(19) United States
(12) Patent Application Publication
(10) Pub. No.: US 2009/0325906 A1
(43) Pub. Date: Dec. 31, 2009

(54) Methods and compositions for therapeutic treatment

(76) Inventors: Wendye Robbins, South San Francisco, CA (US); Ving Lee, Los Altos, CA (US)

Correspondence Address: WILSON, SONSINI, GOODRICH & ROSATI, 650 PAGE MILL ROAD, PALO ALTO, CA, 94304-1050, US

(21) Appl. No.: 12/459,178

(22) Filed: Jun. 26, 2009

Related U.S. Application Data
(60) Provisional application No. 61/076,587, filed on Jun. 27, 2008.

(51) Int. Cl. A61K 31/665; A61K 31/436; A61K 31/352

(52) U.S. Cl. 514/100; 514/291; 514/456

(57) ABSTRACT

Methods and compositions are described for the modulation of hyperglycemia and/or one or more symptoms of hyperglycemia. Methods and compositions are described for the modulation of efflux transporter activity to increase the efflux of calcineurin inhibitors out of a physiological compartment and into an external environment. In particular, the methods and compositions disclosed herein provide for the increase of efflux transporter activity to increase the efflux of calcineurin inhibitor from physiological compartments.

### CROSS-REFERENCE

This application claims the benefit of U.S. Provisional Application No. 61/076,587, filed Jun. 27, 2008; which is incorporated herein by reference in its entirety.

### BACKGROUND OF THE INVENTION

Pharmaceutical agents often cause systemic side-effects rather than a desired localized action.

For instance, Prograf, the market leading immunosuppressant for preventing transplant rejection has been reported to cause hyperglycemia in 20-50% or liver transplant recipients. Other immunosuppressants, such as Cyclosporin, also cause undesirable side effects. As solid organ transplantation is increasing and grafts last longer than they used to—a kidney given in 2003 is expected to last 20 years—there is a need to find methods to decrease side effects that impinge on quality of life of patients.

### SUMMARY OF THE INVENTION

The invention provides methods, compositions, and kits for the use of blood-tissue barrier (BTB) transport protein modulator, e.g., to reduce or eliminate hyperglycemia and/or one or more symptoms of hyperglycemia induced by a calcineurin inhibitor.

In one aspect the invention provides compositions including a calcineurin inhibitor and a BTB transport protein modulator. In some embodiments, the invention provides compositions including an effective amount of a calcineurin inhibitor and an amount of a BTB transport protein modulator sufficient to reduce hyperglycemia and/or one or more symptoms of hyperglycemia induced by the calcineurin inhibitor. In some embodiments of this aspect, the invention provides a composition including a calcineurin inhibitor and a Blood-Tissue barrier (BTB) transport protein modulator, where the BTB transport protein modulator is present in an amount sufficient to reduce hyperglycemia and/or one or more symptoms of hyperglycemia induced by the calcineurin inhibitor. In some embodiments of this aspect, the invention provides a composition including a calcineurin inhibitor and a Blood-Tissue barrier (BTB) transport protein modulator, where the BTB transport protein modulator is present in an amount sufficient to decrease the concentration of the calcineurin inhibitor in a physiological compartment when the composition is administered to an animal.

In some embodiments of this aspect, BTB transport protein includes an ABC transport protein. In some embodiments of the composition, the BTB transport protein modulator in the composition includes a BTB transport protein activator. In some embodiments, the BTB transport protein modulator in the composition includes a modulator of P-gP. In some embodiments, the BTB transport protein modulator in the composition includes a pyrone analog. In some embodiments, the BTB transport protein modulator is a polyphenol. In some embodiments of the invention, the polyphenol includes a flavonoid. In some embodiments, the polyphenol includes quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, epicatechin, or combinations thereof. In some embodiments, the flavonoid is quercetin or a quercetin derivative, or fisetin or fisetin derivative. In some embodiments, the flavonoid is a phosphorylated quercetin or a phosphorylated quercetin derivative, or a phosphorylated fisetin or a phosphorylated fisetin derivative. Preferably, the flavonoid is a phosphorylated quercetin, fisetin or a phosphorylated fisetin.

In some embodiments, the quercetin or quercetin derivative is modified. In some embodiments, the quercetin or quercetin derivative is phosphorylated. In some embodiments, the phosphorylated quercetin is 3′-quercetin phosphate, 4′-quercetin phosphate, 5,7-dideoxyquercetin phosphate, or combinations thereof. In some embodiments, the phosphorylated quercetin is 3′-quercetin phosphate. In some embodiments, the phosphorylated quercetin is 4′-quercetin phosphate. In some embodiments, the phosphorylated quercetin is a mixture of 3′-quercetin phosphate and 4′-quercetin phosphate. In some embodiments, the mixture of phosphorylated quercetin comprises at least 5%, at least 10%, at least 15%, at least 20%, at least 25%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% of 3′-quercetin phosphate. In some embodiments, the mixture of phosphorylated quercetin comprises at least 5%, at least 10%, at least 15%, at least 20%, at least 25%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% of 4′-quercetin phosphate.

In some embodiments, the flavonoid is fisetin or a fisetin derivative. In some embodiments, the fisetin or fisetin derivative is modified. In some embodiments, the modified fisetin or fisetin derivative is phosphorylated. In some embodiments, the fisetin or fisetin derivative is fisetin phosphate. In some embodiments, the phosphorylated fisetin is 3′-fisetin phosphate, 4′-fisetin phosphate or 3-fisetin phosphate.

In some embodiments, the compositions disclosed herein further comprises an oligosaccharide. In some embodiments, the oligosaccharide is a cyclodextrin. In some embodiments, the cyclodextrin is a sulfo-alkyl ether substituted cyclodextrin or a sulfobutyl-ether substituted cyclodextrin. In some embodiments, the cyclodextrin is hydroxypropyl-β-cyclodextrin, hydroxypropyl-γ-cyclodextrin, sulfobutylether-β-cyclodextrin, sulfobutylether-7-β-cyclodextrin, or combinations thereof.

In some embodiments, the calcineurin inhibitor is tacrolimus and the BTB transport protein modulator is quercetin or a quercetin derivative. In some embodiments, the tacrolimus and quercetin or quercetin derivative is present at a molar ratio of 0.001:1 to about 10:1. In some embodiments, the tacrolimus is present at about 0.1-1000 mg and the quercetin or quercetin derivative is present at about 10 to about 1000 mg. In some embodiments, the tacrolimus is present at about 0.5-100 mg and the quercetin or quercetin derivative is present at about 50 to about 500 mg. In some embodiments, the tacrolimus is present at about 5 mg and quercetin or quercetin derivative is present at about 500 mg.

In some embodiments, the calcineurin inhibitor is tacrolimus and the BTB transport protein modulator is fisetin or a fisetin derivative. In some embodiments, the tacrolimus and fisetin or fisetin derivative is present at a molar ratio of 0.001:1 to about 10:1. In some embodiments, the tacrolimus is present at about 0.1-1000 mg and the fisetin or fisetin derivative is present at about 10 to about 1000 mg. In some embodiments, the tacrolimus is present at about 0.5-100 mg and the fisetin or fisetin derivative is present at about 50 to about 500 mg. In some embodiments, the tacrolimus is present at about 5 mg and fisetin or fisetin derivative is present at about 500 mg.

In some embodiments of the compositions of the invention, the BTB transport protein modulator decreases a side effect of the calcineurin inhibitor. In some embodiments of the compositions of the invention, the BTB transport protein modulator decreases or eliminates hyperglycemia or a symptom of hyperglycemia induced by the calcineurin inhibitor. In some embodiments, the symptom is selected from the group consisting of glucosuria, polyphagia, polyuria, polydipsia, loss of consciousness, blurred vision, headaches, coma, ketoacidosis, decrease in blood volume, decrease in renal bloodflow, accelerated lipolysis, weight loss, stomach problems, intestinal problems, poor wound healing, dry mouth, nausea, vomiting, dry skin. itchy skin, impotence, hypeventilation, ketoanemia, fatigue, weakness on one side of the body, hallucinations, impairment in cognitive function, increase sadness, anxiety, recurrent genital infections, increase sugar in urine, retinopathy, nepropathy, arteriosclerotic disorders, cardiac arrhythmia, stupor, susceptibility to infection, neuropathy, nerve damages causing cold feet, nerve damage causing insensitive feet, loss of hair or combinations thereof. In some embodiments, the symptom is glucosuria. In some embodiments of the compositions of the invention, the BTB transport protein modulator decreases a renal side effect. In some embodiments, the renal side effect is nephrotoxicity, renal function impairment, creatinine increase, urinary tract infection, oliguria, cystitis haemorrhagic, hemolytic-uremic syndrome or micturition disorder. In some embodiments of the invention, a kit includes the composition of the invention and instructions for use of the composition.

In another aspect, the invention provides methods utilizing BTB transport protein modulator. In some embodiments of this aspect, the invention provides a method of treating a condition by administering to an animal suffering from the condition an effective amount of a calcineurin inhibitor and an amount of a BTB transport protein modulator sufficient to reduce or eliminate hyperglycemia or a symptom of hyperglycemia without the BTB transport protein modulator when the composition is administered to the animal.

In some embodiments of the methods of the invention, the BTB transport protein modulator is a BTB protein transport activator, where the BTB transport protein activator is present in an amount sufficient to reduce hyperglycemia and/or one or more symptoms of hyperglycemia induced by the calcineurin inhibitor. In some embodiments of this aspect, BTB transport protein includes an ABC transport protein. In some embodiments of the composition, the BTB transport protein modulator includes a BTB transport protein activator. In some embodiments, the BTB transport protein modulator includes a modulator of P-gP. In some embodiments, the BTB transport protein modulator includes a pyrone analog. In some embodiments, the BTB transport protein modulator is a polyphenol. In some embodiments of the invention, the polyphenol includes a flavonoid. In some embodiments, the polyphenol includes quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, epicatechin, or combinations thereof. In some embodiments, the flavonoid is quercetin or a quercetin derivative, or a fisetin or fisetin derivative. In some embodiments, the flavonoid is a phosphorylated quercetin or a phosphorylated quercetin derivative, or a phosphorylated fisetin or a phosphorylated fisetin derivative. Preferably, the flavonoid is a phosphorylated quercetin, fisetin or a phosphorylated fisetin.

In some embodiments of the methods of the invention, the quercetin or quercetin derivative is modified. In some embodiments, the quercetin or quercetin derivative is phosphorylated. In some embodiments, the phosphorylated quercetin is 3′-quercetin phosphate, 4′-quercetin phosphate, 5,7-dideoxyquercetin phosphate, or combinations thereof. In some embodiments, the phosphorylated quercetin is 3′-quercetin phosphate. In some embodiments, the phosphorylated quercetin is 4′-quercetin phosphate. In some embodiments, the phosphorylated quercetin is a mixture of 3′-quercetin phosphate and 4′-quercetin phosphate. In some embodiments, the mixture of phosphorylated quercetin comprises at least 5%, at least 10%, at least 15%, at least 20%, at least 25%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 99.1% or at least 99.9% of 3′-quercetin phosphate. In some embodiments, the mixture of phosphorylated quercetin comprises at least 5%, at least 10%, at least 15%, at least 20%, at least 25%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 99.1% or at least 99.9% of 4′-quercetin phosphate.

In some embodiments of the methods of the invention, the flavonoid is fisetin or a fisetin derivative. In some embodiments, the fisetin or fisetin derivative is modified. In some embodiments, the modified fisetin or fisetin derivative is phosphorylated. In some embodiments, the fisetin or fisetin derivative is fisetin phosphate. In some embodiments, the phosphorylated fisetin is 3′-fisetin phosphate; 4′-fisetin phosphate or 3-fisetin phosphate.

In some embodiments of the methods of the invention, the composition disclosed herein further comprises an oligosaccharide. In some embodiments, the oligosaccharide is a cyclodextrin. In some embodiments, the cyclodextrin is a sulfo-alkyl ether substituted cyclodextrin or a sulfobutyl-ether substituted cyclodextrin. In some embodiments, the cyclodextrin is hydroxypropyl-β-cyclodextrin, hydroxypropyl-γ-cyclodextrin, sulfobutylether-β-cyclodextrin, sulfobutylether-7-β-cyclodextrin, or combinations thereof.

In some embodiments of the methods of the invention, the calcineurin inhibitor is tacrolimus and the BTB transport protein modulator is quercetin or a quercetin derivative. In some embodiments, the tacrolimus and quercetin or quercetin derivative is present at a molar ratio of 0.001:1 to about 10:1. In some embodiments, the tacrolimus is present at about 0.1-1000 mg and the quercetin or quercetin derivative is present at about 10 to about 1000 mg. In some embodiments, the tacrolimus is present at about 0.5-100 mg and the quercetin or quercetin derivative is present at about 50 to about 500 mg. In some embodiments, the tacrolimus is present at about 5 mg and quercetin or quercetin derivative is present at about 500 mg.

In some embodiments of the methods of the invention, the calcineurin inhibitor is tacrolimus and the BTB transport protein modulator is fisetin or a fisetin derivative. In some embodiments, the tacrolimus and fisetin or fisetin derivative is present at a molar ratio of 0.001:1 to about 10:1. In some embodiments, the tacrolimus is present at about 0.1-1000 mg and the fisetin or fisetin derivative is present at about 10 to about 1000 mg. In some embodiments, the tacrolimus is present at about 0.5-100 mg and the fisetin or fisetin derivative is present at about 50 to about 500 mg. In some embodiments, the tacrolimus is present at about 5 mg and fisetin or fisetin derivative is present at about 500 mg.

In some embodiments of methods of the invention, the BTB transport protein modulator decreases a side effect of the calcineurin inhibitor. In some embodiments of the compositions of the invention, the BTB transport protein modulator decreases reduces or eliminates hyperglycemia or a symptom of hyperglycemia induced by the calcineurin inhibitor. In some embodiments, the symptom is selected from the group consisting of glucosuria, polyphagia, polyuria, polydipsia, loss of consciousness, blurred vision, headaches, coma, ketoacidosis, decrease in blood volume, decrease in renal bloodflow, accelerated lipolysis, weight loss, stomach problems, intestinal problems, poor wound healing, dry mouth, nausea, vomiting, dry skin. itchy skin, impotence, hypeventilation, ketoanemia, fatigue, weakness on one side of the body, hallucinations, impairment in cognitive function, increase sadness, anxiety, recurrent genital infections, increase sugar in urine, retinopathy, nepropathy, arteriosclerotic disorders, cardiac arrhythmia, stupor, susceptibility to infection, neuropathy, nerve damages causing cold feet, nerve damage causing insensitive feet, loss of hair or combinations thereof. In some embodiments, the symptom is glucosuria. In some embodiments of the methods of the invention, the BTB transport protein modulator decreases a renal side effect. In some embodiments, the renal side effect is nephrotoxicity, renal function impairment, creatinine increase, urinary tract infection, oliguria, cystitis haemorrhagic, hemolytic-uremic syndrome or micturition disorder. In some embodiments of the invention, a kit includes the composition of the invention and instructions for use of the composition.

In some embodiments of the methods of the invention, the calcineurin inhibitor is present in an amount sufficient to exert a therapeutic effect and the BTB transport protein activator is present in an amount sufficient to decrease a side effect of the calcineurin inhibitor by an average of at least about 5%, compared to the effect without the BTB transport protein activator. In some embodiments, the administration is oral administration. In some embodiments of the methods of the invention, the side effect is induced by the administration of a calcineurin inhibitor. In some embodiments of the methods of the invention, the side effect is hyperglycemia or a symptom induced by the administration of the calcineurin inhibitor. In some embodiments, the symptom is selected from the group consisting of glucosuria, polyphagia, polyuria, polydipsia, loss of consciousness, blurred vision, headaches, coma, ketoacidosis, decrease in blood volume, decrease in renal bloodflow, accelerated lipolysis, weight loss, stomach problems, intestinal problems, poor wound healing, dry mouth, nausea, vomiting, dry skin, itchy skin, impotence, hypeventilation, ketoanemia, fatigue, weakness on one side of the body, hallucinations, impairment in cognitive function, increase sadness, anxiety, recurrent genital infections, increase sugar in urine, retinopathy, nepropathy, arteriosclerotic disorders, cardiac arrhythmia, stupor, susceptibility to infection, neuropathy, nerve damages causing cold feet, nerve damage causing insensitive feet and loss of hair, or combinations thereof. In some embodiments, the symptom is glucosuria. In some embodiments of the methods of the invention, the BTB transport protein modulator decreases a renal side effect. In some embodiments, the renal side effect is nephrotoxicity, renal function impairment, creatinine increase, urinary tract infection, oliguria, cystitis haemorrhagic, hemolytic-uremic syndrome or micturition disorder.

In some embodiments of the methods of the invention, the subject experiences from a condition selected from the group consisting of organ transplant, an autoimmune disease, and an inflammatory disease. In some embodiments, the organ transplant is selected from the group consisting of kidney transplant, pancreas transplant, liver transplant, heart transplant, lung transplant, intestine transplant, pancreas after kidney transplant, and simultaneous pancreas-kidney transplant. In some embodiments of the invention, the autoimmune disease is selected from the group consisting of Lupus nephritis, actopic dermatitis, and psoriasis. In some embodiments, the inflammatory disease is selected from the group consisting of asthma, vulvar lichen sclerosis, chronic allergic contact dermatitis, eczema, vitiligo and ulcerative colitis.

In some embodiments, the administration comprises single or multiple doses of said calcineurin inhibitor and said BTB transport protein modulator in the same dosage form, concurrent administration in separate dosage forms, or separate administration. In some embodiments, the calcineurin inhibitor is administered in an amount sufficient to exert a therapeutic effect and the BTB transport protein modulator is administered in an amount sufficient to decrease hyperglycemia and/or one or more symptoms of hyperglycemia induced by the calcineurin inhibitor by an average of at least about 5%, compared to the hyperglycemia or symptom of hyperglycemia without the BTB transport protein modulator. In some embodiments, therapeutic effect of the calcineurin inhibitor is increased at least about 5%, compared to the therapeutic effect without the BTB transport protein modulator.

In some embodiments of the methods of the invention, the BTB transport protein modulator includes an activator of P-gP. In some embodiments, the BTB transport protein modulator includes a polyphenol. In some embodiments, the polyphenol includes a flavonoid. In some embodiments of the invention, the polyphenol includes quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, or epicatechin. In some embodiments, the flavonoid includes quercetin or other substituted analogs of naturally-occurring (bio)flavonoids. In some embodiments of the invention, the calcineurin inhibitor is tacrolimus or a tacrolimus analog. Examples of tacrolimus analog include meridamycin, 31-O-Demethyl-FK506; L-683,590, L-685,818; 32-O-(1-hydroxyethylindol-5-yl)ascomycin; ascomycin; C18-OH-ascomycin; 9-deoxo-31-O-demethyl-FK506; L-688,617; A-119435; AP1903; rapamycin; dexamethasone-FK506 heterodimer; 13-O-demethyl tacrolimus; and FK 506-dextran conjugate. In some embodiments, the calcineurin inhibitor is tacrolimus.

In some embodiments of the methods of the invention, the concentration of the calcineurin inhibitor is modulated in a pancreatic islet cell comprising administering to a subject in need of treatment with the calcineurin inhibitor an amount of a BTB transport protein modulator sufficient to modify the concentration of said calcineurin inhibitor in said pancreatic islet cell. In some embodiments, the pancreatic islet cell is a β cell. In some embodiments, the BTB transport protein modulator decreases the concentration of the calcineurin inhibitor in the pancreatic islet cell. In some embodiments of the methods of the invention, the BTB transport protein modulator includes an activator of P-gP. In some embodiments, the BTB transport protein modulator includes a pyrone analog. In some embodiments, the BTB transport protein modulator is a polyphenol. In some embodiments, the polyphenol includes a flavonoid. In some embodiments of the invention, the polyphenol includes quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, or epicatechin. In some embodiments, the flavonoid includes quercetin or other substituted analogs of naturally-occurring (bio)flavonoids. In some embodiments of the invention, the calcineurin inhibitor is tacrolimus or a tacrolimus analog. Examples of tacrolimus analog include meridamycin, 31-O-Demethyl-FK506; L-683,590, L-685,818; 32-O-(1-hydroxyethylindol-5-yl)ascomycin; ascomycin; C18-OH-ascomycin; 9-deoxo-31-O-demethyl-FK506; L-688,617; A-119435; AP1903; rapamycin; dexamethasone-FK506 heterodimer; 13-O-demethyl tacrolimus; and FK 506-dextran conjugate. In some embodiments, the calcineurin inhibitor is tacrolimus.

In some embodiments of the methods of the invention, the individual suffers from a condition including organ transplant, an autoimmune disease, and an inflammatory disease. In some embodiments the individual suffers from an organ transplant. In some embodiments, the organ transplant is selected from the group consisting of kidney transplant, pancreas transplant, liver transplant, heart transplant, lung transplant, intestine transplant, pancreas after kidney transplant, and simultaneous pancreas-kidney transplant. In some embodiments the individual suffers from an autoimmune disease. In some embodiments, the autoimmune disease is selected from the group consisting of Lupus nephritis, actopic dermatitis, rheumatoid arthritis, and psoriasis. In some embodiments the individual suffers from an inflammatory disease. In some embodiments, the inflammatory disease is selected from the group consisting of asthma, vulvar lichen sclerosis, chronic allergic contact dermatitis, eczema, vitiligo and ulcerative colitis.

In some embodiments of the method of the invention, the administration includes single or multiple doses of said calcineurin inhibitor and single or multiple doses of said BTB transport protein modulator. In some embodiments of the method of the invention, the administration comprising simultaneous administration of said calcineurin inhibitor and said BTB transport protein modulator in the same dosage form, simultaneous administration in separate dosage forms, or separate administration. In some embodiments of the method of the invention, the administration includes simultaneous administration of the calcineurin inhibitor and the BTB transport protein modulator in the same dosage form. In some embodiments of the method of the invention, the administration is oral administration.

Other objects, features and advantages of the methods and compositions described herein will become apparent from the following detailed description. It should be understood, however, that the detailed description and the specific examples, while indicating specific embodiments, are given by way of illustration only, since various changes and modifications within the spirit and scope of the invention will become apparent to those skilled in the art from this detailed description.

All publications, patents, and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows that quercetin decreases FK506 (tacorlimus or tac, 2 mg/Kg) induced hyperglycemia.

FIG. 2 shows that quercetin decreases FK506 (tacrolimus or tac, 05. mg/Kg) induced hyperglycemia.

FIG. 3 depicts the effect of quercetin on FK 506 inhibition of lymphocyte proliferation in a MLR response at a 0.5:1 R:S ratio (n=3).

FIG. 4 depicts the effect of quercetin on FK 506 inhibition of lymphocyte proliferation in a MLR response at a 1:1 R:S ratio (n=3).

FIG. 5 depicts the effect of quercetin on FK 506 inhibition of lymphocyte proliferation in a MLR response at a 5:1 R:S ratio (n=3).

FIG. 6 depicts the effect of quercetin on FK 506 inhibition of lymphocyte proliferation after concanavalin A (Con A) stimulation (n=3).

FIG. 7 shows the effect of quercetin and tacrolimus on response of mouse spleen cells to concanavalin A at a high cell concentration.

FIG. 8 shows the effect of quercetin and tacrolimus on response of mouse spleen cells to concanavalin A at a low cell concentration.

FIG. 9 shows the effect of quercetin and tacrolimus on response of mouse spleen cells to LPS at a high cell concentration.

FIG. 10 shows the effect of quercetin and tacrolimus on response of mouse spleen cells to LPS at a low cell concentration.

FIG. 11 shows the effect of vehicle treatment on mitogen responses at a high cell concentration.

FIG. 12 shows the effect of vehicle treatment on mitogen responses at a low cell concentration.

FIG. 13 depicts the effect of different doses of quercetin on FK 506 inhibition of lymphocyte proliferation after Con A stimulation at a high cell concentration.

FIG. 14 depicts the effect of different doses of quercetin on FK 506 inhibition of lymphocyte proliferation after Con A stimulation at a low cell concentration.

FIG. 15 depicts a table with the pharmacokinetics parameters of FK 506 in male Lewis rats after 1 mg/kg i.v. administration alone or in combination with i.p, administration of different doses of LNS 0694

FIG. 16 depicts the levels of FK 506 in plasma at different time points after quercetin administration.

### DETAILED DESCRIPTION OF THE INVENTION

Unless defined otherwise, all technical and scientific terms used herein have the same meaning as is commonly understood by one of skill in the art to which this invention belongs. All patents and publications referred to herein are incorporated by reference.

In one aspect, the invention provides compositions and methods utilizing an agent that modulates an effect, e.g., that reduces or eliminates hyperglycemia and/or one or more symptoms of hyperglycemia. In one aspect, the invention provides compositions and methods utilizing an agent that changes the concentration of a calcineurin inhibitor in a physiological compartment, e.g., pancreatic islet cells. In some embodiments, the invention provides compositions and methods utilizing an agent that reduces or eliminates hyperglycemia and/or one or more symptoms of hyperglycemia induced by a calcineurin inhibitor treatment. In some embodiments, the invention provides compositions and methods utilizing a combination of a calcineurin inhibitor and an agent that reduces or eliminates hyperglycemia and/or one or more symptoms of hyperglycemia induced by the calcineurin inhibitor treatment. In some embodiments, the invention provides compositions and methods utilizing a combination of a calcineurin inhibitor and an agent that increases or enhances a therapeutic effect associated with calcineurin inhibitor treatment while decreasing hyperglycemia and/or one or more symptoms of hyperglycemia induced by the calcineurin inhibitor. In some embodiments, the invention provides compositions and methods utilizing a combination of a calcineurin inhibitor and an agent that changes the concentration of a calcineurin inhibitor in a physiological compartment, e.g., pancreatic islet cells or blood. Examples of calcineurin inhibitors include cyclosporin A (CsA), tacrolimus and tacrolimus analogs.

In another aspect, the invention provides compositions and methods utilizing an agent that reduces or eliminates hyperglycemia and/or one or more symptoms of hyperglycemia.

### Blood-Tissue Barrier

In some embodiments, the invention provides methods and compositions that modulate a blood tissue barrier (BTB) transport protein. BTB transport proteins play a role in the maintenance of barrier to foreign molecules and/or removal of substances from spaces (e.g. cells). The barrier can be a boundary between blood and a physiological compartment such as a cell, an organ, or a tissue. The barrier can be a cell membrane or a layer of cells. One example of such a barrier is the blood brain barrier.

A. Blood Brain Barrier

The access to the brain is controlled by at least two barriers, i.e., blood brain barrier (BBB) and blood-cerebrospinal fluid (CSF) barrier. As used herein, the term “blood brain-barrier” can encompass the blood-brain and blood-CSF barriers, unless otherwise indicated.

The blood brain barrier is formed by tight intercellular junctions of brain capillary endothelial cells. The junctions are sealed by zonulae occludentes and tight junctions. The capillaries are covered by a continuous basal membrane enclosing pericytes, an intermittent cell layer, and the outer basal membrane is contacted by astrocytes. The electrical resistance across the endothelium is high, about 1500 to about 2000 Ω/cm².

The blood brain barrier regulates the transfer of substances between circulating blood and brain by facilitated transport and/or facilitated efflux. The interface on both luminal and abluminal surfaces contain physical and metabolic transporter components.

The exchange of substances between circulating blood and brain can be determined by evaluating octanol/H₂O partition coefficient, facilitated transport, and/or facilitated efflux. The methods of measuring blood brain barrier integrity can be used to identify suitable central nervous system modulators for use in the methods and compositions described herein.

Various transporters exist to regulate the rate of brain permeation for compounds with varying lipophilicity. Generally, hydrophilic nutrients, such as glucose and amino acids, are allowed entry into the physiological compartments of the methods and compositions disclosed herein. Conversely, compounds with low lipophilicity are pumped away from the physiological compartments by, for example, xenobiotic efflux transporters. These transporters are preferably modulated by the methods and compositions described herein to prevent entry of compounds and drugs into the central nervous system.

The blood CSF barrier is formed by the tight junctions of the epithelium of the choroid plexus and arachnoid membrane surrounding the brain and spinal cord. It is involved in micronutrient extraction, clearance of metabolic waste, and transport of drugs.

Mechanisms and routes of compounds into and out of the brain include by way of example only, paracellular aqueous pathway for water soluble agents, transcellular lipophilic pathway for lipid soluble agents, transport proteins for glucose, amino acids, purines, etc., specific receptor mediated endocytosis for insulin, transferrin, etc., adsorptive endocytosis for albumin, other plasma proteins, etc., and transporters (e.g., blood-brain barrier transport proteins) such as P-glycoprotein (P-gP), multi-drug resistance proteins (MRP), organic anion transporter (OAT) efflux pumps, gamma-aminobutyric acid (GABA) transporters and other transporters that modulate transport of drugs and other xenobiotics. Methods and compositions of one aspect of the invention may involve modulation of one or more of these transporters. Preferably, the central nervous system modulators affect one or more of these mechanisms and routes to extrude drugs from the central nervous system.

B. Blood-Tissue Barrier Transporters

In some embodiments, the invention provides methods and compositions that modulate ATP Binding Cassette (ABC) transport proteins. ABC transport proteins are a super family of membrane transporters with similar structural features. These transport proteins are widely distributed in prokaryotic and eukaryotic cells. They are critical in the maintenance of a barrier to foreign molecules and removal of waste from privileged spaces, and may be overexpressed in certain glial tumors conferring drug resistance to cytotoxic drugs. 48 members of the super family are described. There are 7 major subfamilies, which include ABC A-G. Subfamilies C, B, and G play a role in transport activity at blood brain barrier and blood-CSF barrier. ABC A substrates include lipids and cholesterol; ABC B transporters include P-glycoprotein (P-gP) and other multi drug resistance proteins (MRPs); ABC C contains MRP proteins; ABC E is expressed in ovary, testis and spleen; and ABC G contains breast cancer resistance protein (BCRP).

Other examples of blood-CSF barrier transporters that can be modulated by methods and compositions of the invention include organic anion transport systems (OAT), P-gP, and the GABA transporters—GAT-1 and GAT2/BGT-1. Substrate compounds for OATs include opiate peptides, including enkephalin and deltorphin II, anionic compounds, indomethacin, salicylic acid and cimetidine. OATs are inhibited by baclofen, tagamet, indomethacin, etc. and transport HVA (dopamine metabolite) and metabolites of norepinephrine, epinephrine, 5-HT3, and histamine.

GABA transporters are Na and Cl dependent, and are specific for GABA, taurine, β alanine, betaine, and nipecotic acid. GAT2 transporters are localized to abluminal and luminal surfaces of capillary endothelial cells. GAT-1 is localized to the outside of neurons and glia. GABA-transporter substrates include lorazepam, midazolam, diazepam, klonazepam and baclofen. Probenicid inhibits luminal membrane GABA transporters from capillary endothelial cells. GAT-1 is inhibited by Tiagabine.

In some embodiments, the invention provides methods and compositions that modulate P-gP, e.g., that activate P-gP. P-gP, also known as ABCB1, forms a protective barrier to pump away by excreting compounds into bile, urine, and intestinal lumen. Three isoforms have been identified in rodents (mdr1a, mdr1b, mdr2) and two in humans (MDR1 and MDR2). It is expressed in epithelium of the brain choroid plexus (which forms the blood-cerebrospinal fluid barrier), as well as on the luminal surface of blood capillaries of the brain (blood-brain barrier) and other organs, tissues or cells known to have Blood-Tissue barriers, such as the placenta, the ovaries, the testes, and pancreatic islet cells.

In the brain, P-gP is expressed in multiple cell types within brain parenchyma including astrocytes and microglia and in luminal plasma membrane of capillary endothelium where it acts as a barrier to entry and efflux pump activity. P-gP transports a wide range of substrates out of cerebral endothelial cells into vascular lumen. P-gP is also expressed in the apical membrane of the choroid plexus and may transport substances into CSF.

P-gP substrates include molecules that tend to be lipophilic, planar molecules or uncharged or positively charged molecules. Non-limiting examples include organic cations, weak organic bases, organic anions and other uncharged compounds, including polypeptides and peptide derivatives, aldosterone, anthracyclines, colchicine, dexamethasone, digoxin, diltiazem, HIV protease inhibitors, loperamide, MTX, morphine, ondansetron, phenyloin and β-blockers. Inhibitors of P-gP include quinidine, verapamil, rifampin, PSC 833 (see Schinkel, J. Clin Invest., 1996, herein incorporated by reference in its entirety), carbamazepine, and amitryptiline.

Multi-drug resistance protein (MRP) substrates include acetaminophen glucuronide, protease inhibitors, methotrexate and ampicillin. Inhibitors of MRP include buthionine sulphoximine, an inhibitor of glutathione biosynthesis.

C. Transport Mechanisms

Transport exchanges are known to involve passive transfer, active transport, facilitated diffusion, phagocytosis and pinocytosis. See, e.g., Pacifici G M, et al., Clin. Pharmacokinet. 28:235-69 (1995), herein incorporated by reference.

Passive Transfer

One embodiment is the modulation of passive transfer of drugs, chemicals and other substances across a blood-tissue barrier. Passive transfer represents the permeation of a molecule through a physical barrier, such as a cell membrane, down its concentration gradient. Passive diffusion does not require the input of energy, is not saturable and is not subject to competitive inhibition. When drugs cross the blood-tissue barrier by passive diffusion, the amount that crosses in any given time is dependent on the concentration of the drug in the circulation, its physicochemical properties and the properties of the blood-tissue barrier that determine how readily the drug will pass.

Passive diffusion is favored for low-molecular weight and highly lipid-soluble drugs that are predominantly un-ionized. The placenta resembles a lipid bilayer membrane, so only the non-protein bound portion of a drug, barring any applicable active-transport mechanisms, is free to diffuse across it.

Facilitated Diffusion

Another embodiment of the methods and compositions disclosed herein is the modulation of facilitated diffusion mechanisms in the blood-tissue barrier. Facilitated diffusion requires the presence of a carrier substance within the blood-tissue barrier. Moreover, the transport of the system becomes saturated at high concentrations relative to the Michaelis-Menten constant (Kₘ) of the transporter. However, transport by this mechanism does not require the input of energy, as opposed to active transport of substances.

Active Transporters

Another embodiment of the methods and compositions disclosed herein is use of modulators in manipulating active transport of drugs, chemicals and other substances across a blood-tissue barrier. Active transport across the blood-tissue barrier, as opposed to facilitated diffusion or passive transport, requires energy, usually in the form of adenosine triphosphate (ATP) or through energy stored in the transmembrane electrochemical gradient provided by Na⁺, Cl⁻ or H⁺. Because of the input of energy, active transport systems may work against a concentration gradient, however, saturation of the transporters can occur.

One embodiment of the methods and compositions disclosed herein is the modulation of the P-gP transporter. The multidrug resistant gene (MDR1) product, P-glycoprotein, is a member of the ATP-binding cassette (ABC) transporter family. The anatomical localization of P-gp in various tumors (where it confers the MDR phenotype) and at the apical/luminal membrane of polarized cells in several normal human tissues with excretory (liver, kidney, adrenal gland) and barrier (intestine, blood-brain barrier, placenta, blood-testis and blood-ovarian barriers) functions suggests for P-gp a physiological role in detoxification and protection of the body against toxic xenobiotics and metabolites by secreting these compounds into bile, urine, and the intestinal lumen and by preventing their accumulation in the brain, testis, and fetus.

One embodiment of the methods and compositions disclosed herein is the modulation of blood-tissue barrier MRP transporters. The MRP family consists of seven members, designated MRP1 to MRP7. For review, see Borst P, et al., J. Natl. Cancer Inst. 92:1295-1302 (2000), herein incorporated by reference. In humans, MRP transporters are expressed in a variety of tissues such as kidney, liver, brain, lung, intestines, testes, peripheral blood mononuclear cells, hepatocytes, and renal proximal tubules.

One embodiment of the methods and compositions disclosed herein is the modulation of blood-tissue barrier BCRP transporters. BCRP, an ATP-driven transporter, is physiologically expressed in a variety of tissues, most abundantly in the liver and intestinal epithelia, the placenta, the blood-brain barrier, and various stem cells. ABCG2 is a plasma membrane glycoprotein, in polarized cell types localizing to the apical regions. BCRP is responsible for rendering tumor cells resistant to chemotherapeutic agents, such as topotecan, mitoxantrone, doxorubicin and daunorubicin. Allen J D, et al., Cancer Res. 59:4237-4241 (1999). BCRP has also been shown to restrict the passage of topotecan and mitoxantrone to the fetus in mice. Jonker J W et al., J. Natl. Cancer Inst. 92:1651-1656 (2000), herein incorporated by reference.

One embodiment is the modulation of monoamine transporters in blood-tissue barrier. Monoamine transporters are neurotransmitter transporters that transfer monoamine neurotransmitters in or out of cells. There are several different monoamine transporters including the dopamine transporter (DAT), the norepinephrine transporter (NET), the serotonin transporter (SERT) and the extraneuronal monoamine transporter (OCT). SERT and NET derive energy from the transmembrane Na⁺ and Cl⁻ electrochemical gradient, and are localized in a variety of tissues. Both SERT and NET transport serotonin, dopamine and norepinephrine from the maternal circulation to the fetus. Drug substrates of the SERT and NET transporters include amphetamines, although cocaine and non-tricyclic antidepressants bind to the SERT and NET transporters with high affinity without being transferred across the membrane.

OCT transportes include OCT1, OCT2, and OCT3. OCT1 and OCT2 are found in the basolateral membrane of hepatocytes, enterocytes, and renal proximal tubular cells. OCT3 has a more widespread tissue distribution and is considered to be the major component of the extraneuronal monoamine transport system (or uptake-2), which is responsible for the peripheral elimination of monoamine neurotransmitters.

One embodiment of the present invention is the modulation of blood-tissue barrier Organic Cation Transporters. OCTN1 and OCTN 2 have been localized is several tissues including kidney, liver and placenta. One embodiment of the methods and compositions disclosed herein is the modulation of monocarboxylate (MCT) and dicarboxylate (NaDC3) transporters. Both MCT (e.g. lactate transport) and NaDC3 (e.g. succinate transport) utilize electrochemical gradients for transport.

### Transporter Modulators (e.g., Activators or Inhibitors)

The invention provides compositions and methods for reducing or eliminating hyperglycemia and/or one or more symptoms of hyperglycemia induced by a calcineurin inhibitor utilizing one or more BTB modulator. Without being limited to any theory it is thought that this BTB modulator modulates the efflux of calcineurin inhibitor out of physiological compartments, e.g. pancreatic islet cells. In some embodiments, such modulators activate and/or increase the efflux by the BTB transport protein, e.g., P-gP transporters on the blood tissue barrier.

Modulators may be any suitable modulator. In some embodiments, modulators useful in the invention are pyrone analogs, including polyphenols, such as flavonoids. Suitable modulators include catechins from green tea, including (−) epicatechin. See Wang, E, et al., Biochem. Biophys. Res. Comm. 297:412-418 (2002); Zhou, S., et al., Drug Metabol. Rev. 36:57-104 (2004), both of which are herein incorporated by reference in its entirety. Other suitable modulators, e.g., P-gP modulators for use herein include flavonols, including, but not limited to, kaempferol, quercetin, fisetin and galangin.

In other embodiments, P-gP transporter modulators may include small molecules, including 2-p-Tolyl-5,6,7,8-tetrahydrobenzo[d]imidazo[2,1-b]thiazole; 1-Carbazol-9-yl-3-(3,5-dimethylpyrazol-1-yl)-propan-2-ol; 2-(4-Chloro-3,5-dimethylphenoxy)-N-(2-phenyl-2H-benzotriazol-5-yl)-acetamide; N-[2-(4-Chloro-phenyl)-acetyl]-N′-(4,7-dimethyl-quinazolin-2-yl)-guanidine; 1-Benzyl-7,8-dimethoxy-3-phenyl-³H-pyrazolo[3,4-c]isoquinoline; N-(3-Benzooxazol-2-yl-4-hydroxyphenyl)-2-p-tolyloxyacetamide; 8-Allyl-2-phenyl-⁸H-1,3a,8-triazacyclopenta[a]indene; 3-(4-Chloro-benzyl)-5-(2-methoxyphenyl)-[1,2,4]oxadiazole; 2-Phenethylsulfanyl-5,6,7,8-tetrahydrobenzo[4,5]thieno[2,3-d]pyrimidin-4-ylamine; (5,12,13-Triaza-indeno[1,2-b]anthracen-13-yl)-acetic acid ethyl ester; 2,2′-(1-phenyl-¹H-1,2,4-triazole-3,5-diyl)bis-phenol; and 2-(2-Chloro-phenyl)-5-(5-methylthiophen-2-yl)-[1,3,4]oxadiazole. See Kondratov, et al., Proc. Natl. Acad. Sci. 98:14078-14083 (2001), herein incorporated by reference in its entirety.

In some embodiments, the invention utilizes a modulator of a BTB transport protein. In some embodiments, the invention utilizes a modulator of a BTB transport protein that is an ABC transport protein. In some embodiments, the invention utilizes a BTB transport protein activator. In some embodiments, the BTB transport protein modulator is a modulator of P-gP, e.g., an activator of P-gP.

One class of compounds useful in the compositions and methods of the invention are pyrone analogs. As used herein and in the appended claims, the singular forms “a,” “and,” and “the” include plural referents unless the context clearly dictates otherwise. Thus, for example, reference to “a compound” includes a plurality of such compounds, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range may vary between 1% and 15% of the stated number or numerical range.

“Acyl” refers to a —(C═O) radical which is attached to two other moieties through the carbon atom. Those groups may be chosen from alkyl, alkenyl, alkynyl, aryl, heterocyclic, heteroaliphatic, heteroaryl, and the like. Unless stated otherwise specifically in the specification, an acyl group is optionally substituted by one or more substituents which independently are: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), —PO₃XY (where X and Y are hydrogen, methyl, ethyl, alkyl, carbohydrate, lithium, sodium or potassium) or —PO₃Z (where Z is calcium, magnesium or iron) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Acyloxy” refers to a R(C═O)O— radical wherein R is alkyl, aryl, heteroaryl or heterocyclyl. Unless stated otherwise specifically in the specification, an acyloxy group is optionally substituted by one or more substituents which independently are: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2) —S(O)ₜN(R^{a})₂ (where t is 1 or 2), —PO₃XY (where X and Y are hydrogen, methyl, ethyl, alkyl, carbohydrate, lithium, sodium or potassium) or —PO₃Z (where Z is calcium, magnesium or iron) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Alkylaryl” refers to an (alkyl)aryl-radical, where alkyl and aryl are as defined herein.

“Aralkyl” refers to an (aryl)alkyl-radical where aryl and alkyl are as defined herein.

“Alkoxy” refers to a (alkyl)O-radical, where alkyl is as described herein and contains 1 to 10 carbons (e.g., C₁-C₁₀ alkyl). Whenever it appears herein, a numerical range such as “1 to 10” refers to each integer in the given range; e.g., “1 to 10 carbon atoms” means that the alkyl group may consist of 1 carbon atom, 2 carbon atoms, 3 carbon atoms, etc., up to and including 10 carbon atoms. In some embodiments, it is a C₁-C₄ alkoxy group. An alkoxy moiety is optionally substituted by one or more of the substituents described as suitable substituents for an alkyl radical.

“Alkyl” refers to a straight or branched hydrocarbon chain radical, having from one to ten carbon atoms (e.g., C₁-C₁₀ alkyl). Whenever it appears herein, a numerical range such as “1 to 10” refers to each integer in the given range; e.g., “1 to 10 carbon atoms” means that the alkyl group may consist of 1 carbon atom, 2 carbon atoms, 3 carbon atoms, etc., up to and including 10 carbon atoms, although the present definition also covers the occurrence of the term “alkyl” where no numerical range is designated. Typical alkyl groups include, but are in no way limited to, methyl, ethyl, propyl, isopropyl, n-butyl, iso-butyl, sec-butyl isobutyl, tertiary butyl, pentyl, isopentyl, neopentyl, hexyl, septyl, octyl, nonyl, decyl, and the like. The alkyl is attached to the rest of the molecule by a single bond, for example, methyl (Me), ethyl (Et), n-propyl, 1-methylethyl (iso-propyl), n-butyl, n-pentyl, 1,1-dimethylethyl (t-butyl), 3-methylhexyl, 2-methylhexyl, and the like. Unless stated otherwise specifically in the specification, an alkyl group is optionally substituted by one or more substituents which independently are: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), —PO₃XY (where X and Y are hydrogen, methyl, ethyl, alkyl, carbohydrate, lithium, sodium or potassium) or —PO₃Z (where Z is calcium, magnesium or iron) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Alkenyl” refers to a straight or branched hydrocarbon chain radical group, containing at least one double bond, and having from two to ten carbon atoms (ie. C₂-C₁₀ alkenyl). Whenever it appears herein, a numerical range such as “2 to 10” refers to each integer in the given range; e.g., “2 to 10 carbon atoms” means that the alkenyl group may consist of 2 carbon atoms, 3 carbon atoms, etc., up to and including 10 carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more substituents which independently are: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), —PO₃XY (where X and Y are hydrogen, methyl, ethyl, alkyl, carbohydrate, lithium, sodium or potassium) or —PO₃Z (where Z is calcium, magnesium or iron) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Alkynyl” refers to a straight or branched hydrocarbon chain radical group, containing at least one triple bond, having from two to ten carbon atoms (ie. C₂-C₁₀ alkynyl). Whenever it appears herein, a numerical range such as “2 to 10” refers to each integer in the given range; e.g., “2 to 10 carbon atoms” means that the alkynyl group may consist of 2 carbon atoms, 3 carbon atoms, etc., up to and including 10 carbon atoms. In certain embodiments, an alkynyl comprises two to eight carbon atoms. In other embodiments, an alkynyl has two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more substituents which independently are: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), —PO₃XY (where X and Y are hydrogen, methyl, ethyl, alkyl, carbohydrate, lithium, sodium or potassium) or —PO₃Z (where Z is calcium, magnesium or iron) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Amine” refers to a —N(R^{a})₂ radical group, where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl. Unless stated otherwise specifically in the specification, an amino group is optionally substituted by one or more substituents which independently are: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), —PO₃XY (where X and Y are hydrogen, methyl, ethyl, alkyl, carbohydrate, lithium, sodium or potassium) or —PO₃Z (where Z is calcium, magnesium or iron) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

An “amide” refers to a chemical moiety with formula —C(O)NR^{a}R^{b} or —NR^{a}C(O)R^{b}, where R^{a} or R^{b} is independently selected from the group consisting of hydrogen, alkyl, cycloalkyl, aryl, heteroaryl (bonded through a ring carbon) and heterocyclic (bonded through a ring carbon). An amide may be an amino acid or a peptide molecule attached to a compound of Formula (I), thereby forming a prodrug. Any amine or carboxyl side chain on the compounds described herein can be amidified. The procedures and specific groups to make such amides are known to those of skill in the art and can readily be found in reference sources such as Greene and Wuts, Protective Groups in Organic Synthesis, 3.sup.rd Ed., John Wiley & Sons, New York, N.Y., 1999, which is incorporated herein by reference in its entirety.

“Aromatic” or “aryl” refers to an aromatic radical with six to fourteen ring carbon atoms (e.g., C₆-C₁₄ aromatic or C₆-C₁₄ aryl). The term includes monocyclic or fused-ring polycyclic (i.e., rings which share adjacent pairs of ring atoms) groups It has at least one ring having a conjugated pi electron system. Whenever it appears herein, a numerical range such as “6 to 14” refers to each integer in the given range; e.g., “6 to 14 ring atoms” means that the aryl group may consist of 6 ring atoms, 7 ring atoms, etc., up to and including 14 ring atoms. Unless stated otherwise specifically in the specification, an aryl moiety is optionally substituted by one or more substituents which are independently: hydroxyl, carboxaldehyde, amine, C₁-C₁₀ alkyl, C₂-C₁₀ alkynyl, C₂-C₁₀alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphonate, aryl, heteroaryl, heterocyclic, C₃-C₁₀cycloalkyl, —CN—OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), —PO₃XY (where X and Y are hydrogen, methyl, ethyl, alkyl, carbohydrate, lithium, sodium or potassium) or —PO₃Z (where Z is calcium, magnesium or iron) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Carboxaldehyde” refers to a —(C═O)H radical.

“Carboxyl” refers to a —(C═O)OH radical.

“Carbohydrate” as used herein, includes, but not limited to, monosaccharides, disaccharides, oligosaccharides, or polysaccharides. Monosaccharide for example includes, but not limited to, aldotrioses such as glyceraldehyde, ketotrioses such as dihydroxyacetone, aldotetroses such as erythrose and threose, ketotetroses such as erythrulose, aldopentoses such as arabinose, lyxose, ribose and xylose, ketopentoses such as ribulose and xylulose, aldohexoses such as allose, altrose, galactose, glucose, gulose, idose, mannose and talose, ketohexoses such as fructose, psicose, sorbose and tagatose, heptoses such as mannoheptulose, sedoheptulose, octoses such as octolose, 2-keto-3-deoxy-manno-octonate, nonoses such as sialoseallose. Disaccharides for example includes, but not limited to, glucorhamnose, trehalose, sucrose, lactose, maltose, galactosucrose, N-acetyllactosamine, cellobiose, gentiobiose, isomaltose, melibiose, primeverose, hesperodinose, and rutinose. Oligosaccharides for example includes, but not limited to, raffinose, nystose, panose, cellotriose, maltotriose, maltotetraose, xylobiose, galactotetraose, isopanose, cyclodextrin (α-CD) or cyclomaltohexaose, β-cyclodextrin (β-CD) or cyclomaltoheptaose and γ-cyclodextrin (γ-CD) or cyclomaltooctaose. Polysaccharide for example includes, but not limited to, xylan, mannan, galactan, glucan, arabinan, pustulan, gellan, guaran, xanthan, and hyaluronan. Some examples include, but not limited to, starch, glycogen, cellulose, inulin, chitin, amylose and amylopectin.

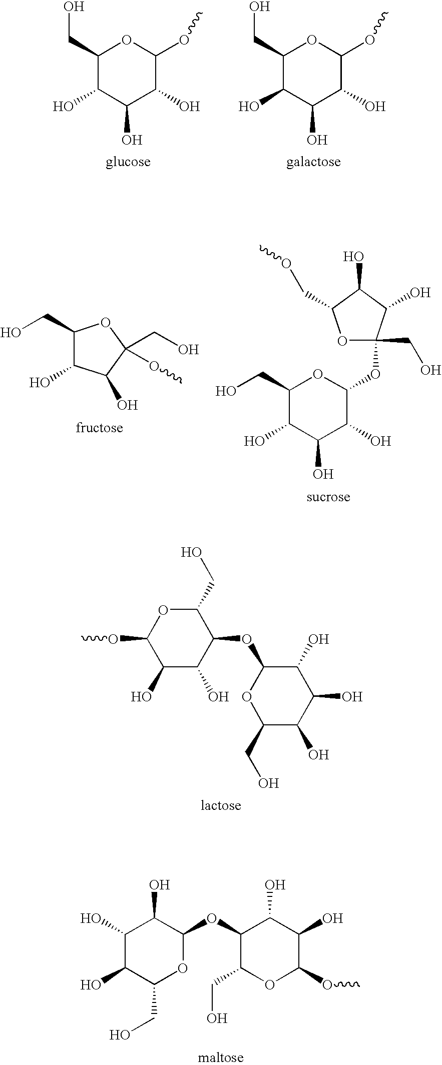

A compound of Formula I having a carbohydrate moiety can be referred to as the pyrone analog glycoside or the pyrone analog saccharide. As used herein, “carbohydrate” further encompasses the glucuronic as well as the glycosidic derivative of compounds of Formula I. Where the phosphonated pyrone analog has no carbohydrate moiety, it can be referred to as the aglycone. Further, where a phenolic hydroxy is derivatized with any of the carbohydrates described above, the carbohydrate moiety is referred to as a glycosyl residue. Unless stated otherwise specifically in the specification, a carbohydrate group is optionally substituted by one or more substituents which are independently: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), —PO₃XY (where X and Y are hydrogen, methyl, ethyl, alkyl, carbohydrate, lithium, sodium or potassium) or —PO₃Z (where Z is calcium, magnesium or iron) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Cyano” refers to a —CN moiety.

“Cycloalkyl” or “carbocyclyl” refers to a monocyclic or polycyclic non-aromatic radical that contains 3 to 10 ring carbon atoms (ie. C₃-C₁₀ cycloalkyl). It may be saturated or unsaturated. Whenever it appears herein, a numerical range such as “3 to 10” refers to each integer in the given range; e.g., “3 to 10 carbon atoms” means that the cycloalkyl group may consist of 3 carbon atoms, etc., up to and including 10 carbon atoms. Illustrative examples of cycloalkyl groups include, but are not limited to the following moieties: cyclopropyl, cyclobutyl, cyclopentyl, cyclohexyl, cycloseptyl, cyclooctyl, cyclononyl, cyclodecyl, norbornyl, and the like. Unless stated otherwise specifically in the specification, a cycloalkyl group is optionally substituted by one or more substituents which are independently: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), —PO₃XY (where X and Y are hydrogen, methyl, ethyl, alkyl, carbohydrate, lithium, sodium or potassium) or —PO₃Z (where Z is calcium, magnesium or iron) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Ester” refers to a chemical radical of formula —COOR, where R is selected from the group consisting of alkyl, cycloalkyl, aryl, heteroaryl (bonded through a ring carbon) and heterocyclic (bonded through a ring carbon). Any hydroxy, or carboxyl side chain on the compounds described herein can be esterified. The procedures and specific groups to make such esters are known to those of skill in the art and can readily be found in reference sources such as Greene and Wuts, Protective Groups in Organic Synthesis, 3.sup.rd Ed., John Wiley & Sons, New York, N.Y., 1999, which is incorporated herein by reference in its entirety. Unless stated otherwise specifically in the specification, an ester group is optionally substituted by one or more substituents which are independently: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), —PO₃XY (where X and Y are hydrogen, methyl, ethyl, alkyl, carbohydrate, lithium, sodium or potassium) or —PO₃Z (where Z is calcium, magnesium or iron) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Fluoroalkyl” refers to an alkyl radical, as defined above, that is substituted by one or more fluoro radicals, for example, trifluoromethyl, difluoromethyl, 2,2,2-trifluoroethyl, 1-fluoromethyl-2-fluoroethyl, and the like. The alkyl part of the fluoroalkyl radical may be optionally substituted as defined above for an alkyl group.

Group “—PO₄XY” refers to —OPO₃XY, and group “—PO₄Z” refers to —OPO₃Z, Group “—OCH₂PO₄XY” refers to —OCH₂OPO₃XY, and group “—OCH₂PO₄Z” refers to —OCH₂OPO₃Z,

“Halo”, “halide”, or, alternatively, “halogen” means fluoro, chloro, bromo or iodo. The terms “haloalkyl,” “haloalkenyl,” “haloalkynyl” and “haloalkoxy” include alkyl, alkenyl, alkynyl and alkoxy structures that are substituted with one or more halo groups or with combinations thereof. For example, the terms “fluoroalkyl” and “fluoroalkoxy” are included in haloalkyl and haloalkoxy groups, respectively, in which the halo is fluorine.

The terms “heteroalkyl” “heteroalkenyl” and “heteroalkynyl” include optionally substituted alkyl, alkenyl and alkynyl radicals and which have one or more skeletal chain atoms selected from an atom other than carbon, e.g., oxygen, nitrogen, sulfur, phosphorus or combinations thereof.

“Heteroaryl” or, alternatively, “heteroaromatic” refers to a 5- to 18-membered aryl group that includes one or more ring heteroatoms selected from nitrogen, oxygen and sulfur, and which may be a monocyclic, bicyclic, tricyclic or tetracyclic fused ring system. Whenever it appears herein, a numerical range such as “5 to 18” refers to each integer in the given range; e.g., “5 to 18 ring atoms” means that the heteroaryl group may consist of 5 ring atoms, 6 ring atoms, etc., up to and including 18 ring atoms. An “N-containing heteroaromatic” or “N-containing heteroaryl” moiety refers to an aromatic group in which at least one of the skeletal atoms of the ring is a nitrogen atom. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzoxazolyl, benzopyranyl, benzopyranonyl, benzofuranyl, benzofuranonyl, benzofurazanyl, benzothiazolyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). Unless stated otherwise specifically in the specification, a heteroaryl moiety is optionally substituted by one or more substituents which are independently: hydroxyl, carboxaldehyde, amine, C₁-C₁₀ alkyl, C₂-C₁₀ alkynyl, C₂-C₁₀alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphonate, aryl, heteroaryl, heterocyclic, C₃-C₁₀cycloalkyl, —CN, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), —PO₃XY (where X and Y are hydrogen, methyl, ethyl, alkyl, carbohydrate, lithium, sodium or potassium) or —PO₃Z (where Z is calcium, magnesium or iron) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Heterocyclyl” or “heterocyclic” refers to a stable 3- to 18-membered non-aromatic ring radical that comprises one to six heteroatoms selected from nitrogen, oxygen and sulfur. Whenever it appears herein, a numerical range such as “3 to 18” refers to each integer in the given range; e.g., “3 to 18 ring atoms” means that the heteroaryl group may consist of 3 ring atoms, 4 ring atoms, etc., up to and including 18 ring atoms. In some embodiments, it is a C₅-C₁₀ heterocyclyl. In some embodiments, it is a C₄-C₁₀ heterocyclyl. In some embodiments, it is a C₃-C₁₀ heterocyclyl. Unless stated otherwise specifically in the specification, the heterocyclyl radical is a monocyclic, bicyclic, tricyclic or tetracyclic ring system, which may include fused or bridged ring systems. The heteroatoms in the heterocyclyl radical may be optionally oxidized. One or more nitrogen atoms, if present, are optionally quaternized. The heterocyclyl radical is partially or fully saturated. The heterocyclyl may be attached to the rest of the molecule through any atom of the ring(s). Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocylyl moiety is optionally substituted by one or more substituents which are independently: hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphonate, aryl, heteroaryl, heterocyclic, C₃-C₁₀cycloalkyl, —CN, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), —PO₃XY (where X and Y are hydrogen, methyl, ethyl, alkyl, carbohydrate, lithium, sodium or potassium) or —PO₃Z (where Z is calcium, magnesium or iron) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Imino” refers to the ═N—H radical.

“Isocyanato” refers to a —N═C═O radical.

“Isothiocyanato” refers to a —N═C═S radical.

“Mercapto” refers to a (alkyl)S— or (H)S— radical.

“Moiety” refers to a specific segment or functional group of a molecule. Chemical moieties are often recognized chemical entities embedded in or appended to a molecule.

“Nitro” refers to the —NO₂ radical.

“Oxa” refers to the —O— radical.

“Oxo” refers to the ═O radical.

“Sulfinyl” refers to a —S(═O)—R radical, where R is selected from the group consisting of alkyl, cycloalkyl, aryl, heteroaryl (bonded through a ring carbon) and heterocyclic (bonded through a ring carbon)

“Sulfonyl” refers to a —S(═O)₂—R radical, where R is selected from the group consisting of alkyl, cycloalkyl, aryl, heteroaryl (bonded through a ring carbon) and heterocyclic (bonded through a ring carbon).

“Sulfonamidyl” refers to a —S(═O)₂—NRR radical, where each R is selected independently from the group consisting of hydrogen, alkyl, cycloalkyl, aryl, heteroaryl (bonded through a ring carbon) and heterocyclic (bonded through a ring carbon).

“Sulfoxyl” refers to a —S(═O)₂OH radical.

“Sulfonate” refers to a —S(═O)₂—OR radical, where R is selected from the group consisting of alkyl, cycloalkyl, aryl, heteroaryl (bonded through a ring carbon) and heterocyclic (bonded through a ring carbon).

“Thiocyanato” refers to a —C═N═S radical.

“Thioxo” refers to the ═S radical.

“Substituted” means that the referenced group may be substituted with one or more additional group(s) individually and independently selected from acyl, alkyl, alkylaryl, cycloalkyl, aralkyl, aryl, carbohydrate, heteroaryl, heterocyclic, hydroxy, alkoxy, aryloxy, mercapto, alkylthio, arylthio, cyano, halo, carbonyl, ester, thiocarbonyl, isocyanato, thiocyanato, isothiocyanato, nitro, perhaloalkyl, perfluoroalkyl, phosphonate, silyl, sulfinyl, sulfonyl, sulfonamidyl, sulfoxyl, sulfonate, and amino, including mono- and di-substituted amino groups, and the protected derivatives thereof. The substituents themselves may be substituted, for example, a cycloakyl substituent may have a halide substituted at one or more ring carbons, and the like. The protecting groups that may form the protective derivatives of the above substituents are known to those of skill in the art and may be found in references such as Greene and Wuts, above.

In this application, 3′-quercetin phosphate is also named as quercetin-3′-O-phosphate. 4′-Quercetin phosphate is also named as quercetin-4′-O-phosphate. 3′-Fisetin phosphate is also named as fisetin-3′-O-phosphate. 4′-Fisetin phosphate is also named as fisetin-4′-O-phosphate. 3-Fisetin phosphate is also named as fisetin-3-O-phosphate.

The compounds presented herein may possess one or more chiral centers and each center may exist in the R or S configuration. The compounds presented herein include all diastereomeric, enantiomeric, and epimeric forms as well as the appropriate mixtures thereof. Stereoisomers may be obtained, if desired, by methods known in the art as, for example, the separation of stereoisomers by chiral chromatographic columns.

The methods and formulations described herein include the use of N-oxides, crystalline forms (also known as polymorphs), or pharmaceutically acceptable salts of compounds having the structure of Formula (I), as well as active metabolites of these compounds having the same type of activity. In addition, the compounds described herein can exist in unsolvated as well as solvated forms with pharmaceutically acceptable solvents such as water, ethanol, and the like. The solvated forms of the compounds presented herein are also considered to be disclosed herein.

Pyrone analogs of Formula I and their pharmaceutically/veterinarily acceptable salt or esters are provided in this invention,

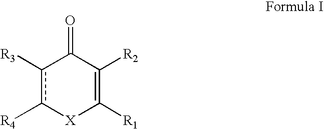

wherein X is O, S, or NR′ wherein R′ is hydrogen, C₁-C₁₀ alkyl, C₂-C₁₀ alkynyl, C₂-C₁₀ alkenyl, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, or C₃-C₁₀ cycloalkyl;

R₁, and R₂ are independently hydrogen, hydroxyl, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, amine, aryl, C₄-C₁₀ heterocyclyl, heteroaryl, C₃-C₁₀ cycloalkyl, —OPO₃WY, —OCH₂PO₄WY, —OCH₂PO₄Z or —OPO₃Z;

R₃ and R₄ are independently hydrogen, hydroxyl, C₁-C₁₀ alkyl, C₂-C₁₀ alkynyl, C₂-C₁₀ alkenyl, carboxyl, carbohydrate, ester, acyloxy, nitro, halogen, C₁-C₁₀aliphatic acyl, C₆-C₁₀ aromatic acyl, C₆-C₁₀ aralkyl acyl, C₆-C₁₀ alkylaryl acyl, alkoxy, amine, aryl, C₄-C₁₀heterocyclyl, heteroaryl, C₃-C₁₀cycloalkyl, —OPO₃WY, —OCH₂PO₄WY, —OCH₂PO₄Z or —OPO₃Z;

or R₃ and R₄ are taken together to form a C₅-C₁₀ heterocyclyl, C₅-C₁₀ cycloalkyl, aryl, or heteroaryl; and W and Y are independently hydrogen, methyl, ethyl, alkyl, carbohydrate, or a cation, and Z is a multivalent cation.

In some embodiments, X is O.

In other embodiments, X is S.

In yet other embodiments, X is NR′.

In some embodiments, R′ is hydrogen. In some embodiments, R′ is unsubstituted C₁-C₁₀ alkyl. In some embodiments, R′ is substituted C₁-C₁₀ alkyl. In some embodiments, R′ is unsubstituted C₂-C₁₀ alkynyl. In some embodiments, R′ is substituted C₂-C₁₀ alkynyl. In some embodiments, R′ is unsubstituted C₂-C₁₀ alkenyl. In some embodiments, R′ is substituted C₂-C₁₀ alkenyl. In some embodiments, R′ is unsubstituted C₁-C₁₀ aliphatic acyl. In some embodiments, R′ is substituted C₁-C₁₀ aliphatic acyl. In some embodiments, R′ is unsubstituted C₆-C₁₀ aromatic acyl. In some embodiments, R′ is substituted C₆-C₁₀ aromatic acyl. In some embodiments, R′ is unsubstituted C₆-C₁₀ aralkyl acyl. In some embodiments, R′ is substituted C₆-C₁₀ aralkyl acyl. In some embodiments, R′ is unsubstituted C₆-C₁₀ alkylaryl acyl. In some embodiments, R′ is substituted C₆-C₁₀ alkylaryl acyl. In some embodiments, R′ is unsubstituted aryl. In some embodiments, R′ is substituted aryl. In some embodiments, R′ is unsubstituted C₃-C₁₀heterocyclyl. In some embodiments, R′ is substituted C₃-C₁₀heterocyclyl. In some embodiments, R′ is unsubstituted heteroaryl. In some embodiments, R′ is substituted heteroaryl. In some embodiments, R′ is unsubstituted C₃-C₁₀ cycloalkyl. In some embodiments, R′ is substituted C₃-C₁₀ cycloalkyl.

In some embodiments, R₁ is hydrogen. In some embodiments, R₁ is optionally substituted C₁-C₁₀ alkyl. hydroxyl. In some embodiments, R₁ is unsubstituted C₁-C₁₀ alkyl. In some embodiments, R₁ is substituted C₁-C₁₀ alkyl. In some embodiments, R₁ is unsubstituted C₁-C₁₀ alkyl. In some other embodiments, R₁ is substituted C₁-C₁₀ alkyl. In some embodiments, R₁ is unsubstituted C₂-C₁₀ alkynyl. In some embodiments, R₁ is substituted C₂-C₁₀ alkynyl. In some embodiments, R₁ is unsubstituted C₂-C₁₀ alkenyl. In some embodiments, R₁ is substituted C₂-C₁₀ alkenyl. In some embodiments, R₁ is carboxyl. In some embodiments, R₁ is unsubstituted carbohydrate. In some embodiments, R₁ is substituted carbohydrate. In some embodiments, R₁ is unsubstituted ester. In some embodiments, R₁ is substituted ester. In some embodiments, R₁ is unsubstituted acyloxy. In some embodiments, R₁ is substituted acyloxy. In some embodiments, R₁ is nitro. In some embodiments, R₁ is halogen. In some embodiments, R₁ is unsubstituted C₁-C₁₀ aliphatic acyl. In some embodiments, R₁ is substituted C₁-C₁₀ aliphatic acyl. In some embodiments, R₁ is unsubstituted C₆-C₁₀ aromatic acyl. In some embodiments, R₁ is substituted C₆-C₁₀ aromatic acyl. In some embodiments, R₁ is unsubstituted C₆-C₁₀ aralkyl acyl. In some embodiments, R₁ is substituted C₆-C₁₀ aralkyl acyl. In some embodiments, R₁ is unsubstituted C₆-C₁₀ alkylaryl acyl. In some embodiments, R₁ is substituted C₆-C₁₀ alkylaryl acyl. In some embodiments, R₁ is unsubstituted alkoxy. In some embodiments, R₁ is substituted alkoxy. In some embodiments, R₁ is unsubstituted amine. In some embodiments, R₁ is substituted amine. In some embodiments, R₁ is unsubstituted aryl. In some embodiments, R₁ is substituted aryl. In some embodiments, R₁ is unsubstituted C₄-C₁₀heterocyclyl. In some embodiments, R₁ is substituted C₄-C₁₀ heterocyclyl. In some embodiments, R₁ is unsubstituted heteroaryl. In some embodiments, R₁ is substituted heteroaryl. In some embodiments, R₁ is unsubstituted C₃-C₁₀ cycloalkyl. In some embodiments, R₁ is substituted C₃-C₁₀ cycloalkyl. In some embodiments, R₁ is —OPO₃WY. In some embodiments, R₁ is —OCH₂PO₄WY. In some embodiments, R₁ is —OCH₂PO₄Z. In some embodiments, R₁ is —OPO₃Z.

In some embodiments, when R₁ is aryl, it is monocyclic. In some embodiments, when R₁ is aryl, it is bicyclic. In some embodiments, when R₁ is heteroaryl, it is monocyclic. In some embodiments, when R₁ is heteroaryl, it is bicyclic.

In some embodiments, R₂ is hydrogen. In some embodiments, R₂ is hydroxyl. In some embodiments, R₂ is optionally substituted C₁-C₁₀ alkyl. In some embodiments, R₂ is unsubstituted C₁-C₁₀ alkyl. In some embodiments, R₂ is substituted C₁-C₁₀ alkyl. In some embodiments, R₂ is unsubstituted C₁-C₁₀ alkyl. In some other embodiments, R₂ is substituted C₁-C₁₀ alkyl. In some embodiments, R₂ is unsubstituted C₂-C₁₀ alkynyl. In some embodiments, R₂ is substituted C₂-C₁₀ alkynyl. In some embodiments, R₂ is unsubstituted C₂-C₁₀ alkenyl. In some embodiments, R₂ is substituted C₂-C₁₀ alkenyl. In some embodiments, R₂ is carboxyl. In some embodiments, R₂ is unsubstituted carbohydrate. In some embodiments, R₂ is substituted carbohydrate. In some embodiments, R₂ is unsubstituted ester. In some embodiments, R₂ is substituted ester. In some embodiments, R₂ is unsubstituted acyloxy. In some embodiments, R₂ is substituted acyloxy. In some embodiments, R₂ is nitro. In some embodiments, R₂ is halogen. In some embodiments, R₂ is unsubstituted C₁-C₁₀ aliphatic acyl. In some embodiments, R₂ is substituted C₁-C₁₀ aliphatic acyl. In some embodiments, R₂ is unsubstituted C₆-C₁₀ aromatic acyl. In some embodiments, R₂ is substituted C₆-C₁₀ aromatic acyl. In some embodiments, R₂ is unsubstituted C₆-C₁₀ aralkyl acyl. In some embodiments, R₂ is substituted C₆-C₁₀ aralkyl acyl. In some embodiments, R₂ is unsubstituted C₆-C₁₀ alkylaryl acyl. In some embodiments, R₂ is substituted C₆-C₁₀ alkylaryl acyl. In some embodiments, R₂ is unsubstituted alkoxy. In some embodiments, R₂ is substituted alkoxy. In some embodiments, R₂ is unsubstituted amine. In some embodiments, R₂ is substituted amine. In some embodiments, R₂ is unsubstituted aryl. In some embodiments, R₂ is substituted aryl. In some embodiments, R₂ is unsubstituted C₄-C₁₀ heterocyclyl. In some embodiments, R₂ is substituted C₄-C₁₀ heterocyclyl. In some embodiments, R₂ is unsubstituted heteroaryl. In some embodiments, R₂ is substituted heteroaryl. In some embodiments, R₂ is unsubstituted C₃-C₁₀ cycloalkyl. In some embodiments, R₂ is substituted C₃-C₁₀ cycloalkyl. In some embodiments, R₂ is —OPO₃WY. In some embodiments, R₂ is —OCH₂PO₄WY. In some embodiments, R₂ is —OCH₂PO₄Z. In some embodiments, R₂ is —OPO₃Z.

In some embodiments, R₃ is hydrogen. In some embodiments, R₃ is optionally substituted C₁-C₁₀ alkyl. hydroxyl. In some embodiments, R₃ is unsubstituted C₁-C₁₀ alkyl. In some embodiments, R₃ is substituted C₁-C₁₀ alkyl. In some embodiments, R₃ is unsubstituted C₁-C₁₀ alkyl. In some other embodiments, R₃ is substituted C₁-C₁₀ alkyl. In some embodiments, R₃ is unsubstituted C₂-C₁₀ alkynyl. In some embodiments, R₃ is substituted C₂-C₁₀ alkynyl. In some embodiments, R₃ is unsubstituted C₂-C₁₀ alkenyl. In some embodiments, R₃ is substituted C₂-C₁₀ alkenyl. In some embodiments, R₃ is carboxyl. In some embodiments, R₃ is unsubstituted carbohydrate. In some embodiments, R₃ is substituted carbohydrate. In some embodiments, R₃ is unsubstituted ester. In some embodiments, R₃ is substituted ester. In some embodiments, R₃ is unsubstituted acyloxy. In some embodiments, R₃ is substituted acyloxy. In some embodiments, R₃ is nitro. In some embodiments, R₃ is halogen. In some embodiments, R₃ is unsubstituted C₁-C₁₀ aliphatic acyl. In some embodiments, R₃ is substituted C₁-C₁₀ aliphatic acyl. In some embodiments, R₃ is unsubstituted C₆-C₁₀ aromatic acyl. In some embodiments, R₃ is substituted C₆-C₁₀ aromatic acyl. In some embodiments, R₃ is unsubstituted C₆-C₁₀ aralkyl acyl. In some embodiments, R₃ is substituted C₆-C₁₀ aralkyl acyl. In some embodiments, R₃ is unsubstituted C₆-C₁₀ alkylaryl acyl. In some embodiments, R₃ is substituted C₆-C₁₀ alkylaryl acyl. In some embodiments, R₃ is unsubstituted alkoxy. In some embodiments, R₃ is substituted alkoxy. In some embodiments, R₃ is unsubstituted amine. In some embodiments, R₃ is substituted amine. In some embodiments, R₃ is unsubstituted aryl. In some embodiments, R₃ is substituted aryl. In some embodiments, R₃ is unsubstituted C₄-C₁₀ heterocyclyl. In some embodiments, R₃ is substituted C₄-C₁₀ heterocyclyl. In some embodiments, R₃ is unsubstituted heteroaryl. In some embodiments, R₃ is substituted heteroaryl. In some embodiments, R₃ is unsubstituted C₃-C₁₀ cycloalkyl. In some embodiments, R₃ is substituted C₃-C₁₀ cycloalkyl. In some embodiments, R₃ is —OPO₃WY. In some embodiments, R₃ is —OCH₂PO₄WY. In some embodiments, R₃ is —OCH₂PO₄Z. In some embodiments, R₃ is —OPO₃Z.

In some embodiments, R₄ is hydrogen. In some embodiments, R₄ is optionally substituted C₁-C₁₀ alkyl. hydroxyl. In some embodiments, R₄ is unsubstituted C₁-C₁₀ alkyl. In some embodiments, R₄ is substituted C₁-C₁₀ alkyl. In some embodiments, R₄ is unsubstituted C₁-C₁₀ alkyl. In some other embodiments, R₄ is substituted C₁-C₁₀ alkyl. In some embodiments, R₄ is unsubstituted C₂-C₁₀ alkynyl. In some embodiments, R₄ is substituted C₂-C₁₀ alkynyl. In some embodiments, R₄ is unsubstituted C₂-C₁₀ alkenyl. In some embodiments, R₄ is substituted C₂-C₁₀ alkenyl. In some embodiments, R₄ is carboxyl. In some embodiments, R₄ is unsubstituted carbohydrate. In some embodiments, R₄ is substituted carbohydrate. In some embodiments, R₄ is unsubstituted ester. In some embodiments, R₄ is substituted ester. In some embodiments, R₄ is unsubstituted acyloxy. In some embodiments, R₄ is substituted acyloxy. In some embodiments, R₄ is nitro. In some embodiments, R₄ is halogen. In some embodiments, R₄ is unsubstituted C₁-C₁₀ aliphatic acyl. In some embodiments, R₄ is substituted C₁-C₁₀ aliphatic acyl. In some embodiments, R₄ is unsubstituted C₆-C₁₀ aromatic acyl. In some embodiments, R₄ is substituted C₆-C₁₀ aromatic acyl. In some embodiments, R₄ is unsubstituted C₆-C₁₀ aralkyl acyl. In some embodiments, R₄ is substituted C₆-C₁₀ aralkyl acyl. In some embodiments, R₄ is unsubstituted C₆-C₁₀ alkylaryl acyl. In some embodiments, R₄ is substituted C₆-C₁₀ alkylaryl acyl. In some embodiments, R₄ is unsubstituted alkoxy. In some embodiments, R₄ is substituted alkoxy. In some embodiments, R₄ is unsubstituted amine. In some embodiments, R₄ is substituted amine. In some embodiments, R₄ is unsubstituted aryl. In some embodiments, R₄ is substituted aryl. In some embodiments, R₄ is unsubstituted C₄-C₁₀ heterocyclyl. In some embodiments, R₄ is substituted C₄-C₁₀ heterocyclyl. In some embodiments, R₄ is unsubstituted heteroaryl. In some embodiments, R₄ is substituted heteroaryl. In some embodiments, R₄ is unsubstituted C₃-C₁₀ cycloalkyl. In some embodiments, R₄ is substituted C₃-C₁₀ cycloalkyl. In some embodiments, R₄ is —OPO₃WY. In some embodiments, R₄ is —OCH₂PO₄WY. In some embodiments, R₄ is —OCH₂PO₄Z. In some embodiments, R₄ is —OPO₃Z.

In some embodiments, R₃ and R₄ are taken together to form an unsubstituted C₅-C₁₀ heterocyclyl. In other embodiments, R₃ and R₄ are taken together to form a substituted C₅-C₁₀ heterocyclyl. In some embodiments, R₃ and R₄ are taken together to form an unsubstituted C₅-C₁₀cycloalkyl. In some embodiments, R₃ and R₄ are taken together to form a substituted C₅-C₁₀cycloalkyl. In some embodiments, R₃ and R₄ are taken together to form an unsubstituted aryl. In some embodiments, R₃ and R₄ are taken together to form a substituted aryl. In some embodiments, R₃ and R₄ are taken together to form an unsubstituted heteroaryl. In some embodiments, R₃ and R₄ are taken together to form a substituted heteroaryl.

In various embodiments, W is hydrogen. In various embodiments, W is unsubstituted methyl. In various embodiments, W is substituted methyl. In various embodiments, W is unsubstituted ethyl. In various embodiments, W is substituted ethyl. In various embodiments, W is unsubstituted alkyl. In various embodiments, W is substituted alkyl. In various embodiments, W is unsubstituted carbohydrate. In various embodiments, W is substituted carbohydrate. In various embodiments, W is potassium. In various embodiments, W is sodium. In various embodiments, W is lithium. In various embodiments, Y is hydrogen. In various embodiments, Y is unsubstituted methyl. In various embodiments, Y is substituted methyl. In various embodiments, Y is unsubstituted ethyl. In various embodiments, Y is substituted ethyl. In various embodiments, Y is unsubstituted alkyl. In various embodiments, Y is substituted alkyl. In various embodiments, Y is unsubstituted carbohydrate. In various embodiments, Y is substituted carbohydrate. In various embodiments, Y is potassium. In various embodiments, Y is sodium. In various embodiments, Y is lithium.

In various embodiments, Z is calcium. In various embodiments, Z is magnesium. In various embodiments, Z is iron.

The 2,3 bond may be saturated or unsaturated in the compounds of Formula I.

In some embodiments of the invention, the pyrone analog of Formula I is of Formula II:

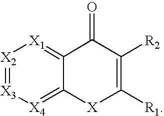

wherein X, R₁, R₂, W, Y, and Z are defined as in Formula I;

X₁, X₂, X₃, and X₄ are independently CR₅, O, S, or N;

each instance of R₅ is independently hydrogen, hydroxyl, carboxaldehyde, amino, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, amine, aryl, C₃-C₁₀heterocyclyl, heteroaryl, C₃-C₁₀ cycloalkyl, —OPO₃WY, —OCH₂PO₄WY, —OCH₂PO₄Z or —OPO₃Z.

In some embodiments, X₁ is CR₅.

In other embodiments, X₁ is O.

In yet other embodiments, X₁ is S.

In further embodiments, X₁ is N.

In some embodiments, X₂ is CR₅.

In other embodiments, X₂ is O.

In yet other embodiments, X₂ is S.

In further embodiments, X₂ is N.

In some embodiments, X₃ is CR₅.

In other embodiments, X₃ is O.

In yet other embodiments, X₃ is S.

In further embodiments, X₃ is N.

In other embodiments, X₄ is CR₅.

In some embodiments, X₄ is O.

In yet other embodiments, X₄ is S.

In some embodiments, X₄ is N.

In some embodiments, X₁, X₂, X₃, and X₄ are CR₅.

In some embodiments, X₁ and X₃ are CR₅ and X₂ and X₄ are N.

In some embodiments, X₂ and X₄ are CR₅ and X₁ and X₃ are N.

In some embodiments, X₂ and X₃ are CR₅ and X₁ and X₄ are N.

In various embodiments, R₁ is one of the following formulae:

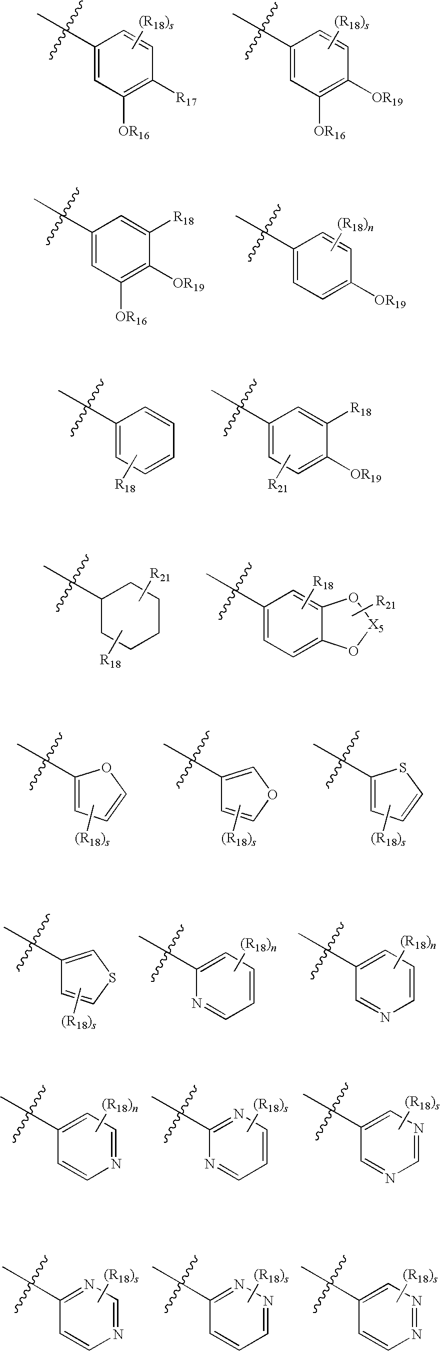

wherein R₁₆ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, C₃-C₁₀ cycloalkyl, —PO₃WY, —CH₂PO₄WY, —CH₂PO₄Z or —PO₃Z;

R₁₇ is hydrogen, hydroxy, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, aryl, C₃-C₁₀ heterocyclyl, heteroaryl, or C₃-C₁₀ cycloalkyl, —OPO₃WY, —OCH₂PO₄WY, —OCH₂PO₄Z or —OPO₃Z; each instance of R₁₈ and R₂₁ is independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀ heterocyclic, C₃-C₁₀ cycloalkyl, —OPO₃WY, —OCH₂PO₄WY, —OCH₂PO₄Z or —OPO₃Z;

R₁₉ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, optionally substituted C₃-C₁₀ cycloalkyl, —PO₃WY, —CH₂PO₄WY, —CH₂PO₄Z or —PO₃Z;

s is an integer of 0, 1, 2, or 3; and

n is an integer of 0, 1, 2, 3, or 4.

In some embodiments, R₁₆ is hydrogen. In some embodiments, R₁₆ is unsubstituted C₁-C₁₀ alkyl. In some embodiments, R₁₆ is substituted C₁-C₁₀ alkyl. In some embodiments, R₁₆ is unsubstituted C₂-C₁₀ alkynyl. In some embodiments, R₁₆ is substituted C₂-C₁₀ alkynyl. In some embodiments, R₁₆ is unsubstituted C₂-C₁₀ alkenyl. In some embodiments, R₁₆ is substituted C₂-C₁₀ alkenyl. In some embodiments, R₁₆ is unsubstituted carbohydrate. In some embodiments, R₁₆ is substituted carbohydrate. In some embodiments, R₁₆ is unsubstituted C₁-C₁₀ aliphatic acyl. In some embodiments, R₁₆ is substituted C₁-C₁₀ aliphatic acyl. In some embodiments, R₁₆ is unsubstituted C₆-C₁₀ aromatic acyl. In some embodiments, R₁₆ is substituted C₆-C₁₀ aromatic acyl. In some embodiments, R₁₆ is unsubstituted C₆-C₁₀ aralkyl acyl. In some embodiments, R₁₆ is substituted C₆-C₁₀ aralkyl acyl. In some embodiments, R₁₆ is unsubstituted C₆-C₁₀ alkylaryl acyl. In some embodiments, R₁₆ is substituted C₆-C₁₀ alkylaryl acyl. In some embodiments, R₁₆ is unsubstituted aryl. In some embodiments, R₁₆ is substituted aryl. In some embodiments, R₁₆ is unsubstituted C₃-C₁₀ heterocyclyl. In some embodiments, R₁₆ is substituted C₃-C₁₀ heterocyclyl. In some embodiments, R₁₆ is unsubstituted heteroaryl. In some embodiments, R₁₆ is substituted heteroaryl. In some embodiments, R₁₆ is unsubstituted C₃-C₁₀ cycloalkyl. In some embodiments, R₁₆ is substituted C₃-C₁₀ cycloalkyl. In some embodiments, R₁₆ is —PO₃WY. In some embodiments, R₁₆ is —CH₂PO₄WY. In some embodiments, R₁₆ is —CH₂PO₄Z. In some embodiments, R₁₆ is —PO₃Z.

In some embodiments, R₁₇ is hydrogen. In some embodiments, R₁₇ is hydroxy. In some embodiments, R₁₇ is carboxaldehyde. In some embodiments, R₁₇ is unsubstituted amine. In some embodiments, R₁₇ is substituted amine. In some embodiments, R₁₇ is unsubstituted C₁-C₁₀ alkyl. In some embodiments, R₁₇ is unsubstituted C₂-C₁₀ alkynyl. In some embodiments, R₁₇ is substituted C₂-C₁₀ alkynyl. In some embodiments, R₁₇ is unsubstituted C₂-C₁₀ alkenyl. In some embodiments, R₁₇ is substituted C₂-C₁₀ alkenyl. In some embodiments, R₁₇ is carboxyl. In some embodiments, R₁₇ is unsubstituted carbohydrate. In some embodiments, R₁₇ is substituted carbohydrate. In some embodiments, R₁₇ is unsubstituted ester. In some embodiments, R₁₇ is substituted ester. In some embodiments, R₁₇ is unsubstituted acyloxy. In some embodiments, R₁₇ is substituted acyloxy. In some embodiments, R₁₇ is nitro. In some embodiments, R₁₇ is halogen. In some embodiments, R₁₇ is unsubstituted C₁-C₁₀ aliphatic acyl. In some embodiments, R₁₇ is substituted C₁-C₁₀ aliphatic acyl. In some embodiments, R₁₇ is unsubstituted C₆-C₁₀ aromatic acyl. In some embodiments, R₁₇ is substituted C₆-C₁₀ aromatic acyl. In some embodiments, R₁₇ is unsubstituted C₆-C₁₀ aralkyl acyl. In some embodiments, R₁₇ is substituted C₆-C₁₀ aralkyl acyl. In some embodiments, R₁₇ is unsubstituted C₆-C₁₀ alkylaryl acyl. n some embodiments, R₁₇ is substituted C₆-C₁₀ alkylaryl acyl. In some embodiments, R₁₇ is unsubstituted alkoxy. In some embodiments, R₁₇ is substituted alkoxy. In some embodiments, R₁₇ is unsubstituted aryl. In some embodiments, R₁₇ is substituted aryl. In some embodiments, R₁₇ is unsubstituted C₃-C₁₀heterocyclyl. In some embodiments, R₁₇ is substituted C₃-C₁₀heterocyclyl. In some embodiments, R₁₇ is unsubstituted heteroaryl. In some embodiments, R₁₇ is substituted heteroaryl. In some embodiments, R₁₇ is unsubstituted C₃-C₁₀ cycloalkyl. In some embodiments, R₁₇ is substituted C₃-C₁₀ cycloalkyl. In some embodiments, R₁₇ is —OPO₃WY. In some embodiments, R₁₇ is —OCH₂PO₄WY. In some embodiments, R₁₇ is —OCH₂PO₄Z. In some embodiments, R₁₇ is —OPO₃Z.

In some embodiments, R₁₈ is hydrogen. In some embodiments, R₁₈ is hydroxy. In some embodiments, R₁₈ is carboxaldehyde. In some embodiments, R₁₈ is unsubstituted amine. In some embodiments, R₁₈ is substituted amine. In some embodiments, R₁₈ is unsubstituted C₁-C₁₀ alkyl. In some embodiments, R₁₈ is unsubstituted C₂-C₁₀ alkynyl. In some embodiments, R₁₈ is substituted C₂-C₁₀ alkynyl. In some embodiments, R₁₈ is unsubstituted C₂-C₁₀ alkenyl. In some embodiments, R₁₈ is substituted C₂-C₁₀ alkenyl. In some embodiments, R₁₈ is carboxyl. In some embodiments, R₁₈ is unsubstituted carbohydrate. In some embodiments, R₁₈ is substituted carbohydrate. In some embodiments, R₁₈ is substituted carbohydrate. In some embodiments, R₁₈ is unsubstituted ester. In some embodiments, R₁₈ is substituted ester. In some embodiments, R₁₈ is unsubstituted acyloxy. In some embodiments, R₁₈ is substituted acyloxy. In some embodiments, R₁₈ is nitro. In some embodiments, R₁₈ is halogen. In some embodiments, R₁₈ is unsubstituted C₁-C₁₀ aliphatic acyl. In some embodiments, R₁₈ is substituted C₁-C₁₀ aliphatic acyl. In some embodiments, R₁₈ is unsubstituted C₆-C₁₀ aromatic acyl. In some embodiments, R₁₈ is substituted C₆-C₁₀ aromatic acyl. In some embodiments, R₁₈ is unsubstituted C₆-C₁₀ aralkyl acyl. In some embodiments, R₁₈ is substituted C₆-C₁₀ aralkyl acyl. In some embodiments, R₁₈ is unsubstituted C₆-C₁₀ alkylaryl acyl. In some embodiments, R₁₈ is substituted C₆-C₁₀ alkylaryl acyl. In some embodiments, R₁₈ is unsubstituted alkoxy. In some embodiments, R₁₈ is substituted alkoxy. In some embodiments, R₁₈ is unsubstituted aryl. In some embodiments, R₁₈ is substituted aryl. In some embodiments, R₁₈ is unsubstituted C₃-C₁₀ heterocyclyl. In some embodiments, R₁₈ is substituted C₃-C₁₀ heterocyclyl. In some embodiments, R₁₈ is unsubstituted heteroaryl. In some embodiments, R₁₈ is substituted heteroaryl. In some embodiments, R₁₈ is unsubstituted C₃-C₁₀ cycloalkyl. In some embodiments, R₁₈ is substituted C₃-C₁₀ cycloalkyl. In some embodiments, R₁₈ is —OPO₃WY. In some embodiments, R₁₈ is —OCH₂PO₄WY. In some embodiments, R₁₈ is —OCH₂PO₄Z. In some embodiments, R₁₈ is —OPO₃Z.

In some embodiments, R₁₉ is hydrogen. In some embodiments, R₁₉ is unsubstituted C₁-C₁₀ alkyl. In some embodiments, R₁₉ is substituted C₁-C₁₀ alkyl. In some embodiments, R₁₉ is unsubstituted C₂-C₁₀ alkynyl. In some embodiments, R₁₉ is substituted C₂-C₁₀ alkynyl. In some embodiments, R₁₉ is unsubstituted C₂-C₁₀ alkenyl. In some embodiments, R₁₉ is substituted C₂-C₁₀ alkenyl. In some embodiments, R₁₉ is unsubstituted carbohydrate. In some embodiments, R₁₉ is substituted carbohydrate. In some embodiments, R₁₉ is unsubstituted C₁-C₁₀ aliphatic acyl. In some embodiments, R₁₉ is substituted C₁-C₁₀ aliphatic acyl. In some embodiments, R₁₉ is unsubstituted C₆-C₁₀ aromatic acyl. In some embodiments, R₁₉ is substituted C₆-C₁₀ aromatic acyl. In some embodiments, R₁₉ is unsubstituted C₆-C₁₀ aralkyl acyl. In some embodiments, R₁₉ is substituted C₆-C₁₀ aralkyl acyl. In some embodiments, R₁₉ is unsubstituted C₆-C₁₀ alkylaryl acyl. In some embodiments, R₁₉ is substituted C₆-C₁₀ alkylaryl acyl. In some embodiments, R₁₉ is unsubstituted aryl. In some embodiments, R₁₉ is substituted aryl. In some embodiments, R₁₉ is unsubstituted C₃-C₁₀ heterocyclyl. In some embodiments, R₁₉ is substituted C₃-C₁₀ heterocyclyl. In some embodiments, R₁₉ is unsubstituted heteroaryl. In some embodiments, R₁₉ is substituted heteroaryl. In some embodiments, R₁₉ is unsubstituted C₃-C₁₀ cycloalkyl. In some embodiments, R₁₉ is substituted C₃-C₁₀ cycloalkyl. In some embodiments, R₁₉ is —PO₃WY. In some embodiments, R₁₉ is —CH₂PO₄WY. In some embodiments, R₁₉ is —CH₂PO₄Z. In some embodiments, R₁₉ is —PO₃Z.

In some embodiments, R₂₁ is hydrogen. In some embodiments, R₂₁ is hydroxy. In some embodiments, R₂, is carboxaldehyde. In some embodiments, R₂₁ is unsubstituted amine. In some embodiments, R₂₁ is substituted amine. In some embodiments, R₂₁ is unsubstituted C₁-C₁₀ alkyl. In some embodiments, R₂₁ is unsubstituted C₂-C₁₀ alkynyl. In some embodiments, R₂₁ is substituted C₂-C₁₀ alkynyl. In some embodiments, R₂₁ is unsubstituted C₂-C₁₀ alkenyl. In some embodiments, R₂₁ is substituted C₂-C₁₀ alkenyl. In some embodiments, R₂₁ is carboxyl. In some embodiments, R₂₁ is unsubstituted carbohydrate. In some embodiments, R₂₁ is substituted carbohydrate. In some embodiments, R₂₁ is unsubstituted ester. In some embodiments, R₂₁ is substituted ester. In some embodiments, R₂, is unsubstituted acyloxy. In some embodiments, R₂₁ is substituted acyloxy. In some embodiments, R₂₁ is nitro. In some embodiments, R₂₁ is halogen. In some embodiments, R₂₁ is unsubstituted C₁-C₁₀ aliphatic acyl. In some embodiments, R₂₁ is substituted C₁-C₁₀ aliphatic acyl. In some embodiments, R₂₁ is unsubstituted C₆-C₁₀ aromatic acyl. In some embodiments, R₂₁ is substituted C₆-C₁₀ aromatic acyl. In some embodiments, R₂₁ is unsubstituted C₆-C₁₀ aralkyl acyl. In some embodiments, R₂₁ is substituted C₆-C₁₀ aralkyl acyl. In some embodiments, R₂₁ is unsubstituted C₆-C₁₀ alkylaryl acyl. In some embodiments, R₂₁ is substituted C₆-C₁₀ alkylaryl acyl. In some embodiments, R₂₁ is unsubstituted alkoxy. In some embodiments, R₂₁ is substituted alkoxy. In some embodiments, R₂₁ is unsubstituted aryl. In some embodiments, R₂₁ is substituted aryl. In some embodiments, R₂₁ is unsubstituted C₃-C₁₀ heterocyclyl. In some embodiments, R₂₁ is substituted C₃-C₁₀ heterocyclyl. In some embodiments, R₂₁ is unsubstituted heteroaryl. In some embodiments, R₂₁ is substituted heteroaryl. In some embodiments, R₂₁ is unsubstituted C₃-C₁₀ cycloalkyl. In some embodiments, R₂₁ is substituted C₃-C₁₀ cycloalkyl. In some embodiments, R₂₁ is —OPO₃WY. In some embodiments, R₂₁ is —OCH₂PO₄WY. In some embodiments, R₂₁ is —OCH₂PO₄Z. In some embodiments, R₂₁ is —OPO₃Z.

In some embodiments, s is an integer of 0. In some embodiments, s is an integer of 1. In some embodiments, s is an integer of 2. In some embodiments, s is an integer of 3.

In some embodiments, n is an integer of 0. In some embodiments, n is an integer of 1. In some embodiments, n is an integer of 2. In some embodiments, n is an integer of 3. In some embodiments, n is an integer of 4.

In various embodiments, W and Y are independently potassium, sodium, or lithium.

In various embodiments, Z is calcium, magnesium or iron.

In various embodiments of the invention, the pyrone analog is of Formulae III, IV, V, or VI as illustrated in Scheme I.

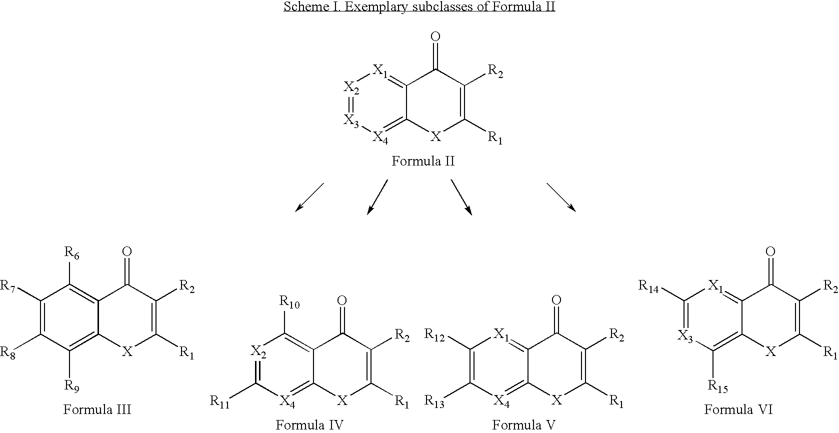

In some embodiments of the invention where the X₁, X₂, X₃, and X₄ of the compounds of Formula II are CR₅, the compound is of Formula III:

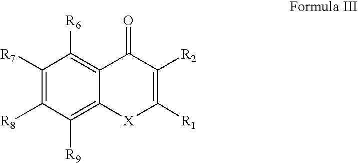

wherein X, R₁, R₂, W, Y, and Z are defined as in Formula I and Formula II;

R₆, R₇, R₈, and R₉ are independently hydrogen, hydroxyl, carboxaldehyde, amino, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, amine, aryl, C₃-C₁₀ heterocyclyl, heteroaryl, C₃-C₁₀ cycloalkyl, —OPO₃WY, —OCH₂PO₄WY, —OCH₂PO₄Z or —OPO₃Z.

In some embodiments, R₆ is hydrogen. In some embodiments, R₆ is hydroxyl. In some embodiments, R₆ is carboxaldehyde. In some embodiments, R₆ is unsubstituted amine. In some embodiments, R₆ is substituted amine. In some embodiments, R₆ is unsubstituted C₁-C₁₀ alkyl. In some embodiments, R₆ is substituted C₁-C₁₀ alkyl. In some embodiments, R₆ is unsubstituted C₂-C₁₀ alkynyl. In some embodiments, R₆ is substituted C₂-C₁₀ alkynyl. In some embodiments, R₆ is unsubstituted C₂-C₁₀ alkenyl. In some embodiments, R₆ is substituted C₂-C₁₀ alkenyl. In some embodiments, R₆ is carboxyl. In some embodiments, R₆ is unsubstituted carbohydrate. In some embodiments, R₆ is substituted carbohydrate. In some embodiments, R₆ is unsubstituted ester. In some embodiments, R₆ is substituted ester. In some embodiments, R₆ is unsubstituted acyloxy. In some embodiments, R₆ is substituted acyloxy. In some embodiments, R₆ is nitro. In some embodiments, R₆ is halogen. In some embodiments, R₆ is unsubstituted C₁-C₁₀ aliphatic acyl. In some embodiments, R₆ is substituted C₁-C₁₀ aliphatic acyl. In some embodiments, R₆ is unsubstituted C₆-C₁₀ aromatic acyl. In some embodiments, R₆ is substituted C₆-C₁₀ aromatic acyl. In some embodiments, R₆ is unsubstituted C₆-C₁₀ aralkyl acyl. In some embodiments, R₆ is substituted C₆-C₁₀ aralkyl acyl. In some embodiments, R₆ is unsubstituted C₆-C₁₀ alkylaryl acyl. In some embodiments, R₆ is substituted C₆-C₁₀ alkylaryl acyl. In some embodiments, R₆ is unsubstituted alkoxy. In some embodiments, R₆ is substituted alkoxy. In some embodiments, R₆ is unsubstituted aryl. In some embodiments, R₆ is substituted aryl. In some embodiments, R₆ is unsubstituted C₃-C₁₀ heterocyclyl. In some embodiments, R₆ is substituted C₃-C₁₀ heterocyclyl. In some embodiments, R₆ is unsubstituted heteroaryl, In some embodiments, R₆ is unsubstituted C₃-C₁₀ cycloalkyl. In some embodiments, R₆ is substituted C₃-C₁₀ cycloalkyl. In some embodiments, R₆ is —OPO₃WY. In some embodiments, R₆ is —OCH₂PO₄WY. In some embodiments, R₆ is —OCH₂PO₄Z. In some embodiments, R₆ is —OPO₃Z.

In some embodiments, R₇ is hydrogen. In some embodiments, R₇ is hydroxyl. In some embodiments, R₇ is carboxaldehyde. In some embodiments, R₇ is unsubstituted amine. In some embodiments, R₇ is substituted amine. In some embodiments, R₇ is unsubstituted C₁-C₁₀ alkyl. In some embodiments, R₇ is substituted C₁-C₁₀ alkyl. In some embodiments, R₇ is unsubstituted C₂-C₁₀ alkynyl. In some embodiments, R₇ is substituted C₂-C₁₀ alkynyl. In some embodiments, R₇ is unsubstituted C₂-C₁₀ alkenyl. In some embodiments, R₇ is substituted C₂-C₁₀ alkenyl. In some embodiments, R₇ is carboxyl. In some embodiments, R₇ is unsubstituted carbohydrate. In some embodiments, R₇ is substituted carbohydrate. In some embodiments, R₇ is unsubstituted ester. In some embodiments, R₇ is substituted ester. In some embodiments, R₇ is unsubstituted acyloxy. In some embodiments, R₇ is substituted acyloxy. In some embodiments, R₇ is nitro. In some embodiments, R₇ is halogen. In some embodiments, R₇ is unsubstituted C₁-C₁₀ aliphatic acyl. In some embodiments, R₇ is substituted C₁-C₁₀ aliphatic acyl. In some embodiments, R₇ is unsubstituted C₆-C₁₀ aromatic acyl. In some embodiments, R₇ is substituted C₆-C₁₀ aromatic acyl. In some embodiments, R₇ is unsubstituted C₆-C₁₀ aralkyl acyl. In some embodiments, R₇ is substituted C₆-C₁₀ aralkyl acyl. In some embodiments, R₇ is unsubstituted C₆-C₁₀ alkylaryl acyl. In some embodiments, R₇ is substituted C₆-C₁₀ alkylaryl acyl. In some embodiments, R₇ is unsubstituted alkoxy. In some embodiments, R₇ is substituted alkoxy. In some embodiments, R₇ is unsubstituted aryl. In some embodiments, R₇ is substituted aryl. In some embodiments, R₇ is unsubstituted C₃-C₁₀heterocyclyl. In some embodiments, R₇ is substituted C₃-C₁₀ heterocyclyl. In some embodiments, R₇ is unsubstituted heteroaryl, In some embodiments, R₇ is unsubstituted C₃-C₁₀ cycloalkyl. In some embodiments, R₇ is substituted C₃-C₁₀ cycloalkyl. In some embodiments, R₇ is —OPO₃WY. In some embodiments, R₇ is —OCH₂PO₄WY. In some embodiments, R₇ is —OCH₂PO₄Z. In some embodiments, R₇ is —OPO₃Z.

In some embodiments, R₈ is hydrogen. In some embodiments, R₈ is hydroxyl. In some embodiments, R₈ is carboxaldehyde. In some embodiments, R₈ is unsubstituted amine. In some embodiments, R₈ is substituted amine. In some embodiments, R₈ is unsubstituted C₁-C₁₀ alkyl. In some embodiments, R₈ is substituted C₁-C₁₀ alkyl. In some embodiments, R₈ is unsubstituted C₂-C₁₀ alkynyl. In some embodiments, R₈ is substituted C₂-C₁₀ alkynyl. In some embodiments, R₈ is unsubstituted C₂-C₁₀ alkenyl. In some embodiments, R₈ is substituted C₂-C₁₀ alkenyl. In some embodiments, R₈ is carboxyl. In some embodiments, R₈ is unsubstituted carbohydrate. In some embodiments, R₈ is substituted carbohydrate. In some embodiments, R₈ is unsubstituted ester. In some embodiments, R₈ is substituted ester. In some embodiments, R₈ is unsubstituted acyloxy. In some embodiments, R₈ is substituted acyloxy. In some embodiments, R₈ is nitro. In some embodiments, R₈ is halogen. In some embodiments, R₈ is unsubstituted C₁-C₁₀ aliphatic acyl. In some embodiments, R₈ is substituted C₁-C₁₀ aliphatic acyl. In some embodiments, R₈ is unsubstituted C₆-C₁₀ aromatic acyl. In some embodiments, R₈ is substituted C₆-C₁₀ aromatic acyl. In some embodiments, R₈ is unsubstituted C₆-C₁₀ aralkyl acyl. In some embodiments, R₈ is substituted C₆-C₁₀ aralkyl acyl. In some embodiments, R₈ is unsubstituted C₆-C₁₀ alkylaryl acyl. In some embodiments, R₈ is substituted C₆-C₁₀ alkylaryl acyl. In some embodiments, R₈ is unsubstituted alkoxy. In some embodiments, R₈ is substituted alkoxy. In some embodiments, R₈ is unsubstituted aryl. In some embodiments, R₈ is substituted aryl. In some embodiments, R₈ is unsubstituted C₃-C₁₀ heterocyclyl. In some embodiments, R₈ is substituted C₃-C₁₀ heterocyclyl. In some embodiments, R₈ is unsubstituted heteroaryl, In some embodiments, R₈ is unsubstituted C₃-C₁₀ cycloalkyl. In some embodiments, R₈ is substituted C₃-C₁₀ cycloalkyl. In some embodiments, R₈ is —OPO₃WY. In some embodiments, R₈ is —OCH₂PO₄WY. In some embodiments, R₈ is —OCH₂PO₄Z. In some embodiments, R₈ is —OPO₃Z.

In some embodiments, R₉ is hydrogen. In some embodiments, R₉ is hydroxyl. In some embodiments, R₉ is carboxaldehyde. In some embodiments, R₉ is unsubstituted amine. In some embodiments, R₉ is substituted amine. In some embodiments, R₉ is unsubstituted C₁-C₁₀ alkyl. In some embodiments, R₉ is substituted C₁-C₁₀ alkyl. In some embodiments, R₉ is unsubstituted C₂-C₁₀ alkynyl. In some embodiments, R₉ is substituted C₂-C₁₀ alkynyl. In some embodiments, R₉ is unsubstituted C₂-C₁₀ alkenyl. In some embodiments, R₉ is substituted C₂-C₁₀ alkenyl. In some embodiments, R₉ is carboxyl. In some embodiments, R₉ is unsubstituted carbohydrate. In some embodiments, R₉ is substituted carbohydrate. In some embodiments, R₉ is unsubstituted ester. In some embodiments, R₉ is substituted ester. In some embodiments, R₉ is unsubstituted acyloxy. In some embodiments, R₉ is substituted acyloxy. In some embodiments, R₉ is nitro. In some embodiments, R₉ is halogen. In some embodiments, R₉ is unsubstituted C₁-C₁₀ aliphatic acyl. In some embodiments, R₉ is substituted C₁-C₁₀ aliphatic acyl. In some embodiments, R₉ is unsubstituted C₆-C₁₀ aromatic acyl. In some embodiments, R₉ is substituted C₆-C₁₀ aromatic acyl. In some embodiments, R₉ is unsubstituted C₆-C₁₀ aralkyl acyl. In some embodiments, R₉ is substituted C₆-C₁₀ aralkyl acyl. In some embodiments, R₉ is unsubstituted C₆-C₁₀ alkylaryl acyl. In some embodiments, R₉ is substituted C₆-C₁₀ alkylaryl acyl. In some embodiments, R₉ is unsubstituted alkoxy. In some embodiments, R₉ is substituted alkoxy. In some embodiments, R₉ is unsubstituted aryl. In some embodiments, R₉ is substituted aryl. In some embodiments, R₉ is unsubstituted C₃-C₁₀ heterocyclyl. In some embodiments, R₉ is substituted C₃-C₁₀ heterocyclyl. In some embodiments, R₉ is unsubstituted heteroaryl, In some embodiments, R₉ is unsubstituted C₃-C₁₀ cycloalkyl. In some embodiments, R₉ is substituted C₃-C₁₀ cycloalkyl. In some embodiments, R₉ is —OPO₃WY. In some embodiments, R₉ is —OCH₂PO₄WY. In some embodiments, R₉ is —OCH₂PO₄Z. In some embodiments, R₉ is —OPO₃Z.

In various embodiments of the invention, the pyrone analog of Formula III is of Formula VII:

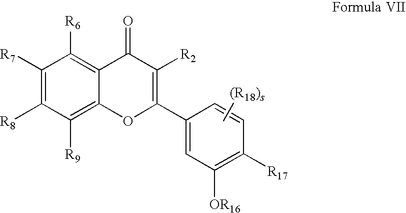

wherein R₂, R₁₆, R₁₇, R₁₈, and s are as defined in Formula II and R₆, R₇, R₈, and R₉ are as defined in Formula III.

In other embodiments of the invention, the pyrone analog of Formula III is a compound of Formula VIII:

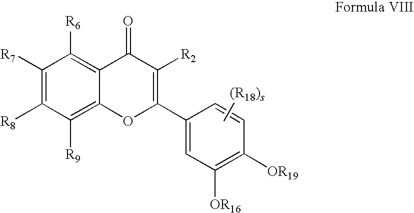

wherein R₂, R₁₆, R₁₈, R₁₉, and s are as defined in Formula II and R₆, R₇, R₈, and R₉ are as defined in Formula III.

In some embodiments of the invention, the pyrone analog of Formula II is of Formula IX:

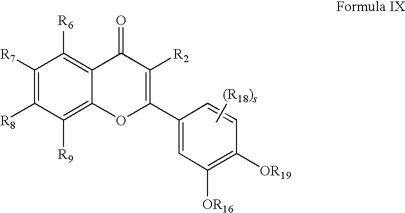

wherein R₂, R₁₆, R₁₈, R₁₉, and s are as defined in Formula II; and

R₆, R₇, R₈, and R₉ are independently hydrogen, carboxaldehyde, amino, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, amine, aryl, C₃-C₁₀ heterocyclyl, heteroaryl, C₃-C₁₀cycloalkyl, —OPO₃WY, —OCH₂PO₄WY, —OCH₂PO₄Z or —OPO₃Z.

In some embodiments, R₆ is hydrogen. In some embodiments, R₆ is carboxaldehyde. In some embodiments, R₆ is unsubstituted amine. In some embodiments, R₆ is substituted amine. In some embodiments, R₆ is unsubstituted C₁-C₁₀ alkyl. In some embodiments, R₆ is substituted C₁-C₁₀ alkyl. In some embodiments, R₆ is unsubstituted C₂-C₁₀ alkynyl. In some embodiments, R₆ is substituted C₂-C₁₀ alkynyl. In some embodiments, R₆ is unsubstituted C₂-C₁₀ alkenyl. In some embodiments, R₆ is substituted C₂-C₁₀ alkenyl. In some embodiments, R₆ is carboxyl. In some embodiments, R₆ is unsubstituted carbohydrate. In some embodiments, R₆ is substituted carbohydrate. In some embodiments, R₆ is unsubstituted ester. In some embodiments, R₆ is substituted ester. In some embodiments, R₆ is unsubstituted acyloxy. In some embodiments, R₆ is substituted acyloxy. In some embodiments, R₆ is nitro. In some embodiments, R₆ is halogen. In some embodiments, R₆ is unsubstituted C₁-C₁₀ aliphatic acyl. In some embodiments, R₆ is substituted C₁-C₁₀ aliphatic acyl. In some embodiments, R₆ is unsubstituted C₆-C₁₀ aromatic acyl. In some embodiments, R₆ is substituted C₆-C₁₀ aromatic acyl. In some embodiments, R₆ is unsubstituted C₆-C₁₀ aralkyl acyl. In some embodiments, R₆ is substituted C₆-C₁₀ aralkyl acyl. In some embodiments, R₆ is unsubstituted C₆-C₁₀ alkylaryl acyl. In some embodiments, R₆ is substituted C₆-C₁₀ alkylaryl acyl. In some embodiments, R₆ is unsubstituted alkoxy. In some embodiments, R₆ is substituted alkoxy. In some embodiments, R₆ is unsubstituted aryl. In some embodiments, R₆ is substituted aryl. In some embodiments, R₆ is unsubstituted C₃-C₁₀ heterocyclyl. In some embodiments, R₆ is substituted C₃-C₁₀ heterocyclyl. In some embodiments, R₆ is unsubstituted heteroaryl, In some embodiments, R₆ is unsubstituted C₃-C₁₀ cycloalkyl. In some embodiments, R₆ is substituted C₃-C₁₀ cycloalkyl. In some embodiments, R₆ is —OPO₃WY. In some embodiments, R₆ is —OCH₂PO₄WY. In some embodiments, R₆ is —OCH₂PO₄Z. In some embodiments, R₆ is —OPO₃Z.

In some embodiments, R₇ is hydrogen. In some embodiments, R₇ is carboxaldehyde. In some embodiments, R₇ is unsubstituted amine. In some embodiments, R₇ is substituted amine. In some embodiments, R₇ is unsubstituted C₁-C₁₀ alkyl. In some embodiments, R₇ is substituted C₁-C₁₀ alkyl. In some embodiments, R₇ is unsubstituted C₂-C₁₀ alkynyl. In some embodiments, R₇ is substituted C₂-C₁₀ alkynyl. In some embodiments, R₇ is unsubstituted C₂-C₁₀ alkenyl. In some embodiments, R₇ is substituted C₂-C₁₀ alkenyl. In some embodiments, R₇ is carboxyl. In some embodiments, R₇ is unsubstituted carbohydrate. In some embodiments, R₇ is substituted carbohydrate. In some embodiments, R₇ is unsubstituted ester. In some embodiments, R₇ is substituted ester. In some embodiments, R₇ is unsubstituted acyloxy. In some embodiments, R₇ is substituted acyloxy. In some embodiments, R₇ is nitro. In some embodiments, R₇ is halogen. In some embodiments, R₇ is unsubstituted C₁-C₁₀ aliphatic acyl. In some embodiments, R₇ is substituted C₁-C₁₀ aliphatic acyl. In some embodiments, R₇ is unsubstituted C₆-C₁₀ aromatic acyl. In some embodiments, R₇ is substituted C₆-C₁₀ aromatic acyl. In some embodiments, R₇ is unsubstituted C₆-C₁₀ aralkyl acyl. In some embodiments, R₇ is substituted C₆-C₁₀ aralkyl acyl. In some embodiments, R₇ is unsubstituted C₆-C₁₀ alkylaryl acyl. In some embodiments, R₇ is substituted C₆-C₁₀ alkylaryl acyl. In some embodiments, R₇ is unsubstituted alkoxy. In some embodiments, R₇ is substituted alkoxy. In some embodiments, R₇ is unsubstituted aryl. In some embodiments, R₇ is substituted aryl. In some embodiments, R₇ is unsubstituted C₃-C₁₀heterocyclyl. In some embodiments, R₇ is substituted C₃-C₁₀heterocyclyl. In some embodiments, R₇ is unsubstituted heteroaryl, In some embodiments, R₇ is unsubstituted C₃-C₁₀ cycloalkyl. In some embodiments, R₇ is substituted C₃-C₁₀ cycloalkyl. In some embodiments, R₇ is —OPO₃WY. In some embodiments, R₇ is —OCH₂PO₄WY. In some embodiments, R₇ is —OCH₂PO₄Z. In some embodiments, R₇ is —OPO₃Z.

In some embodiments, R₈ is hydrogen. In some embodiments, R₈ is hydroxyl. In some embodiments, R₈ is carboxaldehyde. In some embodiments, R₈ is unsubstituted amine. In some embodiments, R₈ is substituted amine. In some embodiments, R₈ is unsubstituted C₁-C₁₀ alkyl. In some embodiments, R₈ is substituted C₁-C₁₀ alkyl. In some embodiments, R₈ is unsubstituted C₂-C₁₀ alkynyl. In some embodiments, R₈ is substituted C₂-C₁₀ alkynyl. n some embodiments, R₈ is unsubstituted C₂-C₁₀ alkenyl. In some embodiments, R₈ is substituted C₂-C₁₀ alkenyl. In some embodiments, R₈ is carboxyl. In some embodiments, R₈ is unsubstituted carbohydrate. In some embodiments, R₈ is substituted carbohydrate. In some embodiments, R₈ is unsubstituted ester. In some embodiments, R₈ is substituted ester. In some embodiments, R₈ is unsubstituted acyloxy. In some embodiments, R₈ is substituted acyloxy. In some embodiments, R₈ is nitro. In some embodiments, R₈ is halogen. In some embodiments, R₈ is unsubstituted C₁-C₁₀ aliphatic acyl. In some embodiments, R₈ is substituted C₁-C₁₀ aliphatic acyl. In some embodiments, R₈ is unsubstituted C₆-C₁₀ aromatic acyl. In some embodiments, R₈ is substituted C₆-C₁₀ aromatic acyl. In some embodiments, R₈ is unsubstituted C₆-C₁₀ aralkyl acyl. In some embodiments, R₈ is substituted C₆-C₁₀ aralkyl acyl. In some embodiments, R₈ is unsubstituted C₆-C₁₀ alkylaryl acyl. In some embodiments, R₈ is substituted C₆-C₁₀ alkylaryl acyl. In some embodiments, R₈ is unsubstituted alkoxy. In some embodiments, R₈ is substituted alkoxy. In some embodiments, R₈ is unsubstituted aryl. In some embodiments, R₈ is substituted aryl. In some embodiments, R₈ is unsubstituted C₃-C₁₀ heterocyclyl. In some embodiments, R₈ is substituted C₃-C₁₀ heterocyclyl. In some embodiments, R₈ is unsubstituted heteroaryl, In some embodiments, R₈ is unsubstituted C₃-C₁₀ cycloalkyl. In some embodiments, R₈ is substituted C₃-C₁₀ cycloalkyl. In some embodiments, R₈ is —OPO₃WY. In some embodiments, R₈ is —OCH₂PO₄WY. In some embodiments, R₈ is —OCH₂PO₄Z. In some embodiments, R₈ is —OPO₃Z.

In some embodiments, R₉ is hydrogen. In some embodiments, R₉ is carboxaldehyde. In some embodiments, R₉ is unsubstituted amine. In some embodiments, R₉ is substituted amine. In some embodiments, R₉ is unsubstituted C₁-C₁₀ alkyl. In some embodiments, R₉ is substituted C₁-C₁₀ alkyl. In some embodiments, R₉ is unsubstituted C₂-C₁₀ alkynyl. In some embodiments, R₉ is substituted C₂-C₁₀ alkynyl. In some embodiments, R₉ is unsubstituted C₂-C₁₀ alkenyl. In some embodiments, R₉ is substituted C₂-C₁₀ alkenyl. In some embodiments, R₉ is carboxyl. In some embodiments, R₉ is unsubstituted carbohydrate. In some embodiments, R₉ is substituted carbohydrate. In some embodiments, R₉ is unsubstituted ester. In some embodiments, R₉ is substituted ester. In some embodiments, R₉ is unsubstituted acyloxy. In some embodiments, R₉ is substituted acyloxy. In some embodiments, R₉ is nitro. In some embodiments, R₉ is halogen. In some embodiments, R₉ is unsubstituted C₁-C₁₀ aliphatic acyl. In some embodiments, R₉ is substituted C₁-C₁₀ aliphatic acyl. In some embodiments, R₉ is unsubstituted C₆-C₁₀ aromatic acyl. In some embodiments, R₉ is substituted C₆-C₁₀ aromatic acyl. In some embodiments, R₉ is unsubstituted C₆-C₁₀ aralkyl acyl. In some embodiments, R₉ is substituted C₆-C₁₀ aralkyl acyl. In some embodiments, R₉ is unsubstituted C₆-C₁₀ alkylaryl acyl. In some embodiments, R₉ is substituted C₆-C₁₀ alkylaryl acyl. In some embodiments, R₉ is unsubstituted alkoxy. In some embodiments, R₉ is substituted alkoxy. In some embodiments, R₉ is unsubstituted aryl. In some embodiments, R₉ is substituted aryl. In some embodiments, R₉ is unsubstituted C₃-C₁₀ heterocyclyl. In some embodiments, R₉ is substituted C₃-C₁₀ heterocyclyl. In some embodiments, R₉ is unsubstituted heteroaryl, In some embodiments, R₉ is unsubstituted C₃-C₁₀ cycloalkyl. In some embodiments, R₉ is substituted C₃-C₁₀ cycloalkyl. In some embodiments, R₉ is —OPO₃WY. In some embodiments, R₉ is —OCH₂PO₄WY. In some embodiments, R₉ is —OCH₂PO₄Z. In some embodiments, R₉ is —OPO₃Z.

In some embodiments of the invention, the pyrone analog of Formula III is of Formula X:

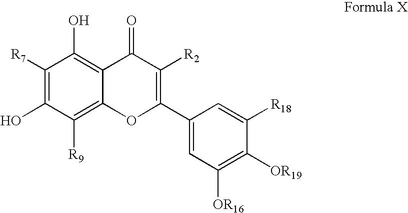

wherein R₂, R₁₆, R₁₈, and R₁₉ are as defined in Formula II and R₇ and R₉ are as defined in Formula III.

In other embodiments of the invention, the pyrone analog of Formula III is of Formula XI:

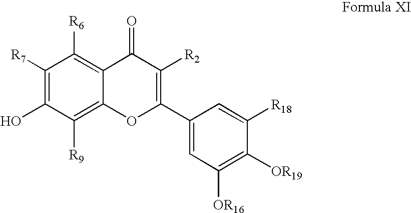

wherein R₂, R₁₆, R₁₈, and R₁₉ are as defined in Formula II and R₆, R₇, and R₉ are as defined in Formula III.

In some embodiments of the invention, compounds of the following Formulae VIII-A, VIII-B, and VIII-C, are useful in the methods of the invention, where each instance of R_{c} and R_{d} is independently hydrogen, —OPO₃WY, —OPO₃Z, —OCH₂OPOWY, or —OCH₂OPO₃Z, where W and Y are hydrogen, methyl, ethyl, alkyl, carbohydrate, lithium, sodium or potassium and Z is calcium, magnesium or iron.

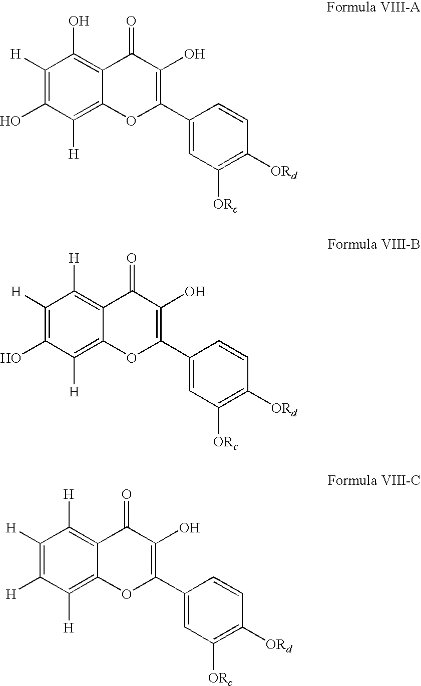

In some embodiments of the invention, for a compound of Formulae VIII-A, VIII-B, or VIII-C, R_{c} and R_{d} are hydrogen. In some embodiments of the invention, for a compound of Formulae VIII-A, VIII-B, or VIII-C, R_{c} is —OPO₃WY and R_{d} is hydrogen. In some embodiments of the invention, for a compound of Formulae VIII-A, VIII-B, or VIII-C, R_{c} is —PO₃WY and R_{d} is —OPO₃WY. In some embodiments of the invention, for a compound of Formulae VIII-A, VIII-B, or VIII-C, R_{c} is a mixture of hydrogen and —PO₃WY and R_{d} is —PO₃WY. In some embodiments of the invention, for a compound of Formulae VIII-A, VIII-B, or VIII-C, R_{c} is hydrogen and R_{d} is a mixture of hydrogen and —OPO₃Z. In some embodiments of the invention, for a compound of Formulae VIII-A, VIII-B, or VIII-C, R_{c} is —OPO₃Z and R_{d} is hydrogen. In some embodiments of the invention, for a compound of Formulae VIII-A, VIII-B, or VIII-C, R_{c} is —OPO₃Z and R_{d} is —OPO₃Z. In some embodiments of the invention, for a compound of Formulae VIII-A, VIII-B, or VIII-C, R_{c} is a mixture of hydrogen and —OPO₃Z and R_{d} is —OPO₃Z. In some embodiments of the invention, for a compound of Formulae VIII-A, VIII-B, or VIII-C, R_{c} is hydrogen and R_{d} is a mixture of hydrogen and —OPO₃Z. In some embodiments of the invention, for a compound of Formulae VIII-A, VIII-B, or VIII-C, R_{c} is —CH₂OPO₃Z and R_{d} is hydrogen. In some embodiments of the invention, for a compound of Formulae VIII-A, VIII-B, or VIII-C, R_{c} is —CH₂OPO₃Z and R_{d} is —CH₂OPO₃Z. In some embodiments of the invention, for a compound of Formulae VIII-A, VIII-B, or VIII-C, R_{c} is a mixture of hydrogen and —CH₂OPO₃Z and R_{d} is —CH₂OPO₃Z. In some embodiments of the invention, for a compound of Formulae VIII-A, VIII-B, or VIII-C, R_{c} is hydrogen and R_{d} is a mixture of hydrogen and —CH₂OPO₃Z.

In other embodiments of the invention, the pyrone analog of Formula III is of Formula XII:

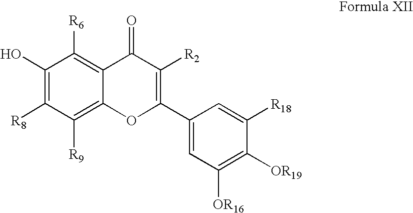

wherein R₂, R₁₆, R₁₈, and R₁₉ are as defined in Formula II and R₆, R₈ and R₉ are as defined in Formula III.

In other embodiments of the invention, the pyrone analog of Formula III is of Formula XIII:

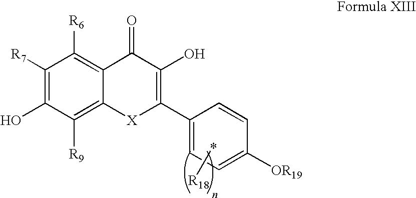

wherein X, R₁₈, and R₁₉ are as defined in Formula II and R₆, R₇, and R₉ are as defined in Formula III.

In some embodiments, the pyrone analog of Formula III is of Formula XIV:

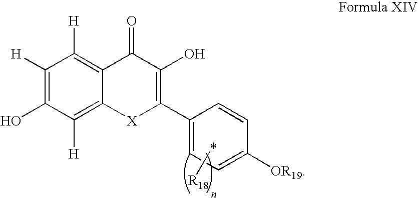

In some embodiments, the pyrone analog of Formula III is of Formula XV:

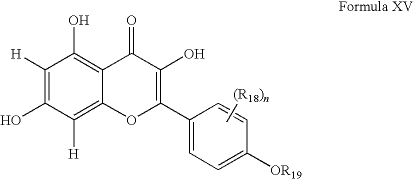

wherein R₁₈, R₁₉, and n are as defined in Formula II.

In some embodiments, the pyrone analog of Formula III is of Formula XVI:

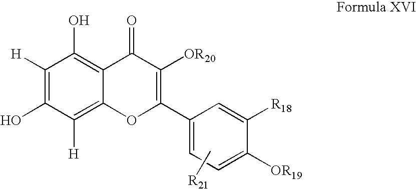

wherein R₁₈, R₁₉, R₂₁, and n are as defined in Formula II;

R₂₀ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, optionally substituted C₃-C₁₀ cycloalkyl, —PO₃WY, —CH₂PO₄WY, —CH₂PO₄Z or —PO₃Z; and

W and Y are independently hydrogen, methyl, ethyl, alkyl, carbohydrate, or a cation, and Z is a multivalent cation.

In some embodiments, R₂₀ is hydrogen. In some embodiments, R₂₀ is unsubstituted C₁-C₁₀ alkyl. In some embodiments, R₂₀ is substituted C₁-C₁₀ alkyl. In some embodiments, R₂₀ is unsubstituted C₂-C₁ alkynyl. In some embodiments, R₂₀ is substituted C₂-C₁₀ alkynyl. In some embodiments, R₂₀ is unsubstituted C₂-C₁₀ alkenyl. In some embodiments, R₂₀ is substituted C₂-C₁₀ alkenyl. In some embodiments, R₂₀ is unsubstituted carbohydrate. In some embodiments, R₂₀ is substituted carbohydrate. In some embodiments, R₂₀ is unsubstituted C₁-C₁₀ aliphatic acyl. In some embodiments, R₂₀ is substituted C₁-C₁₀ aliphatic acyl. In some embodiments, R₂₀ is unsubstituted C₆-C₁₀ aromatic acyl. In some embodiments, R₂₀ is substituted C₆-C₁₀ aromatic acyl. In some embodiments, R₂₀ is unsubstituted C₆-C₁₀ aralkyl acyl. In some embodiments, R₂₀ is substituted C₆-C₁₀ aralkyl acyl. In some embodiments, R₂₀ is unsubstituted C₆-C₁₀ alkylaryl acyl. In some embodiments, R₂₀ is substituted C₆-C₁₀ alkylaryl acyl. In some embodiments, R₂₀ is unsubstituted aryl. In some embodiments, R₂₀ is substituted aryl. In some embodiments, R₂₀ is unsubstituted C₃-C₁₀ heterocyclyl. In some embodiments, R₂₀ is substituted C₃-C₁₀ heterocyclyl. In some embodiments, R₂₀ is unsubstituted heteroaryl. In some embodiments, R₂₀ is substituted heteroaryl. In some embodiments, R₂₀ is unsubstituted C₃-C₁₀ cycloalkyl. In some embodiments, R₂₀ is substituted C₃-C₁₀ cycloalkyl. In some embodiments, R₂₀ is —PO₃WY. In some embodiments, R₂₀ is —CH₂PO₄WY. In some embodiments, R₂₀ is —CH₂PO₄Z. In some embodiments, R₂₀ is —PO₃Z.

In some embodiments, the pyrone analog of Formula III is of Formula XVII:

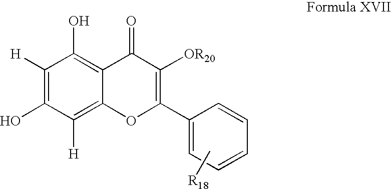

wherein R₁₈ is as defined in Formula II; and

R₂₀ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀heterocyclyl, heteroaryl, optionally substituted C₃-C₁₀cycloalkyl, —PO₃WY, —CH₂PO₄WY, —CH₂PO₄Z or —PO₃Z.

In some embodiments, R₂₀ is hydrogen. In some embodiments, R₂₀ is unsubstituted C₁-C₁₀ alkyl. In some embodiments, R₂₀ is substituted C₁-C₁₀ alkyl. In some embodiments, R₂₀ is unsubstituted C₂-C₁₀ alkynyl. In some embodiments, R₂₀ is substituted C₂-C₁₀ alkynyl. In some embodiments, R₂₀ is unsubstituted C₂-C₁₀ alkenyl. In some embodiments, R₂₀ is substituted C₂-C₁₀ alkenyl. In some embodiments, R₂₀ is unsubstituted carbohydrate. In some embodiments, R₂₀ is substituted carbohydrate. In some embodiments, R₂₀ is unsubstituted C₁-C₁₀ aliphatic acyl. In some embodiments, R₂₀ is substituted C₁-C₁₀ aliphatic acyl. In some embodiments, R₂₀ is unsubstituted C₆-C₁₀ aromatic acyl. In some embodiments, R₂₀ is substituted C₆-C₁₀ aromatic acyl. In some embodiments, R₂₀ is unsubstituted C₆-C₁₀ aralkyl acyl. In some embodiments, R₂₀ is substituted C₆-C₁₀ aralkyl acyl. In some embodiments, R₂₀ is unsubstituted C₆-C₁₀ alkylaryl acyl. In some embodiments, R₂₀ is substituted C₆-C₁₀ alkylaryl acyl. In some embodiments, R₂₀ is unsubstituted aryl. In some embodiments, R₂₀ is substituted aryl. In some embodiments, R₂₀ is unsubstituted C₃-C₁₀ heterocyclyl. In some embodiments, R₂₀ is substituted C₃-C₁₀ heterocyclyl. In some embodiments, R₂₀ is unsubstituted heteroaryl. In some embodiments, R₂₀ is substituted heteroaryl. In some embodiments, R₂₀ is unsubstituted C₃-C₁₀ cycloalkyl. In some embodiments, R₂₀ is substituted C₃-C₁₀ cycloalkyl. In some embodiments, R₂₀ is —PO₃WY. In some embodiments, R₂₀ is —CH₂PO₄WY. In some embodiments, R₂₀ is —CH₂PO₄Z. In some embodiments, R₂₀ is —PO₃Z.

In some embodiments, the pyrone analog of Formula III is of Formula XVIII:

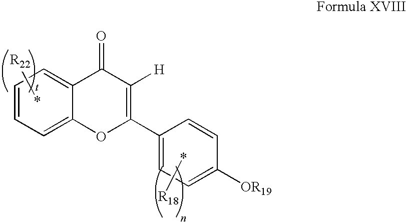

wherein R₁₈ and R₁₉ are as defined in Formula II;

wherein each instance of R₂₂ is independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀ heterocyclic, C₃-C₁₀cycloalkyl, —OPO₃WY, —OCH₂PO₄WY, —OCH₂PO₄Z or —OPO₃Z; and

t is an integer of 0, 1, 2, 3, or 4

In some embodiments, R₂₂ is hydrogen. In some embodiments, R₂₂ is hydroxy. In some embodiments, R₂₂ is carboxaldehyde. In some embodiments, R₂₂ is unsubstituted amine. In some embodiments, R₂₂ is substituted amine. In some embodiments, R₂₂ is unsubstituted C₁-C₁₀ alkyl. In some embodiments, R₂₂ is unsubstituted C₂-C₁₀ alkynyl. In some embodiments, R₂₂ is substituted C₂-C₁₀ alkynyl. In some embodiments, R₂₂ is unsubstituted C₂-C₁₀ alkenyl. In some embodiments, R₂₂ is substituted C₂-C₁₀ alkenyl. In some embodiments, R₂₂ is carboxyl. In some embodiments, R₂₂ is unsubstituted carbohydrate. In some embodiments, R₂₂ is substituted carbohydrate. In some embodiments, R₂₂ is unsubstituted ester. In some embodiments, R₂₂ is substituted ester. In some embodiments, R₂₂ is unsubstituted acyloxy. In some embodiments, R₂₂ is substituted acyloxy. In some embodiments, R₂₂ is nitro. In some embodiments, R₂₂ is halogen. In some embodiments, R₂₂ is unsubstituted C₁-C₁₀ aliphatic acyl. In some embodiments, R₂₂ is substituted C₁-C₁₀ aliphatic acyl. In some embodiments, R₂₂ is unsubstituted C₆-C₁₀ aromatic acyl. In some embodiments, R₂₂ is substituted C₆-C₁₀ aromatic acyl. In some embodiments, R₂₂ is unsubstituted C₆-C₁₀ aralkyl acyl. In some embodiments, R₂₂ is substituted C₆-C₁₀ aralkyl acyl. In some embodiments, R₂₂ is unsubstituted C₆-C₁₀ alkylaryl acyl. In some embodiments, R₂₂ is substituted C₆-C₁₀ alkylaryl acyl. In some embodiments, R₂₂ is unsubstituted alkoxy. In some embodiments, R₂₂ is substituted alkoxy. In some embodiments, R₂₂ is unsubstituted aryl. In some embodiments, R₂₂ is substituted aryl. In some embodiments, R₁₈ is unsubstituted C₃-C₁₀ heterocyclyl. In some embodiments, R₂₂ is substituted C₃-C₁₀ heterocyclyl. In some embodiments, R₂₂ is unsubstituted heteroaryl. In some embodiments, R₂₂ is substituted heteroaryl. In some embodiments, R₂₂ is unsubstituted C₃-C₁₀ cycloalkyl. In some embodiments, R₂₂ is substituted C₃-C₁₀ cycloalkyl. In some embodiments, R₂₂ is —OPO₃WY. In some embodiments, R₂₂ is —OCH₂PO₄WY. In some embodiments, R₂₂ is —OCH₂PO₄Z. In some embodiments, R₂₂ is —OPO₃Z.

In some embodiments, t is an integer of 0. In some embodiments, t is an integer of 1. In some embodiments, t is an integer of 2. In some embodiments, t is an integer of 3. In some embodiments, t is an integer of 4.

In some embodiments, the pyrone analog of Formula III is of Formula XIX:

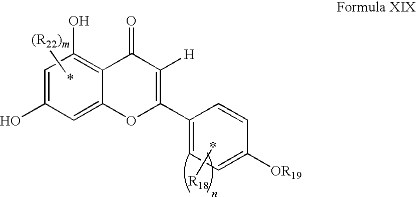

wherein R₁₈ and R₁₉ are as defined in Formula II;

wherein each instance of R₂₂ is independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀ heterocyclic, C₃-C₁₀ cycloalkyl, —OPO₃WY, —OCH₂PO₄WY, —OCH₂PO₄Z or —OPO₃Z; and

m is an integer of 0, 1, or 2.

In some embodiments, m is an integer of 0. In some embodiments, m is an integer of 1. In some embodiments, m is an integer of 2.

In some embodiments, the pyrone analog of Formula III is of Formula XX:

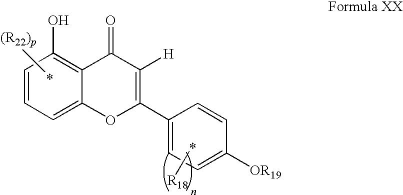

wherein R₁₈ and R₁₉ are as defined in Formula II;

wherein each instance of R₂₂ is independently hydrogen, hydroxyl, carboxaldehyde, amine, C₁-C₁₀ alkyl, C₂-C₁₀ alkynyl, C₂-C₁₀alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀ heterocyclic, C₃-C₁₀ cycloalkyl, —OPO₃WY, —OCH₂PO₄WY, —OCH₂PO₄Z or —OPO₃Z; and

p is an integer of 0, 1, 2 or 3.

In some embodiments, R₂₂ is hydrogen. In some embodiments, R₂₂ is hydroxy. In some embodiments, R₂₂ is carboxaldehyde. In some embodiments, R₂₂ is unsubstituted amine. In some embodiments, R₂₂ is substituted amine. In some embodiments, R₂₂ is unsubstituted C₁-C₁₀ alkyl. In some embodiments, R₂₂ is unsubstituted C₂-C₁₀ alkynyl. In some embodiments, R₂₂ is substituted C₂-C₁₀ alkynyl. In some embodiments, R₂₂ is unsubstituted C₂-C₁₀ alkenyl. In some embodiments, R₂₂ is substituted C₂-C₁₀ alkenyl. In some embodiments, R₂₂ is carboxyl. In some embodiments, R₂₂ is unsubstituted carbohydrate. In some embodiments, R₂₂ is substituted carbohydrate. In some embodiments, R₂₂ is unsubstituted ester. In some embodiments, R₂₂ is substituted ester. In some embodiments, R₂₂ is unsubstituted acyloxy. In some embodiments, R₂₂ is substituted acyloxy. In some embodiments, R₂₂ is nitro. In some embodiments, R₂₂ is halogen. In some embodiments, R₂₂ is unsubstituted C₁-C₁₀ aliphatic acyl. In some embodiments, R₂₂ is substituted C₁-C₁₀ aliphatic acyl. In some embodiments, R₂₂ is unsubstituted C₆-C₁₀ aromatic acyl. In some embodiments, R₂₂ is substituted C₆-C₁₀ aromatic acyl. In some embodiments, R₂₂ is unsubstituted C₆-C₁₀ aralkyl acyl. In some embodiments, R₂₂ is substituted C₆-C₁₀ aralkyl acyl. In some embodiments, R₂₂ is unsubstituted C₆-C₁₀ alkylaryl acyl. In some embodiments, R₂₂ is substituted C₆-C₁₀ alkylaryl acyl. In some embodiments, R₂₂ is unsubstituted alkoxy. In some embodiments, R₂₂ is substituted alkoxy. In some embodiments, R₂₂ is unsubstituted aryl. In some embodiments, R₂₂ is substituted aryl. In some embodiments, R₁₈ is unsubstituted C₃-C₁₀ heterocyclyl. In some embodiments, R₂₂ is substituted C₃-C₁₀ heterocyclyl. In some embodiments, R₂₂ is unsubstituted heteroaryl. In some embodiments, R₂₂ is substituted heteroaryl. In some embodiments, R₂₂ is unsubstituted C₃-C₁₀ cycloalkyl. In some embodiments, R₂₂ is substituted C₃-C₁₀ cycloalkyl. In some embodiments, R₂₂ is —OPO₃WY. In some embodiments, R₂₂ is —OCH₂PO₄WY. In some embodiments, R₂₂ is —OCH₂PO₄Z. In some embodiments, R₂₂ is —OPO₃Z.

In some embodiments, p is an integer of 0. In some embodiments, p is an integer of 1. In some embodiments, p is an integer of 2. In some embodiments, p is an integer of 3.

In some embodiments, the pyrone analog of Formula III is of Formula XXI:

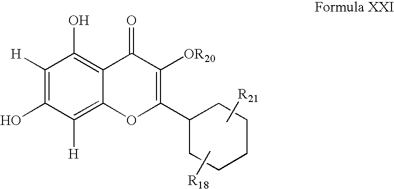

wherein R₁₈ and R₂, are as defined in Formula II; and

R₂₀ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, optionally substituted C₃-C₁₀cycloalkyl, —PO₃WY, —CH₂PO₄WY, —CH₂PO₄Z or —PO₃Z.

In some embodiments, R₂₀ is hydrogen. In some embodiments, R₂₀ is unsubstituted C₁-C₁₀ alkyl. In some embodiments, R₂₀ is substituted C₁-C₁₀ alkyl. In some embodiments, R₂₀ is unsubstituted C₂-C₁₀ alkynyl. In some embodiments, R₂₀ is substituted C₂-C₁₀ alkynyl. In some embodiments, R₂₀ is unsubstituted C₂-C₁₀ alkenyl. In some embodiments, R₂₀ is substituted C₂-C₁₀ alkenyl. In some embodiments, R₂₀ is unsubstituted carbohydrate. In some embodiments, R₂₀ is substituted carbohydrate. In some embodiments, R₂₀ is unsubstituted C₁-C₁₀ aliphatic acyl. In some embodiments, R₂₀ is substituted C₁-C₁₀ aliphatic acyl. In some embodiments, R₂₀ is unsubstituted C₆-C₁₀ aromatic acyl. In some embodiments, R₂₀ is substituted C₆-C₁₀ aromatic acyl. In some embodiments, R₂₀ is unsubstituted C₆-C₁₀ aralkyl acyl. In some embodiments, R₂₀ is substituted C₆-C₁₀ aralkyl acyl. In some embodiments, R₂₀ is unsubstituted C₆-C₁₀ alkylaryl acyl. In some embodiments, R₂₀ is substituted C₆-C₁₀ alkylaryl acyl. In some embodiments, R₂₀ is unsubstituted aryl. In some embodiments, R₂₀ is substituted aryl. In some embodiments, R₂₀ is unsubstituted C₃-C₁₀ heterocyclyl. In some embodiments, R₂₀ is substituted C₃-C₁₀ heterocyclyl. In some embodiments, R₂₀ is unsubstituted heteroaryl. In some embodiments, R₂₀ is substituted heteroaryl. In some embodiments, R₂₀ is unsubstituted C₃-C₁₀ cycloalkyl. In some embodiments, R₂₀ is substituted C₃-C₁₀ cycloalkyl. In some embodiments, R₂₀ is —PO₃WY. In some embodiments, R₂₀ is —CH₂PO₄WY. In some embodiments, R₂₀ is —CH₂PO₄Z. In some embodiments, R₂₀ is —PO₃Z.

In some embodiments, the pyrone analog of Formula III is of Formula XXII:

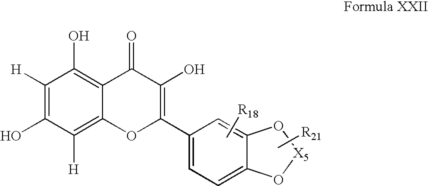

wherein R₁₈ and R₂₁ are as defined in Formula II;

wherein X₅ is a C₁ to C₄ group, optionally interrupted by O, S, NR₂₃, or NR₂₃R₂₃ as valency permits, forming a ring which is aromatic or nonaromatic;

each instance of R₂₃ is independently hydrogen, C₁-C₁₀ alkyl, C₂-C₁₀ alkynyl, C₂-C₁₀ alkenyl, carbohydrate, acyloxy, C₁-C₁₀ aliphatic acyl, C₆-C₁₀ aromatic acyl, C₆-C₁₀ aralkyl acyl, C₆-C₁₀ alkylaryl acyl, alkoxy, aryl, heteroaryl, C₅-C₁₀ heterocyclyl, C₃-C₁₀ cycloalkyl, —PO₃WY, —CH₂PO₄WY, —CH₂PO₄Z or —PO₃Z.

In some embodiments, R₂₃ is hydrogen. In some embodiments, R₂₃ is unsubstituted C₁-C₁₀ alkyl. In some embodiments, R₂₃ is substituted C₁-C₁₀ alkyl. In some embodiments, R₂₃ is unsubstituted C₂-C₁₀ alkynyl. In some embodiments, R₂₃ is substituted C₂-C₁₀ alkynyl. In some embodiments, R₂₃ is unsubstituted C₂-C₁₀ alkenyl. In some embodiments, R₂₃ is substituted C₂-C₁₀ alkenyl. In some embodiments, R₂₃ is unsubstituted acyloxy. In some embodiments, R₂₃ is substituted acyloxy. In some embodiments, R₂₃ is unsubstituted carbohydrate. In some embodiments, R₂₃ is substituted carbohydrate. In some embodiments, R₂₃ is unsubstituted acyloxy. In some embodiments, R₂₃ is substituted acyloxy. In some embodiments, R₂₃ is unsubstituted C₁-C₁₀ aliphatic acyl. In some embodiments, R₂₃ is substituted C₁-C₁₀ aliphatic acyl. In some embodiments, R₂₃ is unsubstituted C₆-C₁₀ aromatic acyl. In some embodiments, R₂₃ is substituted C₆-C₁₀ aromatic acyl. In some embodiments, R₂₃ is unsubstituted C₆-C₁₀ aralkyl acyl. In some embodiments, R₂₃ is substituted C₆-C₁₀ aralkyl acyl. In some embodiments, R₂₃ is unsubstituted C₆-C₁₀ alkylaryl acyl. In some embodiments, R₂₃ is substituted C₆-C₁₀ alkylaryl acyl. In some embodiments, R₂₃ is unsubstituted alkoxy. In some embodiments, R₂₃ is substituted alkoxy. In some embodiments, R₂₃ is unsubstituted aryl. In some embodiments, R₂₃ is substituted aryl. In some embodiments, R₂₃ is unsubstituted C₃-C₁₀ heterocyclyl. In some embodiments, R₂₃ is substituted C₃-C₁₀ heterocyclyl. In some embodiments, R₂₃ is unsubstituted heteroaryl. In some embodiments, R₂₃ is substituted heteroaryl. In some embodiments, R₂₃ is unsubstituted C₃-C₁₀ cycloalkyl. In some embodiments, R₂₃ is substituted C₃-C₁₀ cycloalkyl.

In some embodiments, the pyrone analog of Formula III is of Formula XXIII:

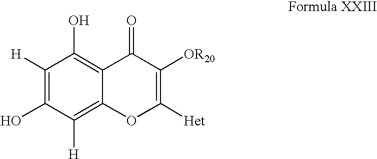

Wherein R₂₀ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, optionally substituted C₃-C₁₀ cycloalkyl, —PO₃WY, —CH₂PO₄WY, —CH₂PO₄Z or —PO₃Z;

Het is a 3 to 10 membered optionally substituted monocyclic or bicyclic heteroaromatic or heterocyclic ring system containing 1, 2, 3, 4, or 5 heteroatoms selected from the group of O, S, and N, with the proviso that no two adjacent ring atoms are O or S, wherein the ring system is unsaturated, partially unsaturated or saturated, wherein any number of the ring atoms have substituents as valency permits which are hydrogen, hydroxyl, carboxyaldehyde, alkylcarboxaldehyde, imino, C₁-C₁₀ alkyl, C₁-C₁₀ alkynyl, C₁-C₁₀ alkenyl, carboxyl, carbohydrate, acyloxy, nitro, halogen, C₁-C₁₀ aliphatic acyl, C₅-C₁₀ aromatic acyl, C₆-C₁₀ aralkyl acyl, C₆-C₁₀ alkylaryl acyl, alkoxy, amine, aryl, heteroaryl, C₅-C₁₀ heterocyclyl, C₅-C₁₀ cycloalkyl, —OPO₃WY, —OCH₂PO₄WY, —OCH₂PO₄Z or —OPO₃Z; and

W and Y are independently hydrogen, methyl, ethyl, alkyl, carbohydrate, or a cation, and Z is a multivalent cation.

In some embodiments, R₂₀ is hydrogen. In some embodiments, R₂₀ is unsubstituted C₁-C₁₀ alkyl. In some embodiments, R₂₀ is substituted C₁-C₁₀ alkyl. In some embodiments, R₂₀ is unsubstituted C₂-C₁₀ alkynyl. In some embodiments, R₂₀ is substituted C₂-C₁₀ alkynyl. In some embodiments, R₂₀ is unsubstituted C₂-C₁₀ alkenyl. In some embodiments, R₂₀ is substituted C₂-C₁₀ alkenyl. In some embodiments, R₂₀ is unsubstituted carbohydrate. In some embodiments, R₂₀ is substituted carbohydrate. In some embodiments, R₂₀ is unsubstituted C₁-C₁₀ aliphatic acyl. In some embodiments, R₂₀ is substituted C₁-C₁₀ aliphatic acyl. In some embodiments, R₂₀ is unsubstituted C₆-C₁₀ aromatic acyl. In some embodiments, R₂₀ is substituted C₆-C₁₀ aromatic acyl. In some embodiments, R₂₀ is unsubstituted C₆-C₁₀ aralkyl acyl. In some embodiments, R₂₀ is substituted C₆-C₁₀ aralkyl acyl. In some embodiments, R₂₀ is unsubstituted C₆-C₁₀ alkylaryl acyl. In some embodiments, R₂₀ is substituted C₆-C₁₀ alkylaryl acyl. In some embodiments, R₂₀ is unsubstituted aryl. In some embodiments, R₂₀ is substituted aryl. In some embodiments, R₂₀ is unsubstituted C₃-C₁₀ heterocyclyl. In some embodiments, R₂₀ is substituted C₃-C₁₀ heterocyclyl. In some embodiments, R₂₀ is unsubstituted heteroaryl. In some embodiments, R₂₀ is substituted heteroaryl. In some embodiments, R₂₀ is unsubstituted C₃-C₁₀ cycloalkyl. In some embodiments, R₂₀ is substituted C₃-C₁₀ cycloalkyl. In some embodiments, R₂₀ is —PO₃WY. In some embodiments, R₂₀ is —CH₂PO₄WY. In some embodiments, R₂₀ is —CH₂PO₄Z. In some embodiments, R₂₀ is —PO₃Z.

In some embodiments, Het is one of the following formulae:

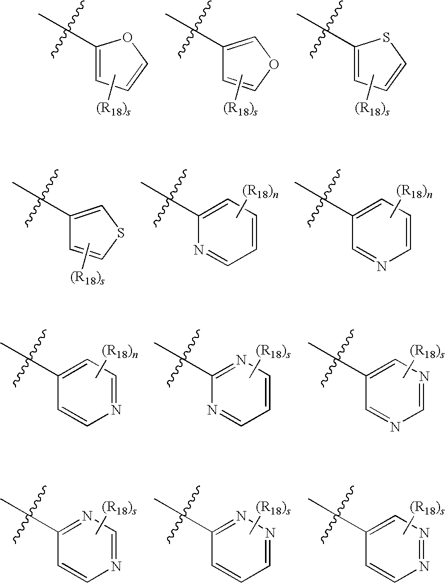

wherein each instance of R₁₈ is independently hydrogen, hydroxyl, carboxaldehyde, amine, C₁-C₁₀ alkyl, C₂-C₁₀ alkynyl, C₂-C₁₀alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀ heterocyclic, C₃-C₁₀ cycloalkyl, —OPO₃WY, —OCH₂PO₄WY, —OCH₂PO₄Z or —OPO₃Z;

s is an integer of 0, 1, 2, or 3; and

n is an integer of 0, 1, 2, 3, or 4.

In some embodiments, R₁₈ is hydrogen. In some embodiments, R₁₈ is hydroxy. In some embodiments, R₁₈ is carboxaldehyde. In some embodiments, R₁₈ is unsubstituted amine. In some embodiments, R₁₈ is substituted amine. In some embodiments, R₁₈ is unsubstituted C₁-C₁₀ alkyl. In some embodiments, R₁₉ is unsubstituted C₂-C₁₀ alkynyl. In some embodiments, R₁₈ is substituted C₂-C₁₀ alkynyl. In some embodiments, R₁₈ is unsubstituted C₂-C₁₀ alkenyl. In some embodiments, R₁₈ is substituted C₂-C₁₀ alkenyl. In some embodiments, R₁₈ is carboxyl. In some embodiments, R₁₈ is unsubstituted carbohydrate. In some embodiments, R₁₈ is substituted carbohydrate. In some embodiments, R₁₈ is substituted carbohydrate. In some embodiments, R₁₈ is unsubstituted ester. In some embodiments, R₁₈ is substituted ester. In some embodiments, R₁₈ is unsubstituted acyloxy. In some embodiments, R₁₈ is substituted acyloxy. In some embodiments, R₁₈ is nitro. In some embodiments, R₁₈ is halogen. In some embodiments, R₁₈ is unsubstituted C₁-C₁₀ aliphatic acyl. In some embodiments, R₁₈ is substituted C₁-C₁₀ aliphatic acyl. In some embodiments, R₁₈ is unsubstituted C₆-C₁₀ aromatic acyl. In some embodiments, R₁₈ is substituted C₆-C₁₀ aromatic acyl. In some embodiments, R₁₈ is unsubstituted C₆-C₁₀ aralkyl acyl. In some embodiments, R₁₈ is substituted C₆-C₁₀ aralkyl acyl. In some embodiments, R₁₈ is unsubstituted C₆-C₁₀ alkylaryl acyl. In some embodiments, R₁₈ is substituted C₆-C₁₀ alkylaryl acyl. In some embodiments, R₁₈ is unsubstituted alkoxy. In some embodiments, R₁₈ is substituted alkoxy. In some embodiments, R₁₈ is unsubstituted aryl. In some embodiments, R₁₈ is substituted aryl. In some embodiments, R₁₈ is unsubstituted C₃-C₁₀ heterocyclyl. In some embodiments, R₁₈ is substituted C₃-C₁₀ heterocyclyl. In some embodiments, R₁₈ is unsubstituted heteroaryl. In some embodiments, R₁₈ is substituted heteroaryl. In some embodiments, R₁₈ is unsubstituted C₃-C₁₀ cycloalkyl. In some embodiments, R₁₈ is substituted C₃-C₁₀ cycloalkyl. In some embodiments, R₁₈ is —OPO₃WY. In some embodiments, R₁₈ is —OCH₂PO₄WY. In some embodiments, R₁₈ is —OCH₂PO₄Z. In some embodiments, R₁₈ is —OPO₃Z.

In some embodiments, n is an integer of 0. In some embodiments, n is an integer of 1. In some embodiments, n is an integer of 2. In some embodiments, n is an integer of 3. In some embodiments, n is an integer of 4.

In some embodiments, s is an integer of 0. In some embodiments, s is an integer of 1. In some embodiments, s is an integer of 2. In some embodiments, s is an integer of 3.

In some embodiments of the invention, the pyrone analog of Formula II is of Formula IV:

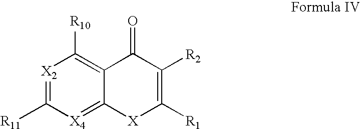

wherein X, X₂, X₄, R₁, and R₂ are as defined for Formula II; and

R₁₀ and R₁₁ are independently hydrogen, hydroxyl, carboxaldehyde, amino, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, amine, aryl, C₃-C₁₀ heterocyclyl, heteroaryl, C₃-C₁₀ cycloalkyl, —OPO₃WY, —OCH₂PO₄WY, —OCH₂PO₄Z or —OPO₃Z.

In some embodiments, R₁₀ is hydrogen. In some embodiments, R₁₀ is hydroxyl. In some embodiments, R₁₀ is carboxaldehyde. In some embodiments, R₁₀ is unsubstituted amine. In some embodiments, R₁₀ is substituted amine. In some embodiments, R₁₀ is unsubstituted C₁-C₁₀ alkyl. In some embodiments, R₁₀ is substituted C₁-C₁₀ alkyl. In some embodiments, R₁₀ is unsubstituted C₂-C₁₀ alkynyl. In some embodiments, R₁₀ is substituted C₂-C₁₀ alkynyl. In some embodiments, R₁₀ is unsubstituted C₂-C₁₀ alkenyl. In some embodiments, R₁₀ is substituted C₂-C₁₀ alkenyl. In some embodiments, R₁₀ is carboxyl. In some embodiments, R₁₀ is unsubstituted carbohydrate. In some embodiments, R₁₀ is substituted carbohydrate. In some embodiments, R₁₀ is unsubstituted ester. In some embodiments, R₁₀ is substituted ester. In some embodiments, R₁₀ is unsubstituted acyloxy. In some embodiments, R₁₀ is substituted acyloxy. In some embodiments, R₁₀ is nitro. In some embodiments, R₁₀ is halogen. In some embodiments, R₁₀ is unsubstituted C₁-C₁₀ aliphatic acyl. In some embodiments, R₁₀ is substituted C₁-C₁₀ aliphatic acyl. In some embodiments, R₁₀ is unsubstituted C₆-C₁₀ aromatic acyl. In some embodiments, R₁₀ is substituted C₆-C₁₀ aromatic acyl. In some embodiments, R₁₀ is unsubstituted C₆-C₁₀ aralkyl acyl. In some embodiments, R₁₀ is substituted C₆-C₁₀ aralkyl acyl. In some embodiments, R₁₀ is unsubstituted C₆-C₁ alkylaryl acyl. In some embodiments, R₁₀ is substituted C₆-C₁₀ alkylaryl acyl. In some embodiments, R₁₀ is unsubstituted alkoxy. In some embodiments, R₁₀ is substituted alkoxy. In some embodiments, R₁₀ is unsubstituted aryl. In some embodiments, R₁₀ is substituted aryl. In some embodiments, R₁₀ is unsubstituted C₃-C₁₀heterocyclyl. In some embodiments, R₁₀ is substituted C₃-C₁₀ heterocyclyl. In some embodiments, R₁₀ is unsubstituted heteroaryl, In some embodiments, R₁₀ is unsubstituted C₃-C₁₀ cycloalkyl. In some embodiments, R₁₀ is substituted C₃-C₁₀ cycloalkyl. In some embodiments, R₁₀ is —OPO₃WY. In some embodiments, R₁₀ is —OCH₂PO₄WY. In some embodiments, R₁₀ is —OCH₂PO₄Z. In some embodiments, R₁₀ is —OPO₃Z.

In some embodiments, R₁₁ is hydrogen. In some embodiments, R₁₁ is hydroxyl. In some embodiments, R₁₁ is carboxaldehyde. In some embodiments, R₁₁ is unsubstituted amine. In some embodiments, R₁₁ is substituted amine. In some embodiments, R₁₁ is unsubstituted C₁-C₁₀ alkyl. In some embodiments, R₁₁ is substituted C₁-C₁₀ alkyl. In some embodiments, R₁₁ is unsubstituted C₂-C₁₀ alkynyl. In some embodiments, R₁₁ is substituted C₂-C₁₀ alkynyl. In some embodiments, R₁₁ is unsubstituted C₂-C₁₀ alkenyl. In some embodiments, R₁₁ is substituted C₂-C₁₀ alkenyl. In some embodiments, R₁₁ is carboxyl. In some embodiments, R₁₁ is unsubstituted carbohydrate. In some embodiments, R₁₁ is substituted carbohydrate. In some embodiments, R₁ is unsubstituted ester. In some embodiments, R₁₁ is substituted ester. In some embodiments, R₁₁ is unsubstituted acyloxy. In some embodiments, R₁₁ is substituted acyloxy. In some embodiments, R₁₁ is nitro. In some embodiments, R₁₁ is halogen. In some embodiments, R₁₁ is unsubstituted C₁-C₁₀ aliphatic acyl. In some embodiments, R₁ is substituted C₁-C₁₀ aliphatic acyl. In some embodiments, R₁₁ is unsubstituted C₆-C₁₀ aromatic acyl. In some embodiments, R₁₁ is substituted C₆-C₁₀ aromatic acyl. In some embodiments, R₁₁ is unsubstituted C₆-C₁₀ aralkyl acyl. In some embodiments, R₁₁ is substituted C₆-C₁₀ aralkyl acyl. In some embodiments, R₁₁ is unsubstituted C₆-C₁₀ alkylaryl acyl. In some embodiments, R₁₁ is substituted C₆-C₁₀ alkylaryl acyl. In some embodiments, R₁₁ is unsubstituted alkoxy. In some embodiments, R₁₁ is substituted alkoxy. In some embodiments, R₁₁ is unsubstituted aryl. In some embodiments, R₁₁ is substituted aryl. In some embodiments, R₁₁ is unsubstituted C₃-C₁₀heterocyclyl. In some embodiments, R₁₁ is substituted C₃-C₁₀ heterocyclyl. In some embodiments, R₁₁ is unsubstituted heteroaryl, In some embodiments, R₁₁ is unsubstituted C₃-C₁₀ cycloalkyl. In some embodiments, R₁₁ is substituted C₃-C₁₀ cycloalkyl. In some embodiments, R₁₁ is —OPO₃WY. In some embodiments, R₁₁ is —OCH₂PO₄WY. In some embodiments, R₁₁ is —OCH₂PO₄Z. In some embodiments, R₁₁ is —OPO₃Z.

In some embodiments of the invention, the pyrone analog of Formula IV is of Formula XXIV or Formula XXV:

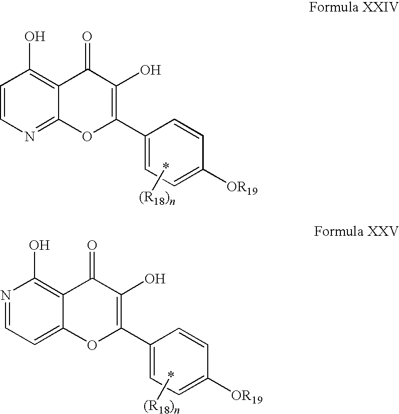

wherein R₁₈, R₁₉, and n are as defined in Formula II.

In some embodiments of the invention, the pyrone analog of Formula IV is of Formula XXVI or Formula XXVII:

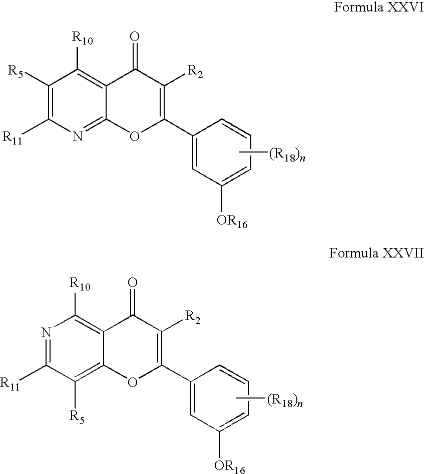

wherein R₂, and R₅ are as defined for Formula II and R₁₀ and R₁₁ are as defined for Formula IV;

R₁₆ is hydrogen, —PO₃WY, —CH₂PO₄WY, —CH₂PO₄Z or —PO₃Z;

wherein each instance of R₁₈ is independently hydrogen, hydroxyl, carboxaldehyde, amine, C₁-C₁₀ alkyl, C₂-C₁₀ alkynyl, C₂-C₁₀alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀ heterocyclic, C₃-C₁₀cycloalkyl, —OPO₃WY, —OCH₂PO₄WY, —OCH₂PO₄Z or —OPO₃Z; and

n is an integer of 0, 1, 2, 3, or 4.

In some embodiments of the invention, the pyrone analog of Formula IV is of Formula XXVIII:

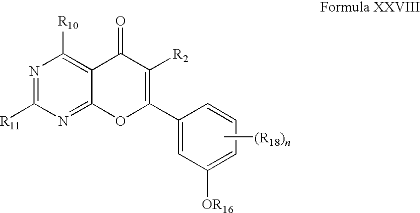

wherein R₂ is as defined for Formula II and R₁₀ and R₁₁ are as defined for Formula IV;

R₁₆ is hydrogen, —PO₃WY, —CH₂PO₄WY, —CH₂PO₄Z or —PO₃Z;

wherein each instance of R₁₈ is independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, C₃-C₁₀ heterocyclic, C₃-C₁₀ cycloalkyl, —OPO₃WY, —OCH₂PO₄WY, —OCH₂PO₄Z or —OPO₃Z; and

n is an integer of 0, 1, 2, 3, or 4.

In some embodiments of the invention, the pyrone analog of Formula II is of Formula V:

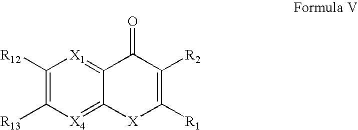

wherein X, X₁, X₄, R₁, and R₂ are as defined for Formula II; and

R₁₂ and R₁₃ are independently hydrogen, hydroxyl, carboxaldehyde, amino, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, amine, aryl, C₃-C₁₀ heterocyclyl, heteroaryl, C₃-C₁₀ cycloalkyl, —OPO₃WY, —OCH₂PO₄WY, —OCH₂PO₄Z or —OPO₃Z.

In some embodiments, R₁₂ is hydrogen. In some embodiments, R₁₂ is hydroxyl. In some embodiments, R₁₂ is carboxaldehyde. In some embodiments, R₁₂ is unsubstituted amine. In some embodiments, R₁₂ is substituted amine. In some embodiments, R₁₂ is unsubstituted C₁-C₁₀ alkyl. In some embodiments, R₁₂ is substituted C₁-C₁₀ alkyl. In some embodiments, R₁₂ is unsubstituted C₂-C₁₀ alkynyl. In some embodiments, R₁₂ is substituted C₂-C₁₀ alkynyl. In some embodiments, R₁₂ is unsubstituted C₂-C₁₀ alkenyl. In some embodiments, R₁₂ is substituted C₂-C₁₀ alkenyl. In some embodiments, R₁₂ is carboxyl. In some embodiments, R₁₂ is unsubstituted carbohydrate. In some embodiments, R₁₂ is substituted carbohydrate. In some embodiments, R₁₂ is unsubstituted ester. In some embodiments, R₁₂ is substituted ester. In some embodiments, R₁₂ is unsubstituted acyloxy. In some embodiments, R₁₂ is substituted acyloxy. In some embodiments, R₁₂ is nitro. In some embodiments, R₁₂ is halogen. In some embodiments, R₁₂ is unsubstituted C₁-C₁₀ aliphatic acyl. In some embodiments, R₁₂ is substituted C₁-C₁₀ aliphatic acyl. In some embodiments, R₁₂ is unsubstituted C₆-C₁₀ aromatic acyl. In some embodiments, R₁₂ is substituted C₆-C₁₀ aromatic acyl. In some embodiments, R₁₂ is unsubstituted C₆-C₁₀ aralkyl acyl. In some embodiments, R₁₂ is substituted C₆-C₁₀ aralkyl acyl. In some embodiments, R₁₂ is unsubstituted C₆-C₁₀ alkylaryl acyl. In some embodiments, R₁₂ is substituted C₆-C₁₀ alkylaryl acyl. In some embodiments, R₁₂ is unsubstituted alkoxy. In some embodiments, R₁₂ is substituted alkoxy. In some embodiments, R₁₂ is unsubstituted aryl. In some embodiments, R₁₂ is substituted aryl. In some embodiments, R₁₂ is unsubstituted C₃-C₁₀ heterocyclyl. In some embodiments, R₁₂ is substituted C₃-C₁₀heterocyclyl. In some embodiments, R₁₂ is unsubstituted heteroaryl, In some embodiments, R₁₂ is unsubstituted C₃-C₁₀ cycloalkyl. In some embodiments, R₁₂ is substituted C₃-C₁₀ cycloalkyl. In some embodiments, R₁₂ is —OPO₃WY. In some embodiments, R₁₂ is —OCH₂PO₄WY. In some embodiments, R₁₂ is —OCH₂PO₄Z. In some embodiments, R₁₂ is —OPO₃Z.

In some embodiments, R₁₃ is hydrogen. In some embodiments, R₁₃ is hydroxyl. In some embodiments, R₁₃ is carboxaldehyde. In some embodiments, R₁₃ is unsubstituted amine. In some embodiments, R₁₃ is substituted amine. In some embodiments, R₁₃ is unsubstituted C₁-C₁₀ alkyl. In some embodiments, R₁₃ is substituted C₁-C₁₀ alkyl. In some embodiments, R₁₃ is unsubstituted C₂-C₁₀ alkynyl. In some embodiments, R₁₃ is substituted C₂-C₁₀ alkynyl. In some embodiments, R₁₃ is unsubstituted C₂-C₁₀ alkenyl. In some embodiments, R₁₃ is substituted C₂-C₁₀ alkenyl. In some embodiments, R₁₃ is carboxyl. In some embodiments, R₁₃ is unsubstituted carbohydrate. In some embodiments, R₁₃ is substituted carbohydrate. In some embodiments, R₁₃ is unsubstituted ester. In some embodiments, R₁₃ is substituted ester. In some embodiments, R₁₃ is unsubstituted acyloxy. In some embodiments, R₁₃ is substituted acyloxy. In some embodiments, R₁₃ is nitro. In some embodiments, R₁₃ is halogen. In some embodiments, R₁₃ is unsubstituted C₁-C₁₀ aliphatic acyl. In some embodiments, R₁₃ is substituted C₁-C₁₀ aliphatic acyl. In some embodiments, R₁₃ is unsubstituted C₆-C₁₀ aromatic acyl. In some embodiments, R₁₃ is substituted C₆-C₁₀ aromatic acyl. In some embodiments, R₁₃ is unsubstituted C₆-C₁₀ aralkyl acyl. In some embodiments, R₁₃ is substituted C₆-C₁₀ aralkyl acyl. In some embodiments, R₁₃ is unsubstituted C₆-C₁₀ alkylaryl acyl. In some embodiments, R₁₃ is substituted C₆-C₁₀ alkylaryl acyl. In some embodiments, R₁₃ is unsubstituted alkoxy. In some embodiments, R₁₃ is substituted alkoxy. In some embodiments, R₁₃ is unsubstituted aryl. In some embodiments, R₁₃ is substituted aryl. In some embodiments, R₁₃ is unsubstituted C₃-C₁₀ heterocyclyl. In some embodiments, R₁₃ is substituted C₃-C₁₀ heterocyclyl. In some embodiments, R₁₃ is unsubstituted heteroaryl, In some embodiments, R₁₃ is unsubstituted C₃-C₁₀ cycloalkyl. In some embodiments, R₁₃ is substituted C₃-C₁₀ cycloalkyl. In some embodiments, R₁₃ is —OPO₃WY. In some embodiments, R₁₃ is —OCH₂PO₄WY. In some embodiments, R₁₃ is —OCH₂PO₄Z. In some embodiments, R₁₃ is —OPO₃Z.

In some embodiments of the invention, the pyrone analog of Formula V is of Formula XXIX or Formula XXX:

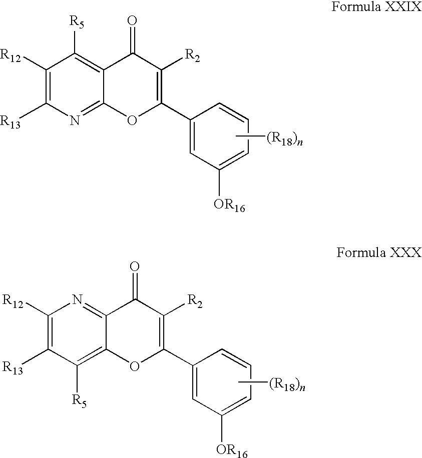

wherein R₂, R₅, R₁₈, and nare as defined for Formula II and R₁₂ and R₁₃ are as defined for Formula V; and

R₁₆ is hydrogen, —PO₃WY, —CH₂PO₄WY, —CH₂PO₄Z or —PO₃Z.

In some embodiments of the invention, the pyrone analog of Formula V is of Formula XXXI:

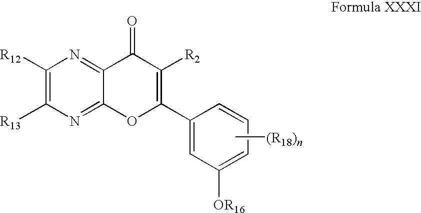

wherein R₂, R₁₈, and n are as defined for Formula II and R₁₂ and R₁₃ are as defined for Formula V; and

R₁₆ is hydrogen, —PO₃WY, —CH₂PO₄WY, —CH₂PO₄Z or —PO₃Z.

In some embodiments of the invention, the pyrone analog of Formula II is of Formula VI:

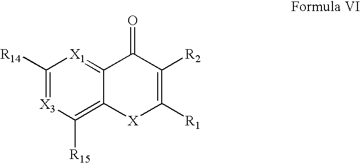

wherein X, X₁, X₃, R₁, and R₂ are as defined for Formula II; and

R₁₄ and R₁₅ are independently hydrogen, hydroxyl, carboxaldehyde, amino, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, amine, aryl, C₃-C₁₀heterocyclyl, heteroaryl, C₃-C₁₀cycloalkyl, —OPO₃WY, —OCH₂PO₄WY, —OCH₂PO₄Z or —OPO₃Z.

In some embodiments, R₁₄ is hydrogen. In some embodiments, R₁₄ is hydroxyl. In some embodiments, R₁₄ is carboxaldehyde. In some embodiments, R₁₄ is unsubstituted amine. In some embodiments, R₁₄ is substituted amine. In some embodiments, R₁₄ is unsubstituted C₁-C₁₀ alkyl. In some embodiments, R₁₄ is substituted C₁-C₁₀ alkyl. In some embodiments, R₁₄ is unsubstituted C₂-C₁₀ alkynyl. In some embodiments, R₁₄ is substituted C₂-C₁₀ alkynyl. In some embodiments, R₁₄ is unsubstituted C₂-C₁₀ alkenyl. In some embodiments, R₁₄ is substituted C₂-C₁₀ alkenyl. In some embodiments, R₁₄ is carboxyl. In some embodiments, R₁₄ is unsubstituted carbohydrate. In some embodiments, R₁₄ is substituted carbohydrate. In some embodiments, R₁₄ is unsubstituted ester. In some embodiments, R₁₄ is substituted ester. In some embodiments, R₁₄ is unsubstituted acyloxy. In some embodiments, R₁₄ is substituted acyloxy. In some embodiments, R₁₄ is nitro. In some embodiments, R₁₄ is halogen. In some embodiments, R₁₄ is unsubstituted C₁-C₁₀ aliphatic acyl. In some embodiments, R₁₄ is substituted C₁-C₁₀ aliphatic acyl. In some embodiments, R₁₄ is unsubstituted C₆-C₁₀ aromatic acyl. In some embodiments, R₁₄ is substituted C₆-C₁₀ aromatic acyl. In some embodiments, R₁₄ is unsubstituted C₆-C₁₀ aralkyl acyl. In some embodiments, R₁₄ is substituted C₆-C₁₀ aralkyl acyl. In some embodiments, R₁₄ is unsubstituted C₆-C₁₀ alkylaryl acyl. In some embodiments, R₁₄ is substituted C₆-C₁₀ alkylaryl acyl. In some embodiments, R₁₄ is unsubstituted alkoxy. In some embodiments, R₁₄ is substituted alkoxy. In some embodiments, R₁₄ is unsubstituted aryl. In some embodiments, R₁₄ is substituted aryl. In some embodiments, R₁₄ is unsubstituted C₃-C₁₀ heterocyclyl. In some embodiments, R₁₄ is substituted C₃-C₁₀ heterocyclyl. In some embodiments, R₁₄ is unsubstituted heteroaryl, In some embodiments, R₁₄ is unsubstituted C₃-C₁₀ cycloalkyl. In some embodiments, R₁₄ is substituted C₃-C₁₀ cycloalkyl. In some embodiments, R₁₄ is —OPO₃WY. In some embodiments, R₁₄ is —OCH₂PO₄WY. In some embodiments, R₁₄ is —OCH₂PO₄Z. In some embodiments, R₁₄ is —OPO₃Z.

In some embodiments, R₁₅ is hydrogen. In some embodiments, R₁₅ is hydroxyl. In some embodiments, R₁₅ is carboxaldehyde. In some embodiments, R₁₅ is unsubstituted amine. In some embodiments, R₁₅ is substituted amine. In some embodiments, R₁₅ is unsubstituted C₁-C₁₀ alkyl. In some embodiments, R₁₅ is substituted C₁-C₁₀ alkyl. In some embodiments, R₁₅ is unsubstituted C₂-C₁₀ alkynyl. In some embodiments, R₁₅ is substituted C₂-C₁₀ alkynyl. In some embodiments, R₁₅ is unsubstituted C₂-C₁₀ alkenyl. In some embodiments, R₁₅ is substituted C₂-C₁₀ alkenyl. In some embodiments, R₁₅ is carboxyl. In some embodiments, R₁₅ is unsubstituted carbohydrate. In some embodiments, R₁₅ is substituted carbohydrate. In some embodiments, R₁₅ is unsubstituted ester. In some embodiments, R₁₅ is substituted ester. In some embodiments, R₁₅ is unsubstituted acyloxy. In some embodiments, R₁₅ is substituted acyloxy. In some embodiments, R₁₃ is nitro. In some embodiments, R₁₃ is halogen. In some embodiments, R₁₃ is unsubstituted C₁-C₁₀ aliphatic acyl. In some embodiments, R₁₅ is substituted C₁-C₁₀ aliphatic acyl. In some embodiments, R₁₅ is unsubstituted C₆-C₁₀ aromatic acyl. In some embodiments, R₁₅ is substituted C₆-C₁₀ aromatic acyl. In some embodiments, R₁₅ is unsubstituted C₆-C₁₀ aralkyl acyl. In some embodiments, R₁₅ is substituted C₆-C₁₀ aralkyl acyl. In some embodiments, R₁₅ is unsubstituted C₆-C₁₀ alkylaryl acyl. In some embodiments, R₁₅ is substituted C₆-C₁₀ alkylaryl acyl. In some embodiments, R₁₅ is unsubstituted alkoxy. In some embodiments, R₁₅ is substituted alkoxy. In some embodiments, R₁₅ is unsubstituted aryl. In some embodiments, R₁₅ is substituted aryl. In some embodiments, R₁₅ is unsubstituted C₃-C₁₀ heterocyclyl. In some embodiments, R₁₅ is substituted C₃-C₁₀ heterocyclyl. In some embodiments, R₁₅ is unsubstituted heteroaryl, In some embodiments, R₁₅ is unsubstituted C₃-C₁₀ cycloalkyl. In some embodiments, R₁₅ is substituted C₃-C₁₀ cycloalkyl. In some embodiments, R₁₅ is —OPO₃WY. In some embodiments, R₁₅ is —OCH₂PO₄WY. In some embodiments, R₁₅ is —OCH₂PO₄Z. In some embodiments, R₁₅ is —OPO₃Z.

In some embodiments of the invention, the pyrone analog of Formula VI is of Formula XXXII or Formula XXXIII:

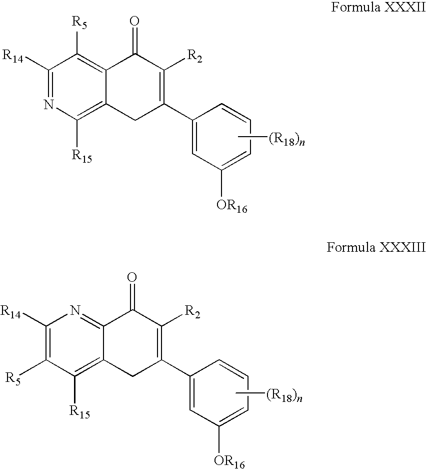

wherein R₂, R₅, R₁₈, and n are as defined for Formula II and R₁₄ and R₁₅ are as defined for Formula VI; and

R₁₆ is hydrogen, —PO₃WY, —CH₂PO₄WY, —CH₂PO₄Z or —PO₃Z.

In some embodiments of the invention, the pyrone analog of Formula VI is of Formula XXXIV:

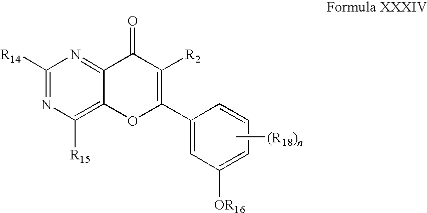

wherein R₂, R₁₈, and n are as defined for Formula II and R₁₄ and R₁₅ are as defined for Formula V; and

R₁₆ is hydrogen, —PO₃WY, —CH₂PO₄WY, —CH₂PO₄Z or —PO₃Z.

One class of compounds useful in the compositions and methods of the invention is polyphenols. Many polyphenols are modulators of BTB transport proteins; however, any suitable polyphenol that reduces or eliminates hyperglycemia and/or one or more symptoms of hyperglycemia induced by administration of a calcineurin inhibitor, no matter what the mechanism, may be used in the compositions and methods of the invention.

A useful class of polyphenols is the flavonoids. Flavonoids, the most abundant polyphenols in the diet, can be classified into subgroups based on differences in their chemical structures. The basic flavonoid structure is shown below (formula XXXV), and its pharmaceutically acceptable salts, esters, prodrugs, analogs, isomers, stereoisomers or tautomers thereof:

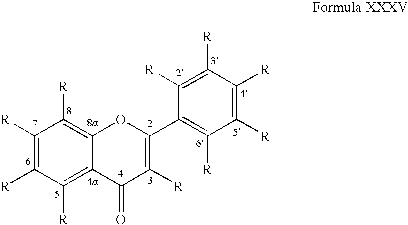

wherein the 2,3 bond may be saturated or unsaturated, and wherein each R can be independently selected from the group consisting of hydrogen, substituted or unsubstituted hydroxyl, substituted or unsubstituted amine, substituted or unsubstituted thiol, substituted or unsubstituted C₁-C₁₀ alkyl, substituted or unsubstituted C₂-C₁₀ alkynyl, substituted or unsubstituted C₂-C₁₀ alkenyl, substituted or unsubstituted aryl, substituted or unsubstituted heteroaryl, substituted or unsubstituted C₃-C₁₀ cycloalkyl, substituted or unsubstituted heterocycloalkyl, substituted or unsubstituted C₁-C₁₀ aliphatic acyl, substituted or unsubstituted C₆-C₁₀ aromatic acyl, substituted or unsubstituted trialkyl silyl, substituted or unsubstituted ether, carbohydrate, and substituted or unsubstituted carbohydrate. “Carbohydrate” as used herein, includes, but is not limited to, monosaccharides, disaccharides, oligosaccharides, or polysaccharides. Monosaccharide for example includes, but not limited to, allose, altrose, mannose, gulose, Idose, glucose, galactose, talose, and fructose. Disaccharides for example includes, but not limited to, glucorhamnose, trehalose, sucrose, lactose, maltose, galactosucrose, N-acetyllactosamine, cellobiose, gentiobiose, isomaltose, melibiose, primeverose, hesperodinose, and rutinose. Oligosaccharides for example includes, but not limited to, raffinose, nystose, panose, cellotriose, maltotriose, maltotetraose, xylobiose, galactotetraose, isopanose, cyclodextrin (α-CD) or cyclomaltohexaose, β-cyclodextrin (β-CD) or cyclomaltoheptaose and γ-cyclodextrin (γ-CD) or cyclomaltooctaose. Polysaccharide for example includes, but not limited to, xylan, mannan, galactan, glucan, arabinan, pustulan, gellan, guaran, xanthan, and hyaluronan. Some examples include, but not limited to, starch, glycogen, cellulose, inulin, chitin, amylose and amylopectin.

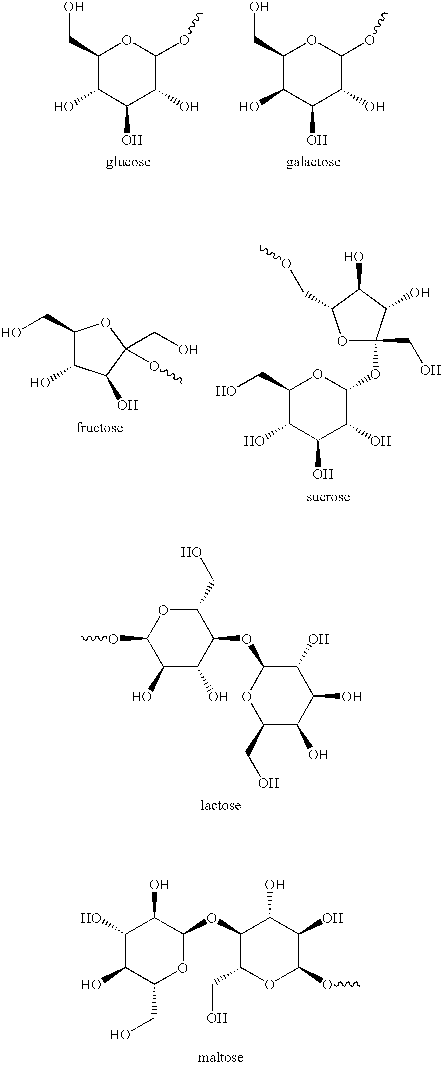

In some embodiments, the invention utilizes a flavonoid where the molecule is planar. In some embodiments, the invention utilizes a flavonoid where the 2-3 bond is unsaturated. In some embodiments, the invention utilizes a flavonoid where the 3-position is hydroxylated. In some embodiments, the invention utilizes a flavonoid where the 2-3 bond is unsaturated and the 3-position is hydroxylated (e.g., flavonols).

In some embodiments, the invention utilizes one or more flavonoids selected from the group consisting of quercetin, isoquercetin, flavone, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, and epicatechin. In some embodiments, the invention utilizes one or more flavonoids selected from the group consisting of quercetin, isoquercetin, apigenin, rhoifolin, galangin, fisetin, morin, rutin, kaempferol, myricetin, naringenin, hesperetin, phloretin, and genistein. Structures of these compounds are well-known in the art. See, e.g., Critchfield et al. (1994) Biochem. Pharmacol 7:1437-1445.

In some embodiments, the invention utilizes a flavonol. In some embodiments, the flavonol is selected from the group consisting of quercetin, fisetin, morin, rutin, myricetin, galangin, and kaempherol, and combinations thereof. In some embodiments, the flavonol is selected from the group consisting of quercetin, fisetin, galangin, and kaempherol, and combinations thereof. In some embodiments, the flavonol is quercetin or a substituted analog thereof. In other embodiments, the flavonol is fisetin or a substituted analog thereof. In some embodiments, the flavonol is galangin or a substituted analog thereof. In some embodiments, the flavonol is kaempherol or a substituted analog thereof. In some embodiments, the flavonol is a phosphorylated quercetin or a phosphorylated quercetin derivative, or a phosphorylated fisetin or a phosphorylated fisetin derivative. Preferably, the flavonol is a phosphorylated quercetin, fisetin or a phosphorylated fisetin.

In some embodiments, the pyrone analog is modified with a phosphate group to increase the solubility of the pyrone analog. The phosphate group can be attached to any suitable part of the pyrone analog. Useful phosphorylated pyrone analogs of the present invention are phosphorylated polyphenols of the structure of formula (XXXVIa) or formula (XXXVIb), or its pharmaceutically or veterinarily acceptable salts, glycosides, esters, or prodrugs:

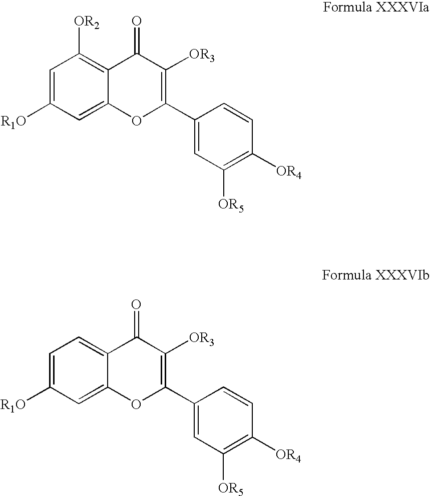

wherein R₁, R₂, R₃, R₄, and R₅ are independently selected from the group of hydrogen, —PO₃XY, and —PO₃Z, wherein X and Y are independently selected from hydrogen, methyl, ethyl, alkyl, carbohydrate, and a cation, wherein Z is a multivalent cation, and wherein at least one of the R₁-R₅ is —PO₃XY, or —PO₃Z.

In some embodiments of the invention, the phosphorylated pyrone analog can comprise a cyclic phosphate. In some embodiments, the invention is a composition comprising a compound of formula (XXXVIIa) or formula (XXXVIIb), or its pharmaceutically or veterinarily acceptable salts, glycosides, esters, or prodrugs:

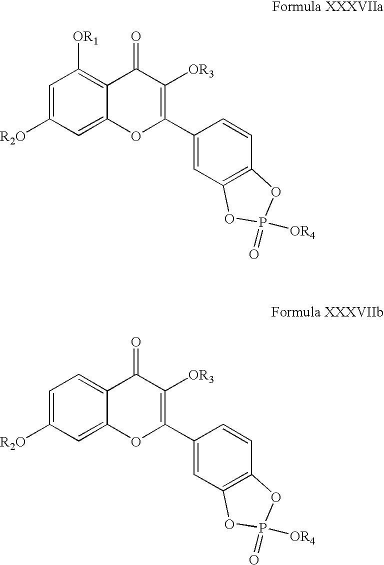

wherein R₁, R₂, and R₃ are each independently selected from the group of hydrogen, —PO₃XY, and —PO₃Z, wherein X and Y are independently selected from hydrogen, methyl, ethyl, alkyl, carbohydrate, and a cation, wherein Z is a multivalent cation, and wherein R4 is selected from the group of hydrogen, methyl, ethyl, alkyl, carbohydrate, and a cation.

Accordingly, in some embodiments, the invention utilizes a phosphorylated flavonoid where the molecule is planar. In some embodiments, the invention utilizes a phosphorylated flavonoid where the 2-3 bond is unsaturated. In some embodiments, the invention utilizes a phosphorylated flavonoid where the 3-position is hydroxylated. In some embodiments, the invention utilizes a phosphorylated flavonoid where the 2-3 bond is unsaturated and the 3-position is hydroxylated (e.g., flavonols).

In some embodiments, the invention utilizes one or more phosphorylated flavonoids selected from the group consisting of phosphorylated quercetin, phosphorylated isoquercetin, phosphorylated flavone, phosphorylated chrysin, phosphorylated apigenin, phosphorylated rhoifolin, phosphorylated diosmin, phosphorylated galangin, phosphorylated fisetin, phosphorylated morin, phosphorylated rutin, phosphorylated kaempferol, phosphorylated myricetin, phosphorylated taxifolin, phosphorylated naringenin, phosphorylated naringin, phosphorylated hesperetin, phosphorylated hesperidin, phosphorylated chalcone, phosphorylated phloretin, phosphorylated phlorizdin, phosphorylated genistein, phosphorylated biochanin A, phosphorylated catechin, and phosphorylated epicatechin. In some embodiments, the invention utilizes one or more phosphorylated flavonoids selected from the group consisting of phosphorylated quercetin, phosphorylated isoquercetin, phosphorylated apigenin, phosphorylated rhoifolin, phosphorylated galangin, phosphorylated fisetin, phosphorylated morin, phosphorylated rutin, phosphorylated kaempferol, phosphorylated myricetin, phosphorylated naringenin, phosphorylated hesperetin, phosphorylated phloretin, and phosphorylated genistein.

In some embodiments, the invention utilizes a phosphorylated flavonol. In some embodiments, the phosphorylated flavonol is selected from the group consisting of phosphorylated quercetin, phosphorylated fisetin, phosphorylated morin, phosphorylated rutin, phosphorylated myricetin, phosphorylated galangin, and phosphorylated kaempherol, and combinations thereof. In some embodiments, the phosphorylated flavonol is selected from the group consisting of phosphorylated quercetin, phosphorylated galangin, phosphorylated fisetin and phosphorylated kaempherol, and combinations thereof. In some embodiments, the phosphorylated flavonol is phosphorylated quercetin or a phosphorylated quercetin derivative. In some embodiments, the phosphorylated flavonol is phosphorylated fisetin or a phosphorylated fisetin derivative. In some embodiments, the phosphorylated flavonol is phosphorylated galangin or a phosphorylated galangin derivative. In some embodiments, the phosphorylated flavonol is phosphorylated kaempherol or a phosphorylated kaempherol derivative.

In some embodiments, the phosphorylated polyphenol comprises a monophosphate, diphosphate, triphosphate, tetraphosphate, or pentaphosphate.

A particularly useful flavonol is quercetin or a quercetin derivative. Quercetin may be used to illustrate formulations and methods useful in the invention; however, it is understood that the discussion of quercetin applies equally to other flavonoids, flavonols, and polyphenols useful in the invention, e.g., fisetin, kaempferol and galangin.

The structure of quercetin is shown below (formula XXXVIII):

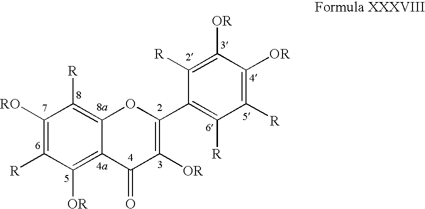

wherein each OR is an OH (i.e., 3-OH, 5-OH, 7-OH, 3′-OH, and 4′-OH) and each R is an H. The numbering of the carbons is the same as in Formula XXXV. This form of quercetin is used in some embodiments of the invention. In addition, metabolites of quercetin, e.g., quercetin 3-O-glucouronide, are encompassed by the term “quercetin” as used herein. The term “quercetin” optionally encompasses glycosides of quercetin, wherein one or more of the R₁-R₅ comprise a carbohydrate.

In some embodiments, quercetin may be modified by derivatizing with at least one phosphate group. The phosphate group can be attached to any suitable part of the quercetin molecule. Examples of quercetin molecules modified by attaching a phosphate group include (Formula XXXIX and XL):

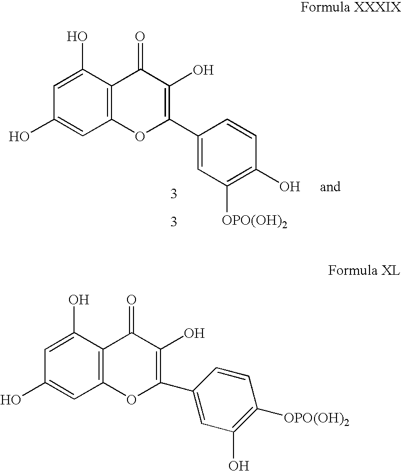

In some embodiments, the quercetin is phosphorylated at the 3′ position (3′-quercetin phosphate). In some embodiments, the quercetin is phosphorylated at the 4′ position (4′-quercetin phosphate). In some embodiments, the quercetin phosphate composition is a mixture of 3′-quercetin phosphate and 4′-quercetin phosphate. In some embodiments, the composition comprises at least 5%, 15%. 20%, 25%, 30%, 35%, 40%, 45%, 50%, 55%, 60%, 65%, 70%, 75%, 80%, 85%, 90%, 95%, 96%, 97%, 98%, 99%, 99.1% or 99.9% 3′-quercetin phosphate. In some embodiments, the composition comprises at least 5%, 15%. 20%, 25%, 30%, 35%, 40%, 45%, 50%, 55%, 60%, 65%, 70%, 75%, 80%, 85%, 90%, 95%, 96%, 97%, 98%, 99%, 99.1% or 99.9% 4′-quercetin phosphate.

In some embodiments, the phosphorylated quercetin is in a carbohydrate-derivatized form, e.g., a phosphorylated quercetin-O-saccharide. Phosphorylated quercetin-O-saccharides useful in the invention include, but are not limited to, phosphorylated quercetin 3-O-glycoside, phosphorylated quercetin 3-O-glucorhamnoside, phosphorylated quercetin 3-O-galactoside, phosphorylated quercetin 3-O-xyloside, and phosphorylated quercetin 3-O-rhamnoside. In some embodiments, the invention utilizes a phosphorylated quercetin 7-O-saccharide.

In some embodiments, the invention utilizes a phosphorylated quercetin aglycone. In some embodiments, a combination of aglycones and carbohydrate-derivatized phosphorylated quercetins is used. It will be appreciated that the various forms of phosphorylated quercetin may have different properties useful in the compositions and methods of the invention, and that the route of administration can determine the choice of forms, or combinations of forms, used in the composition or method. It will also be appreciated that the various forms of quercetin or fisetin, including phosphorylated quercetin or fisetin, vary in the toxicity (or lack thereof) and/or effectiveness in reducing or elimination hyperglycemia and/or one or more symptoms of hyperglycemia induced by a calcineurin inhibitor. For example, certain forms of quercetin or fisetin, e.g. quercetin phosphate, fisetin or fisetin phosphate, may differ by reducing or eliminating renal toxicity induced by the administration of a calcineurin inhibitor. Choice of a single form, or of combinations, including evaluation of the effectiveness and toxicity of a BTB protein modulator is a matter of routine experimentation. For example, preferred embodiments herein include phosphorylated quercetin, fisetin and/or fisetin phosphate based on increased solubility characteristics as well as increased bioavailability.

In some embodiments, quercetin may be modified by attaching an amino acid such as glycine, alanine, dimethyl glycine, sarcosine, aspartic acid, or arginine. The amino acid can be attached to any suitable part of the quercetin molecule.

In some embodiments, fisetin (5 deoxyquercetin; 5 desoxyquercetin; 3,3′,4′,7-tetrahydroxyflavone) or a fisetin derivative may be used in the compositions and formulations disclosed herein. The structure of fisetin is shown below (Formula XXXIX):

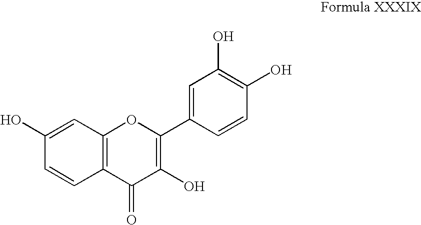

In addition, metabolites of fisetin are encompassed by the term “fisetin” as used herein. The term “fisetin” optionally includes glycosides of fisetin, wherein one or more of the R₁-R₅ comprise a carbohydrate. In some embodiments, fisetin may be modified by derivatizing with at least one phosphate group. The phosphate group can be attached to any suitable part of the fisetin molecule. Examples of fisetin phosphate include 3′-fisetin phosphate (Formula XXXIXa), 4′-fisetin phosphate (Formula XXXIXb), and 3-fisetin phosphate (Formula XXXIXc),

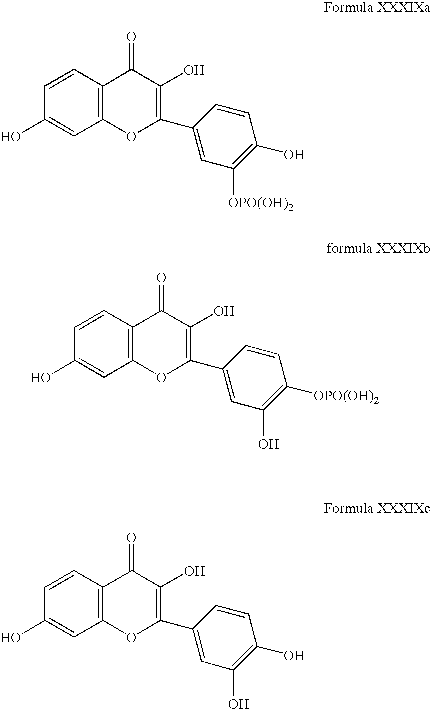

In some embodiments, a fisetin derivative, including 5,7-dideoxyquercetin. In other embodiments, a fisetin derivative is optionally phosphorylated, e.g. fisetin phosphate (5,7 dideoxyquercetin phosphate). In some embodiments, fisetin may be modified by attaching an amino acid such as glycine, alanine, dimethyl glycine, sarcosine, aspartic acid, or arginine. The amino acid can be attached to any suitable part of the fisetin molecule.

In some embodiments, the fisetin is phosphorylated at the 3′ position (3′-fisetin phosphate). In some embodiments, the fisetin is phosphorylated at the 4′ position (4′-fisetin phosphate). In some embodiments, the fisetin phosphate composition is a mixture of 3′-fisetin phosphate and 4′-fisetin phosphate. In some embodiments, the composition comprises at least 5%, 15%. 20%, 25%, 30%, 35%, 40%, 45%, 50%, 55%, 60%, 65%, 70%, 75%, 80%, 85%, 90%, 95%, 96%, 97%, 98%, 99%, 99.1% or 99.9% 3′-fisetin phosphate. In some embodiments, the composition comprises at least 5%, 15%. 20%, 25%, 30%, 35%, 40%, 45%, 50%, 55%, 60%, 65%, 70%, 75%, 80%, 85%, 90%, 95%, 96%, 97%, 98%, 99%, 99.1% or 99.9% 4′-fisetin phosphate.

In some embodiments, a pyrone analog, such as a polyphenol or a polyphenol derivative, is administered with an excipient to increase the solubility of the pyrone analog. In some embodiments, the excipient is an oligosaccharide. In other embodiments, the excipient is a cyclic oligosaccharide, such as cyclodextrin. In some embodiments, the excipient is a sulfo-alkyl ether substituted cyclodextrin, or a sulfobutyl-ether substituted cyclodextrin. In some embodiments, the excipient is hydroxypropyl-β-cyclodextrin, hydroxypropyl-γ-cyclodextrin sulfobutylether-β-cyclodextrin, sulfobutylether-7-β-cyclodextrin, or combinations thereof. In some embodiments, the excipient is Captisol®

In some embodiments, quercetin or a quercetin derivative is administered with an excipient to increase the solubility of the quercetin or quercetin derivative. In some embodiments, the excipient is an oligosaccharide. In other embodiments, the excipient is a cyclic oligosaccharide, such as cyclodextrin. In some embodiments, the excipient is a sulfo-alkyl ether substituted cyclodextrin, or a sulfobutyl-ether substituted cyclodextrin. In some embodiments, the excipient is hydroxypropyl-β-cyclodextrin, hydroxypropyl-γ-cyclodextrin sulfobutylether-β-cyclodextrin, sulfobutylether-7-β-cyclodextrin, or combinations thereof. In some embodiments, the excipient is Captisol®

In some embodiments, fisetin or a fisetin derivative is administered with an excipient to increase the solubility of the fisetin or fisetin derivative. In some embodiments, the excipient is an oligosaccharide. In other embodiments, the excipient is a cyclic oligosaccharide, such as cyclodextrin. In some embodiments, the excipient is a sulfo-alkyl ether substituted cyclodextrin, or a sulfobutyl-ether substituted cyclodextrin. In some embodiments, the excipient is hydroxypropyl-β-cyclodextrin, hydroxypropyl-γ-cyclodextrin, sulfobutylether-β-cyclodextrin, sulfobutylether-7-β-cyclodextrin, or combinations thereof. In some embodiments, the excipient is Captisol®

In some embodiments, the composition comprises quercetin or a quercetin derivative in an amount of from about 0.1% to about 1% (w/v, g/ml); a sulfobutylether-7-β-cyclodextrin in an amount of from about 0.1% to about 5% (w/v); water; and a pH adjusting agent sufficient to adjust the pH of the formulation to from about 6.5 to about 8. In some embodiments the composition further comprises a co-solvent in an amount of from about 1% to about 35% (w/v). In some embodiments the co-solvent is an alcohol. In some embodiments the composition further comprises an effective amount of an antimicrobial preservative. In some embodiments the composition further comprises an effective amount of an antioxidant.

In some embodiments, the composition comprises fisetin or a fisetin derivative in an amount of from about 0.1% to about 1% (w/v); a sulfobutylether-7-β-cyclodextrin in an amount of from about 0.1% to about 5% (w/v); water; and a pH adjusting agent sufficient to adjust the pH of the formulation to from about 6.5 to about 8. In some embodiments the composition further comprises a co-solvent in an amount of from about 1% to about 35% (w/v). In some embodiments the co-solvent is an alcohol. In some embodiments the composition further comprises an effective amount of an antimicrobial preservative. In some embodiments the composition further comprises an effective amount of an antioxidant.

In some embodiments, the composition comprises a solid pharmaceutical formulation comprising a cyclodextrin and a flavonoid. In some embodiments the cyclodextrin is sulfobutylether-7-β-cyclodextrin. In some embodiments the cyclodextrin is Captisol (®). In some embodiments the flavonoid is selected from the group consisting of quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, and epicatechin. In some embodiments the flavonoid is quercetin, galangin, fisetin or kaempferol. In some embodiments the flavonoid is quercetin or fisetin.

In some embodiments the formulation is suitable for oral administration. In some embodiments water is substantially removed from the composition in order to make the solid formulation. In some embodiments the removal of water is performed by a process comprising freeze-drying or lyophilization.

In some embodiments the formulation is suitable for intravenous administration. In some embodiments the molar ratio of quercetin to sulfobutylether-7-β-cyclodextrin is between about 1:1 to about 1:5.

In some embodiments the molar ratio of quercetin to sulfobutylether-7-β-cyclodextrin is between about 1:2 to about 1:4. In some embodiments the weight ratio of quercetin to the sulfobutylether-7-β-cyclodextrin is between about 1:10 to about 1:40. In some embodiments the weight ratio of quercetin to sulfobutylether-7-β-cyclodextrin is between about 1:15 to about 1:20.

In some embodiments, the pharmaceutical composition comprises a flavonoid, a cyclodextrin, a basic amino acid or a sugar-amine and a pharmaceutically or veterinarily acceptable carrier. In some embodiments the basic amino acid is arginine. In some embodiments the basic amino acid is lysine. In some embodiments the sugar-amine is meglumine.

In some embodiments the flavonoid is quercetin, galangin, fisetin or kaempferol. In some embodiments the flavonoid is quercetin or fisetin.

In some embodiments the cyclodextrin is sulfobutylether-7-β-cyclodextrin.

In some embodiments, the cyclodextrin is Captisol®

In some embodiments the flavonoid is quercetin or fisetin, and the cyclodextrin is sulfobutylether-7-β-cyclodextrin.

In some embodiments the carrier comprises water. In some embodiments the sulfobutylether-7-β-cyclodextrin is present at a concentration of about 20% w/v or greater. In some embodiments the sulfobutylether-7-β-cyclodextrin is present at a concentration in a range of about 20% w/v to about 30% w/v. In some embodiments the quercetin is present in a range between about 1 mM to about 50 mM. In some embodiments the quercetin is present in a range between about 2 mM to about 40 mM. In some embodiments the amino acid is arginine. In some embodiments the amino acid is lysine. In some embodiments the pH is greater than about 8.5.

In some embodiments the composition is a dry powder formulation. In some embodiments the molar ratio of the quercetin to the sulfobutylether-7-β-cyclodextrin is between about 1:3 and 1:12.

In some embodiments, a method of preparing a solution of a flavonoid comprises mixing a cyclodextrin, a flavonoid, and a basic amino acid or a sugar-amine with water at a pH greater than 8.5. In some embodiments the method comprises dissolving the cyclodextrin in water to produce a cyclodextrin solution, then mixing the flavonoid and the basic amino acid or sugar-amine with the cyclodextrin solution. In some embodiments the basic amino acid is arginine. In some embodiments the basic amino acid is lysine. In some embodiments the sugar-amine is meglumine. In some embodiments the flavonoid is quercetin, galangin, fisetin or kaempferol. In some embodiments the flavonoid is quercetin or fisetin. In some embodiments the cyclodextrin is sulfobutylether-7-β-cyclodextrin. In some embodiments the flavonoid is quercetin or fisetin, and the cyclodextrin is sulfobutylether-7-β-cyclodextrin.

In some embodiments of the method the sulfobutylether-7-β-cyclodextrin is present at a concentration of about 20% w/v or greater. In some embodiments the sulfobutylether-7-β-cyclodextrin is present at a concentration in a range of about 20% w/v to about 30% w/v. In some embodiments the quercetin is present in a range between about 1 mM to about 50 mM. In some embodiments the quercetin is present in a range between about 2 mM to about 40 mM. In some embodiments the amino acid is arginine. In some embodiments the amino acid is lysine.

In some embodiments, the quercetin or fisetin is in a carbohydrate-derivatized form, e.g., a quercetin-O-saccharide. Quercetin-O-saccharides useful in the invention include, but are not limited to, quercetin 3-O-glycoside, quercetin 3-O-glucorhamnoside, quercetin 3-O-galactoside, quercetin 3-O-xyloside, and quercetin 3-O-rhamnoside. In some embodiments, the invention utilizes a quercetin 7-O-saccharide. In some embodiments, the phosphorylated quercetin or phosphorylated fisetin is in a carbohydrate-derivatized form, e.g., a phosphorylated quercetin-O-saccharide. Phosphorylated quercetin-O-saccharides useful in the invention include, but are not limited to, phosphorylated quercetin 3-O-glycoside, phosphorylated quercetin 3-O-glucorhamnoside, phosphorylated quercetin 3-O-galactoside, phosphorylated quercetin 3-O-xyloside, and phosphorylated quercetin 3-O-rhamnoside. In some embodiments, the invention utilizes a phosphorylated quercetin 7-O-saccharide.

In some embodiments, the invention utilizes a quercetin aglycone or fisetin aglycone. In some embodiments, the invention utilizes a phosphorylated quercetin aglycone or phosphorylated fisetin aglycone. In some embodiments, a combination of aglycones and carbohydrate-derivatized quercetin or fisetin is used. It will be appreciated that the various forms of quercetin or fisetin may have different properties useful in the compositions and methods of the invention, and that the route of administration can determine the choice of forms, or combinations of forms, used in the composition or method. Choice of a single form, or of combinations, is a matter of routine experimentation.

Thus, in some embodiments the invention features a composition or method utilizing quercetin or a quercetin derivative, or fisetin or fisetin derivative, to reduce or eliminate hyperglycemia and/or one or more symptoms of hyperglycemia induced by a calcineurin inhibitor, such as tacrolimus or a tacrolimus analog. In some embodiments, the invention discloses a composition or method utilizing phosphorylated quercetin or a phosphorylated quercetin derivative, or phosphorylated fisetin or a phosphorylated fisetin derivative, to reduce or eliminate hyperglycemia and/or one or more symptoms of hyperglycemia induced by a calcineurin inhibitor, such as tacrolimus or a tacrolimus analog.

In some embodiments, the quercetin or a quercetin derivative, or fisetin or fisetin derivative, is provided in a form for oral consumption. In some embodiments, quercetin-3-O-glycoside is used in an oral preparation of quercetin; in some embodiments, a pharmaceutically acceptable excipient is included in the composition. In some embodiments, quercetin 3-O-glucorhamnoside is used in an oral preparation of quercetin; in some embodiments, a pharmaceutically acceptable excipient is included in the composition. In some embodiments, a combination of quercetin-3-O-glycoside and quercetin 3-O-glucorhamnoside is used in an oral preparation of quercetin; in some embodiments, a pharmaceutically acceptable excipient is included in the composition. Other carbohydrate-derivatized forms of quercetin, or other forms of quercetin which are derivatives as described above, can also be used, based on their oral bioavailability, their metabolism, their incidence of gastrointestinal or other side effects, and other factors known in the art. Determining the bioavailability of quercetin or fisetin in the form of derivatives including aglycones and glycosides is a matter of routine experimentation. See, e.g., Graefe et al., J. Clin. Pharmacol. (2001) 451:492-499; Arts et al. (2004) Brit. J. Nutr. 91:841-847; Moon et al. (2001) Free Rad. Biol. Med. 30:1274-1285; Hollman et al. (1995) Am. J. Clin. Nutr. 62:1276-1282; Jenaelle et al. (2005) Nutr. J. 4:1, and Cermak et al. (2003) J. Nutr. 133: 2802-2807, all of which are incorporated by reference herein in their entirety.

In some embodiments, modified forms of a quercetin or a quercetin derivative, or fisetin or fisetin derivative, is provided in a form for oral consumption. In some embodiments, phosphorylated quercetin-3-O-glycoside or phosphorylated fisetin-3-O-glycoside is used in an oral preparation of quercetin; in some embodiments, a pharmaceutically acceptable excipient is included in the composition. In some embodiments, phosphorylated quercetin 3-O-glucorhamnoside is used in an oral preparation of quercetin; in some embodiments, a pharmaceutically acceptable excipient is included in the composition. In some embodiments, a combination of phosphorylated quercetin-3-O-glycoside or phosphorylated fisetin-3-O-glycoside and phosphorylated quercetin 3-O-glucorhamnoside is used in an oral preparation of quercetin; in some embodiments, a pharmaceutically acceptable excipient is included in the composition. Other carbohydrate-derivatized forms of quercetin, or other forms of phosphorylated quercetin which are derivatives as described above, can also be used, based on their oral bioavailability, their metabolism, their incidence of gastrointestinal or other side effects, and other factors known in the art. Determining the bioavailability of phosphorylated quercetin or phosphorylated fisetin in the form of derivatives including aglycones and glycosides is a matter of routine experimentation. See, e.g., Graefe et al., J. Clin. Pharmacol. (2001) 451:492-499; Arts et al. (2004) Brit. J. Nutr. 91:841-847; Moon et al. (2001) Free Rad. Biol. Med. 30:1274-1285; Hollman et al. (1995) Am. J. Clin. Nutr. 62:1276-1282; Jenaelle et al. (2005) Nutr. J. 4:1, and Cermak et al. (2003) J. Nutr. 133: 2802-2807, all of which are incorporated by reference herein in their entirety.

Oral bioavailability of quercetin O-saccharides is generally superior to that of quercetin aglycones. Similarly, oral bioavailability of fisetin O-saccharides is generally superior to that of fisetin aglycones. The bioavailability of the various components is dependent on 1) the site of carbohydrate moiety or moieties and ii) the pendant sugar unit. In addition it is believed that specific carriers are responsible for the absorption of various quercetin glycosides and fisetin glycosides, as well as specific intestinal betaglucosidases. After distribution in the body, the major metabolite for quercetin, quercetin glucuronide (e.g., quercetin 3-O-glucouronid), is found. Oral bioavailability is sensitive to the presence of food factors.

In compositions for oral delivery of quercetin or fisetin, carbohydrate-derivatized forms (also referred to herein as “quercetin saccharides” or “fisetin saccharides”) are used in some embodiments. In some embodiments, quercetin-3-O-glycoside is used in an oral preparation of quercetin; in some embodiments, a pharmaceutically acceptable excipient is included in the composition. In some embodiments, quercetin 3-O-glucorhamnoside is used in an oral preparation of quercetin; in some embodiments, a pharmaceutically acceptable excipient is included in the composition. In some embodiments, a combination of quercetin-3-O-glycoside and quercetin 3-O-glucorhamnoside is used in an oral preparation of quercetin; in some embodiments, a pharmaceutically acceptable excipient is included in the composition. Other carbohydrate-derivatized forms of quercetin or fisetin, or other forms of quercetin or fisetin which are derivatives as described above, can also be used, based on their oral bioavailability, their metabolism, their incidence of gastrointestinal or other side effects, and other factors known in the art. Determining the bioavailability of quercetin or fiestin in the form of derivatives including aglycones and glycosides is a matter of routine experimentation. See, e.g., Graefe et al., J. Clin. Pharmacol. (2001) 451:492-499; Arts et al. (2004) Brit. J. Nutr. 91:841-847; Moon et al. (2001) Free Rad. Biol. Med. 30:1274-1285; Hollman et al. (1995) Am. J. Clin. Nutr. 62:1276-1282; Jenaelle et al. (2005) Nutr. J. 4:1, and Cermak et al. (2003) J. Nutr. 133: 2802-2807, all of which are incorporated by reference herein in their entirety.

In some embodiments, the invention provides a composition for administration of phosphorylated quercetin or phosphorylated fisetin to an animal to reduce a side effect of a substance, e.g., for the oral delivery of phosphorylated quercetin or phosphorylated fisetin, that contains at least about 1, 5, 10, 20, 30, 40, 50, 60, 70, 80, 90, 95, 99, 99.5, 99.9, or 99.99% phosphorylated quercetin-O-saccharide or phosphorylated fisetin-O-saccharide. In some embodiments, the invention provides a composition for the oral delivery of phosphorylated quercetin or phosphorylated fisetin that contains no more than about 2, 5, 10, 20, 30, 40, 50, 60, 70, 80, 90, 95, 99, 99.5, 99.9, 99.99, or 100% phosphorylated quercetin-O-saccharide or phosphorylated fisetin-O-saccharide. In some embodiments, the invention provides a composition that contains about 1-100% phosphorylated quercetin-O-saccharide or phosphorylated fisetin-O-saccharide, or about 10-100% phosphorylated quercetin-O-saccharide or phosphorylated fisetin-O-saccharide, or about 20-100% phosphorylated quercetin-O-saccharide or phosphorylated fisetin-O-saccharide, or about 50-100% phosphorylated quercetin-O-saccharide or phosphorylated fisetin-O-saccharide, or about 80-100% phosphorylated quercetin-O-saccharide or phosphorylated fisetin-O-saccharide, or about 90-100% phosphorylated quercetin-O-saccharide or phosphorylated fisetin-O-saccharide, or about 95-100% phosphorylated quercetin-O-saccharide or phosphorylated fisetin-O-saccharide, or about 99-100% phosphorylated quercetin-O-saccharide or phosphorylated fisetin-O-saccharide. In some embodiments, the invention provides a composition that contains about 1-90% phosphorylated quercetin-O-saccharide or phosphorylated fisetin-O-saccharide, or about 10-90% phosphorylated quercetin-O-saccharide or phosphorylated fisetin-O-saccharide, or about 20-90% phosphorylated quercetin-O-saccharide or phosphorylated fisetin-O-saccharide, or about 50-90% phosphorylated quercetin-O-saccharide or phosphorylated fisetin-O-saccharide, or about 80-90% phosphorylated quercetin-O-saccharide or phosphorylated fisetin-O-saccharide. In some embodiments, the invention provides a composition that contains about 1-75% phosphorylated quercetin-O-saccharide or phosphorylated fisetin-O-saccharide, or about 10-75% phosphorylated quercetin-O-saccharide or phosphorylated fisetin-O-saccharide, or about 20-75% phosphorylated quercetin-O-saccharide or phosphorylated fisetin-O-saccharide, or about 50-75% phosphorylated quercetin-O-saccharide or phosphorylated fisetin-O-saccharide. In some embodiments, the invention provides a composition that contains about 1-50% phosphorylated quercetin-O-saccharide or phosphorylated fisetin-O-saccharide, or about 10-50% phosphorylated quercetin-O-saccharide or phosphorylated fisetin-O-saccharide, or about 20-50% phosphorylated quercetin-O-saccharide or phosphorylated fisetin-β-saccharide, or about 30-50% phosphorylated quercetin-O-saccharide or phosphorylated fisetin-O-saccharide, or about 40-50% phosphorylated quercetin-O-saccharide or phosphorylated fisetin-O-saccharide. In some embodiments, the invention provides a composition that contains about 140% phosphorylated quercetin-O-saccharide or phosphorylated fisetin-O-saccharide, or about 10-40% phosphorylated quercetin-O-saccharide or phosphorylated fisetin-O-saccharide, or about 20-40% phosphorylated quercetin-O-saccharide or phosphorylated fisetin-O-saccharide, or about 30-40% phosphorylated quercetin-O-saccharide or phosphorylated fisetin-O-saccharide. In some embodiments, the invention provides a composition that contains about 1-30% phosphorylated quercetin-O-saccharide or phosphorylated fisetin-O-saccharide, or about 10-30% phosphorylated quercetin-O-saccharide or phosphorylated fisetin-O-saccharide, or about 20-30% phosphorylated quercetin-O-saccharide or phosphorylated fisetin-O-saccharide. In some embodiments, the invention provides a composition that contains about 1-20% phosphorylated quercetin-O-saccharide or phosphorylated fisetin-O-saccharide, or about 10-20% phosphorylated quercetin-O-saccharide or phosphorylated fisetin-O-saccharide. In some embodiments, the invention provides a composition that contains about 1-10% phosphorylated quercetin-O-saccharide or phosphorylated fisetin-O-saccharide. In some embodiments, the invention provides a composition that contains about 1, 2, 5, 10, 20, 30, 40, 50, 60, 70, 80, 90, 95, 96, 97, 98, or 99% phosphorylated quercetin-O-saccharide or phosphorylated fisetin-O-saccharide.

In some embodiments, the invention provides a composition for administration of quercetin or fisetin to an animal, e.g., for the oral delivery of quercetin or fisetin to reduce hyperglycemia and/or one or more symptoms of hyperglycemia, that contain at least about 1, 5, 10, 20, 30, 40, 50, 60, 70, 80, 90, 95, 99, 99.5, 99.9, or 99.99% quercetin-O-saccharide or fisetin-O-saccharide. In some embodiments, the invention provides a composition for the oral delivery of quercetin that contains no more than about 2, 5, 10, 20, 30, 40, 50, 60, 70, 80, 90, 95, 99, 99.5, 99.9, 99.99, or 100% quercetin-O-saccharide or fisetin-O-saccharide. In some embodiments, the invention provides a composition that contains about 1-100% quercetin-O-saccharide or fisetin-O-saccharide, or about 10-100% quercetin-O-saccharide or fisetin-O-saccharide, or about 20-100% quercetin-O-saccharide or fisetin-O-saccharide, or about 50-100% quercetin-O-saccharide or fisetin-O-saccharide, or about 80-100% quercetin-O-saccharide or fisetin-O-saccharide, or about 90-100% quercetin-O-saccharide or fisetin-O-saccharide, or about 95-100% quercetin-O-saccharide or fisetin-O-saccharide, or about 99-100% quercetin-O-saccharide or fisetin-O-saccharide. In some embodiments, the invention provides a composition that contains about 1-90% quercetin-O-saccharide or fisetin-O-saccharide, or about 10-90% quercetin-O-saccharide or fisetin-O-saccharide, or about 20-90% quercetin-O-saccharide or fisetin-O-saccharide, or about 50-90% quercetin-O-saccharide or fisetin-O-saccharide, or about 80-90% quercetin-O-saccharide or fisetin-O-saccharide. In some embodiments, the invention provides a composition that contains about 1-75% quercetin-O-saccharide or fisetin-O-saccharide, or about 10-75% quercetin-O-saccharide or fisetin-O-saccharide, or about 20-75% quercetin-O-saccharide or fisetin-O-saccharide, or about 50-75% quercetin-O-saccharide or fisetin-O-saccharide. In some embodiments, the invention provides a composition that contains about 1-50% quercetin-O-saccharide or fisetin-O-saccharide, or about 10-50% quercetin-O-saccharide or fisetin-O-saccharide, or about 20-50% quercetin-O-saccharide or fisetin-O-saccharide, or about 30-50% quercetin-O-saccharide or fisetin-O-saccharide, or about 40-50% quercetin-O-saccharide or fisetin-O-saccharide. In some embodiments, the invention provides a composition that contains about 1-40% quercetin-O-saccharide or fisetin-O-saccharide, or about 10-40% quercetin-O-saccharide or fisetin-O-saccharide, or about 20-40% quercetin-O-saccharide or fisetin-O-saccharide, or about 30-40% quercetin-O-saccharide or fisetin-O-saccharide. In some embodiments, the invention provides a composition that contains about 1-30% quercetin-O-saccharide or fisetin-O-saccharide, or about 10-30% quercetin-O-saccharide or fisetin-O-saccharide, or about 20-30% quercetin-β-saccharide or fisetin-O-saccharide. In some embodiments, the invention provides a composition that contains about 1-20% quercetin-O-saccharide or fisetin-O-saccharide, or about 10-20% quercetin-O-saccharide or fisetin-O-saccharide. In some embodiments, the invention provides a composition that contains about 1-10% quercetin-O-saccharide or fisetin-O-saccharide. In some embodiments, the invention provides a composition that contains about 1, 2, 5, 10, 20, 30, 40, 50, 60, 70, 80, 90, 95, 96, 97, 98, or 99% quercetin-O-saccharide or fisetin-O-saccharide.

In some embodiments, the invention provides a composition for administration of phosphorylated quercetin or phosphorylated fisetin to an animal to reduce a side effect of a substance, e.g., for the oral delivery of phosphorylated quercetin or phosphorylated fisetin, that contain at least about 1, 5, 10, 20, 30, 40, 50, 60, 70, 80, 90, 95, 99, 99.5, 99.9, or 99.99% phosphorylated quercetin-3-O-glycoside or phosphorylated fisetin-3-O-glycoside. In some embodiments, the invention provides a composition for the oral delivery of phosphorylated quercetin that contains no more than about 2, 5, 10, 20, 30, 40, 50, 60, 70, 80, 90, 95, 99, 99.5, 99.9, 99.99, or 100% phosphorylated quercetin-3-O-glycoside or phosphorylated fisetin-3-O-glycoside. In some embodiments, the invention provides a composition that contains about 1-100% phosphorylated quercetin-3-O-glycoside or phosphorylated fisetin-3-O-glycoside, or about 10-100% phosphorylated quercetin-3-O-glycoside or phosphorylated fisetin-3-O-glycoside, or about 20-100% phosphorylated quercetin-3-O-glycoside or phosphorylated fisetin-3-O-glycoside, or about 50-100% phosphorylated quercetin-3-O-glycoside or phosphorylated fisetin-3-O-glycoside, or about 80-100% phosphorylated quercetin-3-O-glycoside or phosphorylated fisetin-3-O-glycoside, or about 90-100% phosphorylated quercetin-3-O-glycoside or phosphorylated fisetin-3-O-glycoside, or about 95-100% phosphorylated quercetin-3-O-glycoside or phosphorylated fisetin-3-O-glycoside, or about 99-100% phosphorylated quercetin-3-O-glycoside or phosphorylated fisetin-3-O-glycoside. In some embodiments, the invention provides a composition that contains about 1-90% phosphorylated quercetin-3-O-glycoside or phosphorylated fisetin-3-O-glycoside, or about 10-90% phosphorylated quercetin-3-O-glycoside or phosphorylated fisetin-3-O-glycoside, or about 20-90% phosphorylated quercetin-3-O-glycoside or phosphorylated fisetin-3-O-glycoside, or about 50-90% phosphorylated quercetin-3-O-glycoside or phosphorylated fisetin-3-O-glycoside, or about 80-90% phosphorylated quercetin-3-O-glycoside or phosphorylated fisetin-3-O-glycoside. In some embodiments, the invention provides a composition that contains about 1-75% phosphorylated quercetin-3-O-glycoside or phosphorylated fisetin-3-O-glycoside, or about 10-75% phosphorylated quercetin-3-O-glycoside or phosphorylated fisetin-3-O-glycoside, or about 20-75% phosphorylated quercetin-3-O-glycoside or phosphorylated fisetin-3-O-glycoside, or about 50-75% phosphorylated quercetin-3-O-glycoside or phosphorylated fisetin-3-O-glycoside. In some embodiments, the invention provides a composition that contains about 1-50% phosphorylated quercetin-3-O-glycoside or phosphorylated fisetin-3-O-glycoside, or about 10-50% phosphorylated quercetin-3-O-glycoside or phosphorylated fisetin-3-O-glycoside, or about 20-50% phosphorylated quercetin-3-O-glycoside or phosphorylated fisetin-3-O-glycoside, or about 30-50% phosphorylated quercetin-3-O-glycoside or phosphorylated fisetin-3-O-glycoside, or about 40-50% phosphorylated quercetin-3-O-glycoside or phosphorylated fisetin-3-O-glycoside. In some embodiments, the invention provides a composition that contains about 140% phosphorylated quercetin-3-O-glycoside or phosphorylated fisetin-3-O-glycoside, or about 10-40% phosphorylated quercetin-3-O-glycoside or phosphorylated fisetin-3-O-glycoside, or about 20-40% phosphorylated quercetin-3-O-glycoside or phosphorylated fisetin-3-O-glycoside, or about 3040% phosphorylated quercetin-3-O-glycoside or phosphorylated fisetin-3-O-glycoside. In some embodiments, the invention provides a composition that contains about 1-30% phosphorylated quercetin-3-O-glycoside or phosphorylated fisetin-3-O-glycoside, or about 10-30% phosphorylated quercetin-3-O-glycoside or phosphorylated fisetin-3-O-glycoside, or about 20-30% phosphorylated quercetin-3-O-glycoside or phosphorylated fisetin-3-O-glycoside. In some embodiments, the invention provides a composition that contains about 1-20% phosphorylated quercetin-3-O-glycoside or phosphorylated fisetin-3-O-glycoside, or about 10-20% phosphorylated quercetin-3-O-glycoside or phosphorylated fisetin-3-O-glycoside. In some embodiments, the invention provides a composition that contains about 1-10% phosphorylated quercetin-3-O-glycoside or phosphorylated fisetin-3-O-glycoside. In some embodiments, the invention provides a composition that contains about 1, 2, 5, 10, 20, 30, 40, 50, 60, 70, 80, 90, 95, 96, 97, 98, or 99% phosphorylated quercetin-3-O-glycoside or phosphorylated fisetin-3-O-glycoside.

In some embodiments, the invention provides a composition for administration of fisetin or quercetin to an animal, e.g., for the oral delivery of quercetin or fisetin to reduce hyperglycemia and/or one or more symptoms of hyperglycemia, that contain at least about 1, 5, 10, 20, 30, 40, 50, 60, 70, 80, 90, 95, 99, 99.5, 99.9, or 99.99% quercetin-3-O-glycoside or fisetin-3-O-glycoside. In some embodiments, the invention provides a composition for the oral delivery of quercetin that contains no more than about 2, 5, 10, 20, 30, 40, 50, 60, 70, 80, 90, 95, 99, 99.5, 99.9, 99.99, or 100% quercetin-3-O-glycoside or fisetin-3-O-glycoside. In some embodiments, the invention provides a composition that contains about 1-100% quercetin-3-O-glycoside or fisetin-3-O-glycoside, or about 10-100% quercetin-3-O-glycoside or fisetin-3-O-glycoside, or about 20-100% quercetin-3-O-glycoside or fisetin-3-O-glycoside, or about 50-100% quercetin-3-O-glycoside or fisetin-3-O-glycoside, or about 80-100% quercetin-3-O-glycoside or fisetin-3-O-glycoside, or about 90-100% quercetin-3-O-glycoside or fisetin-3-O-glycoside, or about 95-100% quercetin-3-O-glycoside or fisetin-3-O-glycoside, or about 99-100% quercetin-3-O-glycoside or fisetin-3-O-glycoside. In some embodiments, the invention provides a composition that contains about 1-90% quercetin-3-O-glycoside or fisetin-3-O-glycoside, or about 10-90% quercetin-3-O-glycoside or fisetin-3-O-glycoside, or about 20-90% quercetin-3-O-glycoside or fisetin-3-O-glycoside, or about 50-90% quercetin-3-O-glycoside or fisetin-3-O-glycoside, or about 80-90% quercetin-3-O-glycoside or fisetin-3-O-glycoside. In some embodiments, the invention provides a composition that contains about 1-75% quercetin-3-O-glycoside or fisetin-3-O-glycoside, or about 10-75% quercetin-3-O-glycoside or fisetin-3-O-glycoside, or about 20-75% quercetin-3-O-glycoside or fisetin-3-O-glycoside, or about 50-75% quercetin-3-O-glycoside or fisetin-3-O-glycoside. In some embodiments, the invention provides a composition that contains about 1-50% quercetin-3-O-glycoside or fisetin-3-O-glycoside, or about 10-50% quercetin-3-O-glycoside or fisetin-3-O-glycoside, or about 20-50% quercetin-3-O-glycoside or fisetin-3-O-glycoside, or about 30-50% quercetin-3-O-glycoside or fisetin-3-O-glycoside, or about 40-50% quercetin-3-O-glycoside or fisetin-3-O-glycoside. In some embodiments, the invention provides a composition that contains about 140% quercetin-3-O-glycoside or fisetin-3-O-glycoside, or about 10-40% quercetin-3-O-glycoside or fisetin-3-O-glycoside, or about 20-40% quercetin-3-O-glycoside or fisetin-3-O-glycoside, or about 30-40% quercetin-3-O-glycoside or fisetin-3-O-glycoside. In some embodiments, the invention provides a composition that contains about 1-30% quercetin-3-O-glycoside or fisetin-3-O-glycoside, or about 10-30% quercetin-3-O-glycoside or fisetin-3-O-glycoside, or about 20-30% quercetin-3-O-glycoside or fisetin-3-O-glycoside. In some embodiments, the invention provides a composition that contains about 1-20% quercetin-3-O-glycoside or fisetin-3-O-glycoside, or about 10-20% quercetin-3-O-glycoside or fisetin-3-O-glycoside. In some embodiments, the invention provides a composition that contains about 1-10% quercetin-3-O-glycoside or fisetin-3-O-glycoside. In some embodiments, the invention provides a composition that contains about 1, 2, 5, 10, 20, 30, 40, 50, 60, 70, 80, 90, 95, 96, 97, 98, or 99% quercetin-3-O-glycoside or fisetin-3-O-glycoside.

In some embodiments, the invention provides a composition for administration of phosphorylated quercetin or phosphorylated fisetin to an animal to reduce a side effect of a substance, e.g., for the oral delivery of phosphorylated quercetin or phosphorylated fisetin, that contain at least about 1, 5, 10, 20, 30, 40, 50, 60, 70, 80, 90, 95, 99, 99.5, 99.9, or 99.99% phosphorylated quercetin-3-O-glucorhamnoside or phosphorylated fisetin-3-O-glucorhamnoside. In some embodiments, the invention provides a composition for the oral delivery of phosphorylated quercetin or phosphorylated fisetin that contains no more than about 2, 5, 10, 20, 30, 40, 50, 60, 70, 80, 90, 95, 99, 99.5, 99.9, 99.99, or 100% phosphorylated quercetin-3-O-glucorhamnoside or phosphorylated fisetin-3-O-glucorhamnoside. In some embodiments, the invention provides a composition that contains about 1-100% phosphorylated quercetin-3-O-glucorhamnoside or phosphorylated fisetin-3-O-glucorhamnoside, or about 10-100% phosphorylated quercetin-3-O-glucorhamnoside or phosphorylated fisetin-3-O-glucorhamnoside, or about 20-100% phosphorylated quercetin-3-O-glucorhamnoside or phosphorylated fisetin-3-O-glucorhamnoside, or about 50-100% phosphorylated quercetin-3-O-glucorhamnoside or phosphorylated fisetin-3-O-glucorhamnoside, or about 80-100% phosphorylated quercetin-3-O-glucorhamnoside or phosphorylated fisetin-3-O-glucorhamnoside, or about 90-100% phosphorylated quercetin-3-O-glucorhamnoside or phosphorylated fisetin-3-O-glucorhamnoside, or about 95-100% phosphorylated quercetin-3-O-glucorhamnoside or phosphorylated fisetin-3-O-glucorhamnoside, or about 99-100% phosphorylated quercetin-3-O-glucorhamnoside or phosphorylated fisetin-3-O-glucorhamnoside. In some embodiments, the invention provides a composition that contains about 1-90% phosphorylated quercetin-3-O-glucorhamnoside or phosphorylated fisetin-3-O-glucorhamnoside, or about 10-90% phosphorylated quercetin-3-O-glucorhamnoside or phosphorylated fisetin-3-O-glucorhamnoside, or about 20-90% phosphorylated quercetin-3-O-glucorhamnoside or phosphorylated fisetin-3-O-glucorhamnoside, or about 50-90% phosphorylated quercetin-3-O-glucorhamnoside or phosphorylated fisetin-3-O-glucorhamnoside, or about 80-90% phosphorylated quercetin-3-O-glucorhamnoside or phosphorylated fisetin-3-O-glucorhamnoside. In some embodiments, the invention provides a composition that contains about 1-75% phosphorylated quercetin-3-O-glucorhamnoside or phosphorylated fisetin-3-O-glucorhamnoside, or about 10-75% phosphorylated quercetin-3-O-glucorhamnoside or phosphorylated fisetin-3-O-glucorhamnoside, or about 20-75% phosphorylated quercetin-3-O-glucorhamnoside or phosphorylated fisetin-3-O-glucorhamnoside, or about 50-75% phosphorylated quercetin-3-O-glucorhamnoside or phosphorylated fisetin-3-O-glucorhamnoside. In some embodiments, the invention provides a composition that contains about 1-50% phosphorylated quercetin-3-O-glucorhamnoside or phosphorylated fisetin-3-O-glucorhamnoside, or about 10-50% phosphorylated quercetin-3-O-glucorhamnoside or phosphorylated fisetin-3-O-glucorhamnoside, or about 20-50% phosphorylated quercetin-3-O-glucorhamnoside or phosphorylated fisetin-3-O-glucorhamnoside, or about 30-50% phosphorylated quercetin-3-O-glucorhamnoside or phosphorylated fisetin-3-O-glucorhamnoside, or about 40-50% phosphorylated quercetin-3-O-glucorhamnoside or phosphorylated fisetin-3-O-glucorhamnoside. In some embodiments, the invention provides a composition that contains about 1-40% phosphorylated quercetin-3-O-glucorhamnoside or phosphorylated fisetin-3-O-glucorhamnoside, or about 10-40% phosphorylated quercetin-3-O-glucorhamnoside or phosphorylated fisetin-3-O-glucorhamnoside, or about 20-40% phosphorylated quercetin-3-O-glucorhamnoside or phosphorylated fisetin-3-O-glucorhamnoside, or about 30-40% phosphorylated quercetin-3-O-glucorhamnoside or phosphorylated fisetin-3-O-glucorhamnoside. In some embodiments, the invention provides a composition that contains about 1-30% phosphorylated quercetin-3-O-glucorhamnoside or phosphorylated fisetin-3-O-glucorhamnoside, or about 10-30% phosphorylated quercetin-3-O-glucorhamnoside or phosphorylated fisetin-3-O-glucorhamnoside, or about 20-30% phosphorylated quercetin-3-O-glucorhamnoside or phosphorylated fisetin-3-O-glucorhamnoside. In some embodiments, the invention provides a composition that contains about 1-20% phosphorylated quercetin-3-O-glucorhamnoside or phosphorylated fisetin-3-O-glucorhamnoside, or about 10-20% phosphorylated quercetin-3-O-glucorhamnoside or phosphorylated fisetin-3-O-glucorhamnoside. In some embodiments, the invention provides a composition that contains about 1-10% phosphorylated quercetin-3-O-glucorhamnoside or phosphorylated fisetin-3-O-glucorhamnoside. In some embodiments, the invention provides a composition that contains about 1, 2, 5, 10, 20, 30, 40, 50, 60, 70, 80, 90, 95, 96, 97, 98, or 99% phosphorylated quercetin-3-O-glucorhamnoside.

In some embodiments, the invention provides a composition for administration of quercetin or fisetin to an animal, e.g., for the oral delivery of quercetin or fisetin to reduce hyperglycemia and/or one or more symptoms of hyperglycemia, that contain at least about 1, 5, 10, 20, 30, 40, 50, 60, 70, 80, 90, 95, 99, 99.5, 99.9, or 99.99% quercetin-3-O-glucorhamnoside or fisetin-3-O-glucorhamnoside. In some embodiments, the invention provides a composition for the oral delivery of quercetin that contains no more than about 2, 5, 10, 20, 30, 40, 50, 60, 70, 80, 90, 95, 99, 99.5, 99.9, 99.99, or 100% quercetin-3-O-glucorhamnoside or fisetin-3-O-glucorhamnoside. In some embodiments, the invention provides a composition that contains about 1-100% quercetin-3-O-glucorhamnoside or fisetin-3-O-glucorhamnoside, or about 10-100% quercetin-3-O-glucorhamnoside or fisetin-3-O-glucorhamnoside, or about 20-100% quercetin-3-O-glucorhamnoside or fisetin-3-O-glucorhamnoside, or about 50-100% quercetin-3-O-glucorhamnoside or fisetin-3-O-glucorhamnoside, or about 80-100% quercetin-3-O-glucorhamnoside or fisetin-3-O-glucorhamnoside, or about 90-100% quercetin-3-O-glucorhamnoside or fisetin-3-O-glucorhamnoside, or about 95-100% quercetin-3-O-glucorhamnoside or fisetin-3-O-glucorhamnoside, or about 99-100% quercetin-3-O-glucorhamnoside or fisetin-3-O-glucorhamnoside. In some embodiments, the invention provides a composition that contains about 1-90% quercetin-3-O-glucorhamnoside or fisetin-3-O-glucorhamnoside, or about 10-90% quercetin-3-O-glucorhamnoside or fisetin-3-O-glucorhamnoside, or about 20-90% quercetin-3-O-glucorhamnoside or fisetin-3-O-glucorhamnoside, or about 50-90% quercetin-3-O-glucorhamnoside or fisetin-3-O-glucorhamnoside, or about 80-90% quercetin-3-O-glucorhamnoside or fisetin-3-O-glucorhamnoside. In some embodiments, the invention provides a composition that contains about 1-75% quercetin-3-O-glucorhamnoside or fisetin-3-O-glucorhamnoside, or about 10-75% quercetin-3-O-glucorhamnoside or fisetin-3-O-glucorhamnoside, or about 20-75% quercetin-3-O-glucorhamnoside or fisetin-3-O-glucorhamnoside, or about 50-75% quercetin-3-O-glucorhamnoside or fisetin-3-O-glucorhamnoside. In some embodiments, the invention provides a composition that contains about 1-50% quercetin-3-O-glucorhamnoside or fisetin-3-O-glucorhamnoside, or about 10-50% quercetin-3-O-glucorhamnoside or fisetin-3-O-glucorhamnoside, or about 20-50% quercetin-3-O-glucorhamnoside or fisetin-3-O-glucorhamnoside, or about 30-50% quercetin-3-O-glucorhamnoside or fisetin-3-O-glucorhamnoside, or about 40-50% quercetin-3-O-glucorhamnoside or fisetin-3-O-glucorhamnoside. In some embodiments, the invention provides a composition that contains about 140% quercetin-3-O-glucorhamnoside or fisetin-3-O-glucorhamnoside, or about 10-40% quercetin-3-O-glucorhamnoside or fisetin-3-O-glucorhamnoside, or about 20-40% quercetin-3-O-glucorhamnoside or fisetin-3-O-glucorhamnoside, or about 30-40% quercetin-3-O-glucorhamnoside or fisetin-3-O-glucorhamnoside. In some embodiments, the invention provides a composition that contains about 1-30% quercetin-3-O-glucorhamnoside or fisetin-3-O-glucorhamnoside, or about 10-30% quercetin-3-O-glucorhamnoside or fisetin-3-O-glucorhamnoside, or about 20-30% quercetin-3-O-glucorhamnoside or fisetin-3-O-glucorhamnoside. In some embodiments, the invention provides a composition that contains about 1-20% quercetin-3-O-glucorhamnoside or fisetin-3-O-glucorhamnoside, or about 10-20% quercetin-3-O-glucorhamnoside or fisetin-3-O-glucorhamnoside. In some embodiments, the invention provides a composition that contains about 1-10% quercetin-3-O-glucorhamnoside or fisetin-3-O-glucorhamnoside. In some embodiments, the invention provides a composition that contains about 1, 2, 5, 10, 20, 30, 40, 50, 60, 70, 80, 90, 95, 96, 97, 98, or 99% quercetin-3-O-glucorhamnoside.

In some embodiments, the invention provides a composition for administration of phosphorylated quercetin or fisetin to an animal to reduce a side effect of a substance, e.g., for the oral delivery of phosphorylated quercetin or fisetin, that contain at least about 1, 5, 10, 20, 30, 40, 50, 60, 70, 80, 90, 95, 99, 99.5, 99.9, or 99.99% phosphorylated quercetin aglycone or phosphorylated fisetin aglycone. In some embodiments, the invention provides a composition for the oral delivery of phosphorylated quercetin or fisetin that contains no more than about 2, 5, 10, 20, 30, 40, 50, 60, 70, 80, 90, 95, 99, 99.5, 99.9, 99.99, or 100% phosphorylated quercetin aglycone or phosphorylated fisetin aglycone. In some embodiments, the invention provides a composition that contains about 1-100% phosphorylated quercetin aglycone or phosphorylated fisetin aglycone, or about 10-100% phosphorylated quercetin aglycone or phosphorylated fisetin aglycone, or about 20-100% phosphorylated quercetin aglycone or phosphorylated fisetin aglycone, or about 50-100% phosphorylated quercetin aglycone or phosphorylated fisetin aglycone, or about 80-100% phosphorylated quercetin aglycone or phosphorylated fisetin aglycone, or about 90-100% phosphorylated quercetin aglycone or phosphorylated fisetin aglycone, or about 95-100% phosphorylated quercetin aglycone or phosphorylated fisetin aglycone, or about 99-100% phosphorylated quercetin aglycone or phosphorylated fisetin aglycone. In some embodiments, the invention provides a composition that contains about 1-90% phosphorylated quercetin aglycone or phosphorylated fisetin aglycone, or about 10-90% phosphorylated quercetin aglycone or phosphorylated fisetin aglycone, or about 20-90% phosphorylated quercetin aglycone or phosphorylated fisetin aglycone, or about 50-90% phosphorylated quercetin aglycone or phosphorylated fisetin aglycone, or about 80-90% phosphorylated quercetin aglycone or phosphorylated fisetin aglycone. In some embodiments, the invention provides a composition that contains about 1-75% phosphorylated quercetin aglycone or phosphorylated fisetin aglycone, or about 10-75% phosphorylated quercetin aglycone or phosphorylated fisetin aglycone, or about 20-75% phosphorylated quercetin aglycone or phosphorylated fisetin aglycone, or about 50-75% phosphorylated quercetin aglycone or phosphorylated fisetin aglycone. In some embodiments, the invention provides a composition that contains about 1-50% phosphorylated quercetin aglycone or phosphorylated fisetin aglycone, or about 10-50% phosphorylated quercetin aglycone or phosphorylated fisetin aglycone, or about 20-50% phosphorylated quercetin aglycone or phosphorylated fisetin aglycone, or about 30-50% phosphorylated quercetin aglycone or phosphorylated fisetin aglycone, or about 40-50% phosphorylated quercetin aglycone or phosphorylated fisetin aglycone. In some embodiments, the invention provides a composition that contains about 1-40% phosphorylated quercetin aglycone or phosphorylated fisetin aglycone, or about 10-40% phosphorylated quercetin aglycone or phosphorylated fisetin aglycone, or about 20-40% phosphorylated quercetin aglycone or phosphorylated fisetin aglycone, or about 30-40% phosphorylated quercetin aglycone or phosphorylated fisetin aglycone. In some embodiments, the invention provides a composition that contains about 1-30% phosphorylated quercetin aglycone or phosphorylated fisetin aglycone, or about 10-30% phosphorylated quercetin aglycone or phosphorylated fisetin aglycone, or about 20-30% phosphorylated quercetin aglycone or phosphorylated fisetin aglycone. In some embodiments, the invention provides a composition that contains about 1-20% phosphorylated quercetin aglycone or phosphorylated fisetin aglycone, or about 10-20% phosphorylated quercetin aglycone or phosphorylated fisetin aglycone. In some embodiments, the invention provides a composition that contains about 1-10% phosphorylated quercetin aglycone or phosphorylated fisetin aglycone. In some embodiments, the invention provides a composition that contains about 1, 2, 5, 10, 20, 30, 40, 50, 60, 70, 80, 90, 95, 96, 97, 98, or 99% phosphorylated quercetin aglycone or phosphorylated fisetin aglycone.

In some embodiments, the invention provides a composition for administration of quercetin or fisetin to an animal, e.g., for the oral delivery of quercetin or fisetin to reduce hyperglycemia and/or one or more symptoms of hyperglycemia, that contain at least about 1, 5, 10, 20, 30, 40, 50, 60, 70, 80, 90, 95, 99, 99.5, 99.9, or 99.99% quercetin aglycone or fisetin aglycone. In some embodiments, the invention provides a composition for the oral delivery of quercetin or fisetin that contains no more than about 2, 5, 10, 20, 30, 40, 50, 60, 70, 80, 90, 95, 99, 99.5, 99.9, 99.99, or 100% quercetin aglycone or fisetin aglycone. In some embodiments, the invention provides a composition that contains about 1-100% quercetin aglycone or fisetin aglycone, or about 10-100% quercetin aglycone or fisetin aglycone, or about 20-100% quercetin aglycone or fisetin aglycone, or about 50-100% quercetin aglycone or fisetin aglycone, or about 80-100% quercetin aglycone or fisetin aglycone, or about 90-100% quercetin aglycone or fisetin aglycone, or about 95-100% quercetin aglycone or fisetin aglycone, or about 99-100% quercetin aglycone or fisetin aglycone. In some embodiments, the invention provides a composition that contains about 1-90% quercetin aglycone or fisetin aglycone, or about 10-90% quercetin aglycone or fisetin aglycone, or about 20-90% quercetin aglycone or fisetin aglycone, or about 50-90% quercetin aglycone or fisetin aglycone, or about 80-90% quercetin aglycone or fisetin aglycone. In some embodiments, the invention provides a composition that contains about 1-75% quercetin aglycone or fisetin aglycone, or about 10-75% quercetin aglycone or fisetin aglycone, or about 20-75% quercetin aglycone or fisetin aglycone, or about 50-75% quercetin aglycone or fisetin aglycone. In some embodiments, the invention provides a composition that contains about 1-50% quercetin aglycone or fisetin aglycone, or about 10-50% quercetin aglycone or fisetin aglycone, or about 20-50% quercetin aglycone or fisetin aglycone, or about 30-50% quercetin aglycone or fisetin aglycone, or about 40-50% quercetin aglycone or fisetin aglycone. In some embodiments, the invention provides a composition that contains about 1-40% quercetin aglycone or fisetin aglycone, or about 10-40% quercetin aglycone or fisetin aglycone, or about 20-40% quercetin aglycone or fisetin aglycone, or about 30-40% quercetin aglycone or fisetin aglycone. In some embodiments, the invention provides a composition that contains about 1-30% quercetin aglycone or fisetin aglycone, or about 10-30% quercetin aglycone or fisetin aglycone, or about 20-30% quercetin aglycone or fisetin aglycone. In some embodiments, the invention provides a composition that contains about 1-20% quercetin aglycone or fisetin aglycone, or about 10-20% quercetin aglycone or fisetin aglycone. In some embodiments, the invention provides a composition that contains about 1-10% quercetin aglycone or fisetin aglycone. In some embodiments, the invention provides a composition that contains about 1, 2, 5, 10, 20, 30, 40, 50, 60, 70, 80, 90, 95, 96, 97, 98, or 99% quercetin aglycone or fisetin aglycone.

In some embodiments, the invention provides a composition for administration of quercetin or fisetin to an animal, e.g., for the oral delivery of quercetin or fisetin to reduce hyperglycemia and/or one or more symptoms of hyperglycemia, that contains a combination of phosphorylated quercetin-O-saccharides or phosphorylated fisetin-O-saccharides and/or quercetin-O-saccharides or fisetin-O-saccharides. In some embodiments, the invention provides a composition for administration of quercetin or fisetin to an animal to reduce hyperglycemia and/or one or more symptoms of hyperglycemia induced by a calcineurin inhibitor, by way of example only that contain a combination of quercetin-3-O-glycoside or fisetin-3-O-glycoside and quercetin-3-O-glucorhamnoside or fisetin-3-O-glucorhamnoside for the oral delivery of quercetin or fisetin. In these compositions, the ranges or amounts of the phosphorylated or non-phosphorylated quercetin-O-saccharides or fisetin-O-saccharides, e.g., phosphorylated quercetin-3-O-glycoside or phosphorylated fisetin-3-O-glycoside and phosphorylated quercetin-3-O-glucorhamnoside or phosphorylated fisetin-3-O-glucorhamnoside may be any suitable combination of the ranges or amounts, above.

In some embodiments, the invention provides a composition for administration of quercetin or fisetin to an animal, e.g., for the oral delivery of quercetin or fisetin to reduce hyperglycemia and/or one or more symptoms of hyperglycemia, that contains a combination of one or more quercetin-O-saccharides or fisetin-O-saccharides and quercetin aglycone or fisetin aglycone. In some embodiments, the invention provides a composition for administration of quercetin or fisetin to an animal to reduce hyperglycemia and/or one or more symptoms of hyperglycemia induced by a calcineurin inhibitor, e.g., for the oral delivery of quercetin or fisetin, that contain a combination of quercetin-3-O-glycoside or fisetin-3-O-glycoside and quercetin aglycone or fisetin aglycone. In these compositions, the ranges or amounts of quercetin-3-O-glycoside or fisetin-3-O-glycoside and quercetin aglycone or fisetin aglycone may be any suitable combination of the ranges or amounts, above. In some embodiments, the invention provides a composition for administration of quercetin to an animal to reduce hyperglycemia and/or one or more symptoms of hyperglycemia induced by a calcineurin inhibitor, e.g., for the oral delivery of quercetin or fisetin, that contain a combination of quercetin-3-O-glucorhamnoside or fisetin-3-O-glucorhamnoside and quercetin aglycone or fisetin aglycone. In these compositions, the ranges or amounts of quercetin-3-O-glucorhamnoside or fisetin-3-O-glucorhamnoside and quercetin aglycone or fisetin aglycone may be any suitable combination of the ranges or amounts, above. In some embodiments, the invention provides a composition for administration of quercetin or fisetin to an animal to reduce hyperglycemia and/or one or more symptoms of hyperglycemia induced by a calcineurin inhibitor, e.g., for the oral delivery of quercetin or fisetin, that contain a combination of quercetin-3-O-glycoside or fisetin-3-O-glycoside, quercetin-3-O-glucorhamnoside or fisetin-3-O-glucorhamnoside and quercetin aglycone or fisetin aglycone. In these compositions, the ranges or amounts of quercetin-3-O-glycoside or fisetin-3-O-glycoside, quercetin-3-O-glucorhamnoside or fisetin-3-O-glucorhamnoside and quercetin aglycone or fisetin aglycone may be any suitable combination of the ranges or amounts, above. Other quercetin saccharides or fisetin saccharides, as described herein and as known in the art or developed, may be used as well.

Examples of quercetin derivatives are described in U.S. Appn. No. 60/953,187, filed 31 Jul. 2007, entitled: Polyhydroxylated Aromatic Compositions and Methods, U.S. No. 60/953,188, filed 31 Jul. 2007, entitled: Flavonoid Phosphate Compositions and Methods, and Attorney Docket No. 31423.703.201, entitled: Pyrone Analog Compositions and Methods, and Attorney Docket No. 31423.703.201, entitled: Pyrone Analog Compositions and Methods, Docket No. 31423-716.102, entitled: Soluble Pyrone Analogs Methods and Compositions, and Docket No. 31423.720.201, entitled: Phosphorylated Pyrone Analogs and Methods, all filed on even date herewith, all of which are incorporated by reference herein in their entirety.

In some embodiments, the invention provides a composition for administration of fisetin to an animal, e.g., for the oral delivery of fisetin to reduce hyperglycemia and/or one or more symptoms of hyperglycemia induced by the administration of a calcineurin inhibitor. In some embodiments, the invention provides a composition for administration of fisetin phosphate to an animal, e.g. for the oral delivery of fisetin phosphate to reduce hyperglycemia and/or one or more symptoms of hyperglycemia induced by a calcineurin inhibitor. In some embodiments, the invention provides a composition for administration of fisetin to an animal, e.g. for the oral delivery of fisetin to reduce hyperglycemia and/or one or more symptoms of hyperglycemia induced by a calcineurin inhibitor, that contains a mixture of one or more fisetin and/or fisetin phosphate and/or fisetin derivatives. Forms of fisetin (e.g. aglycone) and amounts for administration are as given herein for quercetin.

In some embodiments the administration is rectal, buccal, intranasal, transdermal, intravenous, intraperitoneal, parenteral, intramuscular, subcutaneous, orally, topical, as an inhalant, or via an impregnated or coated device such as a stent. In some embodiments the administration is intravenous. In some embodiments administration is transdermal. In some embodiments the administration is oral.

In some of these embodiments, a pharmaceutically acceptable excipient is also included.

### Calcineurin Inhibitors

The invention provides compositions and methods, e.g., to reduce or eliminate hyperglycemia and/or one or more symptoms of hyperglycemia induced by a calcineurin inhibitor. In some embodiments, the invention provides compositions and methods to change the concentration of a calcineurin inhibitor in a physiological compartment. In some embodiments, the invention provides compositions and methods to decrease the concentration of a calcineurin inhibitor in a physiological compartment. In some embodiments, the invention provides compositions and methods to decrease the concentration of a calcineurin inhibitor in a pancreatic islet cell. In some embodiments, the compositions and methods retain or enhance a desired effect of the calcineurin inhibitor, e.g., a peripheral effect. The methods and compositions of the invention apply to any calcineurin inhibitor for which it is desired to reduce hyperglycemia and/or one or more symptoms of hyperglycemia induced by a calcineurin inhibitor, e.g., glucosuria. In some embodiments, the compositions and methods of the invention utilize cyclosporin A (CsA). In some embodiments, the compositions and methods of the invention utilize tacrolimus. In some embodiments, the calcineurin inhibitor is tacrolimus analog. In some embodiments, the tacrolimus analog is selected from the group consisting of meridamycin, 31-O-Demethyl-FK506; L-683,590, L-685,818; 32-O-(1-hydroxyethylindol-5-yl)ascomycin; ascomycin; C18-OH-ascomycin; 9-deoxo-31-O-demethyl-FK506; L-688,617; A-119435; AP1903; rapamycin; dexamethasone-FK506 heterodimer; 13-O-demethyl tacrolimus; and FK 506-dextran conjugate.

### Tacrolimus

Tacrolimus, also known as FK506, is the active ingredient in Prograf, one of the leading market immunosuppressants from preventing transplant rejection. Tacrolimus is a macrolide immunosuppressant that can be produced by Streptomyces tsukubaensis. The chemical name is [3S-[3R[E(1S*,3S*,4S*)], 4S*,5R*,8S*,9E,12R*,14R*,15S*,16R*,18S*,19S*,26aR*]]-,6,8,11,12,13,14,15,16,17,18,19,24,25,26,26a-hexadecahydro-5,19-dihydroxy-3-[2-(4-hydroxy-3-methoxycyclohexyl)-1-methylethenyl]-14,16-dimethoxy-4,10,12,18-tetramethyl-8-(2-propenyl)-15,19-epoxy-3H-pyrido[2,1-c][1,4]oxaazacyclotricosine-1,7,20,21(4H,23H)-tetrone, monohydrate. The chemical structure of tacrolimus is:

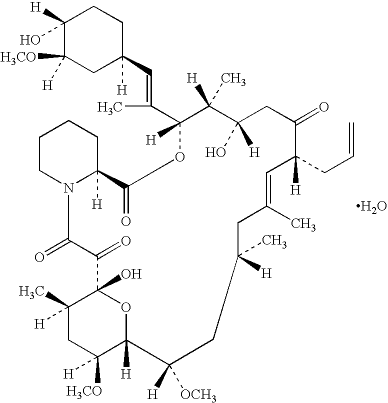

The empirical formula of tacrolimus is C₄₄H₆₉NO₁₂.H₂O (formula weight of 822.03). Early studies demonstrated the immunosuppressive properties of tacrolimus in vitro. At subnanomolar concentrations, tacrolimus was shown to inhibit the proliferation of murine or human T cells stimulated by specific antigens, antibodies to the T cell receptor (TCR)/CD3 complex or mitogenic lectins as well as the generation of cytolytic T cells (CTL) in mixed lymphocyte reactions. In these initial experiments, it became clear that tacrolimus exerts its activity by disrupting calcium signaling events that lead to lymphokine production, similar to CsA, but with 50-100-fold higher potency. Animal models of transplantation confirmed the immunosuppressive properties of tacrolimus and its higher potency over CsA. However, these animal studies also revealed that tacrolimus had major side effects, including neurotoxicity and nephrotoxicity, much like CsA. Despite these toxicity results, trials with tacrolimus were initiated as rescue therapy in human liver transplant patients who did not fare well with CsA treatment. In many of these patients, tacrolimus proved quite beneficial and had a lifesaving effect. These findings were further substantiated upon extended use of the drug in a larger group of patients, providing the impetus for controlled, multi-center clinical trials of tacrolimus as a primary therapy in liver and kidney transplantation. It was demonstrated that tacrolimus-based therapy offers a number of potential advantages over conventional CsA-based treatment, such as a corticosteroid-sparing action and a significant reduction in incidence of both acute and corticosteroid-resistant rejection episodes. Tacrolimus was approved by the FDA for the prophylaxis of liver transplant rejection in 1994 and kidney transplant rejection in 1997.

Tacrolimus prolongs the survival of the host and transplanted graft in animal transplant models of liver, kidney, heart, bone marrow, small bowel and pancreas, lung and trachea, skin, cornea, and limb. In animals, tacrolimus has been demonstrated to suppress some humoral immunity and, to a greater extent, cell-mediated reactions such as allograft rejection, delayed type hypersensitivity, collagen-induced arthritis, experimental allergic encephalomyelitis, and graft versus host disease.

Without being limited to any theory, tacrolimus inhibits T lymphocyte activation, although the exact mechanism is unknown, experimental data suggest that upon formation of a complex with the intracellular protein, FK506-binding protein 12 (FKBP12), the drug selectively inhibits the enzymatic activity of the calcium/calmodulin-dependent protein phosphatase, calcineurin. Engagement of the T cell receptor (TCR) initiates at least two separate signaling pathways driven by Ras/PKC and an elevation of intracellular Ca²⁺. The latter activates calcineurin, composed of a catalytic subunit, a regulatory subunit and of calmodulin. Enzymatically active calcineurin can dephosphorylate the cytoplasmic NFAT family members and cause the dissociation of the inhibitor IkB from NFkB. NFAT and NFkB are then translocated into the nucleus where they can interact with their DNA binding sequences on the IL-2 promoter. To be transcriptionally active, NFAT needs to form a complex with accessory factors, such as AP-1 (fos/jun) contributed by the Ras/PKC pathway. Calcineurin is also thought to regulate the activity of Oct-1 through induction of its co-activators OAP and BOB-1. The complex formed between FKBP12 and tacrolimus impedes access of calcineurin to its substrates and thereby prevents the nuclear translocation or activation of these factors. These factors are thought to initiate gene transcription for the formation of lymphokines (such as interleukin-2, gamma interferon). Calcineurin may also affect the function of the c-jun N-terminal kinase, JNK and Elk-1, which are components of Ras/PKC driven signaling mechanisms. The net result is that T lymphocyte activation is inhibited resulting in immunosuppression.

The greatest limitation to the therapeutic potential of tacrolimus comes from its toxic side effects, which include hyperglycemia. The precise pathophysiological mechanisms of tacrolimus toxicity are still enigmatic, in part because the cells that are actually implicated within the target tissues of this toxicity have not been clearly identified. However, evidence has accumulated that the side effects of tacrolimus arise from the same biochemical mechanisms that underlie its immunosuppressive effects, namely an inhibition of calcineurin activity in various tissues. This is suggested by the fact that the toxicity profile of tacrolimus overlaps with that of CsA and is totally different from that of rapamycin, an immunosuppressant that also binds FKBP12 but unlike tacrolimus it does not inhibit calcineurin. Furthermore, FKBP12-binding analogs of tacrolimus that do not inhibit calcineurin function are devoid of toxicity and the antagonist of FK506-induced immunosuppression, L-685,818, can block FK506-induced toxicity in animal models.

A. Side Effects of Tacrolimus

Liver Transplantation

The principal adverse reactions of Prograf are tremor, headache, diarrhea, hypertension, nausea, and abnormal renal function. These occur with oral and IV administration of Prograf and may respond to a reduction in dosing. Hyperglycemia has also been noted in many patients, requiring insulin therapy in some patients (see tables below).

The incidence of adverse events was determined in two randomized comparative liver transplant trials among 514 patients receiving tacrolimus and steroids and 515 patients receiving a cyclosporine-based regimen (CBIR). The proportion of patients reporting more than one adverse event was 99.8% in the tacrolimus group and 99.6% in the CBIR group. Precautions must be taken when comparing the incidence of adverse events in the U.S. study to that in the European study. The 12-month post-transplant information from the U.S. study and from the European study is presented below. The two studies also included different patient populations and patients were treated with immunosuppressive regimens of differing intensities. Hyperglycemia was reported by 44% of the liver transplant patients taking Prograf in the US study and 33% of the liver transplant patients taking Prograf in the European study. Part of the adverse events reported in ≧15% in tacrolirnus patients (combined study results) are presented below for the two controlled trials in liver transplantation:

LIVER TRANSPLANTATION: ADVERSE EVENTS OCCURRING
IN ≧15% OF PROGRAF-TREATED PATIENTS

| | U.S. STUDY | | EUROPEAN STUDY | |
|---|---|---|---|---|
| | Prograf | CBIR | Prograf | CBIR |
| | (N = 250) | (N = 250) | (N = 264) | (N = 265) |
| Nervous System | | | | |
| Headache | 64% | 60% | 37% | 26% |
| Tremor | 56% | 16% | 48% | 32% |
| Insomnia | 64% | 68% | 32% | 23% |
| Paresthesia | 40% | 30% | 17% | 17% |
| Gastrointestinal | | | | |
| Diarrhea | 72% | 47% | 37% | 27% |
| Nausea | 46% | 37% | 32% | 27% |
| Constipation | 24% | 27% | 23% | 21% |
| LFT Abnormal | 36% | 30% | 6% | 5% |
| Anorexia | 34% | 24% | 7% | 5% |
| Vomiting | 27% | 15% | 14% | 11% |
| Cardiovascular | | | | |
| Hypertension | 47% | 56% | 38% | 43% |
| Urogenital | | | | |
| Kidney Function | 40% | 27% | 36% | 23% |
| Abnormal | | | | |
| Creatinine Increased | 39% | 25% | 24% | 19% |
| BUN Increased | 30% | 22% | 12% | 9% |
| Urinary Tract Infection | 16% | 18% | 21% | 19% |
| Oliguria | 18% | 15% | 19% | 12% |
| Metabolic and | | | | |
| Nutritional | | | | |
| Hyperkalemia | 45% | 26% | 13% | 9% |
| Hypokalemia | 29% | 34% | 13% | 16% |
| Hyperglycemia | 47% | 38% | 33% | 22% |
| Hypomagnesemia | 48% | 45% | 16% | 9% |

Kidney Transplantation

The most common adverse reactions reported were infection, tremor, hypertension, abnormal renal function, constipation, diarrhea, headache, abdominal pain and insomnia. Hyperglycemia was reported by 22% of kidney transplant patients taking Prograf. Part of the adverse events that occurred in ≧15% of Prograf-treated kidney transplant patients are presented below:

KIDNEY TRANSPLANTATION: ADVERSE EVENTS OCCURRING
IN ≧15% OF PROGRAF-TREATED PATIENTS

| | Prograf | CBIR |
|---|---|---|
| | (N = 205) | (N = 207) |
| Nervous System | | |
| Tremor | 54% | 34% |
| Headache | 44% | 38% |
| Insomnia | 32% | 30% |
| Paresthesia | 23% | 16% |
| Dizziness | 19% | 16% |
| Gastrointestinal | | |
| Diarrhea | 44% | 41% |
| Nausea | 38% | 36% |
| Constipation | 35% | 43% |
| Vomiting | 29% | 23% |
| Dyspepsia | 28% | 20% |
| Cardiovascular | | |
| Hypertension | 50% | 52% |
| Chest pain | 19% | 13% |
| Urogenital | | |
| Creatinine Increased | 45% | 42% |
| Urinary Tract Infection | 34% | 35% |
| Metabolic and Nutritional | | |
| Hypophosphatemia | 49% | 53% |
| Hypomagnesemia | 34% | 17% |
| Hyperlipemia | 31% | 38% |
| Hyperkalemia | 31% | 32% |
| Diabetes Mellitus | 24% | 9% |
| Hypokalemia | 22% | 25% |
| Hyperglycemia | 22% | 16% |
| Edema | 18% | 19% |
| Hemic and Lymphatic | | |
| Anemia | 30% | 24% |
| Leukopenia | 15% | 17% |
| Miscellaneous | | |
| Infection | 45% | 49% |
| Peripheral Edema | 36% | 48% |

Heart Transplantation

The more common adverse reactions in Prograf-treated heart transplant recipients were abnormal renal function, hypertension, diabetes mellitus, CMV infection, tremor, hyperglycemia, leukopenia, infection, and hyperlipemia. Part of the adverse events in heart transplant patients in the European trial are presented below:

HEART TRANSPLANTATION: ADVERSE EVENTS OCCURRING
IN ≧15% OF PROGRAF-TREATED PATIENTS

| | Prograf + | CsA + |
|---|---|---|
| COSTART Body System | Azathioprine | Azathioprine |
| COSTART Term | (n = 157) | (n = 157) |
| Cardiovascular System | | |
| Hypertension | 62% | 69% |
| Pericardial effusion | 15% | 14% |
| Body as a Whole | | |
| CMV infection | 32% | 30% |
| Infection | 24% | 21% |
| Metabolic and Nutritional Disorders | | |
| Hyperlipemia | 18% | 27% |
| Diabetes Mellitus | 26% | 16% |
| Hyperglycemia | 23% | 17% |
| Hemic and Lymphatic System | | |
| Leukopenia | 48% | 39% |
| Anemia | 50% | 36% |

In the European study, the cyclosporine trough concentrations were above the pre-defined target range (i.e., 100-200 ng/mL) at Day 122 and beyond in 32-68% of the patients in the cyclosporine treatment arm, whereas the tacrolimus trough concentrations were within the pre-defined target range (i.e., 5-15 ng/mL) in 74-86% of the patients in the tacrolimus treatment arm. Only selected targeted treatment-emergent adverse events were collected in the US heart transplantation study. Those events that were reported at a rate of 15% or greater in patients treated with Prograf and mycophenolate mofetil include the following: any target adverse events (99.1%), hypertension (88.8%), hyperglycemia requiring antihyperglycemic therapy (70.1%), hypertriglyceridemia (65.4%), anemia (hemoglobin<10.0 g/dL) (65.4%), fasting blood glucose>140 mg/dL (on two separate occasions) (60.7%), hypercholesterolemia (57.0%), hyperlipidemia (33.6%), WBCs<3000 cells/mcL (33.6%), serious bacterial infections (29.9%), magnesium<1.2 mEq/L (24.3%), platelet count<75,000 cells/mcL (18.7%), and other opportunistic infections (15.0%). Other targeted treatment-emergent adverse events in Prograf-treated patients occurred at a rate of less than 15%, and include the following: Cushingoid features, impaired wound healing, hyperkalemia, Candida infection, and CMV infection/syndrome.

### Hyperglycemia

Hyperglycemia, hyperglycaemia, or high blood sugar is a condition in which a high amount of glucose circulates in the blood plasma. Glucose levels vary before and after meals, and at various times of day; the definition of normal varies among medical professionals. In general, the normal range for most people (fasting adults) is about 80 to 120 mg/dL or 4 to 7 mmol/L. A subject with a consistent range above 126 mg/dL or 7 mmol/L is generally held to have hyperglycemia, whereas a consistent range below 70 mg/dL or 4 mmol/L is considered hypoglycemic. In fasting adults, blood plasma glucose should not exceed 126 mg/dL or 7 mmol/L. Sustained higher levels of blood sugar cause damage to the blood vessels and to the organs they supply, leading to the complications of diabetes. Chronic hyperglycemia can be measured via the HbA1c test. The definition of acute hyperglycemia varies by study, with mmol/L levels from 8 to 15.

Chronic hyperglycemia that persists even in fasting states is most commonly caused by diabetes mellitus, and in fact chronic hyperglycemia is the defining characteristic of the disease. Acute episodes of hyperglycemia without an obvious cause may indicate developing diabetes or a predisposition to the disorder. This form of hyperglycemia is caused by low insulin levels. These low insulin levels inhibit the transport of glucose across cell membranes therefore causing high blood glucose levels.

Certain eating disorders can produce acute non-diabetic hyperglycemia, as in the binge phase of bulimia nervosa, when the subject consumes a large amount of calories at once, frequently from foods that are high in simple and complex carbohydrates. Certain medications increase the risk of hyperglycemia, including beta blockers, thiazide diuretics, corticosteroids, niacin, pentamidine, protease inhibitors, L-asparaginase, and some antipsychotic agents.

A high proportion of patients suffering an acute stress such as stroke or myocardial infarction may develop hyperglycemia, even in the absence of a diagnosis of diabetes. Human and animal studies suggest that this is not benign, and that stress-induced hyperglycemia is associated with a high risk of mortality after both stroke and myocardial infarction.

Hyperglycemia occurs naturally during times of infection and inflammation. When the body is stressed, endogenous catecholamines are released that—amongst other things—serve to raise the blood glucose levels. The amount of increase varies from person to person and from inflammatory response to response.

The invention provides compositions and methods utilizing an agent that reduces or eliminates hyperglycemia and/or one or more symptoms of hyperglycemia. Typically, the hyperglycemia-decreasing agent is a modulator of a blood tissue barrier (BTB). It will be appreciated that the mechanism of BTB protein modulator for the reduction of hyperglycemia and/or one or more symptoms of hyperglycemia might be through a different mechanism than modulation of a BTB protein transport.

The methods and compositions are useful in the treatment of an animal in need of treatment, where it is desired that hyperglycemia and/or one or more symptoms of hyperglycemia be reduced or eliminated.

The agent causing a decrease in hyperglycemia and/or one or more symptoms of hyperglycemia, e.g., a modulator of a BTB transport protein may be an activator or an inhibitor of the protein. The modulatory effect may be dose-dependent, e.g., some modulators act as activators in one dosage range and inhibitors in another. In some embodiments, a modulator of a BTB transport protein is used in a dosage wherein it acts primarily as an activator.

The BTB transport protein modulator is a BTB activator in some embodiments. In some embodiments the BTB transport protein modulator is a modulator of ATP binding cassette (ABC) transport proteins. In some embodiments the BTB transport protein modulator is a modulator of P-glycoprotein (P-gP).

In some embodiments, compositions of the invention include one or more BTB transport protein modulators. In addition, a BTB transport modulator itself may be metabolized to metabolites that have differing activities in the modulation of one or more BTB transport proteins, and these metabolites are also encompassed by the compositions and methods of the invention.

BTB transport protein modulators of use in the invention include any suitable BTB transport modulators. In some embodiments, the BTB transport protein modulator is one or more pyrone analogs. In other embodiments, the BTB transport protein modulator is one or more polyphenols. In some embodiments, the BTB transport protein modulator is one or more flavonoids. In some embodiments, the BTB transport protein modulator is quercetin or a quercetin derivative. In some embodiments the BTB transport protein modulator is fisetin or a fisetin derivative.

In some embodiments, the pyrone analogs disclosed herein are modified. In some aspects, the modification includes phosphorylation, glycosylation, acylation or combinations thereof. In some embodiments, the phosphorylated pyrone analog is a phosphorylated polyphenol. In other embodiments, the phosphorylated pyrone analog is a phosphorylated flavonoid. In yet another embodiment, the phosphorylated pyrone analog is quercetin or a quercetin derivative. In some embodiments, the phosphorylated pyrone analog is fisetin or a fisetin derivative.

In some embodiments of the invention, the compositions further comprise an oligosaccharide. In some embodiments, the oligosaccharide is a cyclic oligosaccharide. In some embodiments, the oligosaccharide is a cyclodextrin. In some embodiments, the cyclodextrin is a sulfo-alkyl ether substituted cyclodextrin or a sulfobutyl-ether substituted cyclodextrin. In some embodiments, the cyclodextrin is hydroxypropyl-β-cyclodextrin, hydroxypropyl-γ-cyclodextrin, sulfobutylether-β-cyclodextrin, sulfobutylether-7-β-cyclodextrin, or combinations thereof.

In some embodiments the invention provides methods of treatment. In certain embodiments, the invention provides a method of treating a condition by administering to an animal suffering from the condition an amount of a BTB transport protein modulator, e.g., activator, sufficient to reduce or eliminate hyperglycemia and/or one or more symptoms of hyperglycemia. In certain embodiments the invention provides methods of treatment of chronic hyperglycemia, acute hyperglycemia, diabetes mellitus, non-diabetic hyperglycemia, stress-induced hyperglycemia, inflammation-induced hyperglycemia, by administering a modulator of a BTB transport protein, thereby reducing or eliminating hyperglycemia and/or one or more symptoms of hyperglycemia.

In some embodiments, the symptom of hyperglycemia can be glucosuria, polyphagia, polyuria, polydipsia, loss of consciousness, blurred vision, headaches, coma, ketoacidosis, decrease in blood volume, decrease in renal bloodflow, accelerated lipolysis, weight loss, stomach problems, intestinal problems, poor wound healing, dry mouth, nausea, vomiting, dry skin, itchy skin, impotence, hypeventilation, ketoanemia, fatigue, weakness on one side of the body, hallucinations, impairment in cognitive function, increase sadness, anxiety, recurrent genital infections, increase sugar in urine, retinopathy, nepropathy, arteriosclerotic disorders, cardiac arrhythmia, stupor, susceptibility to infection, neuropathy, nerve damages causing cold feet, nerve damage causing insensitive feet and loss of hair, or combinations thereof. In some embodiments, the symptom of hyperglycemia is glucosuria.

A. Hyperglycemia Induced by Calcineurin Inhibitors

As noted above tacrolimus induced hyperglycemia in transplant patients (see side effects section). The incidence of posttransplant diabetes mellitus (PTDM) in renal transplant recipients treated with tacrolimus ranges from 10-30% in western countries and is 31.4% in Japan. PTDM is associated with increased cardiovascular diseases and infection in transplant recipients. With better survival rates, PTDM has been recognized as a more serious complication than previously considered.

The exact mechanism of hyperglycemia associated with tacrolimus in not known, although it might be attributed to accumulation of the drug in the pancreatic islet cells. Without intending to be limited to any theory, it has been suggested that tacrolimus impairs insulin secretion at multiple steps in stimulus-secretion coupling (Uchizono et al. Endocrinology 2004, 145(5): 2264-2272). These authors observed that tacrolimus caused reductions in DNA and insulin contents per islet during 7-d culture. In addition, their experiments showed that tacrolimus time-dependently suppressed glucose-stimulated insulin secretion, and at a therapeutic concentration of 0.01 μmol/liter, it suppressed glucose-stimulated insulin secretion to 32±5% of the control value after 7-d incubation. They further observed that, tacrolimus suppressed insulin secretion stimulated by mitochondrial fuel (combination of L-leucine and L-glutamine, and -ketoisocaproate) and glibenclamide, but not by L-arginine. Their experiments also indicated that tacrolimus suppressed insulin secretion induced by carbachol and by a protein kinase C agonist in the presence or absence of extracellular Ca2+; and that under stringent Ca2+-free conditions, tacrolimus did not affect mastoparan-induced insulin secretion, but suppressed its glucose augmentation. The authors suggested that tacrolimus may impair glucose-stimulated insulin secretion downstream of the rise in intracellular Ca2+ at insulin exocytosis, and that protein kinase C-mediated (Ca2+-dependent and independent) and Ca²+-independent GTP signaling pathways may be involved.

Other mechanisms for the hyperglycemic effect induced by tacrolimus have been proposed. Experiments with animals and human pancreas allograft biopsies indicate that long-term tacrolimus treatment causes cytoplasmic swelling, vacuolization, and apoptosis of β-cells (Hirano et al. 1992, Transplantation 53:889-894 and Drachenberg et al. 1999, Transplantation 68:396-402). In addition, the binding of FK506-binding protein 12.6 (FKBP 12.6) to cyclic ADP-ribose (cADPR), a possible second messenger for glucose-stimulated insulin secretion, in islet microsomes leads to increased Ca2+ release via ryanodine receptor and enhanced insulin secretion (Takasawa at al. 1993, Science 259:370-373 and Okamoto et al. 1997, Diabetologia 40:1485-1491). This pathway is suppressed by tacrolimus suppresses by its binding with FKBP 12.6 (Noguchi et al. 1997, J Biol Chem 272:3133-3136). Moreover, it has been reported that insulin gene transcription is regulated by NFAT, which is activated by Ca2+-dependent calcineurin in β-cells (Lawrence et al. 2001, Mol Endocrinol 15:1758-1767). Tacrolimus suppressed glucose-stimulated insulin gene expression, leading to reduced insulin synthesis and contents. Also, insulin storage β-granules are transported to the cell surface along microtubules, driven by a motor molecule such as kinesin (Balczon et al. 1992, Endocrinology 131:331-336 and Meng et al. 1997, Endocrinology 138:1979-1987). Kinesin heavy chain is activated through dephosphorylation mediated by calcineurin. Suppression of calcineurin activity inhibits dephosphorylation of kinesin heavy chain as well as the second phase of glucose-stimulated insulin secretion. Lastly, it has been reported that glucose-stimulated insulin release is decreased by chronic exposure to tacrolimus due to reduced ATP production and glycolysis derived from reduced glucokinase activity (Radu et al. 2005, Am J Physiol Endocrinol Metab 288: E365-E371)

BREAK The invention provides compositions and methods utilizing an agent that reduces or eliminates hyperglycemia and/or one or more symptoms of hyperglycemia induced by a calcineurin inhibitor treatment. The invention also provides compositions and methods utilizing an agent as described herein that increases a therapeutic effect associated with calcineurin inhibitor treatment. The invention also provides compositions and methods utilizing an agent that changes the concentration in a physiological compartment of a calcineurin inhibitor.

In some embodiments, the invention provides compositions and methods utilizing a combination of a calcineurin inhibitor and an agent that reduces or eliminates hyperglycemia and/or one or more symptoms of hyperglycemia induced by a calcineurin inhibitor treatment. Typically, the hyperglycemia-decreasing agent is a modulator of a blood tissue barrier (BTB). However, it is recognized that the mechanism of action of a particular BTB transport protein modulator in decreasing one or more symptoms as described herein may be different, or in addition to, modulation of a BTB transport protein, and that an agent that has BTB transport protein-modulating activity may nonetheless act by a different mechanism than BTB transport protein modulation. It is also possible for an agent to modulate more than one BTB transport protein, and the overall effect will depend on the summation of all mechanisms by which an agent works.

The methods and compositions are useful in the treatment of an animal in need of treatment, where it is desired that hyperglycemia and/or one or more symptoms of hyperglycemia induced by a calcineurin inhibitor be reduced or eliminated. In embodiments further utilizing a calcineurin inhibitor, the methods and compositions are useful in the treatment of an animal in need of treatment, where it is desired that hyperglycemia and/or one or more symptoms of hyperglycemia induced by a calcineurin inhibitor be reduced or eliminated while one or more of the therapeutic effects (e.g., peripheral effects) of the calcineurin inhibitor are retained or enhanced. In some embodiments, the animal receiving treatment with a calcineurin inhibitor is known or is suspected to have hyperglycemia and/or one or more symptoms of hyperglycemia. In some embodiments, the methods and compositions of the invention utilize an agent that changes the concentration of a calcineurin inhibitor in a physiological compartment.

In some embodiments of the invention, the calcineurin inhibitor is tacrolimus or a tacrolimus analog. Examples of tacrolimus analogs include, but are not limited to, meridamycin, 31-O-Demethyl-FK506; L-683,590, L-685,818; 32-O-(1-hydroxyethylindol-5-yl)ascomycin; ascomycin; C18-OH-ascomycin; 9-deoxo-31-O-demethyl-FK506; L-688,617; A-119435; AP1903; rapamycin; dexamethasone-FK506 heterodimer; 13-O-demethyl tacrolimus; and FK 506-dextran conjugate.

The agent causing a decrease in hyperglycemia and/or one or more symptoms of hyperglycemia induced by the calcineurin inhibitor, and/or an increase in a therapeutic effect of a calcineurin inhibitor, and/or a change in concentration of the calcineurin inhibitor in a physiological compartment, e.g., a modulator of a BTB transport protein may be an activator or an inhibitor of the protein. The modulatory effect may be dose-dependent, e.g., some modulators act as activators in one dosage range and inhibitors in another. In some embodiments, a modulator of a BTB transport protein is used in a dosage wherein it acts primarily as an activator.

Typically, the use of the BTB protein modulator, e.g., activator, results in a decrease in hyperglycemia and/or one or more symptoms of hyperglycemia induced by the calcineurin inhibitor. The therapeutic effect(s) of the calcineurin inhibitor may be decreased, remain the same, or increase; however, in preferred embodiments, if the therapeutic effect is decreased, it is not decreased to the same degree as the hyperglycemia and/or a symptom of hyperglycemia. It will be appreciated that a given calcineurin inhibitor may have more than one therapeutic effect and or one or more symptoms of hyperglycemia, and it is possible that the therapeutic ratio (in this case, the ratio of change in desired effect to change in undesired symptom) may vary depending on which effect is measured. However, at least one therapeutic effect of the calcineurin inhibitor is decreased to a lesser degree than at least one symptom of hyperglycemia induced by the calcineurin inhibitor. In some embodiments, the use of the BTB transport protein modulator does not affect the therapeutic effect(s) of the calcineurin inhibitor.

In addition, in some embodiments, one or more therapeutic effects of the calcineurin inhibitor are enhanced by the use of the calcineurin inhibitor in combination with a BTB transport protein modulator, while hyperglycemia and/or one or more symptoms of hyperglycemia induced by the calcineurin inhibitor are reduced or substantially eliminated. For example, in some embodiments, the immunosuppressant effect of the calcineurin inhibitor is enhanced while hyperglycemia and/or one or more symptoms of hyperglycemia induced by the calcineurin inhibitor is reduced or substantially eliminated.

In some embodiments, the concentration of the calcineurin inhibitor is changed in a physiological compartment by using the calcineurin inhibitor in combination with a BTB transport protein modulator. Examples of physiological compartments include, but are not limited to, blood, kidney and pancreatic islet cells.

Without being bound by theory, and as an example only of a possible mechanism, it is thought that the methods and compositions of the invention operate by reducing or eliminating the concentration of the calcineurin inhibitor from the compartment where the side effect is produced (e.g., pancreatic islet cells, kidney), while retaining or even increasing the effective concentration of the calcineurin inhibitor in the periphery and/or compartment where the therapeutic effect is desired. Calcineurin inhibitors act at least in part by peripheral mechanisms (e.g. inhibition of T lymphocyte activation) and may thus retain some or all of their activity, or even display enhanced therapeutic activity, while at the same time hyperglycemia and/or one or more symptoms of hyperglycemia are reduced or eliminated.

In some embodiments, the BTB transport protein modulator decreases the clearance of the calcineurin inhibitor from the compartment where the calcineurin inhibitor is exerting its therapeutic effect, while hyperglycemia and/or one or more symptoms of hyperglycemia induced by the calcineurin inhibitor are reduced or substantially eliminated. Without being limited to any theory, and as an example only of a possible mechanism, it is thought that the methods and compositions of the invention operate by reducing or eliminating the concentration of the calcineurin inhibitor from the compartment where the calcineurin inhibitor is cleared from the animal (e.g., liver), hence, retaining or even increasing the effective concentration of the calcineurin inhibitor in the periphery and/or compartment where the therapeutic effect is desired.

It will be appreciated that the therapeutic effect and/or inducement of hyperglycemia may be mediated in part or in whole by one or more metabolites of the calcineurin inhibitor, and that a BTB transport modulator that reduces or eliminates the concentration of the calcineurin inhibitor and/or of one or active metabolites of the calcineurin inhibitor in the compartment that produce side effects, while retaining or enhancing the concentration of the calcineurin inhibitor and/or one or more metabolites in the periphery and/or compartment that produces a therapeutic effect, is also encompassed by the methods and compositions of the invention. In addition, a BTB transport modulator itself may be metabolized to metabolites that have differing activities in the modulation of one or more BTB transport modulators, and these metabolites are also encompassed by the compositions and methods of the invention.

In some embodiments the invention provides compositions that include a calcineurin inhibitor and a BTB transport modulator where the calcineurin inhibitor is present in an amount sufficient to exert a therapeutic effect and the BTB transport modulator is present in an amount sufficient to decrease hyperglycemia and/or one or more symptoms of hyperglycemia induced by the calcineurin inhibitor when compared to the hyperglycemia or symptom of hyperglycemia without the BTB transport modulator when the composition is administered to an animal. The decrease in hyperglycemia can be measurable. In some embodiments the invention provides compositions that include a calcineurin inhibitor and a BTB transport protein modulator, where the calcineurin inhibitor is present in an amount sufficient to exert a therapeutic effect and the BTB transport protein modulator is present in an amount sufficient to change the concentration of the calcineurin inhibitor in a physiological compartment when compared to concentration of the calcineurin inhibitor in the physiological compartment without the BTB transport protein modulator, when the composition is administered to an animal. In some embodiments, the BTB transport protein modulator increases the concentration of a calcineurin inhibitor in a physiological compartment where a therapeutic effect is desired (e.g. periphery and/or T cells). In some embodiments, the BTB transport protein modulator decreases the concentration of a calcineurin inhibitor in a physiological compartment where hyperglycemia and/or one or more symptoms of hyperglycemia are produced (e.g. pancreatic islet cells). The change in concentration of the calcineurin inhibitor modulator in a physiological compartment can be measurable. The BTB transport protein modulator is a BTB activator in some embodiments. In some embodiments the BTB transport protein modulator is a modulator of ATP binding cassette (ABC) transport proteins. In some embodiments the BTB transport protein modulator is a modulator of P-glycoprotein (P-gP).

In some embodiments, compositions of the invention include one or more calcineurin inhibitor as well as one or more than one BTB transport protein modulator. One or more of the calcineurin inhibitors may induce one or more symptoms of hyperglycemia which are desired to be decreased.

It will be appreciated that when a BTB transport protein that is the target of the BTB transport modulator is present on the cells where the calcineurin inhibitor is exerting its therapeutic effect, the dosage of the BTB transport modulator may be adjusted such that hyperglycemia and/or one or more symptoms of hyperglycemia induced by the calcineurin inhibitor are reduced without a substantial reduction of the therapeutic effect in the target cells. In some embodiments, it is desirable to inhibit a BTB transport protein present in the cells where the calcineurin inhibitor is exerting its therapeutic effect while activating the same or another BTB transport protein at other site(s) such that hyperglycemia and/or one or more symptoms of hyperglycemia induced by the calcineurin inhibitor are reduced. Therefore, the dosage of the BTB transport modulator may be adjusted such that a BTB transport protein that is the target of the BTB transport modulator is inhibited on the cells where the calcineurin inhibitor is exerting its therapeutic effect, while the same or another BTB transport protein is activated on other site(s) to reduce the side effect of the calcineurin inhibitor.

Compositions of the invention may be prepared in any suitable form for administration to an animal. In some embodiments, the invention provides pharmaceutical compositions.

In some embodiments, the invention provides compositions suitable for oral administration. In some embodiments, compositions are suitable for transdermal administration. In some embodiments, compositions are suitable for injection by any standard route of injection, e.g., intravenous, subcutaneous, intramuscular, or intraperitoneal. Compositions suitable for other routes of administration are also encompassed by the invention, as described herein.

BTB transport protein modulators of use in the invention include any suitable BTB transport modulators. In some embodiments, the BTB transport protein modulator is one or more pyrone analogs. In some embodiments, the BTB transport protein modulator is one or more polyphenols. In some embodiments, the BTB transport protein modulator is one or more flavonoids. In some embodiments, the BTB transport protein modulator is quercetin or a quercetin derivative, or fisetin or a fisetin derivative. In some embodiments, the BTB transport protein modulator is modified, e.g. phosphorylated, glycosylated or acylated. In some embodiments, the BTB transport protein modulator is quercetin phosphate or a derivative, or fisetin phosphate or a derivative.

In some embodiments the invention provides methods of treatment. In certain embodiments, the invention provides a method of treating a condition by administering to an animal suffering from the condition an effective amount of a calcineurin inhibitor and an amount of a BTB transport protein modulator, e.g., activator, sufficient to reduce or eliminate hyperglycemia and/or one or more symptoms of hyperglycemia induced by the calcineurin inhibitor. In some embodiments the BTB transport protein modulator is a BTB transport protein activator. In some embodiments, the calcineurin inhibitor is tacrolimus or a tacrolimus analog. In certain embodiments the invention provides methods of treatment of organ transplant, an autoimmune disease, or an inflammatory disease with a calcineurin inhibitor, by co-administering a modulator of a BTB transport protein in combination with the calcineurin inhibitor, thereby reducing or eliminating hyperglycemia and/or one or more symptoms of hyperglycemia induced by the calcineurin inhibitor. In some embodiments, the invention provides methods for treatment of organ transplant. Example of organ transplant include but are not limited to kidney transplant, pancreas transplant, liver transplant, heart transplant, lung transplant, intestine transplant, pancreas after kidney transplant, and simultaneous pancreas-kidney transplant. In other embodiments, the invention provides methods for the treatment of an autoimmune disease. Examples of autoimmune diseases include, but are not limited to, Rheumatoid Arthritis, Lupus nephritis, actopic dermatitis, and psoriasis. In yet other embodiments, the invention provides methods for the treatment of inflammatory diseases. Examples of inflammatory diseases include, but are not limited to, asthma, vulvar lichen sclerosis, chronic allergic contact dermatitis, eczema, vitiligo and ulcerative colitis

In some embodiments the invention provides methods of decreasing hyperglycemia and/or one or more symptoms of hyperglycemia induced by a calcineurin inhibitor in an animal, e.g. a human, that has received an amount of the calcineurin inhibitor sufficient to produce hyperglycemia by administering to the animal, e.g., human, an amount of a BTB transport protein modulator sufficient to reduce or eliminate hyperglycemia and/or one or more symptoms of hyperglycemia.

In some embodiments, the methods and compositions of the present invention can be used to modulate transport of a variety of calcineurin inhibitors. In some embodiments, the dosage of the calcineurin inhibitor is modulated according to the effect of the transport protein modulator. For instance, less calcineurin inhibitor may be needed to reach optimal effect when co-administered with the transport protein modulator. In other embodiments co-administering the transport protein modulator with a calcineurin inhibitor allows for chronically administering the drug without drug escalation and/or without dependence on the drug. In another embodiment co-administering the transport protein modulator allows for the decrease or elimination of a calcineurin inhibitor from a physiological compartment. In some embodiments, the physiological compartment is a pancreatic islet cell.

In some embodiments the invention provides methods of decreasing hyperglycemia and/or one or more symptoms of hyperglycemia induced by a calcineurin inhibitor in an animal, e.g. a human, that has received an amount of the calcineurin inhibitor sufficient to produce hyperglycemia by administering to the animal, e.g., human, an amount of a BTB transport protein modulator sufficient to reduce or eliminate hyperglycemia and/or one or more symptoms of hyperglycemia. The term “symptom,” as used herein, encompasses any symptom of hyperglycemia. The symptom may be acute or chronic. The symptom may be biochemical, cellular, at the tissue level, at the organ level, at the multi-organ level, or at the level of the entire organism. The symptom may manifest in one or more objective or subjective manners, any of which may be used to measure the effect. For some substances that may be normally or abnormally produced the symptom may be a pathological symptom.

In some embodiments, the symptom of hyperglycemia can be glucosuria, polyphagia, polyuria, polydipsia, loss of consciousness, blurred vision, headaches, coma, ketoacidosis, decrease in blood volume, decrease in renal bloodflow, accelerated lipolysis, weight loss, stomach problems, intestinal problems, poor wound healing, dry mouth, nausea, vomiting, dry skin, itchy skin, impotence, hypeventilation, ketoanemia, fatigue, weakness on one side of the body, hallucinations, impairment in cognitive function, increase sadness, anxiety, recurrent genital infections, increase sugar in urine, retinopathy, nepropathy, arteriosclerotic disorders, cardiac arrhythmia, stupor, susceptibility to infection, neuropathy, nerve damages causing cold feet, nerve damage causing insensitive feet and loss of hair, or combinations thereof.

In another embodiment co-administering the transport protein modulator will allow for a change in concentration of a calcineurin inhibitor in a physiological compartment, e.g. increase of calcineurin inhibitor in the periphery and/or decrease of calcineurin inhibitors in pancreatic islet cells.

If a hyperglycemia or a symptom of hyperglycemia is measured objectively or subjectively (e.g., glucosuria, loss of consciousness, and the like), any suitable method for evaluation of objective or subjective symptom may be used. An example of an objective measure for hyperglycemia is measurement of blood glucose, e.g., fasting glucose. Measurements of blood glucose can be performed by any method known in the art such as the fasting blood sugar or glucose test (FBS), the urine glucose test, the two-hr postprandial blood sugar test (2-h PPBS), the oral glucose tolerance test (OGTT), intravenous glucose tolerance test (IVGTT), glycosylated hemoglobin (HbA1C) or a self-monitoring test of glucose level via home kits. Examples for subjective symptoms include visual and numeric scales and the like for evaluation by an individual. A further example includes sleep latency for measurement of drowsiness, or standard tests for measurement of concentration, mentation, memory, and the like. These and other methods of objective and subjective evaluation of side effects by either an objective observer, the individual, or both, are well-known in the art.

A “therapeutic effect,” as that term is used herein, encompasses a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made. A prophylactic effect includes delaying or eliminating the appearance of a disease or condition, delaying or eliminating the onset of symptoms of a disease or condition, slowing, halting, or reversing the progression of a disease or condition, or any combination thereof.

The term “physiological compartment” as used herein includes physiological structures, such as organs or organ groups or the fetal compartment, or spaces whereby a physiological or chemical barrier exists to exclude compounds or agents from the internal or external portion of the physiological structure or space. Such physiological compartments include the central nervous system, blood and other bodily fluids, the fetal compartment, internal structures contained within organs, such as the ovaries and testes, and cells such as pancreatic islet cells.

### Compositions

In some embodiments, the invention provides compositions that include an agent, e.g., that reduces or eliminates hyperglycemia and/or one or more symptoms of hyperglycemia. In some embodiments, the invention provides compositions that include an agent, e.g., that reduces or eliminates hyperglycemia and/or one or more symptoms of hyperglycemia induced by a calcineurin inhibitor. In some embodiments, the calcineurin inhibitor is co-administered with the agent that reduces hyperglycemia and/or one or more symptoms of hyperglycemia induced by the calcineurin inhibitor. “Co-administration,” “administered in combination with,” and their grammatical equivalents, as used herein, encompasses administration of two or more agents to an animal so that both agents and/or their metabolites are present in the animal at the same time. Co-administration includes simultaneous administration in separate compositions, administration at different times in separate compositions, or administration in a composition in which both agents are present.

In some embodiments, the invention provides compositions containing a combination of a calcineurin inhibitor and an agent, e.g., that reduces or eliminates hyperglycemia and/or one or more symptoms of hyperglycemia induced by the calcineurin inhibitor. In some embodiments, the invention provides compositions containing a combination of a calcineurin inhibitor and an agent that changes the concentration in a physiological compartment of the calcineurin inhibitor. In some embodiments the invention provides pharmaceutical compositions that further include a pharmaceutically acceptable excipient. In some embodiments, the pharmaceutical compositions are suitable for oral administration. In some embodiments, the pharmaceutical compositions are suitable for transdermal administration. In some embodiments, the pharmaceutical compositions are suitable for injection. Other forms of administration are also compatible with embodiments of the pharmaceutical compositions of the invention, as described herein.

In some embodiments, the BTB transport protein is an ABC transport protein. In some embodiments, the BTB transport protein modulator is a BTB transport protein activator. In some embodiments, the BTB transport protein modulator is a BTB transport protein inhibitor. In some embodiments, the BTB transport protein modulator is a modulator of P-gP.

In some embodiments, the BTB transport protein modulator comprises a pyrone analog. In some embodiments, the BTB transport protein modulator is a polyphenol. In other embodiments, a polyphenol which acts to decrease hyperglycemia and/or one or more symptoms of hyperglycemia through a non-BTB transport protein-mediated mechanism, or that acts to lower hyperglycemia and/or one or more symptoms of hyperglycemia through a BTB transport protein-mediated mechanism and a non-BTB transport protein-mediated mechanism, is used. In other embodiments, a polyphenol which acts to lower hyperglycemia and/or one or more symptoms of hyperglycemia induced by a calcineurin inhibitor through a non-BTB transport protein-mediated mechanism, or that acts to lower hyperglycemia and/or one or more symptoms of hyperglycemia induced by a calcineurin inhibitor through a BTB transport protein-mediated mechanism and a non-BTB transport protein-mediated mechanism, is used. In other embodiments, a polyphenol which acts to increase a therapeutic effect of a calcineurin inhibitor through a non-BTB transport protein-mediated mechanism, or that acts to increase a therapeutic effect of a calcineurin inhibitor through a BTB transport protein-mediated mechanism and a non-BTB transport protein-mediated mechanism, is used. In other embodiments, a polyphenol which acts to increase the concentration of a calcineurin inhibitor in a physiological compartment through a non-BTB transport protein-mediated mechanism, or that acts to increase the concentration of a calcineurin inhibitor in a physiological compartment through a BTB transport protein-mediated mechanism and a non-BTB transport protein-mediated mechanism, is used. In other embodiments, a polyphenol which acts to decrease the concentration of a calcineurin inhibitor in a physiological compartment through a non-BTB transport protein-mediated mechanism, or that acts to decrease the concentration of a calcineurin inhibitor in a physiological compartment through a BTB transport protein-mediated mechanism and a non-BTB transport protein-mediated mechanism, is used. In some embodiments utilizing a polyphenol, the polyphenol is a flavonoid. In some embodiments utilizing a polyphenol, the polyphenol is selected from the group consisting of quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, and epicatechin, or combinations thereof. In some embodiments utilizing a polyphenol, the polyphenol is a flavonol. In certain embodiments, the flavonol is selected from the group consisting of quercetin, galangin, fisetin and kaempferol, or combinations thereof. In some embodiments, the flavonol is quercetin or a quercetin derivative. In some embodiments, the flavonol is galangin or a galangin derivative. In some embodiments, the flavonol is kaempferol or a kaempferol derivative. In some embodiments, the flavonol is fisetin or a fisetin derivative.

In some embodiments, the compositions include a modified pyrone analog. In some embodiments, the modified pyrone analog is a phosphorylated polyphenol. In other embodiments, the pyrone analog is a phosphorylated flavonoid, such as a phosphorylated quercetin or quercetin derivative and/or fisetin or fisetin derivative, that acts to decrease hyperglycemia and/or one or more symptoms of hyperglycemia through a non-BTB transport protein-mediated mechanism, or that acts to lower hyperglycemia and/or one or more symptoms of hyperglycemia through a BTB transport protein-mediated mechanism and a non-BTB transport protein-mediated mechanism. In other embodiments, a phosphorylated polyphenol, e.g. phosphorylated flavonoid, such as a phosphorylated quercetin or quercetin derivative and/or fisetin or fisetin derivative, which acts to lower hyperglycemia and/or one or more symptoms of hyperglycemia induced by a calcineurin inhibitor through a non-BTB transport protein-mediated mechanism, or that acts to lower hyperglycemia and/or one or more symptoms of hyperglycemia induced by a calcineurin inhibitor through a BTB transport protein-mediated mechanism and a non-BTB transport protein-mediated mechanism, is used. In other embodiments, a phosphorylated polyphenol, e.g. phosphorylated flavonoid, such as a phosphorylated quercetin quercetin or quercetin derivative and/or fisetin or fisetin derivative, which acts to increase a therapeutic effect of a calcineurin inhibitor through a non-BTB transport protein-mediated mechanism, or that acts to increase a therapeutic effect of a calcineurin inhibitor through a BTB transport protein-mediated mechanism and a non-BTB transport protein-mediated mechanism, is used. In other embodiments, a phosphorylated polyphenol, e.g. phosphorylated flavonoid, such as a phosphorylated quercetin quercetin or quercetin derivative and/or fisetin or fisetin derivative, which acts to increase the concentration of a calcineurin inhibitor in a physiological compartment through a non-BTB transport protein-mediated mechanism, or that acts to increase the concentration of a calcineurin inhibitor in a physiological compartment through a BTB transport protein-mediated mechanism and a non-BTB transport protein-mediated mechanism, is used. In other embodiments, a phosphorylated polyphenol, e.g. phosphorylated flavonoid, such as a phosphorylated quercetin quercetin or quercetin derivative and/or fisetin or fisetin derivative, which acts to decrease the concentration of a calcineurin inhibitor in a physiological compartment through a non-BTB transport protein-mediated mechanism, or that acts to decrease the concentration of a calcineurin inhibitor in a physiological compartment through a BTB transport protein-mediated mechanism and a non-BTB transport protein-mediated mechanism, is used. In some embodiments utilizing a phosphorylated polyphenol, the polyphenol is a flavonoid. In some embodiments utilizing a phosphorylated polyphenol, the polyphenol is selected from the group consisting of quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, and epicatechin. In some embodiments utilizing a phosphorylated polyphenol, the polyphenol is a flavonol. In certain embodiments, the phosphorylated flavonol is selected from the group consisting of phosphorylated quercetin, phosphorylated galangin, phosphorylated fisetin and phosphorylated kaempferol, or combinations thereof. In some embodiments, the phosphorylated flavonol is phosphorylated quercetin or a phosphorylated quercetin derivative. In some embodiments, the phosphorylated polyphenol is phosphorylated fisetin or a phosphorylated fisetin derivative. In some embodiments, the phosphorylated flavonol is phosphorylated galangin or a phosphorylated galangin derivative. In some embodiments, the phosphorylated flavonol is phosphorylated kaempferol or a phosphorylated kaempferol derivative.

In some embodiments, the symptom of hyperglycemia that is reduced is selected from the group consisting of glucosuria, polyphagia, polyuria, polydipsia, loss of consciousness, blurred vision, headaches, coma, ketoacidosis, decrease in blood volume, decrease in renal bloodflow, accelerated lipolysis, weight loss, stomach problems, intestinal problems, poor wound healing, dry mouth, nausea, vomiting, dry skin, itchy skin, impotence, hypeventilation, ketoanemia, fatigue, weakness on one side of the body, hallucinations, impairment in cognitive function, increase sadness, anxiety, recurrent genital infections, increase sugar in urine, retinopathy, nepropathy, arteriosclerotic disorders, cardiac arrhythmia, stupor, susceptibility to infection, neuropathy, nerve damages causing cold feet, nerve damage causing insensitive feet and loss of hair. In some embodiments, the symptom of hyperglycemia is glucosuria.

In some embodiments the calcineurin inhibitor is CsA. In some embodiments the calcineurin inhibitor is tacrolimus. In some embodiments, the calcineurin inhibitor is tacrolimus analog. In some embodiments, the tacrolimus analog is selected from the group consisting of meridamycin, 31-O-Demethyl-FK506; L-683,590, L-685,818; 32-O-(1-hydroxyethylindol-5-yl)ascomycin; ascomycin; C18-OH-ascomycin; 9-deoxo-31-O-demethyl-FK506; L-688,617; A-119435; AP1903; rapamycin; dexamethasone-FK506 heterodimer; 13-O-demethyl tacrolimus; and FK 506-dextran conjugate.

In some embodiments, the invention provides a composition containing a calcineurin inhibitor and a BTB transport protein modulator, where the calcineurin inhibitor is present in an amount sufficient to exert a therapeutic effect and the BTB transport protein modulator is present in an amount sufficient to decrease hyperglycemia and/or one or more symptoms of hyperglycemia induced by the calcineurin inhibitor by a measurable amount, compared to the hyperglycemia or symptom of hyperglycemia without the BTB transport protein modulator, when the composition is administered to an animal. In some embodiments, hyperglycemia and/or one or more symptoms of hyperglycemia induced by the calcineurin inhibitor is decreased by an average of at least about 5, 10, 15, 20, 25, 30, 35, 40, 45, 50, 55, 60, 65, 70, 75, 80, 85, 90, 95, or more than 95%, compared to the hyperglycemia or symptom of hyperglycemia without the BTB transport protein modulator. In some embodiments, hyperglycemia and/or one or more symptoms of hyperglycemia induced by a calcineurin inhibitor is decreased by an average of at least about 5%, compared to the hyperglycemia or symptom of hyperglycemia without the BTB transport protein modulator. In some embodiments, hyperglycemia and/or one or more symptoms of hyperglycemia induced by the calcineurin inhibitor is decreased by an average of at least about 10%, compared to the hyperglycemia or symptom of hyperglycemia without the BTB transport protein modulator. In some embodiments, hyperglycemia and/or one or more symptoms of hyperglycemia induced by a calcineurin inhibitor is decreased by an average of at least about 15%, compared to the hyperglycemia or symptom of hyperglycemia without the BTB transport protein modulator. In some embodiments, hyperglycemia and/or one or more symptoms of hyperglycemia induced by a calcineurin inhibitor is decreased by an average of at least about 20%, compared to the hyperglycemia or symptom of hyperglycemia without the BTB transport protein modulator. In some embodiments, hyperglycemia and/or one or more symptoms of hyperglycemia induced by the calcineurin inhibitor is substantially eliminated compared to the hyperglycemia or symptom of hyperglycemia without the BTB transport protein modulator. “Substantially eliminated” as used herein encompasses no measurable or no statistically significant symptom (one or more symptoms) of hyperglycemia induced by the calcineurin inhibitor, when administered in combination with the BTB transport protein modulator.

Thus, in some embodiments, the invention provides compositions that contain a polyphenol, e.g., a flavonol, including but not limited to a phosphorylated flavonol, and a calcineurin inhibitor, where the calcineurin inhibitor is present in an amount sufficient to exert an therapeutic effect and the polyphenol, e.g., a flavonol, including but not limited to a phosphorylated flavonol, is present in an amount sufficient to decrease hyperglycemia and/or one or more symptoms of hyperglycemia induced by the calcineurin inhibitor by a measurable amount, compared to the hyperglycemia or symptom of hyperglycemia without the polyphenol, e.g., a flavonol, including but not limited to a phosphorylated flavonol, when the composition is administered to an animal. The measurable amount may be an average of at least about 5, 10, 15, 20, 25, 30, 35, 40, 45, 50, 55, 60, 65, 70, 75, 80, 85, 90, 95, or more than 95% as described herein. The symptom of hyperglycemia may be any symptom as described herein. In some embodiments, the symptom of hyperglycemia induced by the calcineurin inhibitor that is reduced glucosuria, polyphagia, polyuria, polydipsia, loss of consciousness, blurred vision, headaches, coma, ketoacidosis, decrease in blood volume, decrease in renal bloodflow, accelerated lipolysis, weight loss, stomach problems, intestinal problems, poor wound healing, dry mouth, nausea, vomiting, dry skin, itchy skin, impotence, hypeventilation, ketoanemia, fatigue, weakness on one side of the body, hallucinations, impairment in cognitive function, increase sadness, anxiety, recurrent genital infections, increase sugar in urine, retinopathy, nepropathy, arteriosclerotic disorders, cardiac arrhythmia, stupor, susceptibility to infection, neuropathy, nerve damages causing cold feet, nerve damage causing insensitive feet and loss of hair. In some embodiments, the symptom of hyperglycemia induced by the calcineurin inhibitor that is glucosuria.

In some embodiments, the invention provides compositions that contain a flavonol that is quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, or epicatechin, or a combination thereof, and a calcineurin inhibitor that is tacrolimus, where the tacrolimus is present in an amount sufficient to exert a therapeutic effect and the flavonol is present in an amount sufficient to decrease hyperglycemia and/or one or more symptoms of hyperglycemia induced by tacrolimus by a measurable amount, compared to the hyperglycemia or symptom of hyperglycemia without the flavonol when the composition is administered to an animal. The measurable amount may be an average of at least about 5, 10, 15, 20, 25, 30, 35, 40, 45, 50, 55, 60, 65, 70, 75, 80, 85, 90, 95, or more than 95% as described herein. The symptom of hyperglycemia may be any symptom as described herein. In some embodiments, the symptom of hyperglycemia induced by the calcineurin inhibitor that is reduced is selected from the group consisting of glucosuria, polyphagia, polyuria, polydipsia, loss of consciousness, blurred vision, headaches, coma, ketoacidosis, decrease in blood volume, decrease in renal bloodflow, accelerated lipolysis, weight loss, stomach problems, intestinal problems, poor wound healing, dry mouth, nausea, vomiting, dry skin, itchy skin, impotence, hypeventilation, ketoanemia, fatigue, weakness on one side of the body, hallucinations, impairment in cognitive function, increase sadness, anxiety, recurrent genital infections, increase sugar in urine, retinopathy, nepropathy, arteriosclerotic disorders, cardiac arrhythmia, stupor, susceptibility to infection, neuropathy, nerve damages causing cold feet, nerve damage causing insensitive feet and loss of hair. In some embodiments, the symptom of hyperglycemia induced by the calcineurin inhibitor that is glucosuria.

In some embodiments, the invention provides compositions that contain a flavonol that is phosphorylated, including phosphorylated quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, or epicatechin, or a combination thereof, and a calcineurin inhibitor that is tacrolimus, where the tacrolimus is present in an amount sufficient to exert a therapeutic effect and the flavonol is present in an amount sufficient to decrease hyperglycemia and/or one or more symptoms of hyperglycemia induced by tacrolimus by a measurable amount, compared to the hyperglycemia or symptom of hyperglycemia without the flavonol when the composition is administered to an animal. The measurable amount may be an average of at least about 5, 10, 15, 20, 25, 30, 35, 40, 45, 50, 55, 60, 65, 70, 75, 80, 85, 90, 95, or more than 95% as described herein. The symptom of hyperglycemia may be any symptom as described herein. In some embodiments, the symptom of hyperglycemia induced by the calcineurin inhibitor that is reduced is selected from the group consisting of glucosuria, polyphagia, polyuria, polydipsia, loss of consciousness, blurred vision, headaches, coma, ketoacidosis, decrease in blood volume, decrease in renal bloodflow, accelerated lipolysis, weight loss, stomach problems, intestinal problems, poor wound healing, dry mouth, nausea, vomiting, dry skin, itchy skin, impotence, hypeventilation, ketoanemia, fatigue, weakness on one side of the body, hallucinations, impairment in cognitive function, increase sadness, anxiety, recurrent genital infections, increase sugar in urine, retinopathy, nepropathy, arteriosclerotic disorders, cardiac arrhythmia, stupor, susceptibility to infection, neuropathy, nerve damages causing cold feet, nerve damage causing insensitive feet and loss of hair. In some embodiments, the symptom of hyperglycemia induced by the calcineurin inhibitor that is glucosuria.

In some embodiments, the invention provides compositions that contain a flavonol that is quercetin or a quercetin derivative, fisetin or a fisetin derivative, galangin or a galangin derivative, or kaempferol or a kaempferol derivative, or combinations thereof, and a calcineurin inhibitor that is tacrolimus, where tacrolimus is present in an amount sufficient to exert a therapeutic effect and the flavonol is present in an amount sufficient to decrease hyperglycemia and/or one or more symptoms of hyperglycemia induced by tacrolimus by a measurable amount, compared to the hyperglycemia or symptom of hyperglycemia without the flavonol when the composition is administered to an animal. The measurable amount may be an average of at least about 5, 10, 15, 20, 25, 30, 35, 40, 45, 50, 55, 60, 65, 70, 75, 80, 85, 90, 95, or more than 95% as described herein. The symptom of hyperglycemia may be any symptom as described herein. The symptom of hyperglycemia may be any symptom as described herein. In some embodiments, the symptom of hyperglycemia induced by the calcineurin inhibitor that is reduced is selected from the group consisting of glucosuria, polyphagia, polyuria, polydipsia, loss of consciousness, blurred vision, headaches, coma, ketoacidosis, decrease in blood volume, decrease in renal bloodflow, accelerated lipolysis, weight loss, stomach problems, intestinal problems, poor wound healing, dry mouth, nausea, vomiting, dry skin, itchy skin, impotence, hypeventilation, ketoanemia, fatigue, weakness on one side of the body, hallucinations, impairment in cognitive function, increase sadness, anxiety, recurrent genital infections, increase sugar in urine, retinopathy, nepropathy, arteriosclerotic disorders, cardiac arrhythmia, stupor, susceptibility to infection, neuropathy, nerve damages causing cold feet, nerve damage causing insensitive feet and loss of hair. In some embodiments, the symptom of hyperglycemia induced by the calcineurin inhibitor that is glucosuria.

In some embodiments, the invention provides compositions that contain a flavonol that is phosphorylated, including phosphorylated quercetin or a phosphorylated quercetin derivative, phosphorylated fisetin or a phosphorylated fisetin derivative, phosphorylated galangin or a phosphorylated galangin derivative, or phosphorylated kaempferol or a phosphorylated kaempferol derivative, or combinations thereof, and a calcineurin inhibitor that is tacrolimus, where tacrolimus is present in an amount sufficient to exert a therapeutic effect and the flavonol is present in an amount sufficient to decrease hyperglycemia and/or one or more symptoms of hyperglycemia induced by tacrolimus by a measurable amount, compared to the hyperglycemia or symptom of hyperglycemia without the flavonol when the composition is administered to an animal. The measurable amount may be an average of at least about 5, 10, 15, 20, 25, 30, 35, 40, 45, 50, 55, 60, 65, 70, 75, 80, 85, 90, 95, or more than 95% as described herein. The symptom of hyperglycemia may be any symptom as described herein. The symptom of hyperglycemia may be any symptom as described herein. In some embodiments, the symptom of hyperglycemia induced by the calcineurin inhibitor that is reduced is selected from the group consisting of glucosuria, polyphagia, polyuria, polydipsia, loss of consciousness, blurred vision, headaches, coma, ketoacidosis, decrease in blood volume, decrease in renal bloodflow, accelerated lipolysis, weight loss, stomach problems, intestinal problems, poor wound healing, dry mouth, nausea, vomiting, dry skin, itchy skin, impotence, hypeventilation, ketoanemia, fatigue, weakness on one side of the body, hallucinations, impairment in cognitive function, increase sadness, anxiety, recurrent genital infections, increase sugar in urine, retinopathy, nepropathy, arteriosclerotic disorders, cardiac arrhythmia, stupor, susceptibility to infection, neuropathy, nerve damages causing cold feet, nerve damage causing insensitive feet and loss of hair. In some embodiments, the symptom of hyperglycemia induced by the calcineurin inhibitor that is glucosuria.

In some embodiments, the invention provides compositions that contains quercetin or a quercetin derivative and tacrolimus where the tacrolimus is present in an amount sufficient to exert a therapeutic effect and the quercetin or quercetin derivative is present in an amount sufficient to decrease hyperglycemia and/or one or more symptoms of hyperglycemia induced by tacrolimus by a measurable amount, compared to the hyperglycemia or symptom of hyperglycemia without the quercetin or quercetin derivative when the composition is administered to an animal. The measurable amount may be an average of at least about 5, 10, 15, 20, 25, 30, 35, 40, 45, 50, 55, 60, 65, 70, 75, 80, 85, 90, 95, or more than 95% as described herein. The symptom of hyperglycemia may be any symptom as described herein. In some embodiments, the symptom of hyperglycemia induced by the calcineurin inhibitor that is reduced is selected from the group consisting of glucosuria, polyphagia, polyuria, polydipsia, loss of consciousness, blurred vision, headaches, coma, ketoacidosis, decrease in blood volume, decrease in renal bloodflow, accelerated lipolysis, weight loss, stomach problems, intestinal problems, poor wound healing, dry mouth, nausea, vomiting, dry skin, itchy skin, impotence, hypeventilation, ketoanemia, fatigue, weakness on one side of the body, hallucinations, impairment in cognitive function, increase sadness, anxiety, recurrent genital infections, increase sugar in urine, retinopathy, nepropathy, arteriosclerotic disorders, cardiac arrhythmia, stupor, susceptibility to infection, neuropathy, nerve damages causing cold feet, nerve damage causing insensitive feet and loss of hair. In some embodiments, the symptom of hyperglycemia induced by the calcineurin inhibitor that is glucosuria.

In some embodiments, the invention provides compositions that contains a modified quercetin or a quercetin derivative, including a phosphorylated quercetin or quercetin derivative, and tacrolimus where the tacrolimus is present in an amount sufficient to exert a therapeutic effect and the modified quercetin or quercetin derivative is present in an amount sufficient to decrease hyperglycemia and/or one or more symptoms of hyperglycemia induced by tacrolimus by a measurable amount, compared to the hyperglycemia or symptom of hyperglycemia without the modified quercetin or quercetin derivative when the composition is administered to an animal. The measurable amount may be an average of at least about 5, 10, 15, 20, 25, 30, 35, 40, 45, 50, 55, 60, 65, 70, 75, 80, 85, 90, 95, or more than 95% as described herein. The symptom of hyperglycemia may be any symptom as described herein. In some embodiments, the symptom of hyperglycemia induced by the calcineurin inhibitor that is reduced is selected from the group consisting of glucosuria, polyphagia, polyuria, polydipsia, loss of consciousness, blurred vision, headaches, coma, ketoacidosis, decrease in blood volume, decrease in renal bloodflow, accelerated lipolysis, weight loss, stomach problems, intestinal problems, poor wound healing, dry mouth, nausea, vomiting, dry skin, itchy skin, impotence, hypeventilation, ketoanemia, fatigue, weakness on one side of the body, hallucinations, impairment in cognitive function, increase sadness, anxiety, recurrent genital infections, increase sugar in urine, retinopathy, nepropathy, arteriosclerotic disorders, cardiac arrhythmia, stupor, susceptibility to infection, neuropathy, nerve damages causing cold feet, nerve damage causing insensitive feet and loss of hair. In some embodiments, the symptom of hyperglycemia induced by the calcineurin inhibitor that is glucosuria.

In some embodiments, the invention provides compositions that contains fisetin or a fisetin derivative and tacrolimus where the tacrolimus is present in an amount sufficient to exert a therapeutic effect and the fisetin or fisetin derivative is present in an amount sufficient to decrease hyperglycemia and/or one or more symptoms of hyperglycemia induced by tacrolimus by a measurable amount, compared to the hyperglycemia or symptom of hyperglycemia without the fisetin or fisetin derivative when the composition is administered to an animal. The measurable amount may be an average of at least about 5, 10, 15, 20, 25, 30, 35, 40, 45, 50, 55, 60, 65, 70, 75, 80, 85, 90, 95, or more than 95% as described herein. The symptom of hyperglycemia may be any symptom as described herein. In some embodiments, the symptom of hyperglycemia induced by the calcineurin inhibitor that is reduced is selected from the group consisting of glucosuria, polyphagia, polyuria, polydipsia, loss of consciousness, blurred vision, headaches, coma, ketoacidosis, decrease in blood volume, decrease in renal bloodflow, accelerated lipolysis, weight loss, stomach problems, intestinal problems, poor wound healing, dry mouth, nausea, vomiting, dry skin, itchy skin, impotence, hypeventilation, ketoanemia, fatigue, weakness on one side of the body, hallucinations, impairment in cognitive function, increase sadness, anxiety, recurrent genital infections, increase sugar in urine, retinopathy, nepropathy, arteriosclerotic disorders, cardiac arrhythmia, stupor, susceptibility to infection, neuropathy, nerve damages causing cold feet, nerve damage causing insensitive feet and loss of hair. In some embodiments, the symptom of hyperglycemia induced by the calcineurin inhibitor that is glucosuria.

In some embodiments, the invention provides compositions that contains a modified fisetin or a fisetin derivative, including a phosphorylated fisetin or fisetin derivative, and tacrolimus where the tacrolimus is present in an amount sufficient to exert a therapeutic effect and the modified fisetin or fisetin derivative is present in an amount sufficient to decrease hyperglycemia and/or one or more symptoms of hyperglycemia induced by tacrolimus by a measurable amount, compared to the hyperglycemia or symptom of hyperglycemia without the modified fisetin or fisetin derivative when the composition is administered to an animal. The measurable amount may be an average of at least about 5, 10, 15, 20, 25, 30, 35, 40, 45, 50, 55, 60, 65, 70, 75, 80, 85, 90, 95, or more than 95% as described herein. The symptom of hyperglycemia may be any symptom as described herein. In some embodiments, the symptom of hyperglycemia induced by the calcineurin inhibitor that is reduced is selected from the group consisting of glucosuria, polyphagia, polyuria, polydipsia, loss of consciousness, blurred vision, headaches, coma, ketoacidosis, decrease in blood volume, decrease in renal bloodflow, accelerated lipolysis, weight loss, stomach problems, intestinal problems, poor wound healing, dry mouth, nausea, vomiting, dry skin, itchy skin, impotence, hypeventilation, ketoanemia, fatigue, weakness on one side of the body, hallucinations, impairment in cognitive function, increase sadness, anxiety, recurrent genital infections, increase sugar in urine, retinopathy, nepropathy, arteriosclerotic disorders, cardiac arrhythmia, stupor, susceptibility to infection, neuropathy, nerve damages causing cold feet, nerve damage causing insensitive feet and loss of hair. In some embodiments, the symptom of hyperglycemia induced by the calcineurin inhibitor that is glucosuria.

In some embodiments, the BTB transport protein modulator is present in an amount sufficient to decrease hyperglycemia and/or one or more symptoms of hyperglycemia induced by the calcineurin inhibitor by a measurable amount and to increase a therapeutic effect of the calcineurin inhibitor by a measurable amount, compared to the hyperglycemia or symptom of hyperglycemia and therapeutic effect without the BTB transport protein modulator, when the composition is administered to an animal. In some embodiments, a therapeutic effect of the calcineurin inhibitor is increased by an average of at least about 5, 10, 15, 20, 25, 30, 35, 40, 45, 50, 55, 60, 65, 70, 75, 80, 85, 90, 95, or more than 95%, compared to the therapeutic effect without the BTB transport protein modulator. In some embodiments, a therapeutic effect of the calcineurin inhibitor is increased by an average of at least about 5%, compared to the therapeutic effect without the BTB transport protein modulator. In some embodiments, a therapeutic effect of the calcineurin inhibitor is increased by an average of at least about 10%, compared to the therapeutic effect without the BTB transport protein modulator. In some embodiments, a therapeutic effect of the calcineurin inhibitor is increased by an average of at least about 15%, compared to the therapeutic effect without the BTB transport protein modulator. In some embodiments, a therapeutic effect of the calcineurin inhibitor is increased by an average of at least about 20%, compared to the therapeutic effect without the BTB transport protein modulator. In some embodiments, a therapeutic effect of the calcineurin inhibitor is increased by an average of at least about 30%, compared to the therapeutic effect without the BTB transport protein modulator. In some embodiments, a therapeutic effect of the calcineurin inhibitor is increased by an average of at least about 40%, compared to the therapeutic effect without the BTB transport protein modulator. In some embodiments, a therapeutic effect of the calcineurin inhibitor is increased by an average of at least about 50%, compared to the therapeutic effect without the BTB transport protein modulator.

Thus, in some embodiments, the invention provides compositions containing a BTB transport protein modulator present in an amount sufficient to decrease hyperglycemia and/or one or more symptoms of hyperglycemia induced by a calcineurin inhibitor by an average of at least about 5% and to increase a therapeutic effect of the calcineurin inhibitor by an average of at least about 5%, compared to the hyperglycemia or symptom of hyperglycemia and therapeutic effect without the BTB transport protein modulator, when the composition is administered to an animal in combination with the calcineurin inhibitor. In some embodiments, the invention provides compositions containing a BTB transport protein modulator present in an amount sufficient to decrease hyperglycemia and/or one or more symptoms of hyperglycemia induced by a calcineurin inhibitor by an average of at least about 10% and to increase a therapeutic effect of the calcineurin inhibitor by an average of at least about 10%, compared to the hyperglycemia or symptom of hyperglycemia and therapeutic effect without the BTB transport protein modulator, when the composition is administered to an animal in combination with the calcineurin inhibitor. In some embodiments, the invention provides compositions containing a BTB transport protein modulator present in an amount sufficient to decrease hyperglycemia and/or one or more symptoms of hyperglycemia induced by a calcineurin inhibitor by an average of at least about 20% and to increase a therapeutic effect of the calcineurin inhibitor by an average of at least about 20%, compared to the hyperglycemia or symptom of hyperglycemia and therapeutic effect without the BTB transport protein modulator, when the composition is administered to an animal in combination with the calcineurin inhibitor. In some embodiments, the invention provides compositions containing a BTB transport protein modulator present in an amount sufficient to decrease hyperglycemia and/or one or more symptoms of hyperglycemia induced by a calcineurin inhibitor by an average of at least about 10% and to increase a therapeutic effect of the calcineurin inhibitor by an average of at least about 20%, compared to the hyperglycemia or symptom of hyperglycemia and therapeutic effect without the BTB transport protein modulator, when the composition is administered to an animal in combination with the calcineurin inhibitor. In some embodiments, the invention provides compositions containing a BTB transport protein modulator present in an amount sufficient to decrease hyperglycemia and/or one or more symptoms of hyperglycemia induced by a calcineurin inhibitor by an average of at least about 10% and to increase a therapeutic effect of the calcineurin inhibitor by an average of at least about 30%, compared to the hyperglycemia or symptom of hyperglycemia and therapeutic effect without the BTB transport protein modulator, when the composition is administered to an animal in combination with the calcineurin inhibitor. In some embodiments, the invention provides compositions containing a BTB transport protein modulator present in an amount sufficient to decrease hyperglycemia and/or one or more symptoms of hyperglycemia induced by a calcineurin inhibitor by an average of at least about 10% and to increase a therapeutic effect of the calcineurin inhibitor by an average of at least about 40%, compared to the hyperglycemia or symptom of hyperglycemia and therapeutic effect without the BTB transport protein modulator, when the composition is administered to an animal in combination with the calcineurin inhibitor. In some embodiments, the invention provides compositions containing a BTB transport protein modulator present in an amount sufficient to decrease hyperglycemia and/or one or more symptoms of hyperglycemia induced by a calcineurin inhibitor by an average of at least about 10% and to increase a therapeutic effect of the calcineurin inhibitor by an average of at least about 50%, compared to the hyperglycemia or symptom of hyperglycemia and therapeutic effect without the BTB transport protein modulator, when the composition is administered to an animal in combination with the calcineurin inhibitor.

In some embodiments, the invention provides compositions containing a pyrone analog. In some embodiments, the invention provides compositions containing a polyphenol, e.g., a flavonol such as quercetin or a quercetin derivative and/or fisetin or a fisetin derivative, present in an amount sufficient to decrease hyperglycemia and/or one or more symptoms of hyperglycemia induced by a calcineurin inhibitor by an average of at least about 5% and to increase a therapeutic effect of the calcineurin inhibitor by an average of at least about 5%, when the composition is administered to an animal in combination with the calcineurin inhibitor, compared to the hyperglycemia or symptom of hyperglycemia and therapeutic effect without the polyphenol, e.g., flavonol such as quercetin or a quercetin derivative and/or fisetin or a fisetin derivative. In some embodiments, the invention provides compositions containing a polyphenol, e.g., a flavonol such as quercetin or a quercetin derivative and/or fisetin or a fisetin derivative, present in an amount sufficient to decrease hyperglycemia and/or one or more symptoms of hyperglycemia induced by a calcineurin inhibitor by an average of at least about 10% and to increase a therapeutic effect of the calcineurin inhibitor by an average of at least about 10%, when the composition is administered to an animal in combination with the calcineurin inhibitor, compared to the hyperglycemia or symptom of hyperglycemia and therapeutic effect when the calcineurin inhibitor is administered without the a polyphenol, e.g., a flavonol such as quercetin or a quercetin derivative and/or a fisetin or a fisetin derivative. In some embodiments, the invention provides compositions containing a polyphenol, e.g., a flavonol such as quercetin or a quercetin derivative and/or fisetin or a fisetin derivative, present in an amount sufficient to decrease a hyperglycemia and/or one or more symptoms of hyperglycemia induced by a calcineurin inhibitor by an average of at least about 20% and to increase a therapeutic effect of the calcineurin inhibitor by an average of at least about 20%, when the composition is administered to an animal in combination with the calcineurin inhibitor, compared to the hyperglycemia or symptom of hyperglycemia and therapeutic effect when the calcineurin inhibitor is administered without the a polyphenol, e.g., a flavonol such as quercetin or a quercetin derivative and/or a fisetin or a fisetin derivative. In some embodiments, the invention provides compositions containing a polyphenol, e.g., a flavonol such as quercetin or a quercetin derivative and/or fisetin or a fisetin derivative, present in an amount sufficient to decrease hyperglycemia and/or one or more symptoms of hyperglycemia induced by a calcineurin inhibitor by an average of at least about 10% and to increase a therapeutic effect of the calcineurin inhibitor by an average of at least about 20%, when the composition is administered to an animal in combination with the calcineurin inhibitor, compared to the hyperglycemia or symptom of hyperglycemia and therapeutic effect when the calcineurin inhibitor is administered without the a polyphenol, e.g., a flavonol such as quercetin or a quercetin derivative and/or fisetin or a fisetin derivative. In some embodiments, the invention provides compositions containing a polyphenol, e.g., a flavonol such as quercetin or a quercetin derivative and/or fisetin or a fisetin derivative, present in an amount sufficient to decrease hyperglycemia and/or one or more symptoms of hyperglycemia induced by a calcineurin inhibitor by an average of at least about 10% and to increase a therapeutic effect of the calcineurin inhibitor by an average of at least about 30%, when the composition is administered to an animal in combination with the calcineurin inhibitor, compared to the hyperglycemia or symptom of hyperglycemia and therapeutic effect when the calcineurin inhibitor is administered without the polyphenol, e.g., a flavonol such as quercetin or a quercetin derivative and/or fisetin or a fisetin derivative. In some embodiments, the invention provides compositions containing a polyphenol, e.g., a flavonol such as quercetin or a quercetin derivative and/or fisetin or a fisetin derivative, present in an amount sufficient to decrease hyperglycemia and/or one or more symptoms of hyperglycemia induced by a calcineurin inhibitor by an average of at least about 10% and to increase a therapeutic effect of the calcineurin inhibitor by an average of at least about 40%, when the composition is administered to an animal in combination with the calcineurin inhibitor, compared to the hyperglycemia or symptom of hyperglycemia and therapeutic effect when the calcineurin inhibitor is administered without the polyphenol, e.g., a flavonol such as quercetin or a quercetin derivative and/or fisetin or a fisetin derivative. In some embodiments, the invention provides compositions containing a polyphenol, e.g., a flavonol such as quercetin or a quercetin derivative and/or fisetin or a fisetin derivative, present in an amount sufficient to decrease hyperglycemia and/or one or more symptoms of hyperglycemia induced by a calcineurin inhibitor by an average of at least about 10% and to increase a therapeutic effect of the calcineurin inhibitor by an average of at least about 50%, when the composition is administered to an animal in combination with the calcineurin inhibitor, compared to the hyperglycemia or symptom of hyperglycemia and therapeutic effect when the calcineurin inhibitor is administered without the a polyphenol, e.g., a flavonol such as quercetin or a quercetin derivative and/or fisetin or a fisetin derivative.

In some embodiments, the invention provides compositions containing a phosphorylated polyphenol, e.g., a phosphorylated flavonol such as phosphorylated quercetin or a quercetin derivative and/or phosphorylated fisetin or a fisetin derivative, present in an amount sufficient to decrease hyperglycemia and/or one or more symptoms of hyperglycemia induced by a calcineurin inhibitor by an average of at least about 5% and to increase a therapeutic effect of the calcineurin inhibitor by an average of at least about 5%, when the composition is administered to an animal in combination with the calcineurin inhibitor, compared to the hyperglycemia or symptom of hyperglycemia and therapeutic effect without the phosphorylated polyphenol, e.g., phosphorylated flavonol such as phosphorylated quercetin or a quercetin derivative and/or phosphorylated fisetin or a fisetin derivative. In some embodiments, the invention provides compositions containing a phosphorylated polyphenol, e.g., a phosphorylated flavonol such as phosphorylated quercetin or a quercetin derivative and/or phosphorylated fisetin or a fisetin derivative, present in an amount sufficient to decrease hyperglycemia and/or one or more symptoms of hyperglycemia induced by a calcineurin inhibitor by an average of at least about 10% and to increase a therapeutic effect of the calcineurin inhibitor by an average of at least about 10%, when the composition is administered to an animal in combination with the calcineurin inhibitor, compared to the hyperglycemia or symptom of hyperglycemia and therapeutic effect when the calcineurin inhibitor is administered without the phosphorylated polyphenol, e.g., a phosphorylated flavonol such as phosphorylated quercetin or a quercetin derivative and/or phosphorylated fisetin or a fisetin derivative. In some embodiments, the invention provides compositions containing a phosphorylated polyphenol, e.g., a phosphorylated flavonol such as phosphorylated quercetin or a quercetin derivative and/or phosphorylated fisetin or a fisetin derivative, present in an amount sufficient to decrease a hyperglycemia and/or one or more symptoms of hyperglycemia induced by a calcineurin inhibitor by an average of at least about 20% and to increase a therapeutic effect of the calcineurin inhibitor by an average of at least about 20%, when the composition is administered to an animal in combination with the calcineurin inhibitor, compared to the hyperglycemia or symptom of hyperglycemia and therapeutic effect when the calcineurin inhibitor is administered without the phosphorylated polyphenol, e.g., a phosphorylated flavonol such as phosphorylated quercetin or a quercetin derivative and/or phosphorylated fisetin or a fisetin derivative. In some embodiments, the invention provides compositions containing a phosphorylated polyphenol, e.g., a phosphorylated flavonol such as phosphorylated quercetin or a quercetin derivative and/or phosphorylated fisetin or a fisetin derivative, present in an amount sufficient to decrease hyperglycemia and/or one or more symptoms of hyperglycemia induced by a calcineurin inhibitor by an average of at least about 10% and to increase a therapeutic effect of the calcineurin inhibitor by an average of at least about 20%, when the composition is administered to an animal in combination with the calcineurin inhibitor, compared to the hyperglycemia or symptom of hyperglycemia and therapeutic effect when the calcineurin inhibitor is administered without the phosphorylated polyphenol, e.g., a phosphorylated flavonol such as phosphorylated quercetin or a quercetin derivative and/or phosphorylated fisetin or a fisetin derivative. In some embodiments, the invention provides compositions containing a phosphorylated polyphenol, e.g., a phosphorylated flavonol such as phosphorylated quercetin or a quercetin derivative and/or phosphorylated fisetin or a fisetin derivative, present in an amount sufficient to decrease hyperglycemia and/or one or more symptoms of hyperglycemia induced by a calcineurin inhibitor by an average of at least about 10% and to increase a therapeutic effect of the calcineurin inhibitor by an average of at least about 30%, when the composition is administered to an animal in combination with the calcineurin inhibitor, compared to the hyperglycemia or symptom of hyperglycemia and therapeutic effect when the calcineurin inhibitor is administered without the phosphorylated polyphenol, e.g., a phosphorylated flavonol such as phosphorylated quercetin or a quercetin derivative and/or phosphorylated fisetin or a fisetin derivative. In some embodiments, the invention provides compositions containing a phosphorylated polyphenol, e.g., a phosphorylated flavonol such as phosphorylated quercetin or a quercetin derivative and/or phosphorylated fisetin or a fisetin derivative, present in an amount sufficient to decrease hyperglycemia and/or one or more symptoms of hyperglycemia induced by a calcineurin inhibitor by an average of at least about 10% and to increase a therapeutic effect of the calcineurin inhibitor by an average of at least about 40%, when the composition is administered to an animal in combination with the calcineurin inhibitor, compared to the hyperglycemia or symptom of hyperglycemia and therapeutic effect when the calcineurin inhibitor is administered without the phosphorylated polyphenol, e.g., a phosphorylated flavonol such as phosphorylated quercetin or a quercetin derivative and/or phosphorylated fisetin or a fisetin derivative. In some embodiments, the invention provides compositions containing a phosphorylated polyphenol, e.g., a phosphorylated flavonol such as phosphorylated quercetin or a quercetin derivative and/or phosphorylated fisetin or a fisetin derivative, present in an amount sufficient to decrease hyperglycemia and/or one or more symptoms of hyperglycemia induced by a calcineurin inhibitor by an average of at least about 10% and to increase a therapeutic effect of the calcineurin inhibitor by an average of at least about 50%, when the composition is administered to an animal in combination with the calcineurin inhibitor, compared to the hyperglycemia or symptom of hyperglycemia and therapeutic effect when the calcineurin inhibitor is administered without the phosphorylated polyphenol, e.g., a phosphorylated flavonol such as phosphorylated quercetin or a quercetin derivative and/or phosphorylated fisetin or a fisetin derivative.

In exemplary embodiments, the invention provides a composition that contains a polyphenol, including modified polyphenols, such as a phosphorylated polyphenol, that is quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, or epicatechin, or combinations thereof, and a calcineurin inhibitor, such as tacrolimus or a tacrolimus analog, where the calcineurin inhibitor is present in an amount sufficient to exert a therapeutic effect, and the polyphenol, including modified polyphenols, such as a phosphorylated polyphenol, is present in an amount effective to decrease hyperglycemia and/or one or more symptoms of hyperglycemia induced by the calcineurin inhibitor by a measurable amount (e.g., an average of at least about 5, 10, 15, 20, or more than 20%, as described herein) and to increase the therapeutic effect of the calcineurin inhibitor by a measurable amount (e.g., an average of at least about 5, 10, 15, 20, or more than 20%, as described herein). The symptom of hyperglycemia may be any symptom as described herein. In some embodiments, the symptom of hyperglycemia induced by the calcineurin inhibitor that is reduced is selected from the group consisting of glucosuria, polyphagia, polyuria, polydipsia, loss of consciousness, blurred vision, headaches, coma, ketoacidosis, decrease in blood volume, decrease in renal bloodflow, accelerated lipolysis, weight loss, stomach problems, intestinal problems, poor wound healing, dry mouth, nausea, vomiting, dry skin, itchy skin, impotence, hypeventilation, ketoanemia, fatigue, weakness on one side of the body, hallucinations, impairment in cognitive function, increase sadness, anxiety, recurrent genital infections, increase sugar in urine, retinopathy, nepropathy, arteriosclerotic disorders, cardiac arrhythmia, stupor, susceptibility to infection, neuropathy, nerve damages causing cold feet, nerve damage causing insensitive feet and loss of hair. In some embodiments, the symptom of hyperglycemia induced by the calcineurin inhibitor that is glucosuria.

In another exemplary embodiments, the invention provides a composition that contains quercetin or a quercetin derivative, including a modified quercetin or quercetin derivative, such as a phosphorylated quercetin or quercetin derivative, and tacrolimus, where tacrolimus is present in an amount sufficient to exert a therapeutic effect, and the quercetin or a quercetin derivative is present in an amount effective to decrease hyperglycemia and/or one or more symptoms of hyperglycemia induced by tacrolimus by a measurable amount (e.g., an average of at least about 5, 10, 15, 20, or more than 20%, as described herein) and to increase the therapeutic effect of tacrolimus by a measurable amount (e.g., an average of at least about 5, 10, 15, 20, or more than 20%, as described herein). The symptom of hyperglycemia may be any symptoms described herein. In some embodiments, the symptom of hyperglycemia induced by the calcineurin inhibitor that is reduced is selected from the group consisting of glucosuria, polyphagia, polyuria, polydipsia, loss of consciousness, blurred vision, headaches, coma, ketoacidosis, decrease in blood volume, decrease in renal bloodflow, accelerated lipolysis, weight loss, stomach problems, intestinal problems, poor wound healing, dry mouth, nausea, vomiting, dry skin, itchy skin, impotence, hypeventilation, ketoanemia, fatigue, weakness on one side of the body, hallucinations, impairment in cognitive function, increase sadness, anxiety, recurrent genital infections, increase sugar in urine, retinopathy, nepropathy, arteriosclerotic disorders, cardiac arrhythmia, stupor, susceptibility to infection, neuropathy, nerve damages causing cold feet, nerve damage causing insensitive feet and loss of hair. In some embodiments, the symptom of hyperglycemia induced by the calcineurin inhibitor that is glucosuria.

In another exemplary embodiments, the invention provides a composition that contains fisetin or a fisetin derivative, including a modified fisetin or fisetin derivative, such as a phosphorylated fisetin or fisetin derivative, and tacrolimus, where tacrolimus is present in an amount sufficient to exert a therapeutic effect, and the fisetin or a fisetin derivative is present in an amount effective to decrease hyperglycemia and/or one or more symptoms of hyperglycemia induced by tacrolimus by a measurable amount (e.g., an average of at least about 5, 10, 15, 20, or more than 20%, as described herein) and to increase the therapeutic effect of tacrolimus by a measurable amount (e.g., an average of at least about 5, 10, 15, 20, or more than 20%, as described herein). The symptom of hyperglycemia may be any symptoms described herein. In some embodiments, the symptom of hyperglycemia induced by the calcineurin inhibitor that is reduced is selected from the group consisting of glucosuria, polyphagia, polyuria, polydipsia, loss of consciousness, blurred vision, headaches, coma, ketoacidosis, decrease in blood volume, decrease in renal bloodflow, accelerated lipolysis, weight loss, stomach problems, intestinal problems, poor wound healing, dry mouth, nausea, vomiting, dry skin, itchy skin, impotence, hypeventilation, ketoanemia, fatigue, weakness on one side of the body, hallucinations, impairment in cognitive function, increase sadness, anxiety, recurrent genital infections, increase sugar in urine, retinopathy, nepropathy, arteriosclerotic disorders, cardiac arrhythmia, stupor, susceptibility to infection, neuropathy, nerve damages causing cold feet, nerve damage causing insensitive feet and loss of hair. In some embodiments, the symptom of hyperglycemia induced by the calcineurin inhibitor that is glucosuria.

In some embodiments, the invention provides a composition containing an calcineurin inhibitor and a blood-tissue barrier (BTB) transport protein modulator, including a modified BTB transport protein modulator, such as a phosphorylated BTB transport protein modulator, where the calcineurin inhibitor is present in an amount sufficient to exert a therapeutic effect and the BTB transport protein modulator is present in an amount sufficient to change the concentration in a physiological compartment of the calcineurin inhibitor by a measurable amount, compared to the concentration of the calcineurin inhibitor in the physiological compartment without the BTB transport protein modulator, when the composition is administered to an animal. In some embodiments, the BTB transport protein modulator decreases the concentration of a calcineurin inhibitor in a physiological compartment where a symptom of hyperglycemia is produced. In some embodiments, the physiological compartment is a pancreatic islet cell. In some embodiments, the concentration of the calcineurin inhibitor is decreased by an average of at least about 5, 10, 15, 20, 25, 30, 35, 40, 45, 50, 55, 60, 65, 70, 75, 80, 85, 90, 95, or more than 95%, compared to the concentration without the BTB transport protein modulator. In some embodiments, the concentration of the calcineurin inhibitor is decreased by an average of at least about 5%, compared to the concentration without the BTB transport protein modulator. In some embodiments, the concentration of the calcineurin inhibitor in a physiological compartment is decreased by an average of at least about 10%, compared to the concentration without the BTB transport protein modulator. In some embodiments, the concentration of the calcineurin inhibitor in a physiological compartment is decreased by an average of at least about 15%, compared to the concentration without the BTB transport protein modulator. In some embodiments, the concentration of the calcineurin inhibitor in a physiological compartment is decreased by an average of at least about 20%, compared to the concentration without the BTB transport protein modulator. In some embodiments, the concentration of a calcineurin inhibitor in a physiological compartment is substantially eliminated compared to the concentration without the BTB transport protein modulator. “Substantially eliminated” as used herein encompasses no measurable or no statistically significant concentration of the calcineurin inhibitor in a physiological compartment, when administered in combination with the BTB transport protein modulator.

Thus, in some embodiments, the invention provides compositions that contain a pyrone analog, including a polyphenol, e.g., a flavonol, including a modified polyphenol, e.g. phosphorylated flavonol, and an calcineurin inhibitor, where the calcineurin inhibitor is present in an amount sufficient to exert an therapeutic effect and the polyphenol, e.g., a flavonol is present in an amount sufficient to decrease the concentration of the calcineurin inhibitor in a physiological compartment by a measurable amount, compared to the concentration without the polyphenol, e.g., a flavonol when the composition is administered to an animal. The measurable amount may be an average of at least about 5%, 10%, 15%, 20%, or more than 20% as described herein. In some embodiments, the physiological compartment is a pancreatic islet cell.

In some embodiments, the invention provides compositions that contain a flavonol, including a modified flavonol, such as a phosphorylated flavonol, that is quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, or epicatechin, or a combination thereof, and/or a modified quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, or epicatechin, or a combination thereof, and a calcineurin inhibitor that is tacrolimus, where tacrolimus is present in an amount sufficient to exert a therapeutic effect and the flavonol is present in an amount sufficient to decrease the concentration of tacrolimus in a physiological compartment by a measurable amount, compared to the concentration without the flavonol when the composition is administered to an animal. The measurable amount may be an average of at least about 5%, 10%, 15%, 20%, or more than 20% as described herein. In some embodiments, the physiological compartment is a pancreatic islet cell.

In some embodiments, the invention provides compositions that contain a flavonol, including a modified flavonol, such as a phosphorylated flavonol, that is quercetin, galangin, fisetinor kaempferol, or combination thereof, and a calcineurin inhibitor that is tacrolimus, where tacrolimus is present in an amount sufficient to exert a therapeutic effect and the flavonol is present in an amount sufficient to decrease the concentration of tacrolimus by a measurable amount, compared to the concentration without the flavonol when the composition is administered to an animal. The measurable amount may be an average of at least about 5%, 10%, 15%, 20%, or more than 20% as described herein. In some embodiments, the physiological compartment is a pancreatic islet cell.

In some embodiments, the invention provides compositions that contain quercetin or a quercetin derivative and tacrolimus where tacrolimus is present in an amount sufficient to exert a therapeutic effect and the quercetin or a quercetin derivative is present in an amount sufficient to decrease the concentration of tacrolimus in a physiological compartment by a measurable amount, compared to the concentration without quercetin or a quercetin derivative when the composition is administered to an animal. The measurable amount may be an average of at least about 5%, 10%, 15%, 20%, or more than 20% as described herein. In some embodiments, the physiological compartment is a pancreatic islet cell.

In some embodiments, the invention provides compositions that contain a modified quercetin or a quercetin derivative and tacrolimus where tacrolimus is present in an amount sufficient to exert a therapeutic effect and the modified quercetin or a quercetin derivative is present in an amount sufficient to decrease the concentration of tacrolimus in a physiological compartment by a measurable amount, compared to the concentration without quercetin or a quercetin derivative when the composition is administered to an animal. The measurable amount may be an average of at least about 5%, 10%, 15%, 20%, or more than 20% as described herein. In some embodiments, the physiological compartment is a pancreatic islet cell.

In some embodiments, the invention provides compositions that contain a phosphorylated quercetin or a quercetin derivative and tacrolimus where tacrolimus is present in an amount sufficient to exert a therapeutic effect and the phosphorylated quercetin or a quercetin derivative is present in an amount sufficient to decrease the concentration of tacrolimus in a physiological compartment by a measurable amount, compared to the concentration without quercetin or a quercetin derivative when the composition is administered to an animal. The measurable amount may be an average of at least about 5%, 10%, 15%, 20%, or more than 20% as described herein. In some embodiments, the physiological compartment is a pancreatic islet cell.

In some embodiments, the invention provides compositions that contain fisetin or a fisetin derivative and tacrolimus where tacrolimus is present in an amount sufficient to exert a therapeutic effect and the fisetin or a fisetin derivative is present in an amount sufficient to decrease the concentration of tacrolimus in a physiological compartment by a measurable amount, compared to the concentration without quercetin or a quercetin derivative when the composition is administered to an animal. The measurable amount may be an average of at least about 5%, 10%, 15%, 20%, or more than 20% as described herein. In some embodiments, the physiological compartment is a pancreatic islet cell.

In some embodiments, the invention provides compositions that contain a modified fisetin or a fisetin derivative and tacrolimus where tacrolimus is present in an amount sufficient to exert a therapeutic effect and the modified fisetin or a fisetin derivative is present in an amount sufficient to decrease the concentration of tacrolimus in a physiological compartment by a measurable amount, compared to the concentration without quercetin or a quercetin derivative when the composition is administered to an animal. The measurable amount may be an average of at least about 5%, 10%, 15%, 20%, or more than 20% as described herein. In some embodiments, the physiological compartment is a pancreatic islet cell.

In some embodiments, the invention provides compositions that contain a phosphorylated fisetin or a fisetin derivative and tacrolimus where tacrolimus is present in an amount sufficient to exert a therapeutic effect and the phosphorylated fisetin or a fisetin derivative is present in an amount sufficient to decrease the concentration of tacrolimus in a physiological compartment by a measurable amount, compared to the concentration without quercetin or a quercetin derivative when the composition is administered to an animal. The measurable amount may be an average of at least about 5%, 10%, 15%, 20%, or more than 20% as described herein. In some embodiments, the physiological compartment is a pancreatic islet cell.

In some embodiments, the invention provides a composition containing a calcineurin inhibitor and a blood-tissue barrier (BTB) transport protein modulator, where the calcineurin inhibitor is present in an amount sufficient to exert a therapeutic effect and the BTB transport protein modulator is present in an amount sufficient to decrease hyperglycemia and/or one or more symptoms of hyperglycemia induced by the calcineurin inhibitor and to increase the concentration of the calcineurin inhibitor in a physiological compartment by a measurable amount, compared to the concentration of the calcineurin inhibitor without the BTB transport protein modulator, when the composition is administered to an animal. Examples of physiological compartments include, but are not limited to, blood, liver, lymph nodes, spleen, Peyer's patches, intestines, lungs, heart, and kidney. In some embodiments, a concentration of the calcineurin inhibitor is increased by an average of at least about 5, 10, 15, 20, 25, 30, 35, 40, 45, 50, 55, 60, 65, 70, 75, 80, 85, 90, 95, or more than 95%, compared to the therapeutic effect without the BTB transport protein modulator. In some embodiments, concentration of the calcineurin inhibitor is increased by an average of at least about 5%, compared to the concentration of the calcineurin inhibitor without the BTB transport protein modulator. In some embodiments, concentration of the calcineurin inhibitor is increased by an average of at least about 10%, compared to the concentration of the calcineurin inhibitor without the BTB transport protein modulator. In some embodiments, concentration of the calcineurin inhibitor is increased by an average of at least about 15%, compared to the concentration of the calcineurin inhibitor without the BTB transport protein modulator. In some embodiments, a concentration of the calcineurin inhibitor is increased by an average of at least about 20%, compared to the concentration of the calcineurin inhibitor without the BTB transport protein modulator. In some embodiments, concentration of the calcineurin inhibitor is substantially increased compared to the concentration of the calcineurin inhibitor without the BTB transport protein modulator.

In some embodiments, the invention provides a composition containing a calcineurin inhibitor and a BTB transport protein modulator, where the calcineurin inhibitor is present in an amount sufficient to exert a therapeutic effect and the BTB transport protein modulator is present in an amount sufficient to decrease hyperglycemia and/or one or more symptoms of hyperglycemia induced by the calcineurin inhibitor and to increase the concentration of the calcineurin inhibitor in blood by a measurable amount, compared to the concentration of the calcineurin inhibitor without the BTB transport protein modulator, when the composition is administered to an animal.

In some embodiments, the invention provides a composition containing a calcineurin inhibitor and a BTB transport protein modulator, where the calcineurin inhibitor is present in an amount sufficient to exert a therapeutic effect and the BTB transport protein modulator is present in an amount sufficient to decrease hyperglycemia and/or one or more symptoms of hyperglycemia induced by the calcineurin inhibitor and to increase the concentration of the calcineurin inhibitor in a lymphoid tissue by a measurable amount, compared to the concentration of the calcineurin inhibitor without the BTB transport protein modulator, when the composition is administered to an animal. Examples of a lymphoid tissue include but are not limited to, thymus, bone marrow, lymph nodes, spleen, Peyer's patches, and lymphatics.

In some embodiments, the invention provides a composition containing a calcineurin inhibitor and a BTB transport protein modulator, where the calcineurin inhibitor is present in an amount sufficient to exert a therapeutic effect and the BTB transport protein modulator is present in an amount sufficient to decrease the concentration of the calcineurin inhibitor in an physiological compartment, such as a pancreatic islet cell, by a measurable amount, compared to the concentration of the calcineurin inhibitor without the BTB transport protein modulator, when the composition is administered to an animal.

In some embodiments, the invention provides a composition containing a calcineurin inhibitor and a BTB transport protein modulator, where the calcineurin inhibitor is present in an amount sufficient to exert a therapeutic effect and the BTB transport protein modulator is present in an amount sufficient to decrease hyperglycemia and/or one or more symptoms of hyperglycemia induced by the calcineurin inhibitor and to decrease the clearance of the calcineurin inhibitor from a physiological compartment where the calcineurin inhibitor exerts a therapeutic effect.

An “average” as used herein is preferably calculated in a set of normal human subjects, this set being at least about 3 human subjects, preferably at least about 5 human subjects, preferably at least about 10 human subjects, even more preferably at least about 25 human subjects, and most preferably at least about 50 human subjects.

In some embodiments, the invention provides a composition that contains a calcineurin inhibitor and a BTB transport protein modulator, e.g. a polyphenol such as a flavonoid, including a modified polyphenol, such as a phosphorylated flavonoid. In some embodiments, the concentration of one or more of the calcineurin inhibitors and/or BTB transport protein modulator, e.g. a polyphenol such as a flavonol, or a modified polyphenol, such as a phosphorylated flavonoid, is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v or v/v.

In some embodiments, the concentration of one or more of the calcineurin inhibitors and/or BTB transport protein modulator, e.g. a polyphenol, such as a flavonoid, including a modified polyphenol, such as a phosphorylated flavonoid is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some embodiments, the concentration of one or more of the calcineurin inhibitors and/or BTB transport protein modulator, e.g. a polyphenol such as a flavonoid, including a modified polyphenol, such as a phosphorylated flavonoid, is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, W/V or V/V.

In some embodiments, the concentration of one or more of the calcineurin inhibitors and/or BTB transport protein modulator, e.g. a polyphenol such as a flavonoid, including a modified polyphenol, such as a phosphorylated flavonoid, is in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v.

In some embodiments, the amount of one or more of the calcineurin inhibitors and/or BTB transport protein modulator, e.g. a polyphenol such as a flavonoid, including a modified polyphenol, such as a phosphorylated flavonoid, is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of one or more of the calcineurin inhibitors and/or BTB transport protein modulator, e.g. a polyphenol such as a flavonoid, including a modified polyphenol, such as a phosphorylated flavonoid, is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

In some embodiments, the amount of one or more of the calcineurin inhibitors and/or BTB transport protein modulator, e.g. a polyphenol such as a flavonoid, including a modified polyphenol, such as a phosphorylated flavonoid, is in the range of 0.0001-10 g, 0.0005-9 g, 0.001-8 g, 0.005-7 g, 0.01-6 g, 0.05-5 g, 0.1-4 g, 0.5-4 g, or 1-3 g.

In some embodiments, compositions of the invention include quercetin or a quercetin derivative and tacrolimus, where quercetin or a quercetin derivative is present in an amount from about 1-1000 mg, or about 10-1000 mg, or about 50-1000 mg, or about 100-1000 mg, or about 1-500 mg, or about 5-500 mg, or about 50-500 mg, or about 100-500 mg, or about 200-1000 mg, or about 200-800 mg, or about 200-700 mg, or about 10 mg, or about 25 mg, or about 50 mg, or about 100 mg, or about 200 mg, or about 250 mg, or about 300 mg, or about 400 mg, or about 500 mg, or about 600 mg, or about 700 mg, or about 800 mg, or about 900 mg, or about 1000 mg, and tacrolimus is present in an amount from 0.01 to 200 mg, or about 0.1-160 mg, or about 0.1, 0.5, 1, 5, 10, 20, 50, 80, or 160 mg. In some embodiments, the compositions disclosed herein include a modified quercetin, such as quercetin phosphate. In some embodiments, the compositions of the invention include quercetin or a quercetin derivative and a pharmaceutical excipient. In some embodiments, the pharmaceutical excipient includes an oligosaccharide excipient, such as a cyclodextrin.

In some embodiments, compositions of the invention include fisetin or a fisetin derivative and tacrolimus, where fisetin or a fisetin derivative is present in an amount from about 1-1000 mg, or about 10-1000 mg, or about 50-1000 mg, or about 100-1000 mg, or about 1-500 mg, or about 5-500 mg, or about 50-500 mg, or about 100-500 mg, or about 200-1000 mg, or about 200-800 mg, or about 200-700 mg, or about 10 mg, or about 25 mg, or about 50 mg, or about 100 mg, or about 200 mg, or about 250 mg, or about 300 mg, or about 400 mg, or about 500 mg, or about 600 mg, or about 700 mg, or about 800 mg, or about 900 mg, or about 1000 mg, and tacrolimus is present in an amount from 0.01 to 200 mg, or about 0.1-160 mg, or about 0.1, 0.5, 1, 5, 10, 20, 50, 80, or 160 mg. In some embodiments, the compositions disclosed herein include a modified fisetin, such as fisetin phosphate. In some embodiments, the compositions of the invention include fisetin or a fisetin derivative and a pharmaceutical excipient. In some embodiments, the pharmaceutical excipient includes an oligosaccharide excipient, such as a cyclodextrin.

In some embodiments, tacrolimus/quercetin or a quercetin derivative, or tacrolimus/modified quercetin, such as phosphorylated quercetin or a phosphorylated quercetin derivative, is present at about 0.1/50 mg (tacrolimus/quercetin). In some embodiments, tacrolimus is present at about 0.1 mg and the quercetin or a quercetin derivative is present at about 100 mg. In some embodiments, tacrolimus is present at about 0.1 mg and the quercetin or a quercetin derivative is present at about 200 mg. In some embodiments, tacrolimus is present at about 0.1 mg and the quercetin or a quercetin derivative is present at about 300 mg. In some embodiments, tacrolimus is present at about 0.1 mg and the quercetin or a quercetin derivative is present at about 1000 mg. In some embodiments, tacrolimus is present at about 0.5 mg and the quercetin or a quercetin derivative is present at about 100 mg. In some embodiments, tacrolimus is present at about 0.5 mg and the quercetin or a quercetin derivative is present at about 250 mg. In some embodiments, tacrolimus is present at about 0.5 mg and the quercetin is present at about 500 mg. In some embodiments, tacrolimus is present at about 0.5 mg and the quercetin or a quercetin derivative is present at about 1000 mg. In some embodiments, tacrolimus is present at about 1 mg and the quercetin or a quercetin derivative is present at about 100 mg. In some embodiments, tacrolimus is present at about 1 mg and the quercetin or a quercetin derivative is present at about 250 mg. In some embodiments, tacrolimus is present at about 1 mg and the quercetin or a quercetin derivative is present at about 500 mg. In some embodiments, tacrolimus is present at about 1 mg and the quercetin or a quercetin derivative is present at about 1000 mg. In some embodiments, tacrolimus is present at about 5 mg and the quercetin or a quercetin derivative is present at about 100 mg. In some embodiments, tacrolimus is present at about 5 mg and the quercetin or a quercetin derivative is present at about 200 mg. In some embodiments, tacrolimus is present at about 5 mg and the quercetin or a quercetin derivative is present at about 300 mg. In some embodiments, tacrolimus is present at about 5 mg and the quercetin or a quercetin derivative is present at about 1000 mg. In some embodiments, tacrolimus is present at about 10 mg and the quercetin or a quercetin derivative is present at about 100 mg. In some embodiments, tacrolimus is present at about 10 mg and the quercetin or a quercetin derivative is present at about 200 mg. In some embodiments, tacrolimus is present at about 10 mg and the quercetin or a quercetin derivative is present at about 300 mg. In some embodiments, tacrolimus is present at about 10 mg and the quercetin or a quercetin derivative is present at about 1000 mg. In some embodiments, tacrolimus is present at about 15 mg and the quercetin or a quercetin derivative is present at about 100 mg. In some embodiments, tacrolimus is present at about 15 mg and the quercetin or a quercetin derivative is present at about 200 mg. In some embodiments, tacrolimus is present at about 15 mg and the quercetin or a quercetin derivative is present at about 300 mg. In some embodiments, tacrolimus is present at about 15 mg and the quercetin or a quercetin derivative is present at about 1000 mg. In some embodiments, the quercetin is in the form of quercetin phosphate. In some embodiments, the compositions of the invention include quercetin or a quercetin derivative and a cyclodextrin such as captisol.

In liquid preparations, tacrolimus can be present at about 1-100 mg/ml, or 1-50 mg/ml, or 1-20 mg/ml, or about 1, 5, 10, or 20 mg/ml and quercetin or a quercetin derivative at about 1-1000 mg/ml, or about 10-1000 mg/ml, or about 50-1000 mg/ml, or about 100-1000 mg/ml, or about 1-500 mg/ml, or about 5-500 mg/ml, or about 50-500 mg/ml, or about 100-500 mg/ml, or about 200-1000 mg/ml, or about 200-800 mg/ml, or about 200-700 mg/ml, or about 10 mg/ml, or about 25 mg/ml, or about 50 mg/ml, or about 100 mg/ml, or about 200 mg/ml, or about 250 mg/ml, or about 300 mg/ml, or about 400 mg/ml, or about 500 mg/ml, or about 600 mg/ml, or about 700 mg/ml, or about 800 mg/ml, or about 900 mg/ml, or about 1000 mg/ml. At higher levels of quercetin or a quercetin derivative, solubility can be enhanced by adjusting the type of diluent. In some embodiments, the quercetin is in the form of quercetin phosphate. In some embodiments, the compositions of the invention include quercetin or a quercetin derivative and a cyclodextrin such as captisol.

In some embodiments, a molar ratio of one or more of the calcineurin inhibitors to the BTB transport protein modulator, e.g. a polyphenol such as a flavonoid, or a modified polyphenol such as a phosphorylated flavonoid, can be 0.0001:1 to 1:1. Without limiting the scope of the invention, the molar ratio of one or more of the calcineurin inhibitors to the BTB transport protein modulator, e.g. a polyphenol such as a flavonoid, or a modified polyphenol such as a phosphorylated flavonoid, can be about 0.0001:1 to about 10:1, or about 0.001:1 to about 5:1, or about 0.01:1 to about 5:1, or about 0.1:1 to about 2:1, or about 0.2:1 to about 2:1, or about 0.5:1 to about 2:1, or about 0.1:1 to about 1:1.

Without limiting the scope of the present invention, the molar ratio of one or more of the calcineurin inhibitors to the flavonoid can be about 0.03×10⁻⁵:1, 0.1×10⁻⁵:1, 0.04×10⁻³:1, 0.03×10⁻⁵:1, 0.02×10⁻⁵:1, 0.01×10⁻³:1, 0.1×10⁻³:1, 0.15×10⁻³:1, 0.2×10⁻³:1, 0.3×10⁻³:1, 0.4×10⁻³:1, 0.5×10⁻³:1, 0.15×10⁻²:1, 0.1×10⁻²:1, 0.2×10⁻²:1, 0.3×10⁻²:1, 0.4×10⁻²:1, 0.5×10⁻²:1, 0.6×10²:1, 0.8×10⁻²:1, 0.01:1, 0.1:1;or 0.2:1 per dose. In one embodiment, the calcineurin inhibitor is tacrolimus. In one embodiment, the flavonoid is quercetin or a quercetin derivative. In some embodiments, the flavonoid is a modified quercetin or a quercetin derivative. In some embodiments, the flavonoid is a phosphorylated quercetin or a quercetin derivative. In one embodiment, the flavonoid is fisetin or a fisetin derivative. In some embodiments, the flavonoid is a modified fisetin or a fisetin derivative. In some embodiments, the flavonoid is a phosphorylated fisetin or a fisetin derivative.

Without limiting the scope of the present invention, the molar ratio of one or more of the calcineurin inhibitors to the BTB transport protein modulator, e.g. a polyphenol such as a flavonoid can be about 0.001:1, 0.002:1, 0.003:1, 0.004:1, 0.005:1, 0.006:1, 0.007:1, 0.008:1, 0.009:1, 0.01:1, 0.02:1, 0.03:1, 0.04:1, 0.05:1, 0.06:1, 0.07:1, 0.08:1, 0.09:1, 0.1:1, 0.2:1, 0.3:1, 0.4:1, 0.5:1, 0.6:1, 0.7:1, 0.8:1, 0.9:1, 1:1, 2:1, 3:1, 4:1, or 5:1 per dose. In one embodiment, the calcineurin inhibitor is tacrolimus. In one embodiment, the flavonoid is quercetin or a quercetin derivative.

A. Pharmaceutical Compositions

The transport protein modulators of the invention are usually administered in the form of pharmaceutical compositions. The drugs described above are also administered in the form of pharmaceutical compositions. When the transport protein modulators and the drugs are used in combination, both components may be mixed into a preparation or both components may be formulated into separate preparations to use them in combination separately or at the same time.

This invention therefore provides pharmaceutical compositions that contain, as the active ingredient, a BTB transport protein modulator or a pharmaceutically acceptable salt and/or coordination complex thereof, and one or more pharmaceutically acceptable excipients, carriers, including inert solid diluents and fillers, diluents, including sterile aqueous solution and various organic solvents, permeation enhancers, solubilizers and adjuvants.

This invention further provides pharmaceutical compositions that contain, as the active ingredient, a BTB transport protein modulator or a pharmaceutically acceptable salt and/or coordination complex thereof, a calcineurin inhibitor or a pharmaceutically acceptable salt and/or coordination complex thereof, and one or more pharmaceutically acceptable excipients, carriers, including inert solid diluents and fillers, diluents, including sterile aqueous solution and various organic solvents, permeation enhancers, solubilizers and adjuvants.

The BTB transport protein modulator and/or the calcineurin inhibitor may be prepared into pharmaceutical compositions in dosages as described herein (see, e.g., Compositions). Such compositions are prepared in a manner well known in the pharmaceutical art.

Pharmaceutical compositions for oral administration In some embodiments, the invention provides a pharmaceutical composition for oral administration containing a combination of a calcineurin inhibitor and an agent that reduces or eliminates hyperglycemia and/or one or more symptoms of hyperglycemia induced by the calcineurin inhibitor, and a pharmaceutical excipient suitable for oral administration. In some embodiments, the agent that reduces or eliminates hyperglycemia and/or one or more symptoms of hyperglycemia induced by the calcineurin inhibitor is a BTB transport protein modulator, e.g. a polyphenol such as a flavonol, as described elsewhere herein.

In some embodiments, the invention provides a solid pharmaceutical composition for oral administration containing:
(i) an effective amount of a calcineurin inhibitor;
(ii) an effective amount of an agent capable of reducing or eliminating hyperglycemia and/or one or more symptoms of hyperglycemia induced by the calcineurin inhibitor; and
(iii) a pharmaceutical excipient suitable for oral administration.

In some embodiments, the composition further contains: (iv) an effective amount of a second calcineurin inhibitor.

In some embodiments, the pharmaceutical composition may be a liquid pharmaceutical composition suitable for oral consumption.

In some embodiments, the calcineurin inhibitor is tacrolimus. In some embodiments, the calcineurin inhibitor is a tacrolimus analog. In some embodiments, the calcineurin inhibitor is CsA. In some embodiments, the agent capable of reducing or eliminating hyperglycemia and/or one or more symptoms of hyperglycemia induced by the calcineurin inhibitor is a BTB transport protein modulator, e.g., a BTB transport protein activator. In some embodiments, the agent capable of reducing or eliminating hyperglycemia and/or one or more symptoms of hyperglycemia induced by the calcineurin inhibitor is a polyphenol, e.g., a flavonoid such as a flavonol.

In some embodiments, the invention provides a solid pharmaceutical composition for oral administration containing:
(i) an effective amount of a calcineurin inhibitor that is tacrolimus, tacrolimus analog or CsA;
(ii) an effective amount of a polyphenol that is quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, or epicatechin; and
(iii) a pharmaceutical excipient suitable for oral administration.

In some embodiments, the composition further contains (iv) an effective amount of a second calcineurin inhibitor.

In some embodiments, the pharmaceutical composition may be a liquid pharmaceutical composition suitable for oral consumption.

In some embodiments, the invention provides a solid pharmaceutical composition for oral administration containing:
(i) an effective amount of a calcineurin inhibitor that is tacrolimus, tacrolimus analog or CsA;
(ii) an effective amount of a polyphenol that is quercetin, galangin, or kaempferol; and
(iii) a pharmaceutical excipient suitable for oral administration.

In some embodiments, the composition further contains (iv) an effective amount of a second calcineurin inhibitor.

In some embodiments, the pharmaceutical composition may be a liquid pharmaceutical composition suitable for oral consumption.

In some embodiments, the invention provides a solid pharmaceutical composition for oral administration containing an effective amount of tacrolimus, an amount of quercetin or a quercetin derivative that is effective in reducing or eliminating a hyperglycemia and/or one or more symptoms of hyperglycemia induced by tacrolimus, and a pharmaceutically acceptable excipient. In some embodiments, the invention provides a liquid pharmaceutical composition for oral administration containing an effective amount of tacrolimus, an amount of quercetin or a quercetin derivative that is effective in reducing or eliminating hyperglycemia and/or one or more symptoms of hyperglycemia induced tacrolimus, and a pharmaceutically acceptable excipient.

In some embodiments, the invention provides a solid pharmaceutical composition for oral administration containing tacrolimus at about 0.01-160 mg, quercetin or a quercetin derivative at about 10-1000 mg and a pharmaceutically acceptable excipient. In some embodiments, the invention provides a liquid pharmaceutical composition for oral administration containing tacrolimus at about 0.1-200 mg/ml, quercetin or a quercetin derivative at about 10-1000 mg/ml and a pharmaceutically acceptable excipient.

Pharmaceutical compositions of the invention suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods of pharmacy, but all methods include the step of bringing the active ingredient into association with the carrier, which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

This invention further encompasses anhydrous pharmaceutical compositions and dosage forms comprising an active ingredient, since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the invention can be prepared using anhydrous or low moisture containing ingredients and low moisture or low humidity conditions. Pharmaceutical compositions and dosage forms of the invention which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

An active ingredient can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the invention to provide tablets that disintegrate when exposed to an aqueous environment. Too much of a disintegrant may produce tablets which may disintegrate in the bottle. Too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

When aqueous suspensions and/or elixirs are desired for oral administration, the essential active ingredient therein may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

The tablet can be prepared for immediate-release. For example, the tablet can be an erodible tablet. A solubilizer, such as captisol when compressed, that erodes rather than disintegrates can be mixed with the active ingredient to form the erodible tablet. Formulation for oral use can also be present as a hard gelatin capsule using suboptimal lyophilization process.

Surfactant which can be used to form pharmaceutical compositions and dosage forms of the invention include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

Within the aforementioned group, preferred ionic surfactants include, by way of example: lecithins, lysolecithin, phospholipids, lysophospholipids and derivatives thereof; carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil; PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In one embodiment, the composition may include a solubilizer to ensure good solubilization and/or dissolution of the calcineurin inhibitor and/or BTB transport protein modulator (e.g., flavonol) and to minimize precipitation of the calcineurin inhibitor and/or BTB transport protein modulator (e.g., flavonol). This can be especially important for compositions for non-oral use, e.g., compositions for injection. A solubilizer may also be added to increase the solubility of the hydrophilic drug and/or other components, such as surfactants, or to maintain the composition as a stable or homogeneous solution or dispersion.

Cyclodextrins and their derivatives can be used to enhance the aqueous solubility of hydrophobic compounds. Cyclodextrins are cyclic carbohydrates derived from starch. The unmodified cyclodextrins differ by the number of glucopyranose units joined together in the cylindrical structure. The parent cyclodextrins typically contain 6, 7, or 8 glucopyranose units and are referred to as alpha-, beta-, and gamma-cyclodextrin respectively. Each cyclodextrin subunit has secondary hydroxyl groups at the 2 and 3-positions and a primary hydroxyl group at the 6-position. The cyclodextrins may be pictured as hollow truncated cones with hydrophilic exterior surfaces and hydrophobic interior cavities. In aqueous solutions, these hydrophobic cavities can incorporate hydrophobic organic compounds, which can fit all, or part of their structure into these cavities. This process, sometimes referred to as inclusion complexation, may result in increased apparent aqueous solubility and stability for the complexed drug. The complex is stabilized by hydrophobic interactions and does not generally involve the formation of any covalent bonds.

Cyclodextrins can be derivatized to improve their properties. Cyclodextrin derivatives that are particularly useful for pharmaceutical applications include the hydroxypropyl derivatives of alpha-, beta- and gamma-cyclodextrin, sulfoalkylether cyclodextrins such as sulfobutylether beta-cyclodextrin, alkylated cyclodextrins such as the randomly methylated beta.-cyclodextrin, and various branched cyclodextrins such as glucosyl- and maltosyl-beta.-cyclodextrin. Chemical modification of the parent cyclodextrins (usually at the hydroxyl moieties) has resulted in derivatives with sometimes improved safety while retaining or improving the complexation ability of the cyclodextrin. The chemical modifications, such as sulfoalkyl ether and hydroxypropyl, can result in rendering the cyclodextrins amorphous rather than crystalline, leading to improved solubility.

Particularly useful cyclodextrin for the present invention are the sulfoalkyl ether derivatives. The sulfoalkyl ether-CDs are a class of negatively charged cyclodextrins, which vary in the nature of the alkyl spacer, the salt form, the degree of substitution and the starting parent cyclodextrin. A particularly useful form of cyclodextrin is sulfobutylether-7-β-cyclodextrin, which is available under the trade name Captisol™ form CyDex, Inc. which has an average of about 7 substituents per cyclodextrin molecule. The anionic sulfobutyl ether substituents improve the aqueous solubility of the parent cyclodextrin. Reversible, non-covalent, complexation of flavonoids with the sulfobutylether-7-β-cyclodextrin cyclodextrin can provide for increased solubility and stability in aqueous solutions. Examples of formulations utilizing cyclodextrin are provided in U.S. Appn. No. 60/953,186, filed 31 Jul. 2007, entitled: Soluble Flavonoid Methods and Pharmaceutical Compositions.

Methods for making aqueous solutions of flavonoids and cyclodextrins that involve mixing the flavonoids and cyclodextrins at high pH, then subsequently reducing the pH. The methods disclosed herein provide a route to make high-concentration aqueous compositions comprising flavonoids and cyclodextrins, for example, comprising sulfobutylether-7-β-cyclodextrin. The compositions can be used as made, or can be further processed, for example by freeze-drying to create a powder composition. These compositions can be used as pharmaceutical compositions to be administered in a variety of ways, for example, intravenously or orally. The ability to have high concentration solutions of these compositions is useful both for the practical processing and manufacturing of pharmaceuticals based on these compositions, and for administering the compositions, where the solubility can be related to bioavailability of the compositions.

In some embodiments, the high solubility aqueous solutions of the invention are stable over time. The stability of the solutions allows them to be stored in some cases for days, weeks or months in liquid form. As used herein, stability with respect to solubility refers to stability with respect to precipitation from solution.

The flavonoid-sulfoalkyl ether compositions of the invention are useful as compositions and method for co-administration with a calcineurin inhibitor. The compositions, for example, can be co-administered with a calcineurin inhibitor to enhance the effectiveness of the calcineurin inhibitor. For example, a sulfobutylether-7-β-cyclodextrin-quercetin aqueous composition, or a sulfobutylether-7-β-cyclodextrin-quercetin derivative aqueous composition of the present invention can reduce or eliminate hyperglycemia and/or one or more symptoms of hyperglycemia induced by the calcineurin inhibitor.

In some embodiments, a method of making aqueous flavonoid solutions comprises mixing a cyclodextrin and the flavonoid at a pH greater than about 11 and subsequently lowering the pH to less than about 9. In some cases, the method allows for the preparation of aqueous solutions with high concentrations of flavonoid. In some cases, the method allows for the production of aqueous compositions with high concentrations of flavonoids.

In one embodiment, a method for forming an aqueous composition comprises a flavonoid comprising: (a) dissolving cyclodextrin in an aqueous solution; (b) adding the flavonoid to the aqueous solution; (c) raising the pH of the aqueous solution to above about pH 11 while mixing the cyclodextrin and flavonoid; and (d) lowering the pH of the aqueous solution to below about pH 9.

In some embodiments, the pH is raised to greater than about pH 11. For example, the pH can be raised to above about 11, 11.2, 11.4, 11.6, 11.8, 12, 12.2, 12.4, 12.6, 12.8, 13, 13.2, 13.4 or above pH 13.4. In general, the pH is raised to bring the flavonoid into solution. In some embodiments the pH is raised to bring as much of the flavonoid into solution as possible without causing significant degradation of the flavonoid. In some embodiments substantially all of the flavonoid is dissolved into solution at the high pH.

In some embodiments, after raising the pH to above pH 11, the pH of the solution is lowered below pH 9. In some embodiments the pH is lowered to below about 8.8, 8.6, 8.5, 8.4, 8.2, 7.8, 7.6, 7.4, 7.2, 7.0, 6.8, 6.5, 6, 5.5, 5, 4.5, 4, 3.5, 3, or less than pH 3. In general, after raising the pH, the pH is lowered to the level at which the aqueous composition will be used or stored. Where the composition is to be used as a pharmaceutical, the pH is lowered to a biologically acceptable pH, usually near neutral pH. In some embodiments, the pH is lowered to between 6 and 9, between 6.5 and 8.5, between about 7.2 and 8.4, between about 7.6 and 8.0, or about pH 7.8.

Some flavonoids are known to be unstable and to degrade in basic solution. For instance, Zheng, et al. J. Pharm. Sci. 94(5), 2005 teaches that while quercetin is stable below pH 3, degradation of quercetin above pH 5 became apparent (see page 1084). Thus, complexation in aqueous solutions between flavonoids and cyclodextrins has generally been carried out at or below neutral pH. For instance, Zheng et al. mix excess quercetin with various cyclodextrins in phosphate buffer at pH 3, mix the mixture for 24 hours, then filter off the undissolved material.

We have found that while flavonoids can degrade in basic solution, aqueous flavonoid-cyclodextrin compositions can be prepared with the present invention with little to no degradation of the flavonoid by minimizing the time during which the flavonoid is above pH 9. In some embodiments the time that the flavonoid is above pH 9 is less than about 60, 40, 30, 20, 15, 10, 5, 4, 3, 2, or less than about one minute. In some embodiments, the time that the flavonoid is above pH 9 is less than about 20 minutes. In some embodiments, the time that the flavonoid is above pH 9 is less than about 15 minutes. In some embodiments, the time that the flavonoid is above pH 9 is less than about 10 minutes. In some embodiments, the time that the flavonoid is above pH 9 is less than about 5 minutes. In some embodiments, the time that the flavonoid is above pH 9 is between about 30 and about 60, between about 20 and about 40, between about 15 and about 20, between about 10 and about 15, between about 5 and about 10, between about 1 and about 5, between about 1 and about 10, between about 2 and about 15, or between about 5 and about 15 minutes.

In the methods of the present invention, the temperature at which flavonoid is above pH 9 is generally kept relatively low. In embodiments of the invention, the temperature at which the flavonoid is above pH 9 is kept below about 50° C., below about 40° C., below about 30° C., below about 28° C., below about 26° C., below about 24° C., below about 22° C., below about 20° C., below about 18° C., below about 16° C., below about 15° C., below about 14° C., below about 12° C., or below about 10° C. In some embodiments the temperature at which the flavonoid is above pH 9 is between about 20° C. and about 30° C., between about 10° C. and about 40° C., between about 20° C. and about 26° C., or between about 23° C. and about 25° C.

Any suitable flavonoid can be used in the present invention. A detailed description of flavonoids is provided herein. In some embodiments of the method, the flavonoid that is used in the method is selected from the group consisting of quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, and epicatechin or mixtures thereof. In some embodiments of the methods, the flavonoid is quercetin, kaempferol, fisetin or galangin or mixtures thereof. In some embodiments, the flavonoid is quercetin or fisetin. In some embodiments, the flavonoid is a derivative of quercetin or fisetin.

The methods of the present invention are useful for flavonoids that are insoluble in water or that are sparingly soluble in water. A flavonoid that is sparingly soluble in water has a low solubility constant or Ks. An example of a sparingly soluble flavonoid is quercetin.

The methods of the present invention are useful for flavonoids having acidic protons. An acidic proton can be removed by base in aqueous solution. In some embodiments, the pKa of the proton is less than 10. In some embodiments the acidic proton will be an —OH group that is attached to an aromatic ring, or a phenol group. The flavonoids can have multiple aromatic —OH groups. In some embodiments, the flavonoid has 3, 4, 5, or 6 acidic protons and/or aromatic —OH groups.

While not being bound by theory, it is known that flavonoids with aromatic —OH protons that are substantially water insoluble or sparingly water soluble can be made more water soluble by raising the pH, due at least in part to the deprotonation of the acidic hydrogen(s), creating a flavonoid anion that will tend to be more soluble in water than flavonoid without the proton removed. Thus, raising the pH to above the pKa of the acidic proton on the flavonoid, can result in higher solubility of the flavonoid at the high pH. In the method of the present invention, the flavonoid, at high pH, is mixed with the cyclodextrin, and then the pH of the aqueous solution is lowered. As the pH of the solution is lowered, the flavonoid becomes less soluble, but instead of precipitating out of solution, the flavonoid appears to form a complex with the cyclodextrin. This method is an effective method for rapidly obtaining a soluble flavonoid-cyclodextrin aqueous composition. Surprisingly, we have found that this method can produce a flavonoid-cyclodextrin aqueous composition in which the flavonoid is soluble at higher concentrations than obtained by conventional means such as sonicating the flavonoid and cyclodextrin below pH 8. This method can be used to obtain high aqueous concentrations of flavonoids with sulfobutylether-7-β-cyclodextrin. In some embodiments, high aqueous concentrations of quercetin or a quercetin derivative with sulfobutylether-7-β-cyclodextrin can be obtained with the methods of the invention.

The methods disclosed herein can be used with any suitable type of cyclodextrin. A more detailed description of cyclodextrins is provided below. The methods of the invention can be used with alpha, beta or gamma cyclodextrins. The methods disclosed herein can be used with modified cyclodextrins such as hydroxypropyl derivatives of alpha-, beta- and gamma-cyclodextrin, sulfoalkylether cyclodextrins such as sulfobutylether beta-cyclodextrin, alkylated cyclodextrins such as the randomly methylated beta.-cyclodextrin, and various branched cyclodextrins such as glucosyl- and maltosyl-beta.-cyclodextrin. In some embodiments, the method is directed at pharmaceutical compositions, in which hydroxypropyl cyclodextrins and sulfoalkyl cyclodextrins can be useful. In some embodiments, sulfobutylether-7-β-cyclodextrin is used.

In some embodiments, the invention provides a composition comprising a flavonoid and a sulfo-alkyl ether substituted cyclodextrin and an aqueous carrier wherein the flavonoid is present in a concentration greater than 0.5 mM, 1 mM, 5 mM, 10 mM, 20 mM, 30 mM, 33 mM, 40 mM, 50 mM, 60 mM, 70 mM, 80 mM or greater than 80 mM. In some embodiments, the invention provides a composition comprising a flavonoid and a sulfobutylether-7-β-cyclodextrin and an aqueous carrier wherein the flavonoid is present in a concentration greater than 0.5 mM, 1 mM, 5 mM, 10 mM, 20 mM, 30 mM, 33 mM, 40 mM, 50 mM, 60 mM, 70 mM, 80 mM or greater than 80 mM. In some embodiments, the invention provides a composition comprising a quercetin or a quercetin derivative and a sulfobutylether-7-β-cyclodextrin and an aqueous carrier wherein the quercetin or a quercetin derivative is present in a concentration greater than 0.5 mM, 1 mM, 5 mM, 10 mM, 20 mM, 30 mM, 33 mM, 40 mM, 50 mM, 60 mM, 70 mM, 80 mM or greater than 80 mM.

In some embodiments, a composition is provided comprising a quercetin or a quercetin derivative and a sulfobutylether-7-β-cyclodextrin and an aqueous carrier wherein the quercetin or a quercetin derivative is present in a concentration greater than 0.5 mM. In some embodiments, the invention provides a composition comprising a quercetin or a quercetin derivative and a sulfobutylether-7-β-cyclodextrin and an aqueous carrier wherein the quercetin or a quercetin derivative is present in a concentration greater than 1 mM. In some embodiments, the invention provides a composition comprising a quercetin or a quercetin derivative and a sulfobutylether-7-β-cyclodextrin and an aqueous carrier wherein the quercetin or a quercetin derivative is present in a concentration greater than 5 mM. In some embodiments, the invention provides a composition comprising a quercetin or a quercetin derivative and a sulfobutylether-7-β-cyclodextrin and an aqueous carrier wherein the quercetin or a quercetin derivative is present in a concentration greater than 10 mM. In some embodiments, the invention provides a composition comprising a quercetin or a quercetin derivative and a sulfobutylether-7-β-cyclodextrin and an aqueous carrier wherein the quercetin or a quercetin derivative is present in a concentration greater than 20 mM. In some embodiments, the invention provides a composition comprising a quercetin or a quercetin derivative and a sulfobutylether-7-β-cyclodextrin and an aqueous carrier wherein the quercetin or a quercetin derivative is present in a concentration greater than 33 mM. In some embodiments, the invention provides a composition comprising a quercetin or a quercetin derivative and a sulfobutylether-7-β-cyclodextrin and an aqueous carrier wherein the quercetin or a quercetin derivative is present in a concentration greater than 40 mM. In some embodiments, the invention provides a composition comprising a quercetin or a quercetin derivative and a sulfobutylether-7-β-cyclodextrin and an aqueous carrier wherein the quercetin or a quercetin derivative is present in a concentration greater than 50 mM. In some embodiments, the invention provides a composition comprising a quercetin or a quercetin derivative and a sulfobutylether-7-β-cyclodextrin and an aqueous carrier wherein the quercetin or a quercetin derivative is present in a concentration greater than 60 mM. In some embodiments, the invention provides a composition comprising a quercetin or a quercetin derivative and a sulfobutylether-7-β-cyclodextrin and an aqueous carrier wherein the quercetin or a quercetin derivative is present in a concentration greater than 80 mM.

In some embodiments, the invention provides a composition comprising a flavonoid and a sulfobutylether-7-β-cyclodextrin and an aqueous carrier wherein the flavonoid is present in a concentration greater than 0.5 mM, 1 mM, 5 mM, 10 mM, 20 mM, 30 mM, 33 mM, 40 mM, 50 mM, 60 mM, 70 mM, 80 mM or greater than 80 mM, wherein the flavonoid is selected from the group consisting of quercetin or a quercetin derivative, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, and epicatechin.

In some embodiments, the molar ratio of flavonoid, e.g. quercetin, to cyclodextrin, e.g. sulfobutylether-7-β-cyclodextrin is between 1:1 and 1:40. In some cases, the molar ratio of flavonoid, e.g. quercetin, to cyclodextrin, e.g. sulfobutylether-7-β-cyclodextrin is between 1:1 and 1:40. In some cases, the molar ratio of flavonoid, e.g. quercetin, to cyclodextrin, e.g. sulfobutylether-7-β-cyclodextrin is between 1:1 and 1:5. In some cases, the molar ratio of flavonoid, e.g. quercetin, to cyclodextrin, e.g. sulfobutylether-7-β-cyclodextrin is between 1:2 and 1:4. In some cases, the molar ratio of flavonoid, e.g. quercetin, to cyclodextrin, e.g. sulfobutylether-7-β-cyclodextrin is between 1:10 and 1:40. In some cases, the molar ratio of flavonoid, e.g. quercetin, to cyclodextrin, e.g. sulfobutylether-7-β-cyclodextrin is between 1:15 and 1:40. In some cases, the molar ratio of flavonoid, e.g. quercetin, to cyclodextrin, e.g. sulfobutylether-7-β-cyclodextrin is between 1:3 and 1:12. In some cases, the molar ratio of flavonoid, e.g. quercetin, to cyclodextrin, e.g. sulfobutylether-7-β-cyclodextrin is between 1:5 and 1:10.

In some embodiments, a method of producing an aqueous solution of a flavonoid comprises mixing a flavonoid, a cyclodextrin, and a basic amino acid or sugar-amine at a pH of about 8.5 or greater. It has been found that the basic amino acid, such as lysine and arginine or a sugar-amine such as meglumine, can act, along with the cyclodextrin, to increase the solubility of the flavonoid in water.

As used in the method, the cyclodextrin is generally present at a level between 10% w/v to 40% w/v in the aqueous solution. In some cases the cyclodextrin is present between 15% and 35%. In some cases the cyclodextrin is present between 20% and 35%. In some cases the cyclodextrin is present between 20% and 35%. In some cases the cyclodextrin is present between 25% and 35%. In some cases the cyclodextrin is present between 30% and 35%. In some cases the cyclodextrin is present at about 10%, about 12%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 38% and about 40% w/v in the aqueous solution. In some cases the cyclodextrin is present in a range of 10% to 15%, 15% to 20%, 20% to 25%, 25% to 30%, 30% to 35%, or 35% to 40% w/v in the aqueous solution. In some cases it is found that having a level of cyclodextrin greater than about 20%, greater than about 25%, or greater than about 30% w/v in the aqueous solution can be used to obtain high solubility of the flavonoid. The cyclodextrin that works in this range can be, for example, a sulfoalkyl cyclodextrins such as sulfobutylether-β-cyclodextrin.

The flavonoid used in the method of producing an aqueous solution comprising the flavonoid, cyclodextrin and amino acid or sugar-amine can be a flavonoid known and/or described herein. The flavonoid can be, for example, quercetin or a quercetin derivative, fisetin or a fisetin derivative, galangin, or kaempferol. In some cases, the method provides the flavonoid, e.g. quercetin or a quercetin derivative, or fisetin or a fisetin derivative, at a concentration in a range between 1 mg/mL and 15 mg/mL, between 3 mg/mL and 14 mg/mL, between 5 mg/mL and 13 mg/mL, between 6 mg/mL and 12 mg/mL, between 8 mg/mL and 12 mg/mL, or between 9 mg/mL and 11 mg/mL. In some cases, the method provides the flavonoid, e.g. quercetin or a quercetin derivative at a concentration of greater than 1 mg/mL, greater than 2 mg/mL, greater than 4 mg/mL, greater than 3 mg/mL, greater than 5 mg/mL, greater than 6 mg/mL, greater than 7 mg/mL, greater than 8 mg/mL, greater than 9 mg/mL, greater than 10 mg/mL, greater than 11 mg/mL, greater than 12 mg/mL, greater than 13 mg/mL, greater than 14 mg/mL, or greater than 15 mg/mL.

In some cases, the method provides the flavonoid e.g. quercetin or a quercetin derivative, or fisetin or a fisetin derivative, at a concentration of greater than about 3 mM, greater than about 6 mM, greater than about 9 mM, greater than about 12 mM, greater than about 15 mM, greater than about 18 mM, greater than about 21 mM, greater than about 24 mM, greater than about 27 mM, greater than about 30 mM, or greater than about 33 mM.

The basic amino acid can be an amino acid having a basic group (in addition to the amine of the amino acid). The basic group can be, for example, an amine group or a guanidinium group. The pKa of the basic group will generally be greater than about 9.5, greater than about 10, greater than about 10.5, greater than about 11, or greater than about 11.5. The pKa of the basic group can be between about 9.5 and about 12, between about 10 and about 11.5, or between about 10.5 and 11.5. The amino acid can be a naturally occurring amino acid or a synthetic amino acid. In some cases it is desirable to use a naturally occurring basic amino acid in a pharmaceutical formulation. In some cases lysine is the amino acid. In some cases arginine is the amino acid. In some cases, both lysine and arginine are added together.

While in most cases, an amino acid is used, in some cases another basic compound can be used in place of the amino acid. For example, in some embodiments, a polyhydroxy compound or a sugar having an amine group (a sugar-amine) can be used in place of the amino acid or in conjunction with the amino acid. In some cases, for example, meglumine (N-Methyl-d-glucamine) can be used in place of the amino acid or in conjunction with the amino acid.

The amount of the amino acid can be the amount required to bring the pH of the solution above about 8.5, above about 8.7, or above about 9.0.

In some cases, the cyclodextrin, e.g. sulfobutylether-β-cyclodextrin, is first dissolved in water, then subsequently, the flavonoid and basic amino acid or sugar-amine are mixed to form the aqueous solution.

In some cases, the flavonoid, e.g. quercetin or a quercetin derivative, will degrade in the basic medium. Therefore, the time of mixing to form the aqueous solution will in some cases be minimized. In some cases, the mixing is done in less than about 1 hour, less than about 30 minutes, less than about 20 minutes, less than about 15 minutes, less than about 10 minutes, or less than about 5 minutes.

The temperature at which the mixing is carried out is generally near room temperature. In some cases, the temperature is between about 20° C. and about 25° C., between about 18° C. and about 28° C., between about 15° C. and about 30° C., between about 10° C. and about 25° C., between about 5° C. and about 20° C.

After the aqueous solution is formed, the pH of the solution can be neutralized by the addition of acid or by the addition of a buffer solution. In some cases, the acid is hydrochloric acid (HCL). The neutralized solution is generally brought to below pH 8.5. In some cases, the pH of the neutralized solution is about 8.5, about 8.4, about 8.2, about 8.0, about 7.8, about 7.6, about 7.4, about 7.2, or about 7.0.

The neutralized solution can then be dried to obtain a dry powder formulation comprising the flavonoid such as quercetin or a quercetin derivative, the cyclodextrin such as sulfobutylether-β-cyclodextrin, and the basic amino acid or sugar-amine. The dry powder can be stored, and can then be re-dissolved in water, for example to produce an intravenous solution. The dry powder can also be formulated as described below into a pharmaceutical formulation suitable for administration via various routes. The powder can be packaged into kits.

In some embodiments, the flavonoid, such as quercetin or a quercetin derivative, the cyclodextrin such as sulfobutylether-β-cyclodextrin, and the basic amino acid or sugar-amine are mixed in methanol. The methanol is then evaporated to yield a mixture which can be subsequently mixed in water to form an aqueous solution of flavonoid of the present invention. While not being bound by theory, the dissolution of the flavonoid in methanol and the subsequent precipitation of the flavonoid along with the cyclodextrin such as sulfobutylether-β-cyclodextrin is believed in some cases to break up the crystallinity of the flavonoid, promoting disruption of the crystalline lattice and fostering interaction with the other components in a manner that facilitates the subsequent dissolution of the flavonoid in water or aqueous solution. In some embodiments, quercetin, for example in the form of quercetin dihydrate, Captisol, and either arginine, lysine, or meglumine are mixed with methanol, the mixture is filtered from undissolved solids, and the solution obtained from filtration is treated in order to remove the methanol to obtain a solid residue. The removal of methanol can be accomplished, for example, by treating with molecular sieves, distillation, evaporation, or lyophilization. The solid residue can be stored or used right away. The solid residue can then be dissolved in water or aqueous solution to produce an aqueous solution of quercetin.

Accordingly, one embodiment is a dry powder formulation comprising the flavonoid such as quercetin or a quercetin derivative, the cyclodextrin such as sulfobutylether-β-cyclodextrin, and the basic amino acid or sugar-amine. In some cases, in the dry powder formulation, the molar ratio of the flavonoid, e.g. quercetin to the basic amino acid or sugar-amine is from about 3:1 to about 1:9. In some cases the molar ratio of the flavonoid, e.g. quercetin to the basic amino acid or sugar-amine is from about 1:1 to about 1:5. In some cases the molar ratio of the flavonoid, e.g. quercetin to the basic amino acid or sugar-amine is about 1:2. In some cases the molar ratio of the flavonoid, e.g. quercetin to the basic amino acid or sugar-amine is from about 1:1 to about 1:5 and the molar ratio of the flavonoid to the cyclodextrin such as sulfobutylether-β-cyclodextrin is about 1:12 to 1:2.

In some cases the molar ratio of the flavonoid, e.g. quercetin to the basic amino acid or sugar-amine is from about 3:1 to about 1:9 and the molar ratio of the flavonoid to the cyclodextrin such as sulfobutylether-β-cyclodextrin is about 1:1 to 1:40. In some cases the molar ratio of the flavonoid, e.g. quercetin to the basic amino acid or sugar-amine is from about 1:1 to about 1:5 and the molar ratio of the flavonoid to the cyclodextrin such as sulfobutylether-β-cyclodextrin is about 1:3 to 1:12. In some cases the molar ratio of the flavonoid, e.g. quercetin to the basic amino acid or sugar-amine is from about 1:1 to about 1:5 and the molar ratio of the flavonoid to the cyclodextrin such as sulfobutylether-β-cyclodextrin is about 1:5 to 1:10. The dry powder can be stored, and can then be re-dissolved in water, for example to produce an intravenous solution. The dry powder can also be formulated as described below into a pharmaceutical formulation suitable for administration via various routes. The powder can be packaged into kits.

In some embodiments the solutions of flavonoid produced by the above method are stable for a long period of time. In some embodiments, by using the methods of the invention, flavonoid solutions at relatively high concentrations can be stable to precipitation for about 5, 10, 20, 30, 45, or 60 minutes, for about 1, 2, 4, 8, 10, 12, 18, or 24 hours, for about 1, 2, 3, 5, 7, or 10 days, for 1, 2, 3, 4, 6 weeks, or for 1, 2, 3, 6, 9, or 12 months or 1, 2 3 or more years. The term “soluble” as used herein means that the flavonoid does not precipitate from the solution. In some embodiments, the soluble solution is substantially clear. In some embodiments the compositions can be stored at low temperature, e.g refrigerated, for the time periods described above without precipitation. For example, a composition of this invention with quercetin at 10 mg/ml in water with sulfobutylether-7-β-cyclodextrin is stable for more than two weeks without precipitation of the quercetin.

In some cases the method allows for the production of flavonoid-sulfoalkyl ether cyclodextrin aqueous compositions that have such a high concentrations that they tend to precipitate out of solution over time. For instance, the compositions may be clear and homogeneous for hours after their production by the methods of the invention, but will tend to precipitate after several hours at room temperature. These meta-stable high concentration solutions can still be useful, for instance if they are used within the time of solubility, or if they are further processed after having been produced at high concentration, for example being freeze-dried, or being diluted into formulations having long shelf life. It is known in the art how to characterize the stability of the fluids under various conditions to determine their usefulness for a given application.

The compositions disclosed herein can be used to make pharmaceutical formulations. In embodiments where the formulations provide a high concentration of the flavonoid in solution, these high concentration solutions can be useful for making pharmaceutical formulations. For example, in some embodiments, a composition with a high concentration of flavonoid and sulfoalkyl ether cyclodextrin can be dried, for example by freeze-drying or lyophilization in order to form a solid, powdered composition for use in a pharmaceutical formulation. The dried powder can then formulated with other components to make a pharmaceutical formulation for any suitable type of administration. For example, in some embodiments the dried powder can be mixed with other ingredients to create an oral formulation. In other embodiments, the dried powder can be made into a solid formulation that can be stored and then subsequently dissolved to produce a pharmaceutical formulation for injection.

In some embodiments, the high concentration form of flavonoid and sulfoalkyl ether cyclodextrin can be made as concentrated stock solution, and subsequently diluted for administration. It can be advantageous to have a high concentration stock solution for ease of manufacturing, storage, and handling.

In some embodiments, the invention provides a pharmaceutical composition that is made using an aqueous composition comprising a flavonoid and a sulfo-alkyl ether substituted cyclodextrin and an aqueous carrier wherein the flavonoid is present in a concentration greater than 0.5 mM, 1 mM, 5 mM, 10 mM, 20 mM, 30 mM, 33 mM, 40 mM, 50 mM, 60 mM, 70 mM, 80 mM or greater than 80 mM.

In some embodiments, the invention provides a pharmaceutical composition made from an aqueous composition comprising a flavonoid and a sulfobutylether-7-β-cyclodextrin and an aqueous carrier wherein the flavonoid is present in a concentration greater than 0.5 mM, 1 mM, 5 mM, 10 mM, 20 mM, 30 mM, 33 mM, 40 mM, 50 mM, 60 mM, 70 mM, 80 mM or greater than 80 mM. In some embodiments, the invention provides a composition comprising a quercetin or a quercetin derivative and a sulfobutylether-7-β-cyclodextrin and an aqueous carrier wherein the quercetin or a quercetin derivative is present in a concentration greater than 0.5 mM, 1 mM, 5 mM, 10 mM, 20 mM, 30 mM, 33 mM, 40 mM, 50 mM, 60 mM, 70 mM, 80 mM or greater than 80 mM.

In some embodiments, the invention provides a pharmaceutical composition made from an aqueous composition comprising a quercetin or a quercetin derivative and a sulfobutylether-7-β-cyclodextrin and an aqueous carrier wherein the quercetin or a quercetin derivative is present in a concentration greater than 0.5 mM. In some embodiments, the invention provides a pharmaceutical composition made from an aqueous composition comprising a quercetin or a quercetin derivative and a sulfobutylether-7-β-cyclodextrin and an aqueous carrier wherein the quercetin or a quercetin derivative is present in a concentration greater than 1 mM. In some embodiments, the invention provides a pharmaceutical composition made from an aqueous composition comprising a quercetin or a quercetin derivative and a sulfobutylether-7-β-cyclodextrin and an aqueous carrier wherein the quercetin or a quercetin derivative is present in a concentration greater than 5 mM. In some embodiments, the invention provides a pharmaceutical composition made from an aqueous composition comprising a quercetin or a quercetin derivative and a sulfobutylether-7-β-cyclodextrin and an aqueous carrier wherein the quercetin or a quercetin derivative is present in a concentration greater than 10 mM. In some embodiments, the invention provides a pharmaceutical composition made from an aqueous composition comprising a quercetin or a quercetin derivative and a sulfobutylether-7-β-cyclodextrin and an aqueous carrier wherein the quercetin or a quercetin derivative is present in a concentration greater than 20 mM. In some embodiments, the invention provides a pharmaceutical composition made from an aqueous composition comprising a quercetin or a quercetin derivative and a sulfobutylether-7-β-cyclodextrin and an aqueous carrier wherein the quercetin or a quercetin derivative is present in a concentration greater than 33 mM. In some embodiments, the invention provides a pharmaceutical composition made from an aqueous composition comprising a quercetin or a quercetin derivative and a sulfobutylether-7-β-cyclodextrin and an aqueous carrier wherein the quercetin or a quercetin derivative is present in a concentration greater than 40 mM. In some embodiments, the invention provides a pharmaceutical composition made from an aqueous composition comprising a quercetin or a quercetin derivative and a sulfobutylether-7-β-cyclodextrin and an aqueous carrier wherein the quercetin or a quercetin derivative is present in a concentration greater than 50 mM. In some embodiments, the invention provides a pharmaceutical composition made from an aqueous composition comprising a quercetin or a quercetin derivative and a sulfobutylether-7-β-cyclodextrin and an aqueous carrier wherein the quercetin or a quercetin derivative is present in a concentration greater than 60 mM. In some embodiments, the invention provides a pharmaceutical composition made from an aqueous composition comprising a quercetin or a quercetin derivative and a sulfobutylether-7-β-cyclodextrin and an aqueous carrier wherein the quercetin or a quercetin derivative is present in a concentration greater than 60 mM. In some embodiments, the invention provides a pharmaceutical composition made from an aqueous composition comprising a quercetin or a quercetin derivative and a sulfobutylether-7-β-cyclodextrin and an aqueous carrier wherein the quercetin or a quercetin derivative is present in a concentration greater than 80 mM.

In some embodiments, the invention provides a pharmaceutical composition made from an aqueous composition comprising a flavonoid and a sulfo-alkyl ether substituted cyclodextrin and an aqueous carrier wherein the flavonoid is present in a concentration greater than 0.5 mM, 1 mM, 5 mM, 10 mM, 20 mM, 30 mM, 33 mM, 40 mM, 50 mM, 60 mM, 70 mM, 80 mM or greater than 80 mM, wherein the flavonoid is selected from the group consisting of quercetin or a quercetin derivative, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, and epicatechin.

In some embodiments, the invention provides a pharmaceutical composition made from an aqueous composition comprising a flavonoid and a sulfobutylether-7-β-cyclodextrin and an aqueous carrier wherein the flavonoid is present in a concentration greater than 0.5 mM, 1 mM, 5 mM, 10 mM, 20 mM, 30 mM, 33 mM, 40 mM, 50 mM, 60 mM, 70 mM, 80 mM or greater than 80 mM, wherein the flavonoid is selected from the group consisting of quercetin or a quercetin derivative, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, and epicatechin.

In some embodiments, the invention provides a pharmaceutical composition made from an aqueous composition comprising a flavonoid and a sulfo-alkyl ether substituted cyclodextrin and an aqueous carrier wherein the flavonoid is present in a concentration greater than 0.5 mM, 1 mM, 5 mM, 10 mM, 20 mM, 30 mM, 33 mM, 40 mM, 50 mM, 60 mM, 70 mM, 80 mM or greater than 80 mM, wherein the administration is rectal, buccal, intranasal, transdermal, intravenous, intraperitoneal, parenteral, intramuscular, subcutaneous, orally, topical, as an inhalant, or via an impregnated or coated device such as a stent. In some embodiments, the invention provides pharmaceutical composition for intravenous administration made from an aqueous composition comprising a flavonoid and a sulfo-alkyl ether substituted cyclodextrin and an aqueous carrier wherein the flavonoid is present in a concentration greater than 0.5 mM, 1 mM, 5 mM, 10 mM, 20 mM, 30 mM, 33 mM, 40 mM, 50 mM, 60 mM, 70 mM, 80 mM or greater than 80 mM. In some embodiments, the pharmaceutical composition for intravenous administration is a solid. In some embodiments, the pharmaceutical composition for intravenous administration is made by removal of water, for example by freeze drying or lyophilization. In some embodiments the pharmaceutical composition for intravenous administration is a liquid.

The pharmaceutical formulation produced from the compositions can be processed and formulated as described herein.

Examples of additional suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, epsilon.-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, δ-valerolactone and isomers thereof, β-butyrolactone and isomers thereof, and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a patient using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

Oral pharmaceutical compositions. In embodiments where the novel formulations provide a high concentration of the flavonoid in solution, these high concentration solutions can be useful for making pharmaceutical formulations. For example, in some embodiments, a composition with a high concentration of flavonoid and sulfoalkyl ether cyclodextrin can be dried, for example by freeze-drying or lyophilization in order to form a solid, powdered composition for use in a pharmaceutical formulation. The dried powder can then formulated with other components to make a pharmaceutical formulation for any type of administration. For example, in some embodiments the dried powder can be mixed with other ingredients to create an oral formulation. Where the oral formulation is made from the aqueous composition of sulfoalkyl ether cyclodextrin-flavonoid, the oral formulation can be a solid formulation that is produced by drying the aqueous composition, for example by freeze-drying or lyophilization. Lyophilization is a freeze-drying process in which water is sublimed from the composition after it is frozen. The particular advantages of the lyophilization process are that biologicals and pharmaceuticals that are relatively unstable in aqueous solution can be dried without elevated temperatures (thereby eliminating the adverse thermal affects) and then stored in the dry state where there are few stability problems. Once the aqueous composition is dried, it can be handled, for example, as a dried powder. The dried powder can be further formulated into oral pharmaceutical compositions as described herein.

Pharmaceutical compositions for injection. In some embodiments, the invention provides a pharmaceutical composition for injection containing a combination of a calcineurin inhibitor and an agent that, e.g., reduces or eliminates hyperglycemia and/or one or more symptoms of hyperglycemia induced by the calcineurin inhibitor, and a pharmaceutical excipient suitable for injection. In some embodiments, the invention provides a pharmaceutical composition for injection containing a combination of a calcineurin inhibitor a cyclodextrin-complexed agent that, e.g. reduces or eliminates hyperglycemia and/or one or more symptoms of hyperglycemia induced by the calcineurin inhibitor, and a pharmaceutical excipient suitable for injection. Components and amounts of agents in the compositions are as described herein.

In some embodiments, the pharmaceutical composition for injection is made using an aqueous composition comprising quercetin or a quercetin derivative, or fisetin or fisetin derivative, a sulfobutylether-7-β-cyclodextrin, and a pharmaceutically or veterinarily acceptable aqueous carrier wherein the quercetin or a quercetin derivative, or fisetin or fisetin derivative, is present in a concentration of greater than 0.5 mM in the composition used to make the formulation. In some embodiments, the pharmaceutical composition for injection is made using an aqueous composition comprising quercetin or a quercetin derivative, or fisetin or fisetin derivative, a sulfobutylether-7-β-cyclodextrin, and a pharmaceutically or veterinarily acceptable aqueous carrier wherein the quercetin or a quercetin derivative is present in a concentration of greater than 1 mM in the composition used to make the formulation. In some embodiments, the pharmaceutical composition for injection is made using an aqueous composition comprising quercetin or a quercetin derivative, or fisetin or fisetin derivative, a sulfobutylether-7-β-cyclodextrin, and a pharmaceutically or veterinarily acceptable aqueous carrier wherein the quercetin or a quercetin derivative, or fisetin or fisetin derivative, is present in a concentration of greater than 5 mM in the composition used to make the formulation. In some embodiments, the pharmaceutical composition for injection is made using an aqueous composition comprising quercetin or a quercetin derivative, or fisetin or fisetin derivative, a sulfobutylether-7-β-cyclodextrin, and a pharmaceutically or veterinarily acceptable aqueous carrier wherein the quercetin or a quercetin derivative, or fisetin or fisetin derivative, is present in a concentration of greater than 10 mM in the composition used to make the formulation. In some embodiments, the pharmaceutical composition for injection is made using an aqueous composition comprising quercetin or a quercetin derivative, or fisetin or fisetin derivative, a sulfobutylether-7-β-cyclodextrin, and a pharmaceutically or veterinarily acceptable aqueous carrier wherein the quercetin or a quercetin derivative, or fisetin or fisetin derivative, is present in a concentration of greater than 15 mM in the composition used to make the formulation. In some embodiments, the pharmaceutical composition for injection is made using an aqueous composition comprising quercetin or a quercetin derivative, or fisetin or fisetin derivative, a sulfobutylether-7-β-cyclodextrin, and a pharmaceutically or veterinarily acceptable aqueous carrier wherein the quercetin or a quercetin derivative, or fisetin or fisetin derivative, is present in a concentration of greater than 20 mM in the composition used to make the formulation. In some embodiments, the pharmaceutical composition for injection is made using an aqueous composition comprising quercetin or a quercetin derivative, or fisetin or fisetin derivative, a sulfobutylether-7-β-cyclodextrin, and a pharmaceutically or veterinarily acceptable aqueous carrier wherein the quercetin or a quercetin derivative, or fisetin or fisetin derivative, is present in a concentration of greater than 30 mM in the composition used to make the formulation. In some embodiments, the pharmaceutical composition for injection is made using an aqueous composition comprising quercetin or a quercetin derivative, or fisetin or fisetin derivative, a sulfobutylether-7-β-cyclodextrin, and a pharmaceutically or veterinarily acceptable aqueous carrier wherein the quercetin or a quercetin derivative, or fisetin or fisetin derivative, is present in a concentration of greater than 33 mM in the composition used to make the formulation. In some embodiments, the pharmaceutical composition for injection is made using an aqueous composition comprising quercetin or a quercetin derivative, or fisetin or fisetin derivative, a sulfobutylether-7-β-cyclodextrin, and a pharmaceutically or veterinarily acceptable aqueous carrier wherein the quercetin or a quercetin derivative, or fisetin or fisetin derivative, is present in a concentration of greater than 40 mM in the composition used to make the formulation. In some embodiments, the pharmaceutical composition for injection is made using an aqueous composition comprising quercetin or a quercetin derivative, or fisetin or fisetin derivative, a sulfobutylether-7-β-cyclodextrin, and a pharmaceutically or veterinarily acceptable aqueous carrier wherein the quercetin or a quercetin derivative, or fisetin or fisetin derivative, is present in a concentration of greater than 50 mM in the composition used to make the formulation. In some embodiments, the pharmaceutical composition for injection is made using an aqueous composition comprising quercetin or a quercetin derivative, or fisetin or fisetin derivative, a sulfobutylether-7-β-cyclodextrin, and a pharmaceutically or veterinarily acceptable aqueous carrier wherein the quercetin or a quercetin derivative, or fisetin or fisetin derivative, is present in a concentration of greater than 60 mM in the composition used to make the formulation. In some embodiments, the pharmaceutical composition for injection is made using an aqueous composition comprising quercetin or a quercetin derivative, or fisetin or fisetin derivative, a sulfobutylether-7-β-cyclodextrin, and a pharmaceutically or veterinarily acceptable aqueous carrier wherein the quercetin or a quercetin derivative, or fisetin or fisetin derivative, is present in a concentration of greater than 80 mM in the composition used to make the formulation.

The forms in which the novel compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the transport protein modulator and/or the calcineurin inhibitor in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

In embodiments where the novel formulations provide a high concentration of the flavonoid in solution, these high concentration solutions can be useful for making pharmaceutical formulations. For example, in some embodiments, a composition with a high concentration of flavonoid and sulfoalkyl ether cyclodextrin can be dried, for example by freeze-drying or lyophilization in order to form a solid, powdered composition for use in a pharmaceutical formulation. The dried powder can then formulated with other components to make a pharmaceutical formulation for any type of administration. For example, in some embodiments the dried powder can be made into a solid formulation that can be stored and then readily dissolved produce a pharmaceutical formulation for injection.

Where the pharmaceutical composition for injection is made from the aqueous composition of sulfoalkyl ether cyclodextrin-flavonoid, pharmaceutical composition for injection can be made into a solid formulation that is produced by drying the aqueous composition, for example by freeze drying or lyophilization. Having a dried, solid formulation can be advantageous for increasing the shelf-life. The solid formulation can then be re-dissolved into solution for injection. The dried powder can be further formulated into pharmaceutical composition for injection as described herein.

Pharmaceutical compositions for topical (e.g., transdermal) delivery. In some embodiments, the invention provides a pharmaceutical composition for transdermal delivery containing a combination of a calcineurin inhibitor and an agent that, e.g., reduces or eliminates one or more symptoms induced by the calcineurin inhibitor, and a pharmaceutical excipient suitable for transdermal delivery. In some embodiments, the agent that e.g., reduces or eliminates one or more symptoms induced by the calcineurin inhibitor is a BTB transport protein modulator, e.g. a polyphenol such as a flavonol, as described elsewhere herein. In some embodiments, the pharmaceutical composition for transdermal delivery is a combination of a calcineurin inhibitor and sulfoalkyl ether cyclodextrin-flavonoid, e.g. sulfobutylether-7-β-cyclodextrin-flavonoid, and a pharmaceutical excipient suitable for transdermal delivery. Components and amounts of agents in the compositions are as described herein.

In some embodiments, the invention provides a pharmaceutical composition for transdermal delivery is an aqueous formulation comprising a flavonoid and a sulfo-alkyl ether substituted cyclodextrin and a pharmaceutically or veterinarily acceptable aqueous carrier wherein the flavonoid is present in a concentration greater than 0.5 mM, 1 mM, 5 mM, 10 mM, 20 mM, 30 mM, 33 mM, 40 mM, 50 mM, 60 mM, 70 mM, 80 mM or greater than 80 mM. In some embodiments, the invention provides a pharmaceutical composition for transdermal delivery is an aqueous formulation comprising quercetin or a quercetin derivative, or fisetin or a fisetin derivative, and a sulfobutylether-7-β-cyclodextrin and a pharmaceutically or veterinarily acceptable aqueous carrier wherein the flavonoid is present in a concentration greater than 0.5 mM, 1 mM, 5 mM, 10 mM, 20 mM, 30 mM, 33 mM, 40 mM, 50 mM, 60 mM, 70 mM, 80 mM or greater than 80 mM. In some embodiments, the pharmaceutical composition for transdermal delivery is made using an aqueous composition comprising a flavonoid, a sulfo-alkyl ether substituted cyclodextrin and a pharmaceutically or veterinarily acceptable aqueous carrier wherein the flavonoid is present in a concentration of greater than 0.5 mM, 1 mM, 5 mM, 10 mM, 20 mM, 30 mM, 33 mM, 40 mM, 50 mM, 60 mM, 70 mM, 80 mM or greater than 80 mM. In some embodiments, the pharmaceutical composition for transdermal delivery is made using an aqueous composition comprising a flavonoid, e.g. quercetin derivative, or fisetin or a fisetin derivative, and a sulfobutylether-7-β-cyclodextrin and a pharmaceutically or veterinarily acceptable aqueous carrier wherein the flavonoid is present in a concentration of greater than 0.5 mM, 1 mM, 5 mM, 10 mM, 20 mM, 30 mM, 33 mM, 40 mM, 50 mM, 60 mM, 70 mM, 80 mM or greater than 80 mM.

Compositions of the present invention can be formulated into preparations in solid, semi-solid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another preferred formulation for use in the methods of the present invention employs transdermal delivery devices (“patches”). Such transdermal patches may be used to provide continuous or discontinuous infusion of the transport protein modulator in controlled amounts, either with or without calcineurin inhibitor. Thus, in some embodiments the invention provides a transdermal patch incorporating a BTB transport protein modulator, e.g., a polyphenol such as a flavonoid (e.g., quercetin or a quercetin derivative). In some embodiments the invention provides a transdermal patch incorporating a BTB transport protein modulator, e.g., a polyphenol such as a flavonoid (e.g., quercetin or a quercetin derivative) in combination with a calcineurin inhibitor, e.g. tacrolimus.

The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

Pharmaceutical compositions for inhalation. Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner.

Other pharmaceutical compositions. Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, N.Y., 1990; Katzung, ed., Basic and Clinical Pharmacology, Ninth Edition, McGraw Hill, 20037ybg; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999); all of which are incorporated by reference herein in their entirety.

B. Kits

The invention also provides kits. The kits include an agent as described herein, in suitable packaging, and written material that can include instructions for use, discussion of clinical studies, listing of side effects, and the like. The kit may further contain a calcineurin inhibitor. In some embodiments, the calcineurin inhibitor and the agent are provided as separate compositions in separate containers within the kit. In some embodiments, the calcineurin inhibitor and the agent are provided as a single composition within a container in the kit. Suitable packaging and additional articles for use (e.g., measuring cup for liquid preparations, foil wrapping to minimize exposure to air, and the like) are known in the art and may be included in the kit. In some embodiments, the kits include a container comprising pharmaceutical formulation that is made using an aqueous composition comprising a flavonoid, a sulfo-alkyl ether substituted cyclodextrin and a pharmaceutically or veterinarily acceptable aqueous carrier wherein the flavonoid is present in a concentration of greater than 0.5 mM, 1 mM, 5 mM, 10 mM, 20 mM, 30 mM, 33 mM, 40 mM, 50 mM, 60 mM, 70 mM, 80 mM or greater than 80 mM in the composition used to make the formulation, and instructions for using the formulation to treat a disorder. In some embodiments, the kits can include a sulfobutylether-7-β-cyclodextrin-flavonoid for example sulfobutylether-7-β-cyclodextrin-quercetin, in suitable packaging, and written material that can include instructions for use, discussion of clinical studies, listing of side effects, and the like.

### Methods

In another aspect, the invention provides methods, including methods of treatment, methods of decreasing or increasing the concentration of a substance in a physiological compartment, and methods of enhancing a therapeutic effect of a substance.

The term “animal” or “animal subject” as used herein includes humans as well as other mammals. The methods generally involve the administration of one or more drugs for the treatment of one or more diseases. Combinations of agents can be used to treat one disease or multiple diseases or to modulate the side-effects of one or more agents in the combination.

The term “treating” and its grammatical equivalents as used herein includes achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

In some embodiments, the invention provides a method of treating a condition by administering to an animal suffering from the condition an effective amount a BTB transport protein activator sufficient to reduce or eliminate hyperglycemia and/or one or more symptoms of hyperglycemia.

In some embodiments, the invention provides a method of treating a condition by administering to an animal suffering from the condition an effective amount of a calcineurin inhibitor and an amount of a BTB transport protein activator sufficient to reduce or eliminate hyperglycemia and/or one or more symptoms of hyperglycemia induced by the calcineurin inhibitor.

In some embodiments, the invention provides a method of treating a condition by administering to an animal suffering from the condition an effective amount of a calcineurin inhibitor and an amount of a BTB transport protein activator sufficient to reduce or eliminate hyperglycemia and/or one or more symptoms of hyperglycemia and to increase a therapeutic effect of the calcineurin inhibitor. In some embodiments, the activator increases a plurality of therapeutic effects of the calcineurin inhibitor.

In some embodiments, the invention provides a method of treating a condition by administering to an animal suffering from the condition an effective amount of a calcineurin inhibitor and an amount of a BTB transport protein activator sufficient to reduce or eliminate hyperglycemia and/or one or more symptoms of hyperglycemia and to decrease or increase the concentration of the calcineurin inhibitor in a physiological compartment.

In some embodiments the animal is a mammal, e.g., a human.

In some embodiments, the invention provides a method of treating a condition by administering to an animal suffering from the condition an effective amount of a calcineurin inhibitor and an amount of a BTB transport protein activator sufficient to increase a therapeutic effect of a calcineurin inhibitor in a physiological compartment.

The calcineurin inhibitor and the BTB transport protein activator are co-administered. “Co-administration,” “administered in combination with,” and their grammatical equivalents, as used herein, encompasses administration of two or more agents to an animal so that both agents and/or their metabolites are present in the animal at the same time. Co-administration includes simultaneous administration in separate compositions, administration at different times in separate compositions, or administration in a composition in which both agents are present. Thus, in some embodiments, the BTB transport protein activator and the calcineurin inhibitor are administered in a single composition. In some embodiments, the calcineurin inhibitor and the BTB transport protein activator are admixed in the composition. Typically, the calcineurin inhibitor is present in the composition in an amount sufficient to produce a therapeutic effect, and the BTB transport protein activator is present in the composition in an amount sufficient to reduce hyperglycemia and/or one or more symptoms of hyperglycemia induced by the calcineurin inhibitor and/or decrease or increase the concentration of the calcineurin inhibitor in a physiological compartment and/or increase a therapeutic effect of the calcineurin inhibitor. In some embodiments, the calcineurin inhibitor is present in an amount sufficient to exert a therapeutic effect and the BTB transport protein activator is present in an amount sufficient to decrease hyperglycemia and/or one or more symptoms of hyperglycemia induced by the calcineurin inhibitor by an average of at least about 5, 10, 15, 20, 25, 30, 40, 50, 60, 70, 80, 90, more than 90%, or substantially eliminate hyperglycemia and/or one or more symptoms of hyperglycemia, compared to the effect without the BTB transport protein activator.

Administration of the agent that reduces or eliminate hyperglycemia and/or one or more symptom of hyperglycemia may be by any suitable means.

Administration of the calcineurin inhibitor and the agent, e.g., that reduces or eliminates hyperglycemia and/or one or more symptoms of hyperglycemia induced by the calcineurin inhibitor may be by any suitable means. If the agents are administered as separate compositions, they may be administered by the same route or by different routes. If the agents are administered in a single composition, they may be administered by any suitable route. In some embodiments, the agents are administered as a single composition by oral administration. In some embodiments, the agents are administered as a single composition by transdermal administration. In some embodiments, the agents are administered as a single composition by injection.

In some embodiments, the agent that reduces or eliminates hyperglycemia and/or one or more symptoms of hyperglycemia is a BTB transport protein modulator, BTB transport protein modulators are as described herein. In some embodiments, a polyphenol is used. In some embodiments, a flavonoid is used. In some embodiments, the flavonoid is quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, or epicatechin. In some embodiments, the flavonoid is quercetin, kaempferol, or galangin. In some embodiments, the flavonoid is quercetin or a quercetin derivative. Dosages are as provided for compositions. Typically, the daily dosage of the BTB transport protein modulator will be about 0.5-100 mg/kg.

The calcineurin inhibitor may be any calcineurin inhibitor described herein. In some embodiments, the calcineurin inhibitor is tacrolimus or a tacrolimus analog, as described herein.

The methods of the invention may be used for treatment of any suitable condition, e.g., chronic hyperglycemia, acute hyperglycemia, diabetes mellitus, non-diabetic hyperglycemia, stress-induced hyperglycemia, inflammation-induced hyperglycemia, organ transplant, an autoimmune disease, and an inflammatory disease.

For example, in some embodiments, the methods of the invention include the treatment of organ transplant recipient to prevent organ rejection by administering to an animal in need of treatment an effective amount of a calcineurin inhibitor, such as tacrolimus, and an effective amount of a BTB protein transport modulator that reduces or eliminates hyperglycemia and/or one or more symptoms of hyperglycemia induced by the calcineurin inhibitor. Example of organ transplant include, but are not limited to, kidney transplant, pancreas transplant, liver transplant, heart transplant, lung transplant, intestine transplant, pancreas after kidney transplant, and simultaneous pancreas-kidney transplant.

In other embodiments, the methods of the invention include the treatment of an autoimmune disease by administering to an animal in need of treatment an effective amount of a calcineurin inhibitor, such as tacrolimus, and an effective amount of a BTB protein transport modulator that reduces or eliminates hyperglycemia and/or one or more symptoms of hyperglycemia induced by the calcineurin inhibitor. Examples of autoimmune diseases include, but are not limited to, Lupus nephritis, actopic dermatitis, and psoriasis.

In yet other embodiments, the methods of the invention include the treatment of inflammatory conditions rejection by administering to an animal in need of treatment an effective amount of a calcineurin inhibitor, such as tacrolimus, and an effective amount of a BTB protein transport modulator that reduces or eliminates hyperglycemia and/or one or more symptoms of hyperglycemia induced by the calcineurin inhibitor. Examples of inflammatory conditions include, but are not limited to, asthma, vulvar lichen sclerosis, chronic allergic contact dermatitis, eczema, vitiligo and ulcerative colitis.

When a calcineurin inhibitor and an agent as described herein are used in combination, any suitable ratio of the two agents, e.g., molar ratio, wt/wt ration, wt/volume ratio, or volume/volume ratio, as described herein, may be used.

In other embodiments, the methods of the invention include the treatment of chronic hyperglycemia by administering to an animal in need of treatment an effective amount of a BTB protein transport modulator that reduces or eliminates hyperglycemia and/or one or more symptoms of hyperglycemia. In some embodiments, the methods of the invention include the treatment of acute hyperglycemia by administering to an animal in need of treatment an effective amount of a BTB protein transport modulator that reduces or eliminates hyperglycemia and/or one or more symptoms of hyperglycemia.

In some embodiments, the methods of the invention include the treatment of diabetes mellitus by administering to an animal in need of treatment an effective amount of a BTB protein transport modulator that reduces or eliminates hyperglycemia and/or one or more symptoms of hyperglycemia.

In some embodiments, the methods of the invention include the treatment of non-diabetic hyperglycemia by administering to an animal in need of treatment an effective amount of BTB protein transport modulator that reduces or eliminates hyperglycemia and/or one or more symptoms of hyperglycemia. Certain eating disorders can produce acute non-diabetic hyperglycemia, as in the binge phase of bulimia nervosa, when the subject consumes a large amount of calories at once, frequently from foods that are high in simple and complex carbohydrates. Certain medications increase the risk of hyperglycemia, including beta blockers, thiazide diuretics, corticosteroids, niacin, pentamidine, protease inhibitors, L-asparaginase, and some antipsychotic agents.

In some embodiments, the methods of the invention include the treatment of stress-induced hyperglycemia by administering to an animal in need of treatment an effective amount of a BTB protein transport modulator that reduces or eliminates hyperglycemia and/or one or more symptoms of hyperglycemia. A high proportion of patients suffering an acute stress such as stroke or myocardial infarction may develop hyperglycemia, even in the absence of a diagnosis of diabetes. Human and animal studies suggest that this is not benign, and that stress-induced hyperglycemia is associated with a high risk of mortality after both stroke and myocardial infarction.

In some embodiments, the methods of the invention include the treatment inflammation-induced hyperglycemia by administering to an animal in need of treatment an effective amount of an effective amount of a BTB protein transport modulator that reduces or eliminates hyperglycemia and/or one or more symptoms of hyperglycemia.

The invention further provides methods of reversing hyperglycemia and/or one or more symptoms of hyperglycemia induced by a calcineurin inhibitor by administering a BTB transport protein activator to an animal that has received an amount of the calcineurin inhibitor sufficient to produce hyperglycemia and/or one or more symptoms of hyperglycemia.

In some embodiments, the invention provides method of preventing, decreasing and/or reversing hyperglycemia and/or one or more symptoms of hyperglycemia induced by a calcineurin inhibitor by administering a BTB transport protein activator to an animal receiving treatment with a calcineurin inhibitor with a known or suspected symptom of hyperglycemia. In some embodiments, the animal, e.g. human, receiving treatment with a calcineurin inhibitor has tested positive for hyperglycemia (e.g. after a fasting glucose test) prior to administering the BTB transport protein activator. In some embodiments, the animal, e.g. human, receiving treatment with a calcineurin inhibitor has displayed one or more symptoms of hyperglycemia as described herein prior to administering the BTB transport protein activator. In some embodiments, the animal, e.g. human, receiving treatment with a calcineurin inhibitor possesses a trait (e.g. genetic trait or physical trait such as obesity) that makes the animal predisposed to hyperglycemia and/or one or more symptoms of hyperglycemia upon treatment with a calcineurin inhibitor; and a BTB transport protein activator is administered to the animal in combination with the calcineurin inhibitor to prevent hyperglycemia and/or one more symptoms of hyperglycemia. For example, a transplant patient undergoing treatment with a calcineurin inhibitor can be prescribed treatment with one or more of the BTB transport activators described herein after testing positive for hyperglycemia from a glucose blood level test such as the fasting glucose test. Alternatively, a transplant patient undergoing treatment with a calcineurin inhibitor that possesses a trait (e.g. genetic trait or physical trait such as obesity) that makes the animal the animal predisposed to hyperglycemia and/or one or more symptoms of hyperglycemia can be prescribed treatment with one or more of the BTB transport activators described herein to prevent hyperglycemia and/or one more symptoms of hyperglycemia, even when the patient is not experiencing hyperglycemia and/or one or more symptoms of hyperglycemia.

In some embodiments, the invention provides a method for reversing hyperglycemia and/or one or more symptoms of hyperglycemia induced by a calcineurin inhibitor in a human by administering to the human an amount of a BTB transport protein modulator sufficient to partially or completely reverse hyperglycemia and/or one or more symptoms of hyperglycemia induced by the calcineurin inhibitor, where the human has received an amount of said calcineurin inhibitor sufficient to produce hyperglycemia and/or one or more symptoms of hyperglycemia. In some embodiments, the human has received an overdose of the calcineurin inhibitor producing the hyperglycemia and/or one or more symptoms of hyperglycemia. In some embodiments, the individual continues to experience peripheral effects of the calcineurin inhibitor. In some embodiments, the BTB transport protein modulator is a polyphenol, such as a flavonoid. In some embodiments, the flavonoid is quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, or epicatechin. In some embodiments, the flavonoid is quercetin or a quercetin derivative. The flavonoid can be administered by any suitable route such as orally or by injection, e.g., intravenously or intraperitoneally, in a dose sufficient to partially or completely reverse hyperglycemia and/or one or more symptoms of hyperglycemia induced by the calcineurin inhibitor. Such a dose in a human can be, e.g., about 0.1-100 g, or about 0.5-50 g, or about 1-20 g, or 1, 2, 3, 4, 5, 6, 7, 8, 9, 10, 12, 14, 16, 18, or 20 g. In general, the dose can be 0.01-1.5 g/kg.

The invention further provides methods of increasing one or more therapeutic effects of a calcineurin inhibitor and decreasing hyperglycemia and/or one or more symptoms of hyperglycemia induced by the calcineurin inhibitor by administering a BTB transport protein activator to an animal that has received an amount of the calcineurin inhibitor sufficient to produce one or more therapeutic effects. In some embodiments, the invention provides a method for increasing a therapeutic effect of a calcineurin inhibitor in a human by administering to the human an amount of a BTB transport protein modulator sufficient increase one or more therapeutic effects of the calcineurin inhibitor, where the human has received an amount of said calcineurin inhibitor sufficient to produce a therapeutic effect. In some embodiments, there is an increase in the therapeutic effect of the calcineurin inhibitor with increase in dose of the BTB transport protein modulator. In some embodiments, there is a window to the increase in the therapeutic effect of the calcineurin inhibitor in which the therapeutic effect increase with increase in dose of the BTB transport protein modulator to a certain point, but then there is a decrease in the therapeutic effect with further increases in dose of the BTB transport protein modulator. In some embodiments, the BTB transport protein modulator is a polyphenol, such as a flavonoid. In some embodiments, the flavonoid is quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, or epicatechin. In some embodiments, the flavonoid is quercetin or a quercetin derivative. The flavonoid can be administered by any suitable route such as orally or by injection, e.g., intravenously or intraperitoneally, in a dose sufficient to increase a therapeutic effect of the calcineurin inhibitor. Such a dose in a human can be, e.g., about 0.1-100 g, or about 0.5-50 g, or about 1-20 g, or 1, 2, 3, 4, 5, 6, 7, 8, 9, 10, 12, 14, 16, 18, or 20 g. In general, the dose can be 0.01-1.5 g/kg. In general the dose can be 0.02-0.5 g/kg. In general the dose can be 0.15-0.5 g/kg.

The invention further provides methods of decreasing or increasing the concentration of a calcineurin inhibitor in a physiological compartment and decreasing hyperglycemia and/or one or more symptoms of hyperglycemia induced by the calcineurin inhibitor by administering a BTB transport protein activator to an animal that has received an amount of the calcineurin inhibitor sufficient to decrease or increase the concentration of a calcineurin inhibitor in a physiological compartment and decrease hyperglycemia and/or one or more symptoms of hyperglycemia induced by the calcineurin inhibitor.

In some embodiments, the invention provides a method for decreasing or increasing the concentration of a calcineurin inhibitor in a physiological compartment and decreasing hyperglycemia and/or one or more symptoms of hyperglycemia induced by the calcineurin inhibitor in a human by administering to the human an amount of a BTB transport protein modulator sufficient to decrease or increase the concentration of a calcineurin inhibitor in a physiological compartment and decrease hyperglycemia and/or one or more symptoms of hyperglycemia induced by the calcineurin inhibitor, where the human has received an amount of said calcineurin inhibitor sufficient for treatment. In some embodiments, the invention provides a method for decreasing or increasing the concentration of tacrolimus or a tacrolimus analog in a physiological compartment in a human by administering to the human an amount of a BTB transport protein modulator sufficient to decrease or increase the concentration of tacrolimus or a tacrolimus analog in a physiological compartment, where the human has received an amount of tacrolimus or a tacrolimus analog sufficient for treatment. In some embodiments, the BTB transport protein modulator is a polyphenol, such as a flavonoid. In some embodiments, the flavonoid is quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, or epicatechin. In some embodiments, the flavonoid is quercetin or a quercetin derivative. Typically, the flavonoid will be administered by injection, e.g., intravenously or intraperitoneally, in a dose sufficient to increase a therapeutic effect of the calcineurin inhibitor. Such a dose in a human can be, e.g., about 0.1-100 g, or about 0.5-50 g, or about 1-20 g, or 1, 2, 3, 4, 5, 6, 7, 8, 9, 10, 12, 14, 16, 18, or 20 g. In general, the dose can be 0.01-1.5 g/kg. In general the dose can be 0.02-0.5 g/kg. In general the dose can be 0.15-0.5 g/kg.

A further aspect of the invention is a method of identifying a transport protein modulator. A drug is administered in an appropriate animal model in the presence and absence of a test compound and the concentration of the drug in a biological sample are measured. The test compound is identified as a transport protein modulator if the concentration of the drug in the biological sample is lower in the presence of the test compound. In some embodiments, the biological sample may be intraventricular samples, amniotic fluid, chorionic samples or brain parenchymal samples. Moreover, the animal model may be a rodent, such as mice or rats, or a primate, horse, dog, sheep, goat, rabbit, or chicken. In other embodiments, the animal model possesses a mutant form of a blood brain transporter.

### Administration

The methods involve the administration of an agent as described herein. For simplicity, administration will be described in terms of reduction of hyperglycemia and/or one or more symptoms of hyperglycemia induced by a calcineurin inhibitor. It is understood that the administration apply equally to other methods described herein.

In some embodiments, a calcineurin inhibitor that produces hyperglycemia and/or one or more symptoms of hyperglycemia is administered in combination with an agent that reduces hyperglycemia and/or one or more symptoms of hyperglycemia induced by the calcineurin inhibitor. In some embodiments, other agents are also administered, e.g., other calcineurin inhibitors. When two or more agents are co-administered, they may be co-administered in any suitable manner, e.g., as separate compositions, in the same composition, by the same or by different routes of administration.

In some embodiments, the agent that reduces or eliminates hyperglycemia and/or one or more symptoms of hyperglycemia is administered in a single dose.

In some embodiments, the agent that reduces or eliminates hyperglycemia and/or one or more symptoms of hyperglycemia is administered in multiple doses. Dosing may be about once, twice, three times, four times, five times, six times, or more than six times per day. In some embodiments, dosing may be about once a month, once every two weeks, once a week, once every other day or any other suitable interval. In one embodiment the calcineurin inhibitor is tacrolimus. In another embodiment the calcineurin inhibitor and the transport protein activator are administered together about once per day to about 6 times per day. In another embodiment the administration of the calcineurin inhibitor and the transport protein activator continues for less than about 7 days. In yet another embodiment the administration continues for more than about 6, 10, 14, 28 days, two months, six months, or one year. In some cases, continuous dosing is achieved and maintained as long as necessary, e.g., in an organ transplant patient, which may require dosing for the rest of their life.

Administration of the agents of the invention may continue as long as necessary. In some embodiments, an agent of the invention is administered for more than about 1, 2, 3, 4, 5, 6, 7, 14, or 28 days. In some embodiments, an agent of the invention is administered for less than about 28, 14, 7, 6, 5, 4, 3, 2, or 1 day. In some embodiments, an agent of the invention is administered chronically on an ongoing basis, e.g., for the treatment of chronic effects.

An effective amount of a transport protein modulator and an effective amount of a calcineurin inhibitor may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer.

The BTB transport protein modulator and the calcineurin inhibitor may be administered in dosages as described herein (see, e.g., Compositions). Dosing ranges for calcineurin inhibitors are known in the art. It is also known in the art that due to intersubject variability in calcineurin inhibitors, such as tacrolimus, pharmacokinetics, individualization of dosing regimen is necessary for optimal therapy. Dosing for the BTB transport modulator may be found by routine experimentation. For a flavonoid, e.g., quercetin or a quercetin derivative, typical daily dose ranges are, e.g. about 1-5000 mg, or about 1-3000 mg, or about 1-2000 mg, or about 1-1000 mg, or about 1-500 mg, or about 1-100 mg, or about 10-5000 mg, or about 10-3000 mg, or about 10-2000 mg, or about 10-1000 mg, or about 10-500 mg, or about 10-200 mg, or about 10-100 mg, or about 20-2000 mg or about 20-1500 mg or about 20-1000 mg or about 20-500 mg, or about 20-100 mg, or about 50-5000 mg, or about 504000 mg, or about 50-3000 mg, or about 50-2000 mg, or about 50-1000 mg, or about 50-500 mg, or about 50-100 mg, about 100-5000 mg, or about 1004000 mg, or about 100-3000 mg, or about 100-2000 mg, or about 100-1000 mg, or about 100-500 mg. In some embodiments, the daily dose of quercetin or a quercetin derivative is about 100, 200, 300, 400, 500, 600, 700, 800, 900, or 1000 mg. In some embodiments, the daily dose of quercetin or a quercetin derivative is 100 mg. In some embodiments, the daily dose of quercetin or a quercetin derivative is 500 mg. In some embodiments, the daily dose of quercetin or a quercetin derivative is 1000 mg. Daily doses may be administered in single or multiple doses. For instance, in some embodiments the BTB transport modulator is administered 3 times per day of an oral dose of 500 mg. In other embodiments the BTB transport modulator is administered 3 times per day of an i.v. dose of 150 mg. Daily doses of quercetin or a quercetin derivative may be administered in the same or separate composition as the calcineurin inhibitor. In some embodiments, the BTB transport protein modulator is in the bloodstream 30 minutes prior to the therapeutic agent. This may be accomplished by administering the BTB transport modulator separately from the calcineurin inhibitor or by administering the BTB transport modulator and calcineurin inhibitor in the same composition that is formulated so that the BTB transport modulator reaches the bloodstream before the calcineurin inhibitor. Daily dose range may depend on the form of flavonoid, e.g., the carbohydrate moieties attached to the flavonoid, and/or factors with which the flavonoid is administered, as described herein. The serum half-life for, e.g., quercetin or a quercetin derivative, is about 19-25 hours, so single dose accuracy is not crucial.

When a BTB transport modulator, e.g., a flavonoid such as quercetin or a quercetin derivative is administered in a composition that comprises one or more calcineurin inhibitors, and the calcineurin inhibitor has a shorter half-life than BTB transport modulator unit dose forms of the calcineurin inhibitor and the BTB transport modulator may be adjusted accordingly. Thus, for example, if quercetin or a quercetin derivative is given in a composition also containing, e.g., calcineurin inhibitor, a typical unit dose form is, e.g., 50 mg calcineurin inhibitor/100 mg quercetin, or 50 mg calcineurin inhibitor/500 mg quercetin. See e.g., Compositions.

When a BTB transport protein that is the target of the BTB transport modulator is present on the cells where the calcineurin inhibitor is exerting its therapeutic effect, unit dose forms of the BTB transport modulator may be adjusted such that hyperglycemia and/or one or more symptoms of hyperglycemia induced by the calcineurin inhibitor are reduced without a substantial reduction of the therapeutic effect.

### EXAMPLES

### Example 1

### Preparation of a sulfobutylether-7-β-cyclodextrin Aqueous Composition

Under an inert atmosphere, 18.7 g of sulfobutylether-7-β-cyclodextrin (Captisol™, CyDex) is dissolved in about 50 ml of deionized (DI) water in a round-bottomed flask with magnetic stirring. The flask is placed in an ice bath. When all of the Captisol is dissolved, 1.24 g of quercetin (Micron Technologies) (equivalent to about 1 g of anhydrous quercetin) is added with stirring. Into the flask, 12 ml of 1 N sodium hydroxide is added over about 5 to 10 minutes. The appearance of the reaction should be clear indicating that both the Captisol and the quercetin are dissolved. Into the flask is then added 10.5 ml of hydrochloric acid over 5 to 10 minutes at a slow enough rate to avoid precipitation. During the addition of the sodium hydroxide and the hydrochloric acid, the temperature is maintained at less than 20° C. DI water is then added to give total volume of 100 ml. This procedure results in a sulfobutylether-7-β-cyclodextrin-quercetin aqueous composition at a concentration of 10 mg/ml (33 mM) in quercetin at a pH of about 7.8. This solution was found to be stable on storage for weeks without precipitation.

In a variation of the above method, 9 ml rather than 10.5 ml of hydrochloric acid is added to make a solution with a pH of about 8.4.

### Example 2

### Solubility of Quercetin with Sulfobutylether-7-β-cyclodextrin and Meglumine

Sulfobutylether-7-β-cyclodextrin (Captisol™) is dissolved in water to form a solution at 30% w/v. To the Captisol solution is added Meglumine at a concentration of 44 mM and Captisol™ at a concentration of about 20 mg/ml. The solution is stirred at room temperature for about 10 minutes. The solution is separated from any excess solids (e.g. by filtration). The concentration of quercetin in the solution is about 9.2 mg/mL.

### Example 3

### Human Study of the Effects of Quercetin (Q) and Tacrolimus on Transplant Patients

An empirical trial on the effects of oral quercetin (Q) on tacrolimus induced hyperglycemia is conducted. Inclusion criteria include patients who have received liver, kidney or heart transplantation, under tacrolimus treatment who demonstrate hyperglycemia and/or one or more symptoms of hyperglycemia. Preferably, these patients have no history of prior transplantation or of hyperglycemia. The Table, below, provides exemplary dosing schemes for tacrolimus.

| | Tacrolimus |
|---|---|
| | Route |
| Population | (Dose) |
| Kidney Transplant | IV |
| | (0.02 mg/kg/12 hr |
| | Oral |
| | (0.3 mg/kg/day) |
| Liver Transplant | IV |
| | (0.05 mg/kg/12 hr) |
| | Oral |
| | (0.3 mg/kg/day |
| Heart Transplant | IV |
| | (0.01 mg/kg/day) |
| | Oral |
| | (0.15 mg/kg/day) |

Due to intersubject variability in tacrolimus pharmacokinetics, individualization of dosing regimen is necessary for optimal therapy. The dose of tacrolimus is adjusted daily to achieve a trough concentration of 15 to 20 and approximately 10 ng/mL in the first 2 weeks and the subsequent 2 weeks, respectively. A blood sample is collected before the morning dose for measuring the concentrations. The tacrolimus whole-blood concentration is measured using the microparticle enzyme immunoassay method, which is known in the art.

Quercetin 100-500 mg per gel capsule is compounded and supplied to all subjects. In some trials, placebo capsules are also compounded. Blood samples are taken at regular intervals, e.g., daily, and blood glucose levels are measured, e.g., fasting glucose test. Sugar levels on the patient can be measured via the HbA1c test. Optionally, subjects are instructed to complete daily diaries for 7-21 days and continue their baseline medications and regular activities. On approximately the 7th day, they are asked to begin twice daily dosing of 2 Q (200-1000 mg) capsules (total daily dose of Q, 400-2000 mg), or an equivalent dosage of placebo, preferably double-blinded (if placebo is used). Diaries are then completed for 7 days.

Individual diaries include rating loss of consciousness, blurred vision, headaches, coma, weight loss, polyphagia, polyuria, polydipsia, stomach problems, intestinal problems, poor wound healing, dry mouth, nausea, vomiting, dry skin, itchy skin, impotence, hypeventilation, fatigue, weakness on one side of the body, hallucinations, impairment in cognitive function, increase sadness, anxiety, recurrent genital infections, increase sugar in urine, retinopathy, nepropathy, arteriosclerotic disorders, cardiac arrhythmia, stupor, susceptibility to infection, neuropathy, cold feet, insensitive feet and loss of hair. Subjects are instructed that concomitant medications should not be altered without speaking with the investigator. Subjects are advised that they will be contacted every day or every other day to assess progress in the trial and any side effects associated with the addition of Quercetin. At the end of the trial, patients are interviewed. They are asked to rate their satisfaction with the study medication (−2-+2) and its ability to modulate hyperglycemia and/or one or more symptoms of hyperglycemia.

If the study has used placebo and is blinded, the blind is broken and statistical comparisons of Quercetin versus placebo are performed.

### Example 4

### Quercetin Decreases Tacrolimus Induced Hyperglycemia

Animals: 8-9 weeks-old rats are used. General procedures for animal care and housing are in accordance with the National Research Council (NRC) Guide for the Care and Use of Laboratory Animals (1996) and the Animal Welfare Standards incorporated in 9 CFR Part 3, 1991.

Treatment: Rats are treated i.v with FK506 and i.p. with Quercetin. Rats receive daily administrations containing either 0.5 mg/kg or 2 mg/kg of FK506 approximately one hour after the start of the light cycle. Rats are treated 30 minutes prior to FK506 treatment with quercetin at three different concentrations, 10 mg/kg, 25 mg/kg or 200 mg/kg. Subsets of 5 rats per group are used for blood sampling at each time point. Blood is collected for glucose measurement on days 0, 5, 9, and 14.

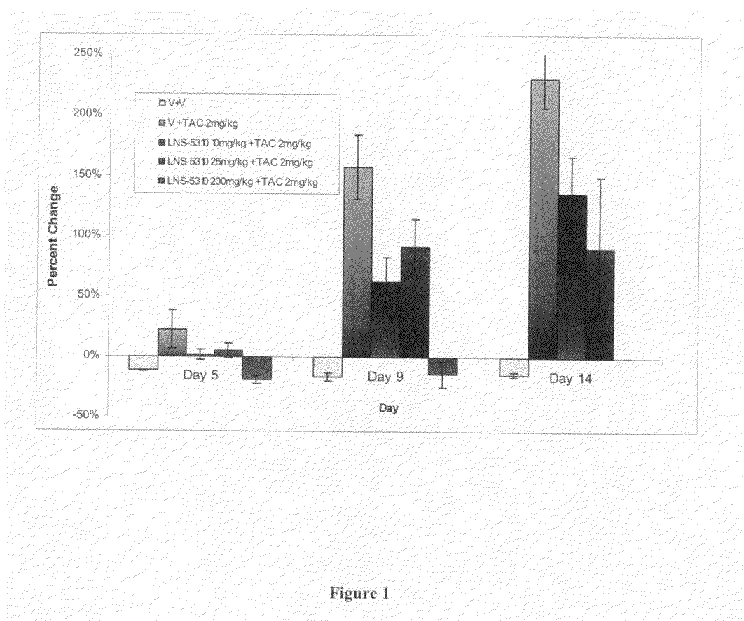

Results: FIG. 1 shows that quercetin decreases FK506 induced hyperglycemia when rats are treated with 2 mg/kg of FK506. At day one the mean for glucose blood level in the rats was 1.38 g/L. By day 5 there is an increase glucose blood levels in the group treated with FK506 when compare to rats treated with vehicle alone. This increase in glucose blood levels is reduced by treatment with quercetin at all concentrations. At day 9 the increase of glucose blood levels is changed 150%, the mean for glucose blood level in rats treated with FK506 was 3.23±0.33 g/L. The increase of glucose blood levels by FK506 is reduced by treatment with quercetin at all concentrations. At 10 mg/kg and 25 mg/kg the increase of blood glucose is reduced to 60% and 90%, respectively. However, at 200 mg/kg the percent change is reduced to the levels similar to rats treated with vehicle alone. Similar results are observed at day 14 are the mean for glucose blood level in rats treated with FK506 was 4.15±0.30 g/L, over a 200 percent change.

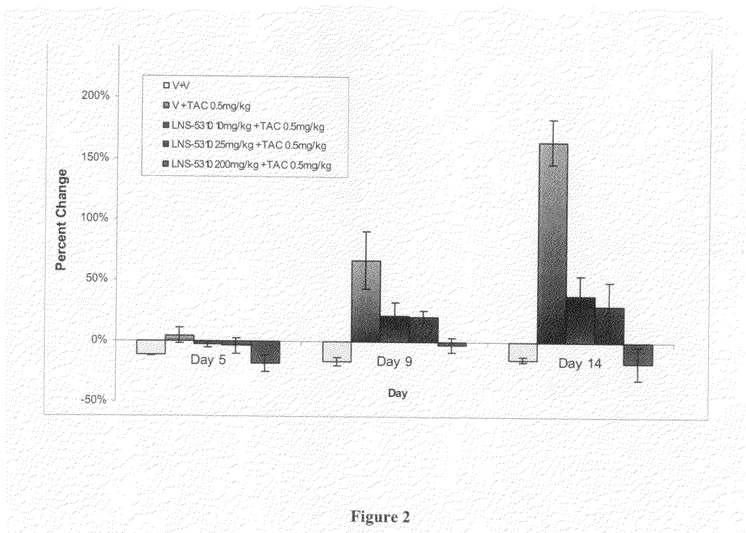

FIG. 2 shows that quercetin decreases FK506 induced hyperglycemia when rats are treated with 0.5 mg/kg of FK506. At day one the mean for glucose blood level in the rats is 1.38 g/L. By day 5 there is a slight increase in the group treated with FK506 when compare to rats treated with vehicle alone or FK506 in combination with quercetin. By days 9 and 14, however, there is a greater increase in the percent change of glucose blood level. The mean for glucose blood level at day 9 and 14 in rats treated with FK506 is 2.33±0.33 g/L and was 3.69±0.25 g/L, respectively. The increase of glucose blood levels by FK506 is reduced by treatment with quercetin at all concentrations with a more dramatic effect observed at 200 mg/kg of quercetin were the glucose blood levels were similar to rats treated with vehicle alone.

These results indicate that quercetin decreases FK506 induced hyperglycemia.

### Example 5

### BTB Transport Protein Activator Increases Tacrolimus Efficacy

Animals: 8-9 weeks-old Lewis and Brown Norway male rats are obtained from Charles River Laboratories. General procedures for animal care and housing are in accordance with the National Research Council (NRC) Guide for the Care and Use of Laboratory Animals (1996) and the Animal Welfare Standards incorporated in 9 CFR Part 3, 1991.

Treatment: Lewis rats are treated with different single doses of LNS 0694 i.p. 30 minutes prior to single i.v. injections of tacrolimus at a concentration of 1 mg/kg as described in the table below.

| | LNS 0694™ | |
|---|---|---|
| | (BTB Transport | |
| | Protein Activator) | FK506 |
| | Treatment | Treatment |
| Group | (IP) | (IV) |
| 1 | Baseline | — |
| | (Untreated Control) | |
| 2 | 50 mg/kg | 1 mg/kg |
| 3 | 150 mg/kg | 1 mg/kg |
| 4 | 300 mg/kg | 1 mg/kg |
| 5 | — | 1 mg/kg |

Dose calculations (mg/kg) are based on the individual body weight measured on the day of treatment.

Spleens are collected at 4 hr after administration of FK506 for use in the in vitro mixed lymphocyte reaction (MLR) and Con A assays. In addition to treated Lewis rats, spleens are collected from 5 Brown Norway rats (not treated) which are used for the in vitro assay.

Mixed lymphocyte reactions: A single cell suspension of the spleen of each rat on test (LEW, responder) is prepared with a Dounce homogenizer and wash-medium. The cell suspension is depleted of red blood cells (treatment with NH4CL/Tris buffer) and washed twice with wash medium before resuspending in complete medium (CM; RPMI 1640 with 5% heat-inactivated (30 minutes at 56° C.) normal rat serum (from Lewis rats), 2 mM GlutaMAX, 100 U/ml penicillin and 100 μg/ml streptomycin mixture, and 55 μM 2-mercaptoethanol). A single cell suspension of splenocytes from five Brown Norway rats (BN, stimulators) is prepared with the same method. The BN splenocytes are pooled and irradiated with 1,500-2,000 rad (cesium source) before use.

Varying numbers of responder cells are mixed with a constant number of stimulators (105) in 96-well, U-bottom cell culture plates to give a final responder:stimulator (R:S) ratio of 10:1, 5:1, 1:1 and 0.5:1 in 200 μl CM. Control wells for each cell suspension contained 10⁵ responders alone in medium and 105 pooled, irradiated stimulators alone in medium (separate wells). For a positive proliferative response, each responder (10⁵) is treated with 2.5 μg/ml Con A.

Cultures are incubated at 37±° 1 C. for 72±2 hours in 5±1% CO₂ humidified air. Each well is pulsed with 1 μCi tritiated thymidine for 18±2 hours before automatic harvesting and analysis in a liquid scintillation counter.

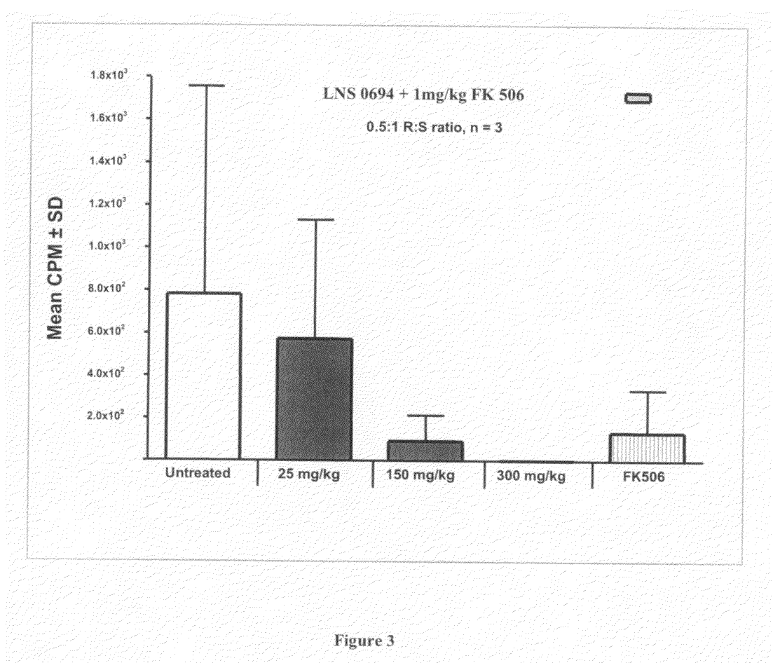

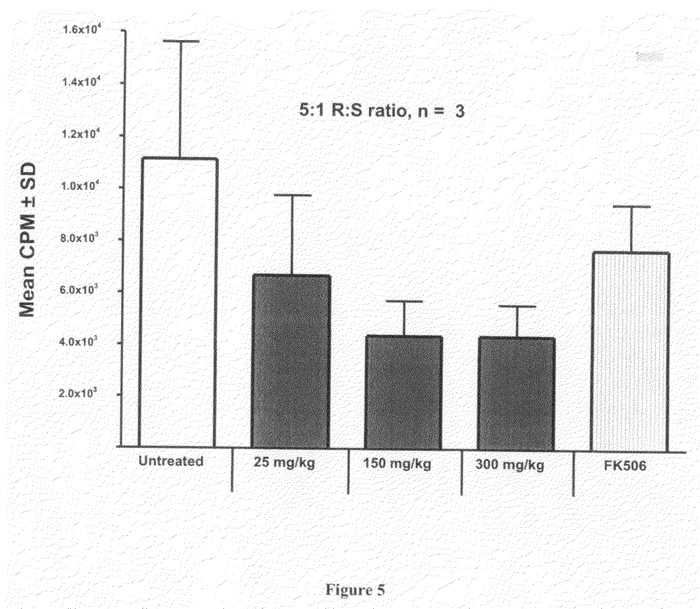

The results of the MLR assays [thymidine incorporation as counts per minute (CPM)] are expressed as mean±SD. Results are shown in FIGS. 3-6.

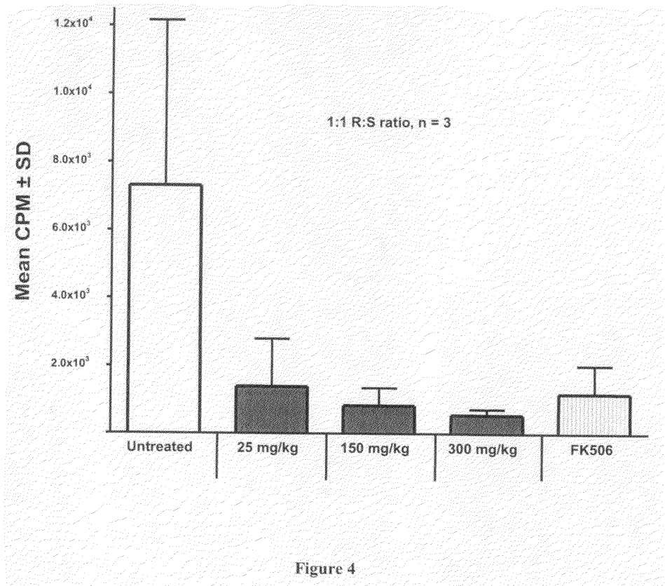

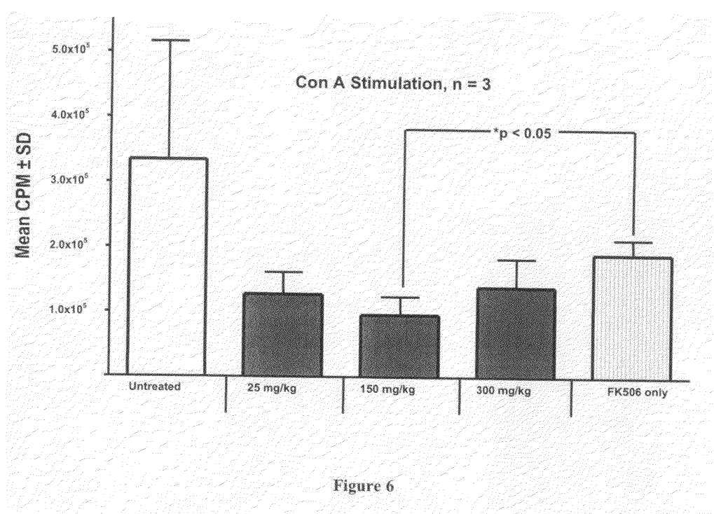

Results: The effect of BTB transport protein activator on FK 506-inhibitory effects on lymphocyte proliferation is evaluated by mixing LEW, responder and allogeneic BN, stimulators at three different ratios. As shown in FIGS. 3-5, untreated LEW, responders' proliferation increases with increases in R:S ratio (FIG. 3-5). As expected, FK 506 inhibited MLR. FK 506 exhibits a stronger inhibitory effect at lower R:S ratios (See FIGS. 3 and 4). LNS 0694 increases FK 506 inhibitory effect in a dose-dependent manner. As shown in FIG. 6, LNS 0694 also increases FK 506 inhibitory effect when the LEW, responders are activated with Con A.

These results suggest that FK 506 efficacy is enhanced when combined with a BTB transport protein modulator.

### Example 6

### BTB Transport Protein does not Impair Tacrolimus Induced T Cell Suppression In Vitro

Animals: 8-9 weeks-old Lewis and Brown Norway male rats are obtained from Charles River Laboratories. General procedures for animal care and housing is in accordance with the National Research Council (NRC) Guide for the Care and Use of Laboratory Animals (1996) and the Animal Welfare Standards incorporated in 9 CFR Part 3, 1991.

Spleens are collected for use in in vitro Con A and LPS assays.

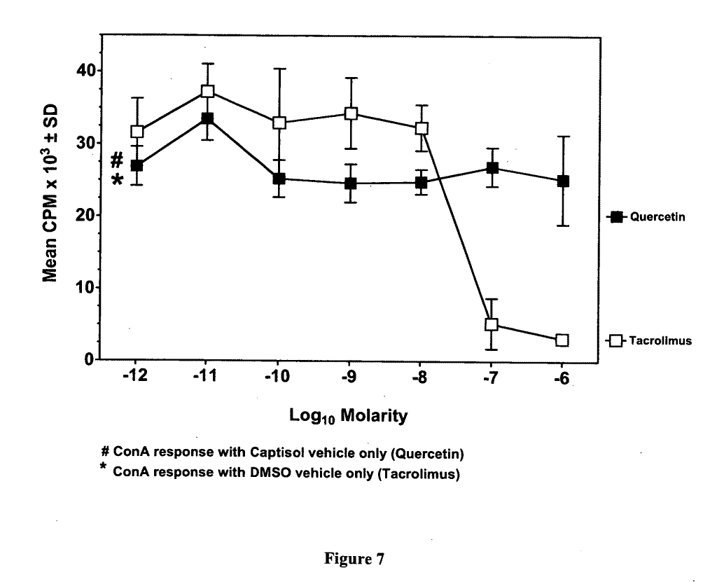

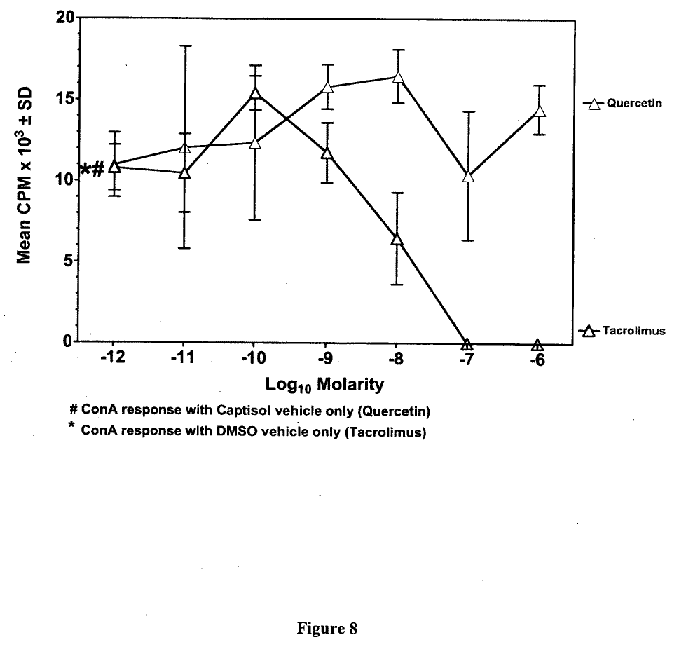

Results: FIGS. 7 and 8 show the effect of quercetin and tacrolimus on response of mouse spleen cells to Con A at a high (1.6×10⁶ cells/well) and low (8×10⁵ cells/well) cell concentration, respectively. As expected, tacrolimus inhibits Con A-induced proliferation in a dose dependent manner in cultures at high and low cell concentration. Quercetin has no significant effect in the response of cells to Con A at a high or low cell concentration.

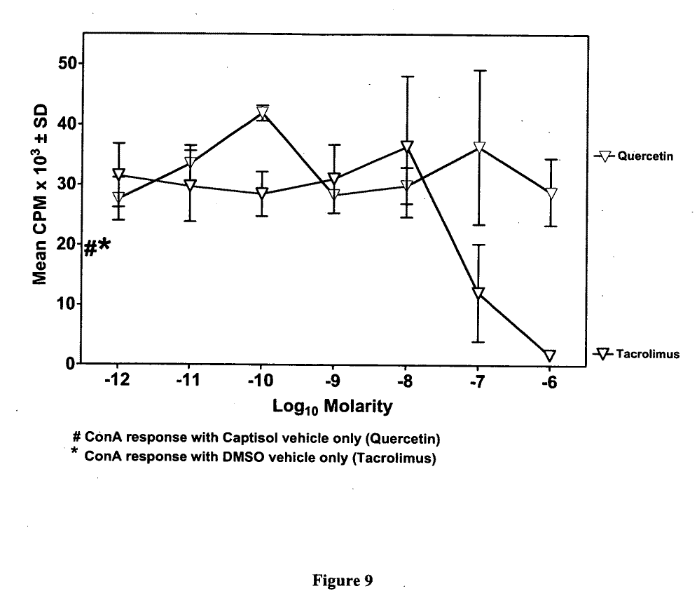

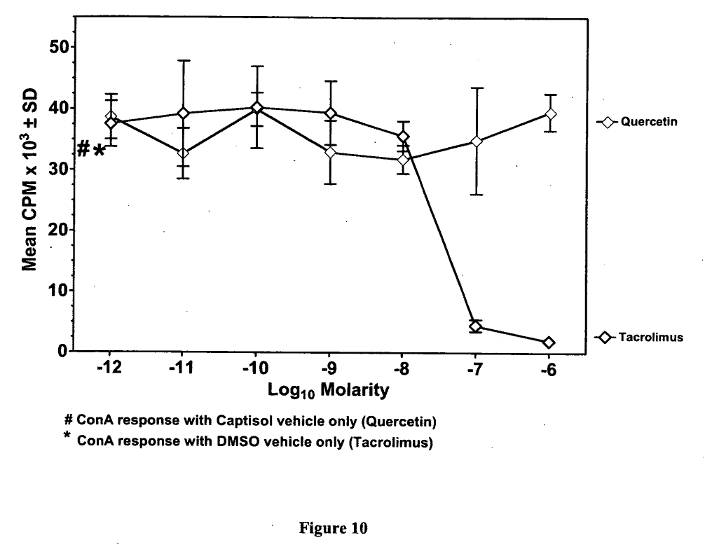

FIGS. 9 and 10 show the effect of quercetin and tacrolimus on response of mouse spleen cells to LPS at a high (1.6×10⁶ cells/well) and low (8×10⁵ cells/well) cell concentration, respectively. Like in the Con A assay, tacrolimus inhibits LPS-induced proliferation in a dose dependent manner both in high and low cell concentration cultures. Quercetin has no significant effect in the response of cells to LPS at a high or low cell concentration.

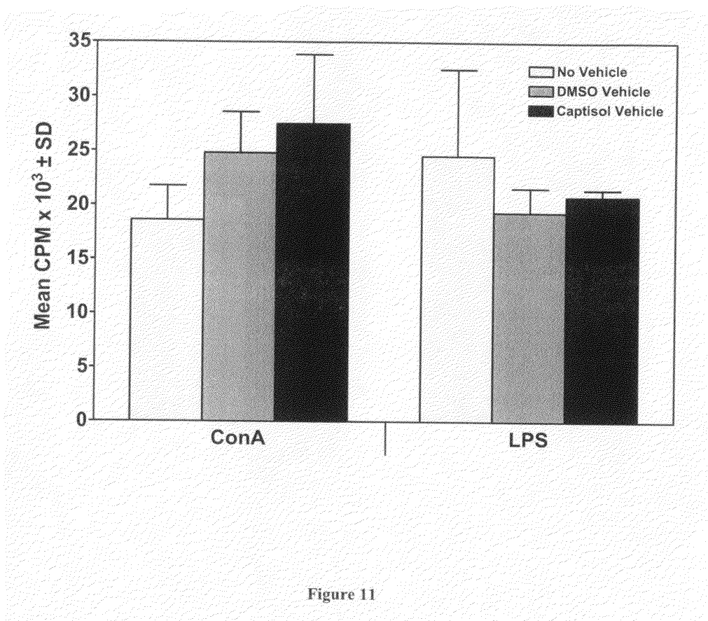

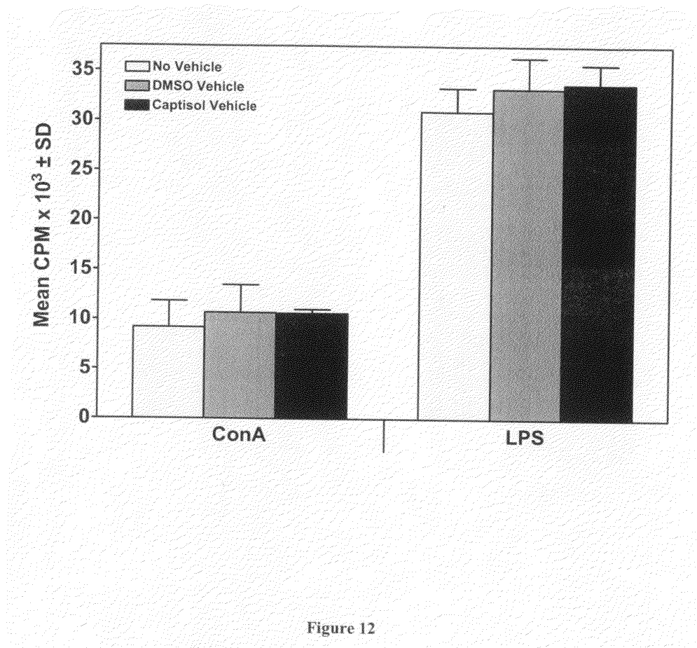

FIGS. 11 and 12 show the effect of vehicle treatment on mitogen responses at a high (1.6×10⁶ cells/well) and low (8×10⁵ cells/well) cell concentration, respectively. FIGS. 11 and 12 show no significant difference between no vehicle and either DMSO or captisol vehicle for either mitogen.

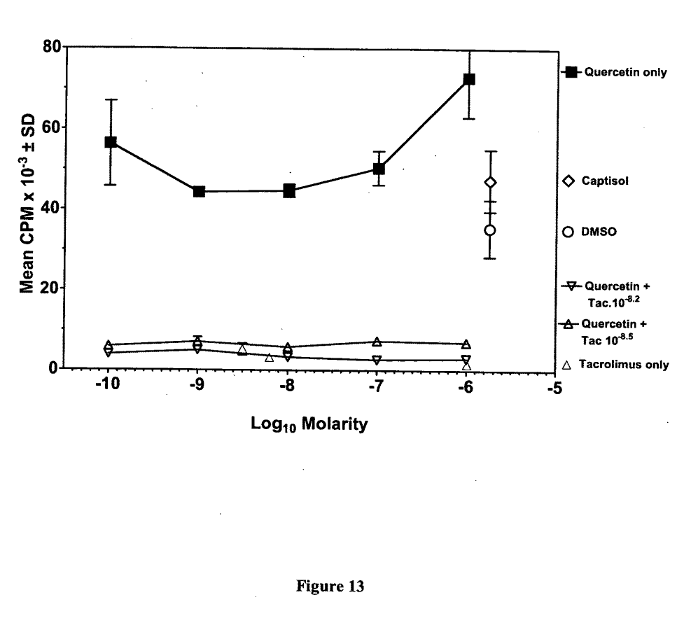

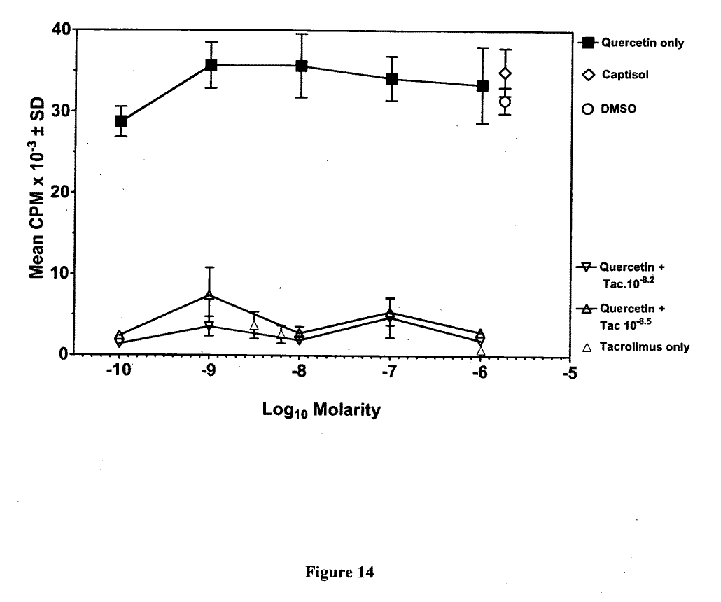

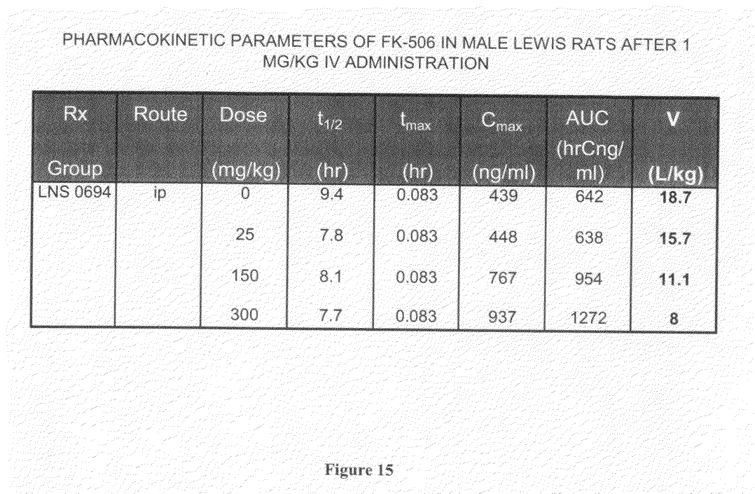

Spleen cells are treated with Con A in the presence of vehicle, tacrolimus, quercetin or two different concentrations of tacrolimus (10^{-8.2} and 10^{-8.5} M) and increasing doses of quercetin at a high cell concentration (FIG. 13) or at a low cell concentration (FIG. 14). FIG. 15 shows no significant difference between cultures treated with tacrolimus and cultures treated with tacrolimus and quercetin. Quercetin does not impair tacrolimus induced cell suppression in vitro at either concentration of tacrolimus. There is no significance difference between the effect of quercetin on tacrolimus induced cell suppression in vitro at the different concentrations used for quercetin. The same results are observed in cultures with low cell concentration (FIG. 14).

Taken together these results demonstrate that quercetin does not alter the effect of tacrolimus on cells.

### Example 7

### BTB Transport Protein Modulator Increase Peripheral Bioavailability and Decrease the Volume of Distribution of Tacrolimus

Animals: 8-9 weeks-old Lewis and Brown Norway male rats are obtained from Charles River Laboratories. General procedures for animal care and housing is in accordance with the National Research Council (NRC) Guide for the Care and Use of Laboratory Animals (1996) and the Animal Welfare Standards incorporated in 9 CFR Part 3, 1991.

Treatment: Lewis rats are treated as described in example 3. Subsets of 3 Lewis rats per group are used for blood sampling at each time point (Groups 2-5) as described in the table below.

Pharmacokinetics Group (n = 9)

| Parameter: | 3 rats/group | 3 rats/group | 3 rats/group |
|---|---|---|---|
| Pharmacokinetics | 5 min | 15 min | 30 min |
| (whole blood | 1 hr | 4 hr | 8 hr |
| drug levels) | 2 hr | 6 hr | 24 hr |
| Penetration of | 2 hr | — | — |
| Blood-Tissue | | | |
| barrier | | | |
| (brain drug | | | |
| levels) | | | |

The brains are harvested from one subset of 3 rats per group at 2 hr after FK506 treatment.

Plasma Blood Sample Collection: Whole blood is collected from the retro-orbital sinus under 60:40 CO₂:O₂ anesthesia using EDTA as the anticoagulant. Sample collection is performed at 9 time points: 5, 15, and 30 min and 1, 2, 4, 6, 8, & 24 hr after administration of FK 506. Three samples are collected from each rat per group (Groups 2-5). The first 2 samples are collected under anesthesia, after which the animal regains consciousness and is retained until the next collection interval. The third/last sample is also collected under anesthesia, however the animal was euthanatized prior to anesthetic recovery.

Whole blood with EDTA anticoagulant is collected from 3 naïve rats and stored frozen at 80° C. (±10° C.). These samples serve as baseline PK samples. Additional blood samples are collected approximately 4 days prior to study start for method development.

Whole blood samples with EDTA anticoagulant are collected from 4 rats (to total ≧25 ml), stored on wet ice, and delivered to PK staff for method development. Brains are collected from 3 of these rats immediately following blood collection.

Whole blood samples without an anticoagulant are collected from a different set of 2 rats and processed to obtain a total ≧7 ml of serum. Serum samples are stored on wet ice and developed as described below. The sample volume is 500 μL.

Whole blood samples are placed on dry ice after collection and stored frozen at 80° C. (±10° C.) until analysis.

Drug levels are determined in collected whole blood samples using a bioanalytical method developed to detect parent drug levels.

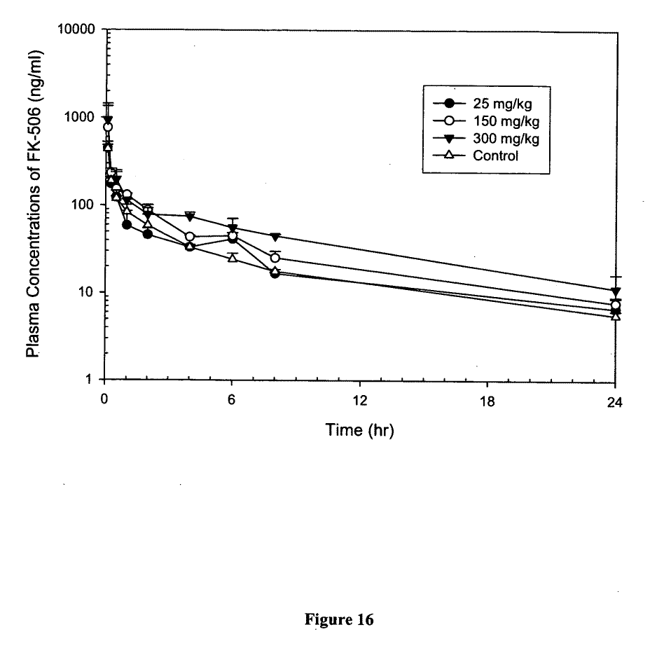

Results are shown in FIGS. 15 and 16.

Results: The pharmacokinetics parameters of FK 506 are determined in male Lewis rats after 1 mg/kg i.v. administration alone or in combination with i.p, administration of different doses of LNS 0694 (See FIG. 15).

FIG. 16 shows the plasma concentration of FK 506 at different time points after 1 mg/kg i.v. administration alone or in combination with i.p, administration of different doses of LNS 0694. Results in FIGS. 15 and 16 demonstrate that LNS 0694 increases the peripheral bioavailability of FK506 in a dose dependent manner.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A composition comprising an amount of a calcineurin inhibitor capable of inducing hyperglycemia and/or one or more symptoms of hyperglycemia and an amount of a blood tissue barrier (BTB) transport protein modulator sufficient to reduce hyperglycemia and/or one or more symptoms of hyperglycemia induced by the calcineurin inhibitor.

2. The composition of claim 1 wherein said calcineurin inhibitor is tacrolimus.

3. The composition of claim 1 wherein said calcineurin inhibitor is a tacrolimus analog.

4. The composition of claim 3 wherein said tacrolimus analog is selected from the group consisting of meridamycin, 31-O-Demethyl-FK506; L-683,590, L-685,818; 32-O-1-hydroxyethylindol-5-ylascomycin; ascomycin; C18-OH-ascomycin; 9-deoxo-31-O-demethyl-FK506; L-688,617; A-119435; AP1903; rapamycin; dexamethasone-FK506 heterodimer; 13-O-demethyl tacrolimus; and FK 506-dextran conjugate.

5. The composition of claim 1 wherein the BTB transport protein modulator is a BTB transport protein activator.

6. The composition of claim 1 wherein the BTB transport protein modulator modulates an ABC transport protein.

7. The composition of claim 6 wherein the ABC transport protein is P-gP.

8. The composition of claim 1 wherein said BTB transport protein modulator is a flavonoid or flavonoid derivative.

9. The composition of claim 8 wherein said flavonoid or flavonoid derivative is selected from the group consisting of quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, and epicatechin.

10. The composition of claim 9 wherein said flavonoid or flavonoid derivative is quercetin or a quercetin derivative.

11. The composition of claim 10, wherein said quercetin or quercetin derivative is 5,7-dideoxyquercetin.

12. The composition of claim 10, wherein said quercetin or quercetin derivative is phosphorylated.

13. The composition of claim 12, wherein said phosphorylated quercetin is 3′-quercetin phosphate, 4′-quercetin phosphate, 5,7-dideoxyquercetin phosphate, or a combination thereof.

14. The composition of claim 12, wherein said phosphorylated quercetin is 3-quercetin phosphate.

15. The composition of claim 12, wherein said phosphorylated quercetin is 4′-quercetin phosphate.

16. The composition of claim 12, wherein said phosphorylated quercetin is a mixture of 3′-quercetin phosphate and 4′-quercetin phosphate.

17. The composition of claim 16, wherein said phosphorylated quercetin mixture comprises at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 99.1% or at least 99.9% of 3′-quercetin phosphate.

18. The composition of claim 16, wherein said phosphorylated quercetin mixture comprises at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 99.1% or at least 99.9% of 4′-quercetin phosphate.

19. The composition of claim 9, wherein said flavonoid or flavonoid derivative is fisetin or a fisetin derivative.

20. The composition of claim 19, wherein said fisetin or fisetin derivative is phosphorylated.

21. The composition of claim 20, wherein said phosphorylated fisetin is 3′-fisetin phosphate, 4′-fisetin phosphate, 3-fisetin phosphate or a combination thereof.

22. The composition of claim 1, further comprising an oligosaccharide.

23. The composition of claim 22, wherein said oligosaccharide is a cyclic oligosaccharide.

24. The composition of claim 22, wherein said oligosaccharide is a cyclodextrin.

25. The composition of claim 24, wherein said cyclodextrin is a sulfo-alkyl ether substituted cyclodextrin or a sulfobutyl-ether substituted cyclodextrin.

26. The composition of claim 24, wherein said cyclodextrin is hydroxypropyl-β-cyclodextrin, hydroxypropyl-γ-cyclodextrin, sulfobutylether-β-cyclodextrin, sulfobutylether-7-γ-cyclodextrin, Captisol or combinations thereof.

27. The composition of claim 1 wherein said calcineurin inhibitor is tacrolimus and said BTB transport protein modulator is quercetin or a quercetin derivative.

28. The composition of claim 1 wherein said calcineurin inhibitor is tacrolimus and said BTB transport protein modulator is fisetin or a fisetin derivative.

29. The composition of claim 27 wherein tacrolimus and quercetin or a quercetin derivative are present in a molar ratio of about 0.001:1 to about 10:1.

30. The composition of claim 27 wherein tacrolimus is present at about 0.1-1000 mg and quercetin or a quercetin derivative is present at about 10 to about 1000 mg.

31. The composition of claim 30 wherein tacrolimus is present at about 0.5-100 mg and quercetin or a quercetin derivative is present at about 50 to about 500 mg.

32. The composition of claim 31 wherein tacrolimus is present at about 5 mg and quercetin or a quercetin derivative is present at about 500 mg.

33. The composition of claim 1 wherein the hyperglycemia or a symptom of hyperglycemia induced by said calcineurin inhibitor is capable of being decreased by an average of at least about 5% compared to the hyperglycemia or symptom of hyperglycemia without the BTB transport protein modulator, when the composition is administered to an animal.

34. The composition of claim 1 wherein said symptom is selected from the group consisting of glucosuria, polyphagia, polyuria, polydipsia, loss of consciousness, blurred vision, headaches, coma, ketoacidosis, decrease in blood volume, decrease in renal bloodflow, accelerated lipolysis, weight loss, stomach problems, intestinal problems, poor wound healing, dry mouth, nausea, vomiting, dry skin. itchy skin, impotence, hypeventilation, ketoanemia, fatigue, weakness on one side of the body, hallucinations, impairment in cognitive function, increase sadness, anxiety, recurrent genital infections, increase sugar in urine, retinopathy, nepropathy, arteriosclerotic disorders, cardiac arrhythmia, stupor, susceptibility to infection, neuropathy, nerve damages causing cold feet, nerve damage causing insensitive feet, loss of hair and a combination thereof.

35. The composition of claim 34 wherein said symptom is glucosuria.

36. A kit comprising the composition of claim 1 and instructions for use of the composition.

37. A method of decreasing or preventing the appearance of hyperglycemia and/or one or more symptoms of hyperglycemia induced by a calcineurin inhibitor comprising:
administering to a subject receiving treatment with said calcineurin inhibitor with known or suspected hyperglycemia an amount of a blood tissue barrier (BTB) transport protein modulator sufficient to prevent, reduce or eliminate hyperglycemia and/or one or more symptoms of hyperglycemia induced by said calcineurin inhibitor.

38. The method of claim 37 wherein said calcineurin inhibitor is tacrolimus.

39. The method of claim 37 wherein said calcineurin inhibitor is a tacrolimus analog.

40. The method of claim 39 wherein said tacrolimus analog is selected from the group consisting of meridamycin, 31-O-Demethyl-FK506; L-683,590, L-685,818; 32-O-1-hydroxyethylindol-5-ylascomycin; ascomycin; C18-OH-ascomycin; 9-deoxo-31-O-demethyl-FK506; L-688,617; A-119435; AP1903; rapamycin; dexamethasone-FK506 heterodimer; 13-O-demethyl tacrolimus; and FK 506-dextran conjugate.

41. The method of claim 37 wherein the BTB transport protein modulator is a BTB transport protein activator.

42. The method of claim 37 wherein the BTB transport protein modulator modulates an ABC transport protein.

43. The method of claim 42 wherein the ABC transport protein is P-gP.

44. The method of claim 37 wherein said BTB transport protein modulator is a flavonoid or flavonoid derivative.

45. The method of claim 44 wherein said flavonoid or flavonoid derivative is selected from the group consisting of quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, and epicatechin.

46. The method of claim 45 wherein said flavonoid or flavonoid derivative is quercetin or a quercetin derivative.

47. The method of claim 46, wherein said quercetin or quercetin derivative is 5,7-dideoxyquercetin.

48. The method of claim 46, wherein said quercetin or quercetin derivative is phosphorylated.

49. The composition of claim 48, wherein said phosphorylated quercetin is 3′-quercetin phosphate, 4′-quercetin phosphate, 5,7-dideoxyquercetin phosphate, or a combination thereof.

50. The method of claim 48, wherein said phosphorylated quercetin is 3′-quercetin phosphate.

51. The method of claim 48, wherein said phosphorylated quercetin is 4′-quercetin phosphate.

52. The method of claim 48, wherein said phosphorylated quercetin is a mixture of 3′-quercetin phosphate and 4′-quercetin phosphate.

53. The method of claim 52, wherein said phosphorylated quercetin mixture comprises at least 5% of 3′-quercetin phosphate.

54. The c method of claim 52, wherein said phosphorylated quercetin mixture comprises at least 5% of 4′-quercetin phosphate.

55. The method of claim 44, wherein said flavonoid or flavonoid derivative is fisetin or a fisetin derivative.

56. The method of claim 55, wherein said fisetin or fisetin derivative is phosphorylated.

57. The method of claim 56, wherein said phosphorylated fisetin is 3′-fisetin phosphate, 4′-fisetin phosphate, 3-fisetin phosphate or a combination thereof.

58. The method of claim 37, further comprising an oligosaccharide.

59. The method of claim 58, wherein said oligosaccharide is a cyclodextrin.

60. The method of claim 59, wherein said cyclodextrin is a sulfo-alkyl ether substituted cyclodextrin or a sulfobutyl-ether substituted cyclodextrin.

61. The method of claim 59, wherein said cyclodextrin is hydroxypropyl-β-cyclodextrin, hydroxypropyl-γ-cyclodextrin, sulfobutylether-β-cyclodextrin, sulfobutylether-7-β-cyclodextrin, or combinations thereof.

62. The method of claim 37 wherein said calcineurin inhibitor is tacrolimus and said BTB transport protein modulator is quercetin or a quercetin derivative.

63. The method of claim 37 wherein said calcineurin inhibitor is tacrolimus and said BTB transport protein modulator is fisetin or a fisetin derivative.

64. The method of claim 37 wherein said symptom is selected from the group consisting of glucosuria, polyphagia, polyuria, polydipsia, loss of consciousness, blurred vision, headaches, coma, ketoacidosis, decrease in blood volume, decrease in renal bloodflow, accelerated lipolysis, weight loss, stomach problems, intestinal problems, poor wound healing, dry mouth, nausea, vomiting, dry skin, itchy skin, impotence, hypeventilation, ketoanemia, fatigue, weakness on one side of the body, hallucinations, impairment in cognitive function, increase sadness, anxiety, recurrent genital infections, increase sugar in urine, retinopathy, nepropathy, arteriosclerotic disorders, cardiac arrhythmia, stupor, susceptibility to infection, neuropathy, nerve damages causing cold feet, nerve damage causing insensitive feet and loss of hair, and a combination thereof.

65. The method of claim 64 wherein said symptom is glucosuria.

66. The method of claim 37 wherein said subject experiences from a condition selected from the group consisting of organ transplant, an autoimmune disease, and an inflammatory disease.

67. The method of claim 66 wherein said condition is organ transplant.

68. The method of claim 67 wherein said organ transplant is selected from the group consisting of kidney transplant, pancreas transplant, liver transplant, heart transplant, lung transplant, intestine transplant, pancreas after kidney transplant, and simultaneous pancreas-kidney transplant.

69. The method of claim 66 wherein said condition is an autoimmune disease.

70. The method of claim 69 wherein said autoimmune disease is selected from the group consisting of Lupus nephritis, actopic dermatitis, and psoriasis.

71. The method of claim 66 wherein said condition is an inflammatory disease.

72. The method of claim 71 wherein said inflammatory disease is selected from the group consisting of asthma, vulvar lichen sclerosis, chronic allergic contact dermatitis, eczema, vitiligo and ulcerative colitis.

73. The method of claim 37 wherein said administration comprises single or multiple doses of said calcineurin inhibitor and single or multiple doses of said BTB transport protein modulator.

74. The method of claim 37 wherein said administration comprises co-administration of said calcineurin inhibitor and said BTB transport protein modulator in the same dosage form, concurrent administration in separate dosage forms, or separate administration.

75. The method of claim 74 wherein said administration comprises co-administration of said calcineurin inhibitor and said BTB transport protein modulator in the same dosage form.

76. The method of claim 37 wherein the molar ratio of the amount of the calcineurin inhibitor administered to the amount of BTB transport protein modulator administered is about 0.001:1 to about 10:1.

77. The method of claim 37 wherein the calcineurin inhibitor is administered in an amount sufficient to exert a therapeutic effect and the BTB transport protein modulator is administered in an amount sufficient to decrease hyperglycemia and/or one or more symptoms of hyperglycemia induced by the calcineurin inhibitor by an average of at least about 5%, compared to the hyperglycemia or symptom of hyperglycemia without the BTB transport protein modulator.

78. The method of claim 37 wherein a therapeutic effect of the calcineurin inhibitor is increased at least about 5%, compared to the therapeutic effect without the BTB transport protein modulator.

79. A method of modifying the concentration of a calcineurin inhibitor in a pancreatic islet cell comprising administering to a subject in need of treatment with said calcineurin inhibitor an amount of a BTB transport protein modulator sufficient to modify the concentration of said calcineurin inhibitor in said pancreatic islet cell.

80. The method of claim 79 wherein said pancreatic islet cell is a β cell.

81. The method of claim 79 wherein said BTB transport protein modulator decreases the concentration of said calcineurin inhibitor in said pancreatic islet cell.

82. The method of claim 79 wherein said BTB transport protein modulator is a BTB protein transport activator.

83. The method of claim 79 wherein said BTB transport protein modulator modulates an ABC transport protein.

84. The method of claim 83 wherein said ABC transport protein is P-gP.

85. The method of claim 79 wherein said BTB transport protein modulator is a flavonoid or flavonoid derivative.

86. The method of claim 85 wherein said flavonoid or flavonoid derivative is selected from the group consisting of quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, and epicatechin.

87. The method of claim 86 wherein said flavonoid or flavonoid derivative is quercetin or a quercetin derivative.

88. The composition of claim 87, wherein said quercetin or quercetin derivative is 5,7-dideoxyquercetin.

89. The composition of claim 87, wherein said quercetin or quercetin derivative is phosphorylated.

90. The composition of claim 89, wherein said phosphorylated quercetin is 3′-quercetin phosphate, 4′-quercetin phosphate, 5,7-dideoxyquercetin phosphate, or a combination thereof.

91. The composition of claim 89, wherein said phosphorylated quercetin is 3′-quercetin phosphate.

92. The composition of claim 89, wherein said phosphorylated quercetin is 4′-quercetin phosphate.

93. The composition of claim 89, wherein said phosphorylated quercetin is a mixture of 3′-quercetin phosphate and 4′-quercetin phosphate.

94. The composition of claim 93, wherein said phosphorylated quercetin mixture comprises at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 99.1% or at least 99.9% of 3′-quercetin phosphate.

95. The composition of claim 93, wherein said phosphorylated quercetin mixture comprises at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 99.1% or at least 99.9% of 4′-quercetin phosphate.

96. The composition of claim 86, wherein said flavonoid or flavonoid derivative is fisetin or a fisetin derivative.

97. The composition of claim 96, wherein said fisetin or fisetin derivative is phosphorylated.

98. The composition of claim 97, wherein said phosphorylated fisetin is 3′-fisetin phosphate, 4′-fisetin phosphate, 3-fisetin phosphate or a combination thereof.

99. The composition of claim 79, further comprising an oligosaccharide.

100. The composition of claim 99, wherein said oligosaccharide is a cyclodextrin.

101. The composition of claim 100, wherein said cyclodextrin is a sulfo-alkyl ether substituted cyclodextrin or a sulfobutyl-ether substituted cyclodextrin.

102. The composition of claim 100, wherein said cyclodextrin is hydroxypropyl-β-cyclodextrin, hydroxypropyl-γ-cyclodextrin, sulfobutylether-β-cyclodextrin, sulfobutylether-7-β-cyclodextrin, or combinations thereof.

103. The method of claim 79 wherein said calcineurin inhibitor is tacrolimus or tacrolimus analog.

104. The method of claim 103 wherein said tacrolimus analog is selected from the group consisting of meridamycin, 31-O-Demethyl-FK506; L-683,590, L-685,818; 32-O-(1-hydroxyethylindol-5-yl)ascomycin; ascomycin; C18-OH-ascomycin; 9-deoxo-31-O-demethyl-FK506; L-688,617; A-119435; AP1903; rapamycin; dexamethasone-FK506 heterodimer; 13-O-demethyl tacrolimus; and FK 506-dextran conjugate.

105. A method of decreasing or preventing the appearance of hyperglycemia and/or one or more symptoms of hyperglycemia comprising administering to a subject with a known or suspected symptom of hyperglycemia an amount of a BTB transport protein modulator sufficient to prevent, reduce or eliminate hyperglycemia and/or one or more symptoms of hyperglycemia.

106. The method of claim 105 wherein said symptom is selected from the group consisting of glucosuria, polyphagia, polyuria, polydipsia, loss of consciousness, blurred vision, headaches, coma, ketoacidosis, decrease in blood volume, decrease in renal bloodflow, accelerated lipolysis, weight loss, stomach problems, intestinal problems, poor wound healing, dry mouth, nausea, vomiting, dry skin, itchy skin, impotence, hypeventilation, ketoanemia, fatigue, weakness on one side of the body, hallucinations, impairment in cognitive function, increase sadness, anxiety, recurrent genital infections, increase sugar in urine, retinopathy, nepropathy, arteriosclerotic disorders, cardiac arrhythmia, stupor, susceptibility to infection, neuropathy, nerve damages causing cold feet, nerve damage causing insensitive feet, loss of hair, and a combination thereof.

107. The method of claim 106 wherein said symptom is glucosuria.

108. The method of claim 105 wherein said BTB transport protein modulator is a BTB protein transport activator.

109. The method of claim 105 wherein said BTB transport protein modulator modulates an ABC transport protein.

110. The method of claim 109 wherein said ABC transport protein is P-gP.

111. The method of claim 105 wherein said BTB transport protein modulator is a flavonoid or flavonoid derivative.

112. The method of claim 111 wherein said flavonoid or flavonoid derivative is selected from the group consisting of quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, and epicatechin.

113. The method of claim 112 wherein said flavonoid or flavonoid derivative is quercetin or a quercetin derivative.

114. The composition of claim 113, wherein said quercetin or quercetin derivative is 5,7-dideoxyquercetin.

115. The composition of claim 113, wherein said quercetin or quercetin derivative is phosphorylated.

116. The composition of claim 115, wherein said phosphorylated quercetin is 3′-quercetin phosphate, 4′-quercetin phosphate, 5,7-dideoxyquercetin phosphate, or a combination thereof.

117. The composition of claim 115, wherein said phosphorylated quercetin is 3-quercetin phosphate.

118. The composition of claim 115, wherein said phosphorylated quercetin is 4′-quercetin phosphate.

119. The composition of claim 115, wherein said phosphorylated quercetin is a mixture of 3′-quercetin phosphate and 4′-quercetin phosphate.

120. The composition of claim 119, wherein said phosphorylated quercetin mixture comprises at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 99.1% or at least 99.9% of 3′-quercetin phosphate.

121. The composition of claim 119, wherein said phosphorylated quercetin mixture comprises at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 99.1% or at least 99.9% of 4′-quercetin phosphate.

122. The composition of claim 111, wherein said flavonoid or flavonoid derivative is fisetin or a fisetin derivative.

123. The composition of claim 122, wherein said fisetin or fisetin derivative is phosphorylated.

124. The composition of claim 123, wherein said phosphorylated fisetin is 3′-fisetin phosphate, 4′-fisetin phosphate, 3-fisetin phosphate or a combination thereof.

125. The composition of claim 105, further comprising an oligosaccharide.

126. The composition of claim 125, wherein said oligosaccharide is a cyclodextrin.

127. The composition of claim 126, wherein said cyclodextrin is a sulfo-alkyl ether substituted cyclodextrin or a sulfobutyl-ether substituted cyclodextrin.

128. The composition of claim 126, wherein said cyclodextrin is hydroxypropyl-β-cyclodextrin, hydroxypropyl-γ-cyclodextrin, sulfobutylether-β-cyclodextrin, sulfobutylether-7-β-cyclodextrin, or combinations thereof.
